(19) United States
(12) Patent Application Publication
(10) Pub. No.: US 2025/0313819 A1
(43) Pub. Date: Oct. 9, 2025

(54) THERAPEUTIC CRISPR/CAS9 GENE EDITING APPROACHES TO THE C9ORF72 REPEAT EXPANSION MUTATION IN IPSCS

(71) Applicants: THE REGENTS OF THE UNIVERSITY OF CALIFORNIA, Oakland, CA (US); The J. David Gladstone Institutes, a testamentary trust established under the Will of J. David Glads, San Francisco, CA (US)

(72) Inventors: Claire Clelland, San Francisco, CA (US); Bruce Conklin, San Francisco, CA (US)

(21) Appl. No.: 18/864,778

(22) PCT Filed: May 12, 2023
(86) PCT No.: PCT/US2023/066966

§ 371 Date: Nov. 11, 2024

Related U.S. Application Data
(60) Provisional application No. 63/341,341, filed on May 12, 2022.

(51) Int. Cl. C12N 9/22; A61P 25/28; C12N 15/11

(52) U.S. Cl. CPC C12N 9/226; A61P 25/28; C12N 15/111; C12N 2310/20

(57) ABSTRACT

There are provided in vitro and in vivo methods of editing the C9ORF72 repeat expansion mutation using a nuclease to edit a nucleic acid in which the expansion is found. An exemplary method uses a Cas-9 editing system. Guide nucleic acids for editing the repeat expansion mutation are provided. Also provided is a method of mitigating or eliminating symptoms arising in a subject due to the presence of the mutation in the subject's genome.

### CROSS REFERENCES TO RELATED APPLICATIONS

The present disclosure claims priority to U.S. Provisional Patent Application No. 63/341,341 filed May 12, 2022, which is hereby incorporated by reference.

This application is related to United States Provisional Patent Application entitled “THERAPEUTIC CRISPR/CAS9 GENE EDITING APPROACHES TO THE C9ORF72 REPEAT EXPANSION MUTATION IN IPSCS” (Attorney Docket No.: 061818-5531-PR), filed on an even date herewith, the entire disclosure of which is incorporated herein by reference for all purposes.

### STATEMENT REGARDING FEDERALLY SPONSORED RESEARCH OR DEVELOPMENT

This invention was made with government support under grants K08 NS112330, EY028249, AG072052, HL145795 awarded by The National Institutes of Health. The government has certain rights in the invention.

### BACKGROUND OF THE INVENTION

Age-related neurodegenerative diseases, including dementias and motor neuron diseases, are leading contributors to death, disability and health care expenditure worldwide¹⁻⁶. Heterozygous expansion of a GGGGCC repeat in a single allele of the C9orf72 gene is the most frequent known genetic cause of both FTD and ALS⁷⁻⁹ (C9FTD/ALS). Targeting the mutant C9orf72 gene itself is the most parsimonious and potentially the most powerful therapeutic intervention. While antisense oligonucleotide (ASO) therapy showed promise in pre-clinical studies¹⁰⁻¹⁴, the inability of a phase I ASO trial in C9-ALS patients¹⁵ demonstrates the need for more targeted approaches. Gene editing offers the advantage that a single intervention could potentially be curative/preventative¹⁶.

Expression of the C9orf72 mutant repeat expansion is thought to cause disease through the generation of toxic products derived from the repeat expansion itself. RNA harboring the mutant repeat expansion may disrupt normal RNA processing by sequestering RNA-binding proteins¹⁷⁻²⁰ and production of toxic dipeptide repeats through repeat-associated non-canonical (RAN) translation²⁰⁻²⁹. Hapolinsufficiency has been proposed as an additional or alternative mechanism of disease³⁰⁻³³, but this is unlikely to the major contributor to C9FTD/ALS. The most compelling evidence against this hypothesis is that large-scale population sequencing³⁴ and clinical sequencing suggest that C9orf72 heterozygous loss-of-function mutations do not contribute to C9FTD/ALS³⁵. Secondly, knock-out mouse models have an autoimmune phenotype but lack neurologic disease³⁶⁻³⁹ Loss of C9orf72 function may indeed exacerbate toxic gain-of-function^{38,40}. We therefore hypothesized that gene editing strategies that remove or silence the repeat expansion would arrest or reverse cellular pathology.

CRISPR gene editing holds promise to cure or arrest monogenic disease, if we know which edit will be curative at the cellular level, and can achieve such an edit reliably, safely and effectively. C9orf72 is the leading genetic cause both frontotemporal dementia (FTD) and amyotrophic lateral sclerosis (ALS). A method of editing the C9orf72 repeat expansion mutation for the ability to correct pathology in neurons derived from patient iPSCs would provide a significant advance in the understanding of the origins, and pathologies associated with these conditions, and open pathways to treating these conditions.

### BRIEF SUMMARY OF THE INVENTION

Quite surprisingly, the present invention provides an efficacious and safe CRISPR-based method of editing the C9orf72 repeat expansion mutation, and first in class guide RNAs of use in carrying out this method. The method and guide RNAs provide critical tools for gene therapy targeting the C9orf72 repeat expansion mutation, which can normalize RNA abnormalities and TDP-43 pathology. In various embodiments, the present invention provides various methods of accomplishing this gene therapy.

Though clearly a valuable goal and target, selection of an appropriate method for gene therapy of the C9orf72 repeat expansion mutation is not immediately apparent. The most apparent strategy, editing to remove the repeat-expansion itself⁴¹⁻⁴³ risks off-target editing at >2500 homologous off-targets throughout the genome⁴⁴, thus risking cellular death from DNA damage. Other editing approaches disrupted nearby regulatory regions on both the normal and diseased allele⁴¹, which is undesirable as homozygous knockout causes early lethality in mice³⁶⁻³⁸ Finally, editing strategies that utilize homology directed repair⁴³ are inefficient in post-mitotic cells⁴⁵.

In various embodiments, the present invention provides approaches to targeting the C9orf72 repeat expansion mutation using gene therapy. Exemplary approaches include directly targeting the mutation (bi-allelic excision of the repeat expansion region), allele-specific excision of the mutant allele leaving the normal allele intact, and bi-allelic excision of a regulator region (exon 1A) controlling expression of the mutation. All three approaches normalize RNA abnormalities and TDP-43 pathology. Surprisingly, only repeat excision and allele-specific excision completely eliminated pathologic dipeptide repeats. Accordingly, in various embodiments, the invention provides methods of gene therapy targeting the C9orf72 repeat expansion mutation using a member selected from repeat excision, allele-specific excision and a combination thereof.

In various embodiments, the present invention provides CRISPR approaches to gene correction using patient iPSCs.

Additional objects and embodiments of the present invention will be better understood from the Detailed Description that follows. The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A, 1B, 1C, and 1D collectively illustrate editing efficiencies of three therapeutic editing approaches to correct the C9orf72 mutation in non-disease control and patient iPSC lines. (A) The mutant repeat expansion of the C9orf72 gene lies between alternative start sites, exon 1a and exon 1b in the 5′ UTR. Only the allele expressing the repeat expansion causes disease. (B) Three proof-of-concept CRISPR gene editing approaches to correct the C9orf72 mutation. Circle A: bi-allelic removal of the repeat expansion region. Circle B: 21 kb excision of the mutant allele containing the repeat expansion and transcriptional start site. This approach leaves the normal allele intact. Circle C: Bi-allelic excision of exon 1A that controls expression of the repeat expansion sense strand. (C) Summary table of editing approaches and their expected excision band size on the mutant and WT alleles. (D) Editing efficiencies were determined by PCR or single-molecule sequencing across the excision site. Each experiment contained 3 biologic replicates of either 48 hand-picked or 96 single-cell sorted clones. Sanger sequencing was used to confirm clone excision in surviving clones except for repeat expansion excision from the C9-patient cell line, which instead used single-molecule sequencing across the repeat region. Editing efficiencies were significantly different between patient and control lines for REx and 1Ax but not HET excisions (2-way ANOVA F (2, 11)=9.115, p<0.001; *p<0.05, **p<0.01 using Sidaks multiple comparison post-hoc test). Error bars=SEM.

FIGS. 2A, 2B, 2C, 2D, 2E, 2F, 2G, 2H, 2I, and 2J collectively illustrate C9orf72 gene expression after gene editing. RNA and protein in 2-week old hNIL neurons were measured across unedited and edited patient and control lines. (A) Exon spanning ddPCR probes were used to quantify the two dominant mRNA variants: exon 1A-long to exon 2 (RNA variant 3, V3) and exon 1B to exon 2 (RNA variant 2, V2). Exon 1A-short-containing RNA (RNA variant 1, V1) was not detected in the samples (data not shown). (B) Exon-spanning ddPCR probes that differed by a single nucleotide corresponding to a coding SNP were used in the patient line to quantify RNA derived from each allele. The probes targeted rs10757668 C/T. The C SNP was phased to the repeat-containing allele using single-molecule PacBio sequencing. Only exon 1A transcripts from the C-allele contain the sense repeat expansion. (C, D) ddPCR quantification of exon1A-exon2 (V3) RNA (blue), exon 1B-containing (V2) RNA (green) and exon2-exon3 total RNA (orange) in isogenic lines from a C9-patient (C) or WT-control (D). Less than 1% of detected RNA compared to RNA from UBE2D2 housekeeping gene derived from exon-1A in both cell lines; the majority of the total RNA came from exon-1B-containing transcript (V2). A significant gap was detected between measured variant RNA (exon-1A+exon-1B-containing transcript) and total transcript that was only present in lines harboring a repeat expansion (C9-unedited, HET(Ref)x) (paired t-test corrected for multiple tests, FDR<5%, *=p<0.01). (E, F) Use of exon 1AL-exon 2 (E) and exon 1B-exon 2 (F) spanning ddPCR probes that differed by a single nucleotide corresponding to a coding SNP to detect RNA expression from each allele. Surprisingly the majority of exon 1A-containing RNA (V3) derived from the mutant allele in the C9 unedited line (E) and this was normalized by repeat expansion excision (E, REx). As expected, heterozygous excision of one allele resulted in expression off of only the preserved allele (E, F: HET(Alt)x and HET(Ref)x). Exon 1B-containing transcripts found to predominantly arise from the WT allele in the unedited patient line (F, C9-unedited, blue) but this imbalance was corrected by repeat expansion excision (REx) or excision of 1Ax. Excision of exon 1A abolished expression exon-1A containing transcripts (E) and bi-allelelic gene knock-out abolished all transcripts (E,F). (G-J) Quantifying C9orf72 protein expression in unedited and edited patient and control lines. Interesting none of the allele-specific heterozygous excision changed total C9orf72 protein across both cell lines (1-way ANOVA: C9: F(5,12)=94.81, p<0.0001; WT: F(4,10)=32.98, p<0.0001; Dunnet's multiple comparison test *p<. 05, ****p<0.0001). Only excision of exon 1A in the WT line (H,J) and bi-allelic gene knock-out (G-J) significantly decreased C9orf72 protein level. Error bars=SEM.

FIGS. 3A, 3B and 3C collectively illustrate sense dipeptide repeat expression (DPR) is corrected by three therapeutic gene editing approaches whereas antisense DPR expression is only corrected by removing the repeat expansion. (A) Schematic of expression of sense and antisense repeat expansion in RNA and through non-canonical repeat-associated non-AUG (RAN) translation to form mutant dipeptide proteins. (B, C) Measuring 2 DPRs across unedited and edited C9-patient cell lines compared to KO control using MSD sandwich ELISA. (B) Poly-GA was only detected in lines expressing the sense mutant repeat expansion (C9-unedited, C9-HET(Ref)x) (1-way ANOVA F (4,10)=10.12, p<0.001; Dunnet's multiple comparisons test **p<0.01). Although the repeat expansion remains in the DNA, excision of exon 1A (C9-1Ax) halted the expression of poly-GA suggesting silencing of sense strand expression. (C) Poly-GP was detected in lines in which the repeat expansion remains in the DNA (C9-unedited, C9-HET(Ref)x, and C9-1Ax) (1-way ANOVA F (4,10)-19.66, p<0.0001; Dunnet's multiple comparisons test *p<0.5, **p<0.01) suggesting excision of exon 1A does eliminate mutant RNA/protein expression from the anti-sense strand. Error bars=SEM.

FIGS. 4A, 4B, 4C and 4D collectively illustrate three editing approaches correct abnormal loss of nuclear TDP43 in 7 week old neurons. (A, B) Distinguishing loss of nuclear TDP43 (pink arrow) from nuclear TDP (yellow arrow) in 7-week old induced neurons which were untreated (A) or treated with 1 uM proteosome inhibitor MG132 (B). (C) Observation of a non-significant trend toward increase in loss of nuclear TDP43 in unedited C9-patient after MG132 treatment (two-tailed t-test, p=0.1). (B, D) Comparing edited patient cell lines to the unedited patient cell line after MG132 treatment. All edits decreased loss of nuclear TDP43 (2-way ANOVA F (5,12)=12.01, p<0.001, *p<0.05, **p<0.001). Each experiment contained 3 biologic replicates (separate wells). Error bars=SEM.

FIGS. 5A, 5B, 5C, 5D, 5E, 5F, 5G, 5H, 5I, 5J, 5K, 5L, 5M, 5N, 50, 5P, 5Q, 5R, and 5S collectively illustrate Pacific Biosciences (PacBio) single molecule sequencing to determine the repeat size in 8 iPSC lines. Because the C9orf72 repeat expansion does not amplify by PCR over ˜60 repeats, it is not possible to size the repeat expansion by traditional sequencing methods. (A) Schematic of the pipeline used to generate the library for single molecule sequencing. Enriching the gene region of interest by using CRISPR gRNA to excise a segment of DNA containing the repeat expansion region. Barcoding allowed to multiplexing of samples to reduce costs. (B) Sequencing 3-5 ug of DNA from WT-control iPSCs and 7 iPSCs from patients harboring expansions of the C9orf72 mutation. Quantification of repeat number initiated by counting the number of GGCCCC repeats just after an anchor (CGCCC) 5′ to the repeat region. On target reads were calculated from total reads that fully sequenced the excised genomic region (including the repeat region and flanking DNA) 3 times (=3 pass criteria). Repeat lengths and associated read counts are reported for each allele of each cell line and compared to repeat length estimated by Southern blot. Repeat lengths estimated by Southern blot were comparable to mean repeat lengths determined by single molecule PacBio sequencing. (C) Southern blot of WT-control and patient DNA from iPSCs. After EcoR/Xbal digestion, a loading control fragment (1.05 kb), WT allele (1.33 kb) and expanded repeats were detected. Southern blot required 20 ug of input DNA (vs. 3-5 ug input for PacBio sequencing) and a sample with 14 ug (P6) failed Southern blot, demonstrating the insensitivity of Southern blot. (D-S) Sequencing traces for each cell line. (D, F, H, J, L, N, P, R) show single molecule sequencing traces. Each horizontal line depicts one sequenced molecule of DNA. Blue color depicts on target sequencing, grey color depicts sequencing error. Each molecule is anchored to an adjacent, non-repeat region (CGCCC) which is not included in the total repeat count. Y-axis=CCS reads. (E, G, I, K, M, O, Q, S) Historgrams showing frequency of repeat count by CCS read.

FIG. 6 illustrates Guide nucleic acid and primer sequences used to generate and verify, respectively, each edited cell line. Excision size of the corresponding region of the WT allele is provided for each line. Expected amplicon size for each set of PCR primers is also provided. The sequences under gRNA are used to generate the lines. The primer sequences are the primers used to confirm the lines. The excision primers are to detect excision bands. The cut site primers are to detect any remaining WT alleles that were not excised and also indels that might have formed.

FIG. 7 illustrates predicted off-targets for eachguide nucleic acid. Using CRISPOR (Homo sapiens-USCS December 2013 (GRCh38/hg38) to predict off-targets for each gRNA for spCas9. Off-targets are displayed as a function of mismatch (0-1-2-3-4) and in NT next to the PAM site.

FIGS. 8A, 8B, 8C, 8D, 8E, and 8F collectively illustrate C9orf72 RNA quantification of edited C9-patient and WT-control lines. (A, B) Use of exon-spanning PCR primers to quantify RNA variant incorporating exon 1a (variant 3), exon 1b (variant 2) or total RNA (exon 2-3 spanning). Variant 1 (1a-short) could not be detected above background noise in any of the cell lines (data not shown). (C, D) ddPCR quantification of exon1A-exon2 (V3) RNA (blue), exon 1B-containing (V2) RNA (green) and exon2-exon3 total RNA (orange) in isogenic lines from a C9-patient (C) or WT-control (D). These data are depicted in FIG. 2 C, D and repeated here to illustrate total contribution from each transcript variant. A significant gap was detected between exon-1B-containing transcript and total transcript that was only present in lines harboring a repeat expansion (C9-unedited, HET(Ref)x) (mixed models F (10,36)=5.6, p<0.0001; Tukey's post-hoc test *p<0.05, **p<0.005). This gap was closed in C9-corrected lines (C9-REx, HET(Alt)x, 1Ax) and all WT lines) (WT: mixed models F (8.29)=38.9, p<0.0001). (E, F) Quantification of the gap between detectable RNA variants (1A+1B-containing transcripts) vs total measured RNA (exon 2-3 containing transripts) in C9-patient (E) and WT (F) lines. Only C9-lines expressing the repeat expansion (C9-unedited, HET(Ref)x) significantly differed from 0 (0=no gap between measured variant and total transcript) (one-sample t-test corrected for multiple comparisons, *=p<0.01). PCR probes for each exon-spanning RNA target and ddPCR probes for each allele-specific RNA target are shown in FIG. 2A.

FIG. 9 illustrates ddPCR probes used in FIG. 2 and FIG. 8.

FIG. 10 illustrates nine commercially available C9orf72 antibodies tested are not specific for C9orf72 in iPSC-derived neurons by immunocytochemistry. Commercially available C9orf72 antibodies were not specific for C9orf72 found by comparing staining patterns in knock-out lines (WT-KO and C9-KO) to unedited cells (WT-unedited and C9-unedited). Blue=DAPI. Green=staining from antibodies tested in table FIG. 11. Scale bar=100 uM.

FIG. 11 illustrates C9orf72 antibodies, and their concentrations, tested corresponding to FIG. 10.

FIGS. 12A and 12B collectively illustrate two of 10 antibodies tested were specific for dipeptide repeats (DPRs) from the C9orf72 mutant line compared to KO control. (A) Schematic of expression of sense and antisense repeat expansion in RNA and through non-canonical repeat-associated non-AUG (RAN) translation to form mutant dipeptide proteins. 5 total DPRs are formed: poly-GR and poly-GA from the sense strand, poly-PR and poly-PA from the antisense strand and poly-GP from both the sense and antisense strands. (B) Testing 10 antibodies raised against DPRs in varying combinations and concentrations using sandwich ELISA on the MSD platform. Concentrations of capture, detect and lysate from 2-week old neurons induced from a C9-patient iPSC line harboring ˜195 repeats or 21 kb KO of the C9orf72 gene, including the transcriptional start site, the repeat region and exons 1-3. Antibody combinations that generated a ratio of signal from the C9-patient vs KO line greater than 2 (highlighted in green) were used to generate FIG. 3 (the corresponding conditions used are highlighted in grey). Most antibodies generated noise that was similar between KO and C9-patient lines (ratio ˜1).

FIGS. 13A and 13B collectively illustrate no difference in loss of nuclear TDP43 in edited C9-patient lines without treatment. (A) Distinguishing loss of nuclear TDP43 (pink arrow) from nuclear TDP (yellow arrow) in 7-week old induced neurons which received no proteosome inhibitor treatment (untreated) across all C9-patient edited lines. (B) There was not a significant effect of genotype on nuclear TDP43 quantification (1-way ANOVA F(5,12)=2.222, p=0.12; *p<0.05). Each experiment contained 3 biologic replicates (separate wells). Error bars=SEM.

FIGS. 14A, 14B, 14C, 14D, 14E, and 14F collectively illustrate construction of the C9-REx cell line. (A) Position of the gRNAs (indicated by scissors) and excision primers (purple errors) used to create and verify, respectively, excision of the repeat expansion in the C9orf72 gene in a patient cell line. (B) Line had a band at ˜500 bp using excision primers and clean Sanger sequencing (C) cut sites indicated by pink arrows). the repeat region fails amplification, thus these data do not indicate whether the line had a homozygous excision of the repeat region or a heterozygous excision of the WT allele only. Single-molecule sequencing (D) was used to determine that that the clone was pure, with a 26 bp excision of the mutant allele (using SNPs to differentiate alleles, indicated by blue arrows). (E) Allele count of PacBio sequencing data shows both alleles were equally covered by sequencing. (F) The cell line had a normal karyotype.

FIGS. 15A, 15B, 15C, 15D, 15E, 15F, 15G, and 15H collectively illustrate construction of the C9-Het(Mut)x cell line. (A) Position of the gRNAs (indicated by scissors) and excision and cut site primers (purple errors) used to create and verify, respectively, ˜22 kb excision of the mutant allele of the C9orf72 gene in a patient cell line. SNPs phased to the repeat expansion (blue dots) were used to target the mutant allele. Presence of an excision band (B) and preservation of bands at both the 5′ (C) and 3′ (D) cut sites indicates the line is a heterozygous excision. Corresponding clean Sanger sequencing (D-G) shows the clone is pure. (pink arrow—cut site; blue arrow—misaligned Sanger sequencing). (H) The cell line had a normal karyotype.

FIGS. 16A, 16B, 16C, 16D, 16E, and 16F collectively illustrate construction of the C9-1Ax cell line. (A) Position of the gRNAs (indicated by scissors) and excision primers (purple errors) used to create and verify, respectively, excision of exon 1A of the C9orf72 gene in a patient cell line. (B) Presence of an excision band and absence of a WT band C9-1 Ax indicates the line is homozygous. WT-unedited, C9-unedited and WT-1Ax serve as negative and positive controls. (C) Sanger sequencing shows the excision cut sites (pink arrows). (D) Single-molecule sequencing revealed 227 bp excision on the WT allele and a 354 bp excision on the mutant allele (blue arrow shows the repeat expansion). (E) Total alleles sequenced by single molecule sequencing showed a modest preference for the WT allele, as expected. (F) The cell line had a normal karyotype.

FIGS. 17A and 17B collectively illustrate efficiency of excision of the mutant C9orf72 through guide nucleic acids targeting allele specific sequences. (A) The on-targeting editing rate for each 5′ guide nucleic acid (as labeled by guide names 1, 2, 3, 4, 5, and 6) in combination with each 3′ guide nucleic acid (as labeled by guide names A, B, C, and D) with individual Replicate values were summarized and compared. The tested guide pairs include A1-A6, B1-B6, C1-C6 and D1-D6. (B) The on-target editing efficiency heat map for the combinations A1-A6, B1-B6, C1-C6 and D1-D6 were illustrated with each block representing the average editing efficiency across replicates.

FIGS. 18A and 18B collectively illustrate AAV vector DNA sequence for AAV-spCas9.

FIGS. 19A and 19B collectively illustrate AAV vector DNA sequence for AAV-ALT.

FIGS. 20A and 20B collectively illustrate AAV vector DNA sequence for AAV-REX.

### DETAILED DESCRIPTION OF THE INVENTION

### Introduction

The CRISPR/Cas9 system is a highly specific genome editing tool and newly engineered Cas9 variants are capable of distinguishing alleles differing by even a single base pair⁴⁶⁻⁵³. CRISPR-Cas9 was used to edit the C9orf72 locus in patient and non-diseased control iPSCs to generate 11 isogenic lines across two genetic backgrounds.

Selected embodiments of the present invention emerged from examination of three approaches to editing the C9orf72 locus: (1) targeting the mutation itself (repeat expansion excision), (2) allele-specific excision of the mutant allele leaving the normal allele intact and (3) excision of a regulatory region (exon 1A) that controls expression of the mutation sense-strand. Single-molecule sequencing was used to size the repeat expansion in 7 patient lines, to phase the mutation to nearby SNPs and to determine the outcome of edits involving the repeat expansion or that were otherwise indeterminable from Sanger sequencing. Robust editing and outcome measurement tools lay the groundwork to investigate gene-editing approaches for monogenic disease in human iPSCs and derived cell-types relevant to disease, and are applicable to any monogenic disease, particularly other repeat expansion disorders.

Three strategies for correcting the C9orf72 repeat expansion mutation in patient iPSCs were investigated. Each strategy capitalized on Cas9's ability to cut DNA, which aligns with technologies that are closest to clinical prime-time^{61,62}. Two of the three approaches (repeat expansion excision and excision of the mutant allele) were found to correct RNA abnormalities, preserve protein levels, and correct dipeptide repeat and TDP43 pathology in iPSC-derived neurons from a patient line harboring ˜200 repeats. As an alternative approach, silencing the expression of the repeat expansion without removing it from the DNA by excising exon 1A was performed. While this approach successfully restored the RNA profile and ameliorated TDP43 pathology, surprisingly, it did not eliminate poly-GP DPRs. Interestingly, both successful approaches, repeat expansion and allele-specific excisions, included removing the repeat expansion.

Provided herein are compositions and methods relating to treatment of disorders attributable to the C9orf72 repeat expansion mutation in human genome. Exemplary diseases treatable by the composition and methods of the invention include both frontotemporal dementia (FTD) and amyotrophic lateral sclerosis (ALS) caused by C9orf72 repeat expansion mutation in human genome.

### Definitions

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “oligonucleotide” refers to a polynucleotide of between 3 and 100 nucleotides of single- or double-stranded nucleic acid (e.g., DNA, RNA, or a modified nucleic acid). However, for the purposes of this disclosure, there is no upper limit to the length of an oligonucleotide. Oligonucleotides are also known as “oligomers” or “oligos” and may be isolated from genes, transcribed (in vitro and/or in vivo), or chemically synthesized. The terms “polynucleotide” and “nucleic acid” should be understood to include, as applicable to the embodiments being described, single-stranded (such as sense or antisense) and double-stranded polynucleotides.

A “stem-loop structure” refers to a nucleic acid having a secondary structure that includes a region of nucleotides which are known or predicted to form a double strand (step portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact base-pairing. Thus, the stem may include one or more base mismatches. Alternatively, the base-pairing may be exact, i.e. not include any mismatches.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule: guanine (G) can also base pair with uracil (U). For example, G/U base-pairing is partially responsible for the degeneracy (i.e., redundancy) of the genetic code in the context of tRNA anti-codon base-pairing with codons in mRNA. Thus, in the context of this disclosure, a guanine (G) is considered complementary to both a uracil (U) and to an adenine (A). For example, when a G/U base-pair can be made at a given nucleotide position of a protein-binding segment (e.g., dsRNA duplex) of a subject guide nucleic acid molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). The temperature and wash solution salt concentration may be adjusted as necessary according to factors such as length of the region of complementation and the degree of complementation.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Exemplary methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a subject Cas9/guide nucleic acid complex and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (Ka) of less than 10-6 M, less than 10-7 M, less than 108 M, less than 10-9 M, less than 1010 M, less than 1011 M, less than 1012 M, less than 10-13 M, less than 1014 M, or less than 10-15 M. “Affinity” refers to the strength of binding, increased binding affinity being correlated with a lower Ka.

By “binding domain” it is meant a protein domain that is able to bind non-covalently to another molecule. A binding domain can bind to, for example, a DNA molecule (a DNA-binding domain), an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various methods and computer programs (e.g., BLAST, T-COFFEE, MUSCLE, MAFFT, etc.), available over the world wide web at sites including ncbi.nlm nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10.

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g. tRNA, rRNA, microRNA (miRNA), a “non-coding” RNA (ncRNA), a guide nucleic acid, etc.).

A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide nucleic acid) or a coding sequence (e.g., Cas9 polypeptide, or Cas9 polypeptide) and/or regulate translation of an encoded polypeptide.

As used herein, a “promoter sequence” is a DNA regulatory region capable of binding RNA polymerase and initiating transcription of a downstream (3′ direction) coding or non-coding sequence. For purposes of the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present disclosure.

The term “Untranslated Regions (UTRs)” as used herein applied to untranslated regions (UTRs) of a gene are transcribed but not translated. The 5′UTR starts at the transcription start site and continues to the start codon but does not include the start codon; whereas the 3′UTR starts immediately following the stop codon and continues until the transcriptional termination signal.

The term “in cis” as used herein refers to regions of DNA on the same chromosome as a reference gene.

The term “naturally-occurring” or “unmodified” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is wild type (and naturally occurring).

“Heterologous,” as used herein, means a nucleotide or polypeptide sequence that is not found in the native nucleic acid or protein, respectively. For example, in a chimeric Cas9 protein, the RNA-binding domain of a naturally-occurring bacterial Cas9 polypeptide (or a variant thereof) may be fused to a heterologous polypeptide sequence (i.e. a polypeptide sequence from a protein other than Cas9 or a polypeptide sequence from another organism). The heterologous polypeptide sequence may exhibit an activity (e.g., enzymatic activity) that will also be exhibited by the chimeric Cas9 protein (e.g., methyltransferase activity, acetyltransferase activity, kinase activity, ubiquitinating activity, etc.). A heterologous nucleic acid sequence may be linked to a naturally-occurring nucleic acid sequence (or a variant thereof) (e.g., by genetic engineering) to generate a chimeric nucleotide sequence encoding a chimeric polypeptide. As another example, in a fusion variant Cas9 polypeptide, a variant Cas9 polypeptide may be fused to a heterologous polypeptide (i.e. a polypeptide other than Cas9), which exhibits an activity that will also be exhibited by the fusion variant Cas9 polypeptide. A heterologous nucleic acid sequence may be linked to a variant Cas9 polypeptide (e.g., by genetic engineering) to generate a nucleotide sequence encoding a fusion variant polypeptide.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Alternatively, DNA sequences encoding RNA (e.g., guide nucleic acid) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “vector” or “expression vector” is a replicon, such as plasmid, phage, virus, or cosmid, to which another DNA segment, i.e. an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression.

The terms “recombinant expression vector,” or “DNA construct” are used interchangeably herein to refer to a DNA molecule comprising a vector and one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: S0169-409X(12)00283-9. doi: 10.1016/j.addr.2012.09.023), and the like.

The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

By “cleavage” it is meant the breakage of the covalent backbone of a target nucleic acid molecule (e.g., RNA, DNA). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. In certain embodiments, a complex comprising a guide nucleic acid and a Cas9 polypeptide is used for targeted cleavage of a single stranded target nucleic acid (e.g., ssRNA, ssDNA).

“Nuclease” and “endonuclease” are used interchangeably herein to mean an enzyme which possesses catalytic activity for nucleic acid cleavage (e.g., ribonuclease activity (ribonucleic acid cleavage), deoxyribonuclease activity (deoxyribonucleic acid cleavage), etc.).

By “cleavage domain” or “active domain” or “nuclease domain” of a nuclease it is meant the polypeptide sequence or domain within the nuclease which possesses the catalytic activity for nucleic acid cleavage. A cleavage domain can be contained in a single polypeptide chain or cleavage activity can result from the association of two (or more) polypeptides. A single nuclease domain may consist of more than one isolated stretch of amino acids within a given polypeptide.

A “target nucleic acid” as used herein is a polynucleotide (e.g., RNA, DNA) that includes a “target site”, “target sequence” or “targeting segment.” The terms “target site”, “target sequence” or “targeting segment.” are used interchangeably herein to refer to a nucleic acid sequence present in a target nucleic acid to which a targeting segment of a subject guide nucleic acid will bind, provided sufficient conditions for binding exist. Suitable hybridization conditions include physiological conditions normally present in a cell. For a double stranded target nucleic acid, the strand of the target nucleic acid that is complementary to and hybridizes with the guide nucleic acid is referred to as the “complementary strand”; while the strand of the target nucleic acid that is complementary to the “complementary strand” (and is therefore not complementary to the guide nucleic acid) is referred to as the “noncomplementary strand” or “non-complementary strand”. In cases where the target nucleic acid is a single stranded target nucleic acid (e.g., single stranded DNA (ssDNA), single stranded RNA (ssRNA)), the guide nucleic acid is complementary to and hybridizes with single stranded target nucleic acid. “target sequence” refers to a sequence to which a guide sequence is designed to have complementarity, where hybridization between a target sequence and a guide sequence promotes the formation of an engineered nuclease complex. A target sequence may comprise any polynucleotide, such as DNA, RNA, or a DNA-RNA hybrid. A target sequence can be located in the nucleus or cytoplasm of a cell. A target sequence can be located in vitro or in a cell-free environment.

A nucleic acid molecule that binds to the Cas9 polypeptide and targets the polypeptide to a specific location within the target nucleic acid is referred to herein as a “guide nucleic acid”. When the guide nucleic acid is an RNA molecule, it can be referred to as a “guide RNA” or a “gRNA”. A subject guide nucleic acid comprises two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule. For example, in some cases the protein-binding segment (described below) of a guide nucleic acid is one nucleic acid molecule (e.g., one RNA molecule) and the protein-binding segment therefore comprises a region of that one molecule. In other cases, the protein-binding segment (described below) of a guide nucleic acid comprises two separate molecules that are hybridized along a region of complementarity.

A “PAM” as used herein, denotes the protospacer adjacent motif (PAM), which is a typically 2-6 base pair DNA sequence immediately proximal to the DNA sequence targeted by the nuclease (protospacer). Depending on the CRISPR system, a PAM sequence can be positioned either 5′ or 3′ relative to the protospacer sequence. Type V CRISPR-Cas systems show a specificity towards 5′ PAM sequences that are T-rich. In contrast, Cas9, a Type II Cas, has specificity for a 3′ G-rich PAM sequence.

A “PAMmer” as used herein, denotes a single stranded oligonucleotide (as defined above) (e.g., DNA, RNA, a modified nucleic acid (described below), etc.) that hybridizes to a single stranded target nucleic acid (thus converting the single stranded target nucleic acid into a double stranded target nucleic acid at a desired position), and provides a protospacer adjacent motif (PAM) sequence, thus converting the single stranded target nucleic acid into a target for binding and/or cleavage by a Cas9 polypeptide. A PAMmer includes a PAM sequence and at least one of: an orientation segment (which is positioned 3′ of the PAM sequence), and a specificity segment (which is positioned 5′ of the PAM sequence). A specificity segment has a nucleotide sequence that is complementary to a first target nucleotide sequence in a target nucleic acid (i.e., the sequence that is targeted by the specificity segment), where the first target nucleotide sequence overlaps (in some cases 100%) with the sequence targeted by the targeting segment of the guide nucleic acid. In other words, the specificity segment is complementary with (and hybridizes to) the target site of the target nucleic acid. In some cases, a PAMmer having a specificity segment is referred to herein as a “5′ extended PAMmer.” An orientation segment has a nucleotide sequence that is complementary to a second target nucleotide sequence in a target nucleic acid (i.e., the sequence that is targeted by the orientation segment). In some cases, a subject PAMmer includes a PAM sequence and an orientation segment, but does not include a specificity segment. In some cases, a subject PAMmer includes a PAM sequence and a specificity segment, but does not include an orientation segment.

Throughout the description below, when referring to the components (e.g., a PAMmer, a guide nucleic acid, a Cas9 polypeptide, etc.) of subject compositions and methods, terms describing the components can also be provided as nucleic acids encoding the component. For example, when a composition or method includes a Cas9 polypeptide, it is understood that the Cas9 can be provided as the actual polypeptide or as a nucleic acid (DNA or RNA) encoding the same. Likewise, when a composition or method includes a PAMmer, it is understood that the PAMmer can be provided as the actual PAMmer or as a nucleic acid (DNA) encoding the same. For example, in some cases a PAMmer is DNA, in some cases a PAMmer is a modified nucleic acid, and in some cases a PAMmer is RNA, in which case the term “PAMmer” can be provided as the actual RNA PAMmer but also can be provided as a DNA encoding the RNA PAMmer. Likewise, when a composition or method includes a guide nucleic acid, it is understood that the guide nucleic acid can be provided as the actual guide nucleic acid or as a nucleic acid (DNA) encoding the guide nucleic acid. For example, in some cases a guide nucleic acid is a modified nucleic acid, in some cases a guide nucleic acid is a DNA/RNA hybrid molecule, and in some cases a guide nucleic acid is RNA, in which case the guide nucleic acid can be provided as the actual guide RNA or as a DNA (e.g., plasmid) encoding the guide RNA.

A “host cell” or “target cell” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a nucleic acid, and include the progeny of the original cell which has been transformed by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject bacterial host cell is a genetically modified bacterial host cell by virtue of introduction into a suitable bacterial host cell of an exogenous nucleic acid (e.g., a plasmid or recombinant expression vector) and a subject eukaryotic host cell is a genetically modified eukaryotic host cell (e.g., a mammalian germ cell), by virtue of introduction into a suitable eukaryotic host cell of an exogenous nucleic acid.

The term “stem cell” is used herein to refer to a cell (e.g., plant stem cell, vertebrate stem cell) that has the ability both to self-renew and to generate a differentiated cell type (see Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells (described below) can differentiate into lineage-restricted progenitor cells (e.g., mesodermal stem cells), which in turn can differentiate into cells that are further restricted (e.g., neuron progenitors), which can differentiate into end-stage cells (i.e., terminally differentiated cells, e.g., neurons, cardiomyocytes, etc.), which play a characteristic role in a certain tissue type, and may or may not retain the capacity to proliferate further. Stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny.

Stem cells of interest include pluripotent stem cells (PSCs). The term “pluripotent stem cell” or “PSC” is used herein to mean a stem cell capable of producing all cell types of the organism.

PSCs of animals can be derived in a number of different ways. For example, embryonic stem cells (ESCs) are derived from the inner cell mass of an embryo (Thomson et. al, Science. 1998 Nov. 6; 282 (5391): 1145-7) whereas induced pluripotent stem cells (iPSCs) are derived from somatic cells (Takahashi et. al, Cell. 2007 Nov. 30; 131 (5): 861-72; Takahashi et. al, Nat Protoc. 2007; 2 (12): 3081-9; Yu et. al, Science. 2007 Dec. 21; 318 (5858): 1917-20. Epub 2007 Nov. 20). Because the term PSC refers to pluripotent stem cells regardless of their derivation, the term PSC encompasses the terms ESC and iPSC, as well as the term embryonic germ stem cells (EGSC), which are another example of a PSC. PSCs may be in the form of an established cell line, they may be obtained directly from primary embryonic tissue, or they may be derived from a somatic cell. PSCs can be target cells of the methods described herein.

By “induced pluripotent stem cell” or “iPSC” it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPSCs may be found in, for example, U.S. Patent Publication Nos. US20090047263, US20090068742, US20090191159, US20090227032, US20090246875, and US20090304646, the disclosures of which are incorporated herein by reference. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

By “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e. ectoderm, mesoderm and endoderm. For example, somatic cells would include both neurons and neural progenitors, the latter of which may be able to naturally give rise to all or some cell types of the central nervous system but cannot give rise to cells of the mesoderm or endoderm lineages.

By “mitotic cell” it is meant a cell undergoing mitosis. Mitosis is the process by which a eukaryotic cell separates the chromosomes in its nucleus into two identical sets in two separate nuclei. It is generally followed immediately by cytokinesis, which divides the nuclei, cytoplasm, organelles and cell membrane into two cells containing roughly equal shares of these cellular components.

By “post-mitotic cell” it is meant a cell that has exited from mitosis, i.e., it is “quiescent”, i.e. it is no longer undergoing divisions. This quiescent state may be temporary, i.e. reversible, or it may be permanent.

By “meiotic cell” it is meant a cell that is undergoing meiosis. Meiosis is the process by which a cell divides its nuclear material for the purpose of producing gametes or spores. Unlike mitosis, in meiosis, the chromosomes undergo a recombination step which shuffles genetic material between chromosomes. Additionally, the outcome of meiosis is four (genetically unique) haploid cells, as compared with the two (genetically identical) diploid cells produced from mitosis.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired, particularly humans.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Disclosed herein are methods and compositions for genome engineering, including genome engineering to study and treat a frontotemporal degeneration (FTD) or amyotrophic lateral sclerosis (ALS). It is now recognized that the C9orf72 gene is the most common gene causing hereditary FTD, ALS and ALS with FTD. The invention describes genomic editing of any target cell such that there is a favorable change in the expression of C9orf72 genes, which in turn results in treatment of a disease in a subject in need thereof. Non-limiting examples of diseases include neurological diseases (e.g., FTD, ALS, etc.), cancers, and the like. Additionally, delivery of altered stem cells in a transplant altered to express a desired protein product can be similarly beneficial in a disease. Also described are cell lines and organisms with altered gene expression. Described below are genes to be targeted by the CRISPR/Cas system using the sgRNAs of the invention. Mammalian gene locations as described are relative to the UCSC Genome Brower created by the Genome Bioinformatics Group of UC Santa Cruz, software copyright the Regents of the University of California. Human genomic coordinates are provided in the GRCh37/hg19 assembly of the human genome, and correspond to numbers on a double stranded DNA. Thus, any position described by a genomic coordinate corresponds to either the (+) or Watson strand, or may specify its corresponding (−) or Crick strand.

### THE EMBODIMENTS

In an exemplary embodiment, the invention provides one or more guide RNA sequence(s) active in a CRISPR system. In various embodiments, the CRISPR system edits the C9orf72 repeat expansion mutation in humans. In an exemplary embodiment the guide RNA targets and hybridizes to a site in cis with the C9orf72 repeat expansion mutation. In an exemplary embodiment, the guide RNA targets and hybridizes to a site in cis with the wild type allele of the C9orf72 gene. In an exemplary embodiment, the guide RNA targets and hybridizes to a site in cis with the mutant allele of the C9orf72 gene.

In various embodiments, the invention provides a guide nucleic acid having at least about 90%, about 91%, 92%, 93%, 94%, 95%, 96%, 97%, 98% or at least about 99% sequence homology to a sequence selected from the group consisting of SEQ ID NOs: 1-2, 5-6, 13-14, 21-22, 29-30, 37-38, 45-48, 55-712, 731-740, and 749-1410. In various embodiments, the invention provides a guide nucleic acid at least about 90%, about 91%, 92%, 93%, 94%, 95%, 96%, 97%, 98% or at least about 99% sequence homology to a sequence selected from the group consisting of SEQ ID NOs: 1-2, 5-6, 13-14, 21-22,29-30, 37-38, 45-48, 55-712, 731-746, and 749-1410, which binds to hybridizes to a site in cis with at least one allele of the C9orf72 gene in a manner appropriate to form a substrate for Cas9.

In various embodiments, the guide nucleic acid is a component of an expression vector.

In an exemplary embodiment, the invention provides a host cell containing one or more guide sequences having at least about 90%, about 91%, 92%, 93%, 94%, 95%, 96%, 97%, 98% or at least about 99% sequence homology to a sequence selected from the group consisting of SEQ ID NOs: 1-2, 5-6, 13-14, 21-22, 29-30, 37-38, 45-48, 55-712, 731-746, and 749-1410. In an exemplary embodiment, the host cell is a component of a functional organism (e.g., a human).

In various embodiments, the one or more guide sequence is internal to a host cell and is contained within a delivery vehicle, e.g., a viral plasmid, a lipid delivery particle or the like.

In one embodiment, the compositions include a nucleic acid sequence comprising a sequence encoding a CRISPR-associated endonuclease and one or more guide RNAs, wherein the guide RNA is complementary to a target site in cis with the C9orf72 repeat expansion mutation. In some embodiments this nucleic acid is contained within an expression vector. In one embodiment, the compositions include a CRISPR-associated endonuclease polypeptide and one or more guide RNAs, wherein the guide RNA is complementary to a target site in cis with the C9orf72 repeat expansion mutation. In one embodiment, the guide nucleic acid sequence is having at least about 90%, about 91%, 92%, 93%, 94%, 95%, 96%, 97%, 98% or at least about 99% sequence homology to a sequence selected from the group consisting of SEQ ID NOs: 1-2, 5-6, 13-14, 21-22, 29-30, 37-38, 45-48, 55-712, 731-746, and 749-1410.

Also provided are nucleic acids encoding a CRISPR-Cas ribonucleoprotein (RNP) complex for correcting a C9orf72 GC repeat expansion mutation comprising a sequence of a guide nucleic acid having at least about 90%, about 91%, 92%, 93%, 94%, 95%, 96%, 97%, 98% or at least about 99% sequence homology to a sequence selected from the group consisting of SEQ ID NOs: 1-2, 5-6, 13-14, 21-22, 29-30, 37-38, 45-48, 55-712, 731-746, and 749-1410, wherein the nucleic acid is delivered to a target site through a carrier, e.g., a functional carrier.

Also provided herein are methods of correcting the C9orf72 GC repeat expansion mutation in a host cell. An exemplary method includes administering to a host cell a guide nucleic acid of the invention and such auxiliary sequences and enzymes as are necessary to correct the C9orf72 GC repeat expansion mutation in a host cell.

In one embodiment, a first guide nucleic acid targets and hybridizes to a sequence upstream of the C9orf72 GC repeat expansion region. In some embodiments, a second guide nucleic acid targets and hybridizes to a sequence downstream of the C9orf72 GC repeat expansion region. In various embodiments, a first guide nucleic acid targets and hybridizes to a sequence upstream of the C9orf72 GC repeat expansion region and a second guide nucleic acid targets and hybridizes to a sequence downstream of the C9orf72 GC repeat expansion region. An exemplary first guide nucleic acid sequence comprises a sequence having at least about 90%, about 91%, 92%, 93%, 94%, 95%, 96%, 97%, 98% or at least about 99% sequence homology to SEQ ID NO: 1, or SEQ ID NO. 731. An exemplary second guide nucleic acid sequence comprises a sequence having at least about 90%, about 91%, 92%, 93%, 94%, 95%, 96%, 97%, 98% or at least about 99% sequence homology to a SEQ ID NO: 2, or SEQ ID NO. 732.

An exemplary method further comprises:
a. excising a region containing a C9orf72 GC repeat expansion in a mutant allele by cleaving one or both strands of DNA at a first target nucleic acid sequence and at a second target nucleic acid sequence with the endonuclease (e.g., Cas9); and
b. excising a region in the normal allele by cleaving one or both strands of DNA at a first target nucleic acid sequence and at a second target nucleic acid sequence with the endonuclease (e.g. Cas9).

Also provided herein is a method of correcting the C9orf72 GC repeat expansion mutation in a host cell. The method includes administering to the host cell an endonuclease (e.g., Cas9) and one, two or more guide nucleic acids. In one embodiment, a first guide nucleic acid targets and hybridizes to a sequence upstream of the exon 1A at the C9orf72 locus, and a second guide nucleic acid targets and hybridizes to a sequence downstream of the exon 1B at the C9orf72 locus. An exemplary first guide nucleic acid sequence comprises a sequence having at least about 90%, about 91%, 92%, 93%, 94%, 95%, 96%, 97%, 98% or at least about 99% sequence homology to SEQ ID NO: 21, or SEQ ID NO: 737, and the second guide nucleic acid sequence comprises a sequence having at least about 90%, about 91%, 92%, 93%, 94%, 95%, 96%, 97%, 98% or at least about 99% sequence homology to SEQ ID NO: 22, or SEQ ID NO: 738. The method further comprises excising a region containing exon 1A, exon 1B and at least a portion of the GC repeat expansion in the mutant allele by cleaving one or both strands of DNA at first and second target nucleic acid sequences with the endonuclease (e.g., Cas9).

In exemplary embodiments, the invention provides methods of correcting the C9orf72 GC repeat expansion mutation in a host cell comprising treating of host cell with endonuclease and one, two or more guide nucleic acids in which a first guide nucleic acid targets and hybridizes to a sequence upstream of a transcriptional start site at the C9orf72 locus, and a second guide nucleic acid targets and hybridizes to a sequence downstream of the transcriptional start site at the C9orf72 locus. The first guide nucleic acid sequence comprises a sequence having at least about 90%, about 91%, 92%, 93%, 94%, 95%, 96%, 97%, 98% or at least about 99% sequence homology to SEQ ID NO: 5, or SEQ ID NO: 733 and the second guide nucleic acid sequence comprises a sequence having at least about 90%, about 91%, 92%, 93%, 94%, 95%, 96%, 97%, 98% or at least about 99% sequence homology to SEQ ID NO: 6, or SEQ ID NO: 734. In various embodiments, the method further comprises the steps of:
a. excising a region that contains the transcriptional start site in the mutant allele by cleaving one or both strands of DNA at a first target nucleic acid sequence and at a second target nucleic acid sequence with the endonuclease (e.g., Cas9);
b. excising a region that contains the transcriptional start site in the normal allele by cleaving one or both strands of DNA at a first target nucleic acid sequence and a second target nucleic acid sequence with the endonuclease (e.g., Cas9); and
c. thereby changing expression level of the C9orf72 gene in the host cell.

Also provided herein are a population of engineered cells, wherein a C9orf72 GC repeat expansion mutation in the cells has been corrected by any of the methods disclosed herein.

Also provided herein are methods of treating C9orf72 GC repeat expansion associated diseases in a subject, comprising administering a population of engineered cells, wherein a C9orf72 GC repeat expansion mutation in the cells has been corrected by any of the methods disclosed above.

### 1. C9orf72 GC Repeat Expansion

In various embodiments, the present invention is directed to in vivo or in vitro systems for use as novel therapeutics and, in some embodiments, methods of treatment, that can be used for the treatment of neurodegenerative diseases, disorders and conditions. In some embodiments, an in vivo or in vitro system described herein treats a disease, disorder, and/or condition associated with a C9orf72 locus. An exemplary disease, disorder or condition is associated with a hexanucleotide repeat extension sequence at the locus. An exemplary disease is a neurodegenerative disorder (e.g., ALS and/or FTD).

In some embodiments, a hexanucleotide repeat expansion mutation sequence comprises at least one, e.g., at least about three, at least about five, at least about ten, at least about fifteen, at least about twenty, at least about thirty, at least about forty, at least about fifty, at least about sixty, at least about seventy, at least about eighty, at least about ninety or at least about one-hundred, or at least a thousand contiguous, repeats of the hexanucleotide sequence.

In some embodiments, a human hexanucleotide expansion sequence span (and optionally encompasses) all or one or more portions of exons 1A and/or exon 1B of a human C9orf72 gene.

### 2. The CRISPR/Cas System

Exemplary compositions of the invention include a CRISPR-associated endonuclease, e.g., Cas9, one or more guide RNAs complementary to and capable of hybridizing to a target site in cis with the C9orf72 repeat expansion mutation. In some embodiments, there is provided a nucleic acid encoding a CRISPR-associated endonuclease, e.g., Cas9, and one or more guide RNAs complementary to and capable of hybridizing to a target site in cis with the C9orf72 repeat expansion mutation.
a. CRISPR-Associated Endonuclease

The compositions of the invention can include a CRISPR-associated endonuclease and/or a nucleic acid encoding a CRISPR-associated endonuclease.

Exemplary CRISPR-associated endonucleases include type II CRISPR/Cas system endonucleases, having endonuclease activity to cut target DNA. In an exemplary embodiment, the endonuclease provided is a Cas9 polypeptide. Cas9 is guided by a mature CRISPR (Clustered Regularly Interspaced Short Palindromic Repeats) RNA (crRNA) that contains about 20 base pairs (bp) of unique target sequence (called spacer) and a trans-activated small RNA (tracrRNA) that serves as a guide for ribonuclease III-aided processing of pre-crRNA. The crRNA: tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM).

In an exemplary embodiment, there is provided a Cas9 polypeptide. By “Cas9 polypeptide” or “site-directed polypeptide” or “site-directed Cas9 polypeptide” is meant a polypeptide that binds RNA (e.g., the protein binding segment of a guide nucleic acid) and is targeted to a specific sequence (a target site) in a target nucleic acid. A Cas9 polypeptide as described herein is targeted to a target site by the guide nucleic acid to which it is bound. The guide nucleic acid comprises a sequence complementary to a target sequence within the target nucleic acid, thus targeting the bound Cas9 polypeptide to a specific location within the target nucleic acid (the target sequence) (e.g., stabilizing the interaction of Cas9 with the target nucleic acid). Naturally occurring Cas9 polypeptides bind a guide nucleic acid, and are thereby directed to a specific sequence within a target nucleic acid (a target site), and cleave the target nucleic acid (e.g., cleave dsDNA to generate a double strand break, cleave ssDNA, cleave ssRNA, etc.). A subject Cas9 polypeptide comprises two portions, an RNA-binding portion and an activity portion. An RNA-binding portion interacts with a subject guide nucleic acid. An activity portion exhibits site-directed enzymatic activity (e.g., nuclease activity, activity for DNA and/or RNA methylation, activity for DNA and/or RNA cleavage, activity for histone acetylation, activity for histone methylation, activity for RNA modification, activity for RNA-binding, activity for RNA splicing etc.). In some cases the activity portion exhibits reduced nuclease activity relative to the corresponding portion of a wild type Cas9 polypeptide. In some cases, the activity portion is enzymatically inactive.

In some embodiments, the Cas9 polypeptide is a naturally occurring polypeptide (e.g., naturally occurs in bacterial and/or archaeal cells). In various embodiments, the Cas9 polypeptide is not a naturally occurring polypeptide (e.g., the Cas9 polypeptide is a variant Cas9 polypeptide, a chimeric polypeptide as discussed below, and the like). In some embodiments, the Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Psuedomona aeruginosa, Escherichia coli, or other sequenced bacteria genomes and archaea, or other prokaryotic microorganisms. In selected embodiments, the wild type Streptococcus pyrogenes Cas9 sequence is modified. For exemplary Cas9 nucleases, the nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GI: 669193757; KM099232.1 GI: 669193761; or KM099233.1 GI:669193765. Alternatively, the Cas9 nuclease sequence can be, for example, the sequence contained within a commercially available vector such as PX330 or PX260 from Addgene (Cambridge, Mass.). In some embodiments, the Cas9 endonuclease has an amino acid sequence that is a variant or a fragment of any of the Cas9 endonuclease sequences of Genbank accession numbers KM099231.1 GI:669193757; KM099232.1 GI: 669193761; or KM099233.1 GI: 669193765 or Cas9 amino acid sequence of PX330 or PX260 (Addgene, Cambridge, Mass.).

Exemplary CRISPR-associated endonucleases include Cas polypeptides from Type V CRISPR systems. In one embodiment, the endonuclease is Cpfl. Cpfl is a single RNA-guided endonuclease that, in contrast to Type II systems, lacks tracrRNA. In fact, Cpf1-associated CRISPR arrays are processed into mature crRNAS without the requirement of an additional trans-activating tracrRNA. Useful Cpfl Protein include, without limitation, the Cpfl Protein disclosed in U.S. Pat. No. 9,790,490B2, U.S. Pat. No. 9,745,562B2, U.S. Pat. No. 11,268,082B2, U.S. Pat. No. 11,286,478B2, US20190010481A1, or US20190062735A1. In some embodiments, the endonuclease is Cas12a (type V-A), Cas12b (type V-B), and Cas12e (type V-E), or Cas12J (type V-J).

Exemplary CRISPR-associated endonucleases include CasX Proteins. A CasX polypeptide (this term is used interchangeably with the term “CasX protein”) can bind and/or modify (e.g., cleave, nick, methylate, demethylate, etc.) a target nucleic acid and/or a polypeptide associated with target nucleic acid (e.g., methylation or acetylation of a histone tail) (e.g., in some cases the CasX protein includes a fusion partner with an activity, and in some cases the CasX protein provides nuclease activity). Useful CasX Protein include, without limitation, the CasX Protein disclosed in U.S. Pat. No. 10,570,415B2, U.S. Pat. No. 11,268,082B2, US20190367924A1, US20180362590A1, US20210198330A1, or US20190093091A1.

Exemplary CRISPR-associated endonucleases include other Cas polypeptides that are naturally occurring, non-naturally occurring, or under developments. In some embodiments, the endonuclease is a Cas polypeptide from two classes (Class I and Class II) Cas polypeptides that are subdivided into at least 6 types (I-VI). Useful CRISPR-associated endonucleases include, without limitation, Cas polypeptides disclosed in U.S. Pat. No. 10,808,245B2, U.S. Pat. No. 11,225,659B2, U.S. Pat. No. 11,168,324B2, or US20210301288A1.

Assays used to determine whether a protein has an RNA-binding portion interacting with a subject guide nucleic acid are any convenient binding assay testing for binding between a protein and a nucleic acid. Exemplary assays include binding assays (e.g., gel shift assays) that include adding a guide nucleic acid and a Cas9 polypeptide to a target nucleic acid. In some cases, a PAMmer is also added (e.g., in some cases when the target nucleic acid is a single stranded nucleic acid).

Assays to determine whether a protein has an activity portion (e.g., to determine if the polypeptide has nuclease activity cleaving a target nucleic acid) can be any convenient nucleic acid cleavage assay that tests for nucleic acid cleavage. Exemplary assays include cleavage assays involving adding a guide nucleic acid and a Cas9 polypeptide to a target nucleic acid. In some cases, a PAMmer is also added (e.g., in some cases when the target nucleic acid is a single stranded nucleic acid).

b. Guide Nucleic Acid for Gene Editing

In an exemplary embodiment, the invention provides a guide nucleic acid, e.g., a nucleic acid having at least about 90%, about 91%, 92%, 93%, 94%, 95%, 96%, 97%, 98% or at least about 99% sequence homology to a sequence selected from SEQ ID NO: 1-2, 5-6, 13-14, 21-22, 29-30, 37-38, 45-48, 55-712, 731-746, and 749-1410, as shown in Table 1-2, and Table 8-9. A nucleic acid molecule that binds to a Cas polypeptide and targets the polypeptide to a specific location within the target nucleic acid is referred to herein as a “guide nucleic acid”. When the guide nucleic acid is an RNA molecule, it can be referred to as a “guide RNA” or a “gRNA”. A subject guide nucleic acid comprises two segments, a first segment (referred to herein as a “targeting segment”); and a second segment (referred to herein as a “protein-binding segment”).

### (i). Targeting Segment

The first segment (targeting segment) of an exemplary guide nucleic acid comprises a nucleotide sequence complementary to a target site in cis with the C9orf72 repeat expansion mutation. In other words, the targeting segment of an exemplary guide nucleic acid interacts with a target site in cis with the C9orf72 gene (e.g., a single stranded RNA (ssRNA) and/or a single stranded DNA (ssDNA)) in a sequence-specific manner via hybridization (i.e., base pairing). As such, the nucleotide sequence of the targeting segment may vary and can determine the location within the target nucleic acid that the guide nucleic acid and the target nucleic acid will interact. The targeting segment of a subject guide nucleic acid can be modified (e.g., by genetic engineering) to hybridize to any desired sequence (target site) in cis with the C9orf72 gene. In some embodiments, the target site is in cis with a region comprising at least one hexanucleotide repeats (GGGGCC; or G4C2), e.g., at least about three, at least about five, at least about ten, at least about fifteen, at least about twenty, at least about thirty, at least about forty, at least about fifty, at least about sixty, at least about seventy, at least about eighty, at least about ninety or at least about one-hundred, or at least a thousand contiguous, repeats of the hexanucleotide sequence.

An exemplary targeting segment can have a length of from about 12 nucleotides to about 100 nucleotides. For example, the targeting segment can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the targeting segment can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt.

An exemplary nucleotide sequence (the targeting sequence) of the targeting segment that is complementary to a nucleotide sequence (target site) in cis with the C9orf72 repeat expansion mutation can have a length of 12 nt or more. For example, the targeting sequence of the targeting segment that is complementary to a target site of the target nucleic acid can have a length of 12 nt or more, 15 nt or more, 18 nt or more, 19 nt or more, 20 nt or more, 25 nt or more, 30 nt or more, 35 nt or more or 40 nt. For example, an exemplary targeting sequence of the targeting segment that is complementary to a target sequence of the target nucleic acid can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt.

### (ii). Protein-Binding Segment

As an illustrative, non-limiting example, a protein-binding segment of a guide nucleic acid that comprises two separate molecules can comprise (i) base pairs 40-75 of a first molecule (e.g., RNA molecule, DNA/RNA hybrid molecule) that is 100 base pairs in length; and (ii) base pairs 10-25 of a second molecule (e.g., RNA molecule) that is 50 base pairs in length.

In an exemplary embodiment, the protein-binding segment (or “protein-binding sequence”) used in present invention is a sequence interacting with a Cas polypeptide. Useful protein-binding sequences include, without limitation, the Cas polypeptide-binding sequences disclosed in U.S. Pat. No. 11,261,439B2, U.S. Pat. No. 9,738,908B2, U.S. Pat. No. 10,920,221B2, US20210180055A1 or US20200291370. In an exemplary embodiment, the “protein-binding segment” comprises a duplex formed by a crRNA comprising rGrUrU rUrUrA rGrArG rCrUrA rUrGrC rU (Seq ID NO: 722) and a tracrRNA. Useful tracrRNA sequences include, without limitation, the tracrRNA sequences disclosed in U.S. Pat. No. 10,711,258B2, US20190032131A1, US20180200387A1, US20210017518A1, US20190032052A1 or US20220047722A1. In an exemplary embodiment, the “protein-binding sequence” of a single guide RNA (sgRNA) comprises rG rUrUrU rUrArG rArGrC rUrArG rArArA rUrArG rCrArA rGrUrU rArArA rArUrA rArGrG rCrUrA rGrUrC rCrGrU rUrArU rCrArA rCrUrU rGrArA rArArA rGrUrG rGrCrA rCrCrG rArGrU rCrGrG rUrGrC mU*mU*mU*rU (SEQ ID NO: 723) as shown in Table 5.

Site-specific binding and/or cleavage of the target nucleic acid can occur at locations determined by base-pairing complementarity between the guide nucleic acid and the target nucleic acid. The protein-binding segment of a subject guide nucleic acid comprises two complementary stretches of nucleotides that hybridize to one another to form a double stranded RNA duplex (dsRNA duplex).

A subject guide nucleic acid and a subject Cas polypeptide form a complex (i.e., bind via non-covalent interactions). The guide nucleic acid provides target specificity to the complex by comprising a nucleotide sequence that is complementary to a sequence of a target nucleic acid. The Cas polypeptide of the complex provides the site-specific activity. In other words, the Cas polypeptide is guided to a target nucleic acid sequence (e.g. a target sequence in a chromosomal nucleic acid; a target sequence in an extrachromosomal nucleic acid, e.g. an episomal nucleic acid, a minicircle, an ssRNA, an ssDNA, etc.; a target sequence in a mitochondrial nucleic acid; a target sequence in a plasmid; etc.) by virtue of its association with the protein-binding segment of the guide nucleic acid. In various embodiments, the invention provides a complex between a guide nucleic acid of the invention and a Cas polypeptide.

(iii). Additional Sequences

In some embodiments, a guide nucleic acid comprises an additional segment or segments (in some cases at the 5′ end, in some cases the 3′ end, in some cases at either the 5′ or 3′ end, in some cases embedded within the sequence (i.e., not at the 5′ and/or 3′ end), in some cases at both the 5′ end and the 3′ end, in some cases embedded and at the 5′ end and/or the 3′ end, etc). For example, a suitable additional segment can comprise a 5′ cap (e.g., a 7-methylguanylate cap (m7G)); a 3′ polyadenylated tail (i.e., a 3′ poly(A) tail); a ribozyme sequence (e.g. to allow for self-cleavage of a guide nucleic acid (or component of a guide nucleic acid, e.g., a targeter, an activator, etc.) and release of a mature PAMmer in a regulated fashion); a riboswitch sequence (e.g., to allow for regulated stability and/or regulated accessibility by proteins and protein complexes); a sequence that forms a dsRNA duplex (i.e., a hairpin)); a sequence that targets an RNA to a subcellular location (e.g., nucleus, mitochondria, chloroplasts, and the like); a modification or sequence that provides for tracking (e.g., a direct label (e.g., direct conjugation to a fluorescent molecule (i.e., fluorescent dye)), conjugation to a moiety that facilitates fluorescent detection, a sequence that allows for fluorescent detection; a modification or sequence that provides a binding site for proteins (e.g., proteins that act on DNA, including transcriptional activators, transcriptional repressors, DNA methyltransferases, DNA demethylases, histone acetyltransferases, histone deacetylases, proteins that bind RNA (e.g., RNA aptamers), labeled proteins, fluorescently labeled proteins, and the like); a modification or sequence that provides for increased, decreased, and/or controllable stability; and combinations thereof.

### 3. Method of Use

An exemplary method of cleaving any desired sequence (target site) in cis with the C9orf72 repeat expansion mutation includes contacting a target nucleic acid with a Cas polypeptide, a guide nucleic acid (e.g., a dual guide RNA, a single guide RNA, an RNA/DNA hybrid guide RNA, etc.), and a PAMmer.

In some embodiments, the designed gRNAs are those having the fewest overall off-target binding events, including those with no predicted off-target matches to the exact sequence and no predicted off-target within the first 2 bases of the PAM. In some embodiments, the gRNAs are designed and evaluated by a bioinformatic tool. In an exemplary embodiment, the bioinformatic tool is AlleleAnalyzer. In one exemplary embodiment, the bioinformatic tool is CRISPOR.

In some embodiments, the target sequence for gene editing is a sequence between two alternative non-coding 5′UTR start sites, exon 1A and exon 1B. In some embodiments, the target sequence for gene editing is a sequence lying upstream of the non-coding 5′UTR start sites exon 1A. In some embodiments, the target sequence for gene editing is a sequence lying downstream of the non-coding 5′UTR start sites exon 1B.

In some embodiments, the GC repeat expansion mutation is knocked-out or silenced. In some embodiments, the C9orf72 mutation is knocked-out through cleaving at two target sites in cis with the C9orf72 repeat expansion mutation. In some embodiments, the C9orf72 mutation is knocked-out through cleaving at two or more target sites in cis with the C9orf72 repeat expansion mutation. In some embodiments, the C9orf72 mutation knockout is facilitated by two guide nucleic acids, e.g. gRNAs. In some embodiment, the C9orf72 mutation knockout is conducted by two or more guide nucleic acids, e.g. gRNAs. Methods and compositions disclosed herein may comprise multiple guide nucleic acids, wherein each guide nucleic acid has a different guide sequence, thereby targeting a different target sequence. In such cases, multiple guide nucleic acids can be using in multiplexing, wherein multiple targets in cis with the C9orf72 repeat expansion mutation are targeted simultaneously.

In an exemplary embodiment, a first gRNA provides target specificity to the complex by incorporating a nucleotide sequence complementary to a first target site. A second gRNA provides target specificity to the complex by incorporating a nucleotide sequence complementary to a second target site. Each guide nucleic acid and a subject Cas polypeptide form a complex, providing the site-specific activity, binding and/or modifying (e.g., cleave, methylate, demethylate, etc.) the target sequence (target site) in cis with the C9orf72 repeat expansion mutation. In an exemplary embodiment, the target site is located in a region between 25 kbp upstream and 28 kbp downstream of a transcription start site of the C9orf72 gene. The cleavage of one or both strands in or near (e.g. within 1, 2, 3, 4, 5, 6, 7, 8, 9, 10, 20, 50, or more base pairs from) the target sequence can occur within a target sequence, 5′ of the target sequence, upstream of a target sequence, 3′ of the target sequence, or downstream of a target sequence. In some embodiments, the nucleic acid-guided nuclease directs cleavage of one or both strands at the location of a target sequence, such as within the target sequence and/or within the complement of the target sequence. In some embodiments, the nucleic acid-guided nuclease directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, at least more than one GC repeat expansion region at the C9orf72 locus is excised. In some embodiments, the excision of one or more GC repeat expansion region at the C9orf72 locus is biallelic. In an exemplary embodiment, the method of correcting the C9orf72 GC repeat expansion mutation in a host cell includes administering to the host cell a system comprising an endonuclease and two or more guide nucleic acids, wherein a first guide nucleic acid targets and hybridizes with a sequence upstream of the GC repeat expansion region, and a second guide nucleic acid targets and hybridizes with a sequence downstream of the GC repeat expansion region, wherein the first guide nucleic acid sequence comprising a sequence having at least about 90%, about 91%, 92%, 93%, 94%, 95%, 96%, 97%, 98% or at least about 99% sequence homology to SEQ ID NO. 1, or SEQ ID NO. 731, and the second guide nucleic acid sequence comprising a sequence having at least about 90%, about 91%, 92%, 93%, 94%, 95%, 96%, 97%, 98% or at least about 99% sequence homology to SEQ ID NO: 2, or SEQ ID NO. 732. An exemplary method further comprises:
a. excising a region containing a GC repeat expansion in the mutant allele by cleaving one or both strands of DNA at a first target nucleic acid sequence and at a second target nucleic acid sequence with the endonuclease; and
b. excising a region in the normal allele by cleaving one or both strands of DNA at a first target nucleic acid sequence and at a second target nucleic acid sequence with the endonuclease.

In some embodiments, the excision of a GC repeat expansion region at the C9orf72 locus is monoallelic. In some embodiments, the GC repeat expansion mutant allele is excised while the normal allele of the genome is maintained. In some embodiments, the GC repeat expansion mutant allele is excised by a Cas9 that can distinguish between alleles differing by a single nucleotide. In an exemplary embodiment, the method of correcting the C9orf72 repeat expansion mutation in a host cell comprises administering to a host cell an endonuclease and one, two or more guide nucleic acids. A first guide nucleic acid targets and hybridizes to a sequence upstream of the exon 1A at the C9orf72 locus, wherein a second guide nucleic acid targets and hybridizes to a sequence downstream of the exon 1B at the C9orf72 locus, wherein the first guide nucleic acid sequence comprises a sequence having at least about 90%, about 91%, 92%, 93%, 94%, 95%, 96%, 97%, 98% or at least about 99% sequence homology to SEQ ID NO: 21, or SEQ ID NO. 737, and the second guide nucleic acid sequence comprises a sequence having at least about 90%, about 91%, 92%, 93%, 94%, 95%, 96%, 97%, 98% or at least about 99% sequence homology to SEQ ID NO: 22, or SEQ ID NO. 738, and further comprising excising a region that contains exon 1A, exon 1B and at least a portion of GC repeats expansion in the mutant allele by cleaving a one or both strands of DNA at a first target nucleic acid sequence and at a second target nucleic acid sequence with the endonuclease.

In some embodiments, a GC repeat expansion mutant allele is silenced by excising a regulatory region. In some methods, a control sequence can be inactivated such that it no longer functions as a regulatory sequence. As used herein, “regulatory sequence” can refer to any nucleic acid sequence that effects the transcription, translation, or accessibility of a nucleic acid sequence. Examples of regulatory sequences include a promoter, a transcription terminator, and an enhancer. In some embodiments, the regulatory region lies in exon 1A which includes a transcriptional start site and controls the expression of the C9orf72 sense-transcript harboring the mutation.

In one embodiment, methods of correcting the C9orf72 GC repeat expansion mutation in a host cell comprises administering to a host cell an endonuclease and two or more guide nucleic acids, wherein a first guide nucleic acid targets and hybridizes to a sequence upstream of a transcriptional start site at the C9orf72 locus, and a second guide nucleic acid targets and hybridizes to a sequence downstream of the transcriptional start site at the C9orf72 locus. The first guide nucleic acid sequence comprises a sequence having at least about 90%, about 91%, 92%, 93%, 94%, 95%, 96%, 97%, 98% or at least about 99% sequence homology to SEQ ID NO: 5, or SEQ ID NO. 733, wherein the second guide nucleic acid sequence comprising having at least about 90%, about 91%, 92%, 93%, 94%, 95%, 96%, 97%, 98% or at least about 99% sequence homology to SEQ ID NO: 6, or SEQ ID NO. 734. An exemplary method of the invention further comprises the steps of:
a. excising a region that contains the transcriptional start site in the mutant allele by cleaving one or both strands of DNA at a first target nucleic acid sequence and at a second target nucleic acid sequence with the endonuclease;
b. excising a region that contains the transcriptional start site in the normal allele by cleaving one or both strands of DNA at a first target nucleic acid sequence and a second target nucleic acid sequence with the endonuclease; and
c. changing expression level of the C9orf72 gene in the host cells.

In some embodiments, the methods for correcting the C9orf72 GC repeat expansion mutation are selected from the group consisting of C9orf72 GC repeat expansion biallelic excision, C9orf72 GC repeat expansion mutation monoallelic excision, C9orf72 expression modification, and a combination thereof.

The effectiveness of editing the GC repeat expansion can be determined by art-recognized assays. For example, to assay for an agent-induced alteration in the level of mRNA transcripts or corresponding polynucleotides, nucleic acid contained in a sample is first extracted according to standard methods in the art. For instance, mRNA can be isolated using various lytic enzymes or chemical solutions according to the procedures set forth in Green and Sambrook (2014), or extracted by nucleic-acid-binding resins following the accompanying instructions provided by the manufacturers. The mRNA contained in the extracted nucleic acid sample is then detected by amplification procedures or conventional hybridization assays (e.g. Northern blot analysis) according to methods widely known in the art or based on the methods exemplified herein. In some embodiments, the mRNA contained in the extracted nucleic acid sample is then detected by Droplet Digital PCR (ddPCR).

In some embodiments, single molecule sequencing of a genomic region containing the repeat region is used to size the entirety of the C9orf72 GC rich repetitive region and evaluate the editing results of C9orf72 GC repeat expansion region.

### 4. Delivery System

In an exemplary embodiment, one or more guide nucleic acid in the form of RNA or encoded on a DNA expression cassette can be introduced into a host cell. In an exemplary embodiment, the guide nucleic acid may be provided in the cassette with as one or more polynucleotides, which may be contiguous or non-contiguous in the cassette. In specific embodiments, the guide nucleic acid is provided in the cassette as a single contiguous polynucleotide.

In some embodiments, one or more vectors driving expression of one or more components of a targetable nuclease system are introduced into a host cell or in vitro. For example, a nucleic acid-guided nuclease and a guide nucleic acid can each be operably linked to separate regulatory elements on separate vectors. Alternatively, two or more of the elements expressed from the same or different regulatory elements, may be combined in a single vector, with one or more additional vectors providing any components of the targetable nuclease system not included in the first vector. Targetable nuclease system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a nucleic acid-guided nuclease and one or more guide nucleic acids. In some embodiments, a nucleic acid-guided nuclease and one or more guide nucleic acids are operably linked to and expressed from the same promoter. In other embodiments, one or more guide nucleic acids or polynucleotides encoding the one or more guide nucleic acids are introduced into a cell or in vitro environment already comprising a nucleic acid-guided nuclease or polynucleotide sequence encoding the nucleic acid-guided nuclease.

When multiple different guide sequences are used, a single expression construct may be used to target nuclease activity to multiple different, corresponding target sequences within a cell or in vitro. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell or in vitro.

A nucleic acid-guided nuclease and one or more guide nucleic acids can be delivered either as DNA or RNA. In some aspect, the invention provides methods comprising delivering one or more polynucleotides, such as or one or more vectors or linear polynucleotides as described herein, one or more transcripts thereof, and/or one or proteins transcribed therefrom, to a host cell.

In an exemplary embodiment, the delivery system is a viral vector delivery system. Viral vector delivery systems include DNA and RNA viruses. In an exemplary embodiment, the delivery system is a lentiviral vector delivery system. Lentiviral vectors are retroviral vectors able to transduce or infect non-dividing cells and typically produce high viral titers. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Other useful viral vector delivery systems include, without limitation, the viral vectors disclosed in Anderson, Science 256:808-813 (1992); Nabel & Feigner, TIBTECH 11:211-217 (1993); Mitani & Caskey, TIBTECH 11:162-166 (1993); Dillon. TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer & Perricaudet, British Medical Bulletin 51 (1): 31-44 (1995); Haddada et al., in Current Topics in Microbiology and Immunology Doerfler and Bohm (eds) (1995); and Yu et al., Gene Therapy 1:13-26 (1994).

In some embodiments, the delivery system is a non-viral delivery system. Methods of non-viral delivery of nucleic acids include yeast systems, lipofection, microinjection, biolistics, virosomes, liposomes, immunoliposomes, polycation or lipid: nucleic acid conjugates, naked DNA, virions, artificial virions, agent-enhanced uptake of DNA, electroporation, cell permeable peptides, nanoparticles, nanowires (Shalek et al., Nano Letters, 2012), exosomes or molecular trojan horses liposomes (Pardridge et al., Cold Spring Harb Protoc; 2010; doi: 10.1101/pdb.prot5407).

Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In some embodiments, the endonuclease and gRNA are delivered via the system as disclosed in US20210139892A, comprising a) a modified RNA-binding protein (RBP) comprising: i) an RBP; and ii) one or more endosomolytic peptides (ELPs) covalently linked, directly or via a linker, to the RBP; and b) a modified cargo RNA complexed to the RBP, wherein the modified cargo RNA comprises a cargo RNA modified to include one or more RBP binding sites that are bound by the RBP present in the modified RBP.

In an exemplary embodiment, the endonuclease and gRNA are delivered as ribonucleoprotein (RNP) (e.g., a RNP comprising a site-directed modifying polypeptide, such as a Cas9 RNP or a Cpf1 RNP) via a system as described in U.S. Pat. No. 10,851,367B2 or under development. In some embodiments of such a composition, the ribonucleoprotein (e.g., a RNP comprising a site-directed modifying polypeptide, such as a Cas9 RNP or a Cpf1 RNP) is co-incubated with the endosomal escape agent to form the composition. In some embodiments, the ribonucleoprotein (e.g., a RNP comprising a site-directed modifying polypeptide, such as a Cas9 RNP or a Cpf1 RNP) or the endosomal escape agent is conjugated to an antibody or a fragment thereof. In some embodiments, the ribonucleoprotein (e.g., a RNP comprising a site-directed modifying polypeptide, such as a Cas9 RNP or a Cpf1 RNP) is modified to include glycosylation sites. In some embodiments, the ribonucleoprotein (e.g., a RNP comprising a site-directed modifying polypeptide, such as a Cas9 RNP or a Cpf1 RNP) is modified to include transduction or translocation domains.

### 5. Pharmaceutical Compositions

Exemplary compounds and compositions of the present invention are useful for treating C9orf72 GC repeat expansion mutation associated diseases, conditions and/or disorders; therefore, another embodiment of the present invention is a pharmaceutical composition comprising a therapeutically effective amount of a compound, containing composition of the present invention and a pharmaceutically acceptable excipient, diluent or carrier. The compounds of the present invention (including the compositions and processes used therein) may also be used in the manufacture of a medicament for the therapeutic applications described herein.

The pharmaceutical compositions of this invention may be in liquid solutions (e.g., injectable and infusible solutions). The preferred form depends on the intended mode of administration and therapeutic application, and is readily determinable by one of ordinary skill in the art. Typical pharmaceutical compositions are in the form of injectable or infusible solutions, such as pharmaceutical compositions similar to those used for passive immunization of humans. One mode of administration is parenteral (e.g., intravenous, subcutaneous, intraperitoneal, intramuscular, intradermal, and intrasternally) or by infusion techniques, in the form of sterile injectable liquid or olagenous suspensions. As will be appreciated by the skilled artisan, the route and/or mode of administration will vary depending upon the desired results. In a preferred embodiment, the compound or composition is administered by intravenous infusion or injection. In another preferred embodiment, the compound or composition is administered by intramuscular or subcutaneous injection.

In some embodiments, the pharmaceutical composition further includes cells produced by such methods for treating C9orf72 GC repeat expansion associated diseases in a recipient patient, and organisms comprising or produced from such cells. In some embodiments, there is provided a pharmaceutical composition comprising any of the elements disclosed herein for producing a population of any of the engineered cells described herein for treating C9orf72 GC repeat expansion associated diseases in a recipient patient described herein and a pharmaceutically acceptable additive, carrier, diluent or excipient. In some embodiments, the engineered cell is a pluripotent stem cell. In many embodiments, the engineered cell is an induced pluripotent stem cells. The pharmaceutical compositions of the invention may include a therapeutically effective amount or a prophylactically effective amount of compound of the invention. In preparing the pharmaceutical composition, the therapeutically effective amount of the compound present in the pharmaceutical composition can be determined, for example, by taking into account the desired dose volumes and mode(s) of administration, the nature and severity of the condition to be treated, and the age and size of the subject.

Dosage regimens can also be adjusted to provide the optimum desired response (e.g., a therapeutic or prophylactic response) by administering several divided doses to a subject over time or the dose can be proportionally reduced or increased as indicated by the exigencies of the therapeutic situation. It is especially advantageous to formulate parenteral pharmaceutical compositions in dosage unit form for ease of administration and uniformity of dosage.

Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention is dictated by and directly dependent on (a) the unique characteristics of the compound or portion and the particular therapeutic or prophylactic effect to be achieved, and (b) the limitations inherent in the art of compounding such an antibody for the treatment of sensitivity in individuals.

### EXAMPLES

### Example 1: Therapeutic CRISPR/Cas9 Gene Editing Approaches to the C9-Orf72 Repeat Expansion Mutation in Patient iPSCs

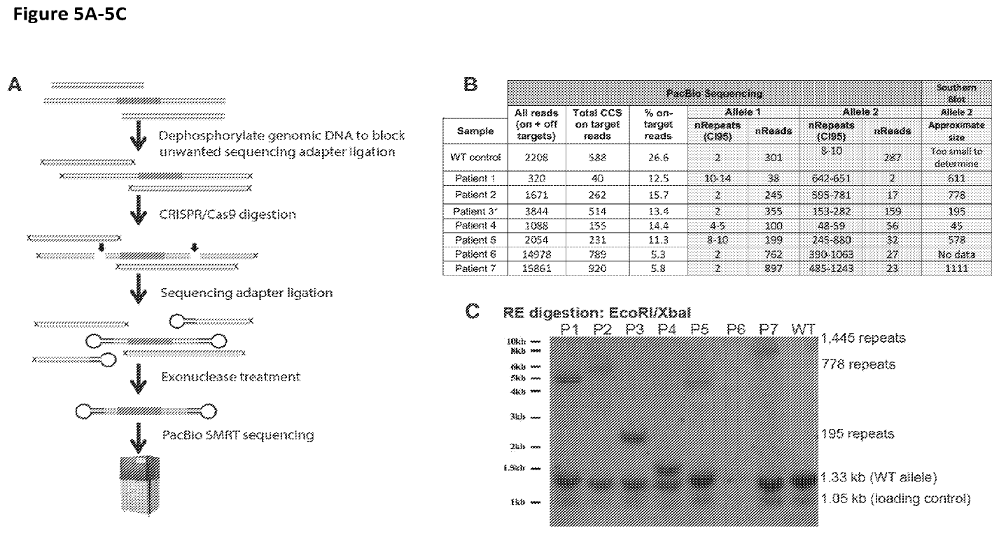

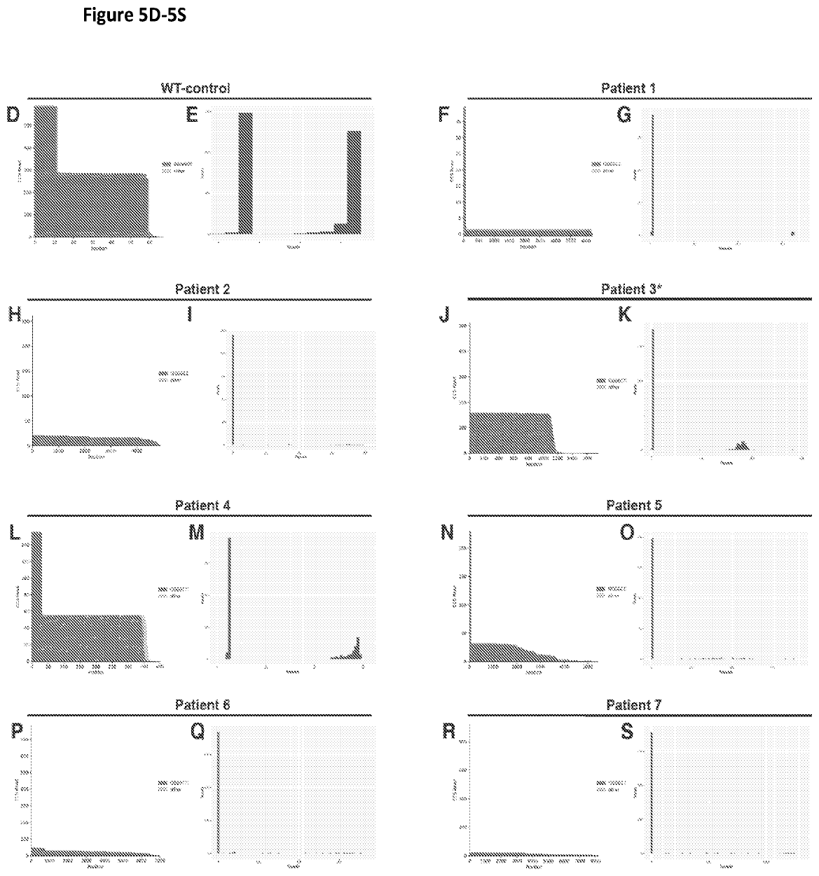

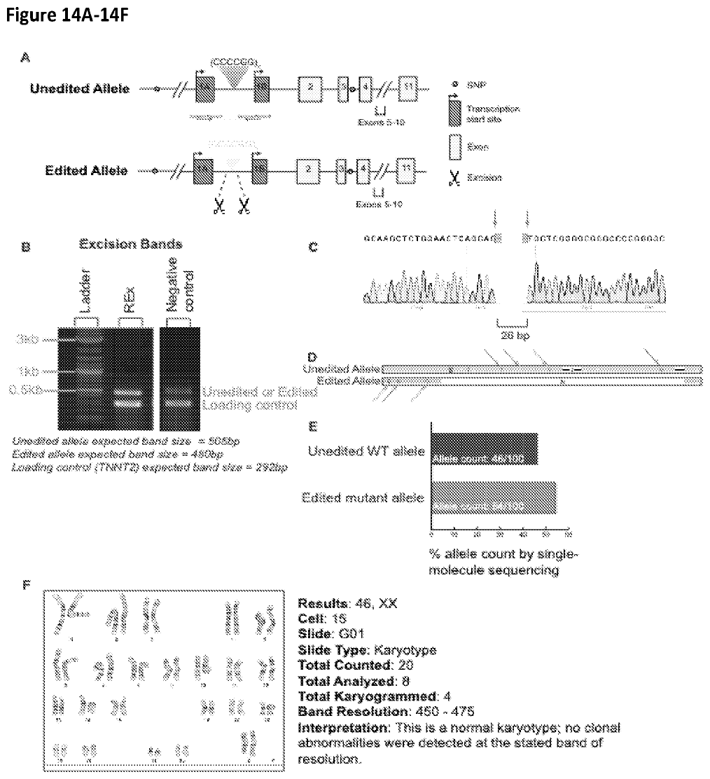

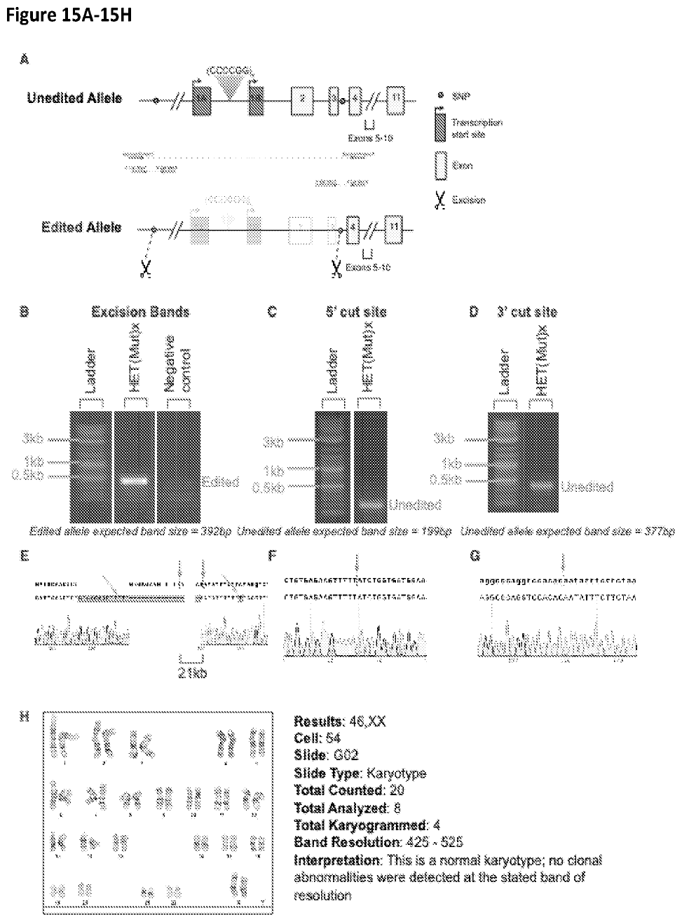

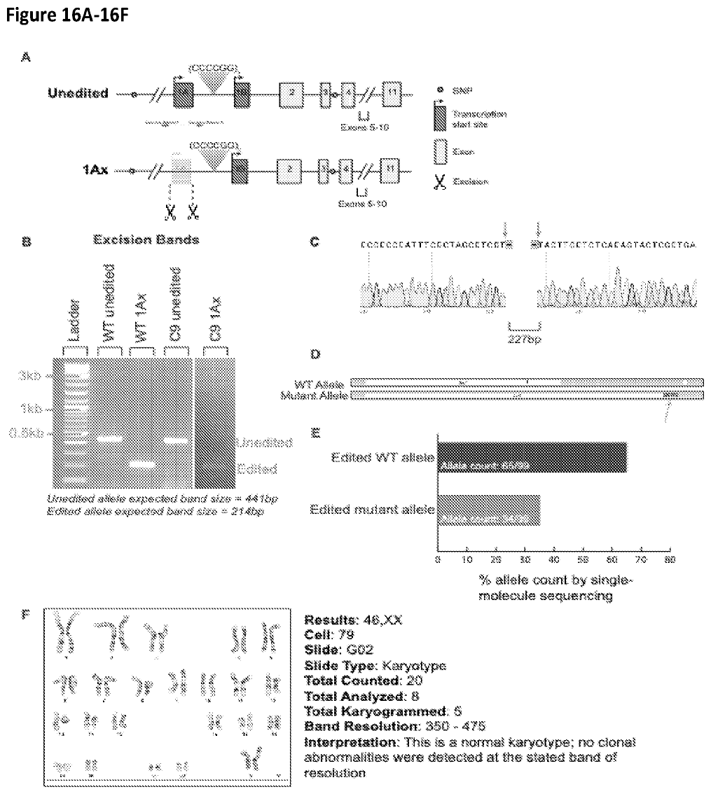

Patient lines were collected from previously published or publicly available sources^{30,41,54,55} Currently, long-range PCR⁵⁶ and Southern blot⁵⁷ are used to clinically diagnose repeat expansion mutations in the C9orf72 gene. Sizing the repeat expansion above ˜100 repeats is not possible using traditional sequencing techniques that require amplification because amplification fails across GC rich repetitive DNA regions. Patients can have C9orf72 repeats into the thousands! Single molecule sequencing has been demonstrated to traverse the expanded repeats of C9orf72 in plasmid⁵⁸ and human tissue^{59,60} PacBio single-molecule sequencing of DNA from patient iPSC lines was developed to size the repeat expansion and phase it to surrounding SNPs (FIG. 5, FIG. 14D). By excising a 3.6-10 kb genomic region centering the repeat expansion using Cas9 cutting of naked DNA, allowed enriching the target genomic loci of interest without amplification (FIG. 5A). Single-molecule sequencing was critical to determining the editing outcomes involving the C9orf72 repeat regions (FIG. 14), and was also useful in determining editing events that differed on each allele (FIG. 16). From these data, a patient cell line was chosen with ˜200 repeats and an advantageous coding SNP in the splice acceptor of exon 2 which was exploited for measuring RNA from each allele.

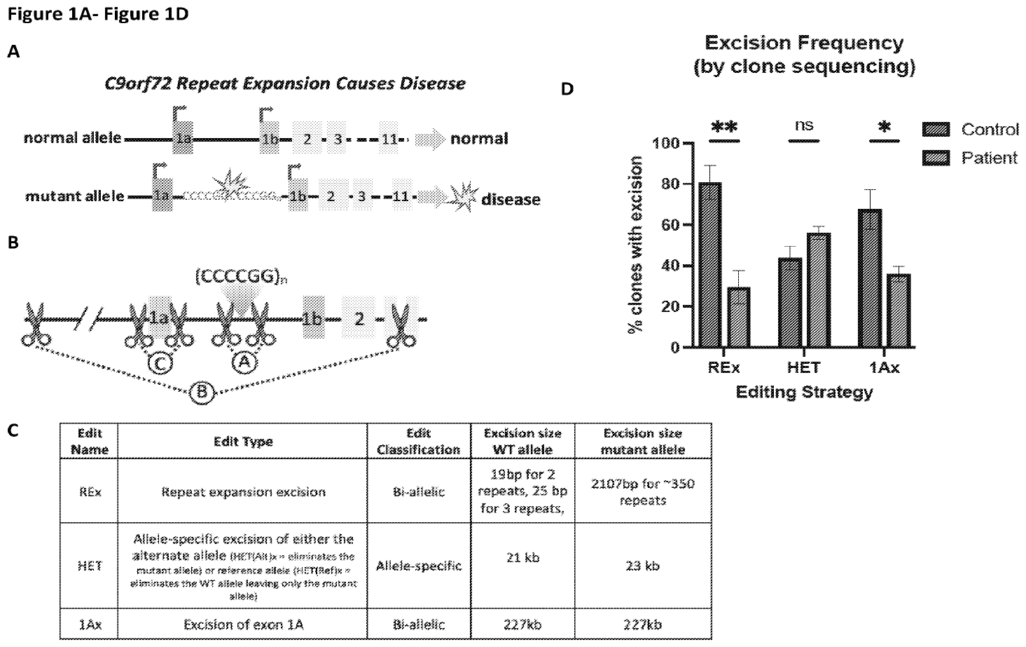

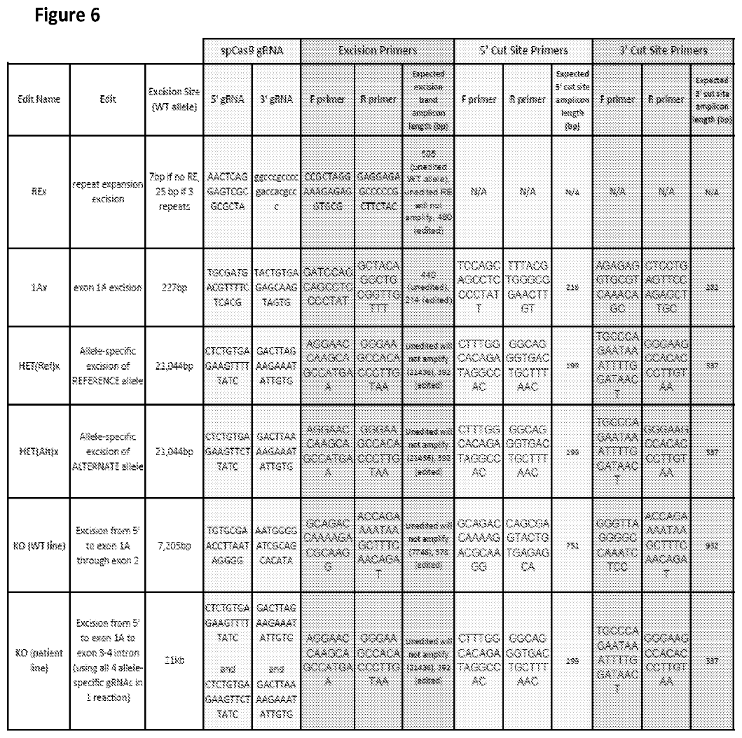

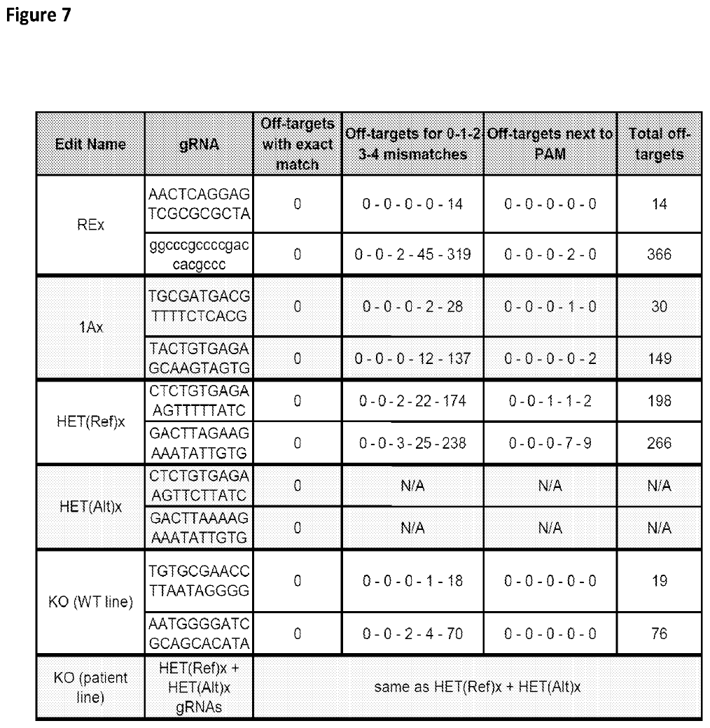

The C9orf72 mutation lies in the non-coding 5′UTR between two alternative start sites, exon 1A and exon 1B2 (FIG. 1A). It was hypothesized that gene editing strategies that can remove or silence the repeat expansion would be curative at the cellular level. Three editing approaches were compared to correcting the C9orf72 mutation in a patient and non-diseased control cell line (FIG. 1B, C). Each of these approaches capitalizes on the ability of Cas9 to induce double-stranded breaks (cuts) in DNA, which aligns with the most-developed Cas9 technology currently employed in clinical trials⁶¹⁻⁶³. gRNAs (FIG. 6, Table 8) were designed with the fewest overall off-targets (FIG. 7), including with no predicted off-target matches to the exact sequence and no predicted off-target within the first 2 bases of the PAM, as predicted by CRISPOR. The first approach excised the repeat expansion region (REx, FIG. 1C). Given numerous predicted off-targets throughout the genome, it is not safe to cut within the repeat region itself; instead cuts were made just 5′ and 3′ to the repeat region (FIG. 1B, circle A). Given high homology in this area, it is not possible to target a single allele, therefore this excision was designed to be bi-allelic. Interestingly, in our patient cell line, the excision occurred only on the mutant allele (FIG. 14) by chance, leaving intact the native two repeats on the WT allele. The second approach was to excise the mutant allele, leaving the normal allele intact. Newer versions of Cas9 can distinguish between alleles that differ by a single nucleotide. By targeting SNPs in cis with the mutation, the mutant allele becomes targetable, even if the mutation itself is not. AlleleAnalyzer⁶⁵, an open source bioinformatics tool, was used to design gRNA pairs that would result in allele-specific nuclease sites in the C9orf72 gene based on common heterozygous polymorphisms from reference data of >2500 human genomes from around the world⁶⁶. All gRNA pairs designed to edit C9orf72 are shown in Table 1-2 and Table 8-9. We chose a pair of gRNAs (FIG. 6, Table 1, Table 8) that span the repeat expansion and that cover the maximum number of individuals in the representative global cohort with the lowest off-target predictions. ed 21 kb of the mutant allele (alternate allele, ALT) including the repeat expansion through exon 3 (FIG. 1B, circle B, FIG. 1C, HET) was excised. In addition, the complementary 21 kb excision of the WT allele (reference allele, REF) was made leaving the only the mutant allele intact as a control. The third approach was to leave the mutation in the DNA but silence its expression by excising a regulatory region. As a proof-of-concept, we excised one such regulatory region, exon 1A (FIG. 1B, circle C, FIG. 1C 1Ax) which includes a transcriptional start site and controls the expression of the C9orf72 sense-transcript harboring the mutation. The REx, HET and 1Ax excisions in a non-diseased WT line to examine effects of each approach on the normal (non-diseased) cellular expression of the C9orf72 gene were also made. As additional controls, homozygous knock-outs of the gene in our patient and WT lines, comprised of bi-allelic 21 kb and 7 kb excisions, respectively were made (FIG. 6, Table 1).

Editing efficiency (FIG. 1D) was measured by PCR and sequencing across the edited locus in 3 independent experiments per edit. Each experiment derived from 48 hand-picked or 96 single-cell sorted clones. The efficiency of all editing was between 21 and 92% in iPSCs. Editing near the repeat region (REx, 1Ax) was found to be significantly less efficient in the patient lines containing a expanded repeat region compared to a WT line with fewer than 10 repeats. The hypothesis was that methylation of the repeat region and promoter⁶²⁻⁶⁹ in patient lines accounts for less accessibility of the loci to Cas enzymes and therefore lower editing efficiency. Interestingly, a large 21 kb excision was surprisingly efficient (30-59%) and did not differ between patient and WT lines. It is important to note that these observed efficiencies are based on gRNA derived from computational predictions (highest on-target with lowest off-target rate) but have not been optimized experimentally for efficiency.

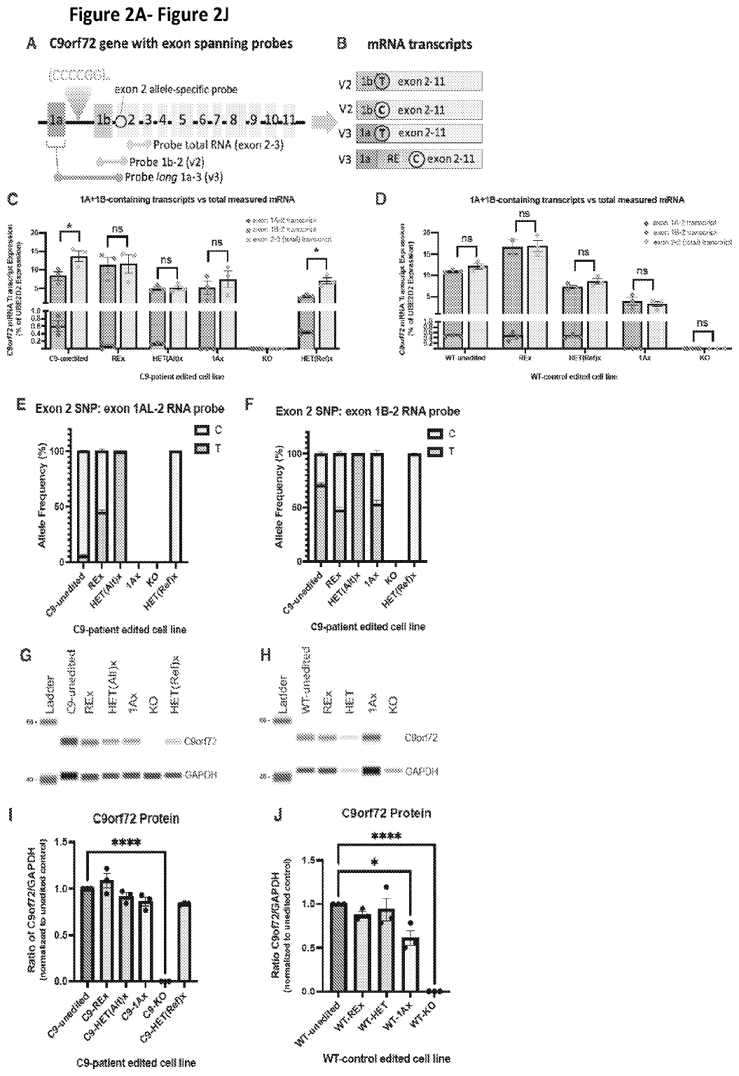

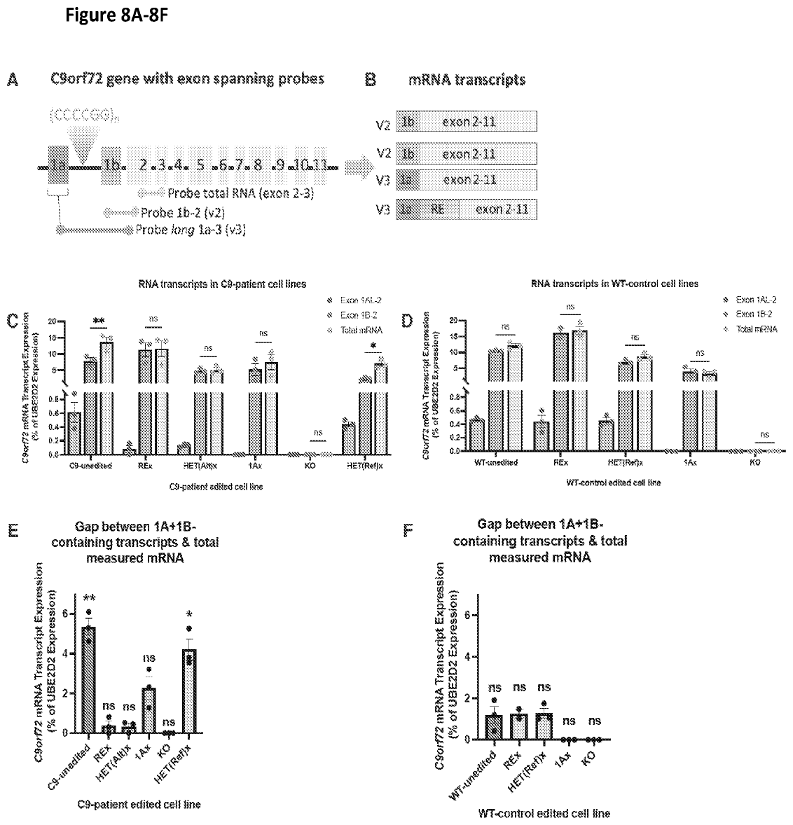

Next, the effect of each of the edits was evaluated in a patient and control line on C9orf72 RNA and protein expression levels. Using ddPCR, the two major splice isoforms of C9orf72 were quantified using exon spanning probes that cross either the exon 1A-exon2 (variant 3) or exon1B-exon 2 (variant 2) junctions (FIG. 2A, B). We were not able to detect short isoform (variant 1) in the lines, consistent with its low expression in human tissue⁷⁰⁻⁷¹. Total mRNA was additionally quantified using a probe targeting the exon 2-exon 3 junction. The majority of the total RNA derived from exon 1b-containing transcripts across all lines, with exon 1A-containing transcript comprising only a small proportion of total transcript (FIG. 2C, D). A gap was noted between measured total mRNA (exon 2-exon 3-containing transcripts) and exon 1A- and exon 1B containing transcripts only in lines containing the repeat expansion (C9-unedited, HET(Ref)x) but not in mutation-corrected patient lines (REx, HET(Alt)x, 1Ax) (FIG. 2C, FIG. 8C, E) or any of the WT lines (FIG. 2D, FIG. 7D, F). It was hypothesized that this gap is comprised of 1A-transcript that retains the repeat expansion resulting from sense expression of the mutation, which would not be measurable due to inability to amplify repetitive RNA and also because primers targeting exon 1A and exon 2 would be too far apart form an amplicon. Interestingly, a decrease in 1A expression was also observed in each of the correct mutant lines compared to the unedited patient line (FIG. 2C, FIG. 8C), but no effect of editing on 1A-expression in the WT lines (FIG. 2D, FIG. 8D), suggesting the possibility of upregulation of normal 1A transcription in the diseased state.

Lying between exons 1A and 1B, the sense repeat expansion is expressed from exon 1A; therefore only exon 1A-containing transcripts have the possibility of expressing the mutation (FIG. 2B). Using single-molecule sequencing, an advantageous coding C SNP (rs10757668) in the exon 2 splice acceptor to the repeat expansion was phased. Using probes that differed by a single nucleotide targeting this SNP, the percentage of expression of 1A- and 1B-containing transcripts derived from each allele (FIG. 2E, F) was determined. Just as with the ddPCR probes in FIG. 2C, D, transcripts with retained repeat expansions/introns as our probes are exon-exon spanning were not detected. Surprisingly, most (>90%) exon-1A containing transcript derived from the mutant allele in the unedited patient lines (FIG. 2E), suggesting at least some normal splicing of the mutant transcript. The imbalance was corrected by repeat expansion excision. Together with the decreased in 1A-containing transcript levels after gene correction, these data suggest an upregulation of exon 1A transcripts off of the mutant allele, which implicates transcriptional upregulation of the mutation as a possible biological driver of disease. In contrast to exon 1A regulation, exon 1B-containing transcripts derived predominantly (>68%) from the WT allele, which was restored to nearly 50% with repeat expansion excision (FIG. 2F). Excision of exon 1A also restored bi-allelic expression of exon 1B-containing transcripts, which suggests that the mere presence of the repeat expansion in the DNA does not solely account for altered 1B-containing transcript expression. As expected, excision of either allele resulted in elimination of expression from that allele.

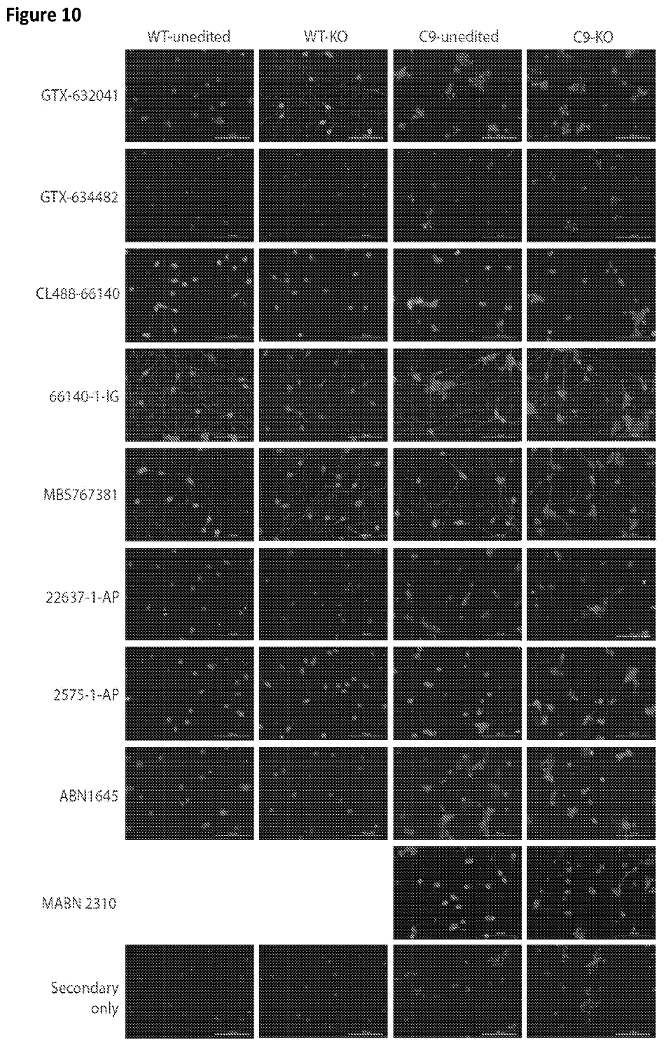

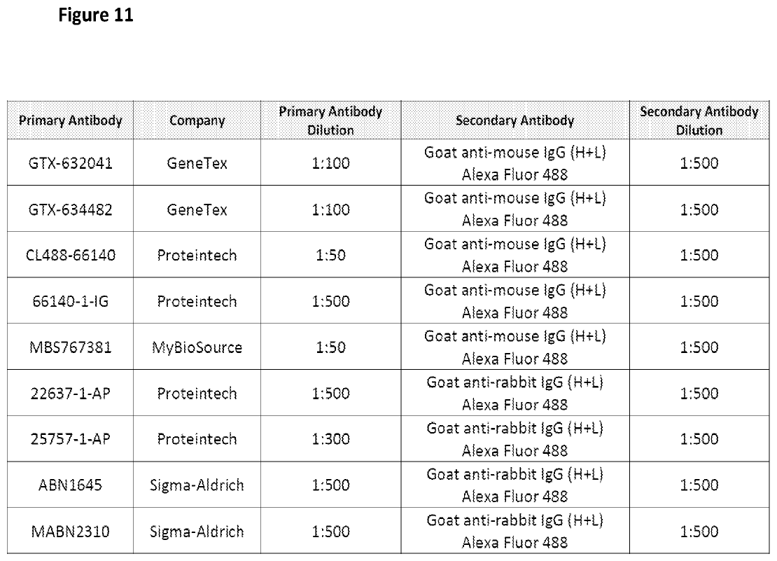

C9orf72 protein was quantified using the Simple Western system (WES). antibody specificity was validated using the knock-out line. It is important to note that this antibody cannot distinguish between protein derived from exon-1A and exon 1B-containing transcripts as these transcripts produce an identical protein isoform. None of the therapeutic edits (REX, HETx, 1Ax) reduced the C9orf72 protein levels in the patient line (FIG. 2G, I), and only exon 1A-excision reduced C9orf72 expression in the WT line (FIG. 2H, J). These results indicate that the C9orf72 protein is also regulated post-transcriptionally, which is advantageous for gene therapy as even major alterations of the gene (such as removal of an entire allele) does not alter total protein levels in cells. Additionally, nine commercially available C9orf72 antibodies were tested for immunocytochemistry and did not find any that were specific for C9orf72 (i.e., they either had no signal or showed signal in our 2 KO lines) (FIG. 10, FIG. 11).

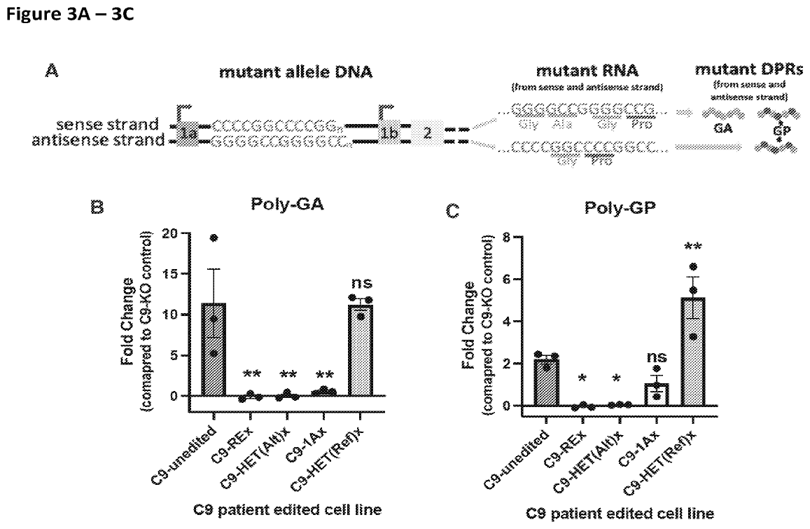

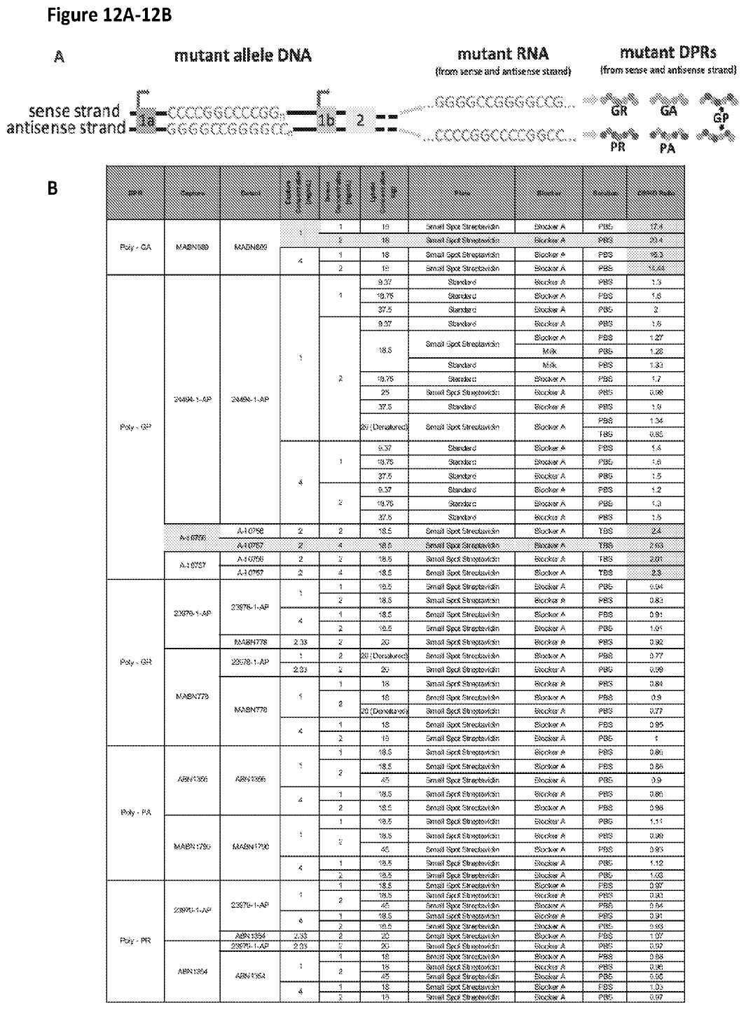

The ability of therapeutic edits to reverse the pathology caused by the C9orf72 mutation. C9orf72 is transcribed off of both the sense and anti-sense strands (FIG. 3A) was evaluated. The data suggest that sense transcription of the mutation starts from exon 1A, since excision of exon 1A closed the gap in “undetectable” sense transcript (FIG. 2C, FIG. 8). It is unknown where anti-sense transcription initiates. The mutant repeat expansion is translated through non-canonical RAN translation from transcripts derived from both and sense and anti-sense strands to form 5 dipeptide repeat proteins (DPRs) which are thought to be toxic (FIG. 3A, FIG. 12). 10 antibodies targeting each of these DPRs were evaluated using MSD's sandwich ELISA and found 2 antibody combinations that could reliably detect the presence of poly-GA and poly-GP DPRs above background level defined by out KO line (FIG. 12B). Poly-GA expression was eliminated by each of the therapeutic edits (C9-REx, HET(Alt)x, 1Ax) but unchanged by excision of the WT allele (C9-HET(Ref)x)) (FIG. 3B). Poly-GP was also eliminated by removal of the repeat expansion (REx, HET(Alt)x) but not excision of exon 1A (1Ax) (FIG. 3C), owing to anti-sense transcription of the repeat expansion. Excision of the WT allele more than doubled the amount of poly-GP expression (HET(Ref)x), indicating that regulation of the mutation itself is dynamic and worthy of further exploration (FIG. 3C).

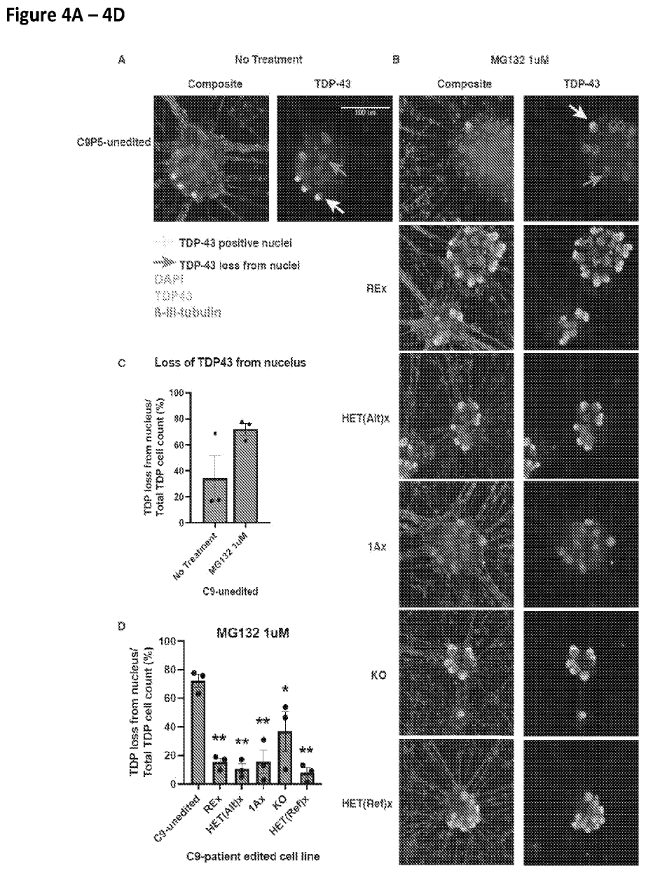

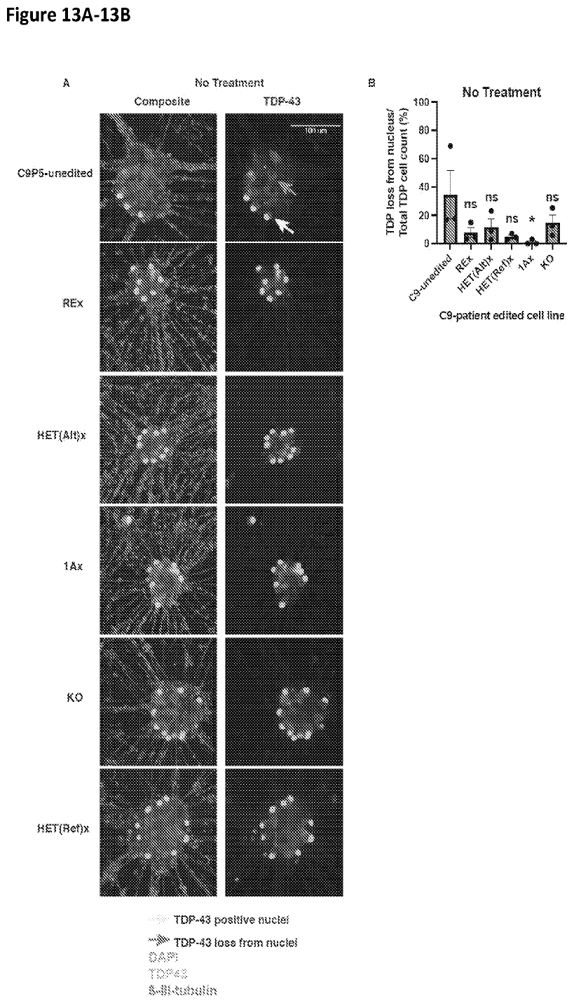

The pathological hallmark of C9-FTD/ALS is loss of TDP-43 expression from nuclei and TDP-43 aggregation in the cytoplasm of affected neurons²². These events are thought to be independent. Loss of nuclear TDP43 was detected in aged 7-week old neurons derived from the unedited patient cell line (FIG. 4A, pink arrow, FIG. 4C) which showed a non-significant trend toward increase compared to our edited lines (FIG. 13A, B). This effect was non-significantly amplified after 17-hour treatment with a proteosome inhibitor MG132 (FIG. 4B, C). After a 16 hour treatment with 1 uM MG132 of 7-week old neurons derived from all of the isogenic patient lines, a significant effect of genotype of nuclear loss of TDP43 (FIG. 4C, D) was found. Whereas 72% of TDP43-positive neurons in our unedited patient line had lost nuclear TDP43 after treatment with MG132, this rate was on average <20% in each of the therapeutically edited lines (REx, HET(Alt)x, 1Ax). Interestingly the KO line had the highest rate of nuclear loss of TDP43 compared to our other edited lines (FIG. 4D) whereas removal of either the mutant or WT allele did not show this finding, suggesting the TDP43 pathological changes may be impact by complete, but not partial, loss of C9orf72 expression.

### Discussion

Three strategies were investigated for correcting the C9orf72 repeat expansion mutation in patient iPSCs. Each strategy capitalized on Cas9's ability to cut DNA, which aligns with technologies that are closest to clinical prime-time⁶¹⁻⁶⁷. Two that two of the three approaches (repeat expansion excision and excision of the mutant allele) were found to correct RNA abnormalities, preserved protein levels, and correct dipeptide repeat and TDP43 pathology in iPSC-derived neurons from a patient line harboring ˜200 repeats. As an alternative approach, silencing the expression of the repeat expansion without removing it from the DNA by excising exon 1A was attempted. While this approach successfully restored the RNA profile and ameliorated TDP43 pathology, it did not eliminate poly-GP DPRs. Interestingly, both of the successful approaches, repeat expansion and allele-specific excisions, included removing the repeat expansion.

### Methods

Cell line generation, maintenance and determination of editing efficiencies. iPSC generated by others^{41-54,55} from patients harboring the C9orf72 mutation and a control cell line without mutation (WTC²²) was used. iPSCs were maintained in mTesR plus, passaging at 60-80% confluency. All cell lines had a normal karyotype and negative monthly mycloplasma testing.

We first knocked-in the inducible motor neuron transcription factor transgene cassette⁴⁹⁻⁷² in the CLYBL safe-harbor locus of a C9-patient line using spCas9 and ATGTTGGAAGGATGAGGAAA (SEQ ID NO: 713; 747) gRNA. This transgene includes human NGN2, ISL1, LHX3 (hNIL) under the TET operator and is inducible by doxycycline, mCherry (for positive selection) and neomycin antibiotic resistance (for negative fluorescence). Red-fluorescing cells were sorted via FACS to isolate single, live cells. As shown in Table 3, each resulting clonal cell line was analyzed for incorporation of the transgene in the CLYBL locus by PCR (left homology arm junction primers CAGACAAGTCAGTAGGGCCA (SEQ ID NO: 714) and AGAAGACTTCCTCTGCCCTC (SEQ ID NO: 715)) with preservation of one of the alleles (CLYBL wild-type primers TGACTAAACACTGTGCCCCA (SEQ ID NO: 716) and AGGCAGGATGAATTGGTGGA (SEQ ID NO: 717)). We used Copy Number Variation (CNV) ddPCR to pick a clone with a single transgene insertion of the hNIL plasmid (Nemomycin primers CATGGCTGATGCAATGCG (SEQ ID NO: 718) and TCGCTTGGTGGTCGAATG (SEQ ID NO: 719), probe FAM; Primers UBE2D2—Bio-Rad 10031255, probe HEX) to mitigate the risk of integration of the transgene at genomic loci other than CLYBL.

To engineer each iPSC line HiFi spCas9 protein (Macolabs, UC Berkeley) and two gRNAs (FIG. 6, Table 1, Table 8) were used to create an excision. gRNAs were designed to have no exact off-target matches and the lowest predicted off-targets using CRISPOR (Homo sapiens-USCS December 2013 (GRCh38/hg38)) 45 Cas9-gRNA RNP (spCas9 (40 μM), sgRNA (100 uM)) was delivered by nucleofection (Lonza AAF-1002B, Lonza AAF-1002X, Pulse Code=DS138) to 350,000 iPSCs suspended in 20 μl of P3 Buffer. The cells were recovered with mTesR plus supplemented with ROCK1 inhibitor (Selleckchem S1049) at 10 μM and Clone R (Stemcell 05888). Approximately 50% of iPSCs died within the first 24 hours of electroporation, as expected. Following a 48-72 hour recovery, the pool of edited cells was collected and either hand-picked 48 clones or sorted single live cells via FACS to a single well on a 96-well plate. Single cell sorting was performed using a BD FACSAria Fusion (Beckton Dickinson) by the Gladstone Flow Cytometry Core. The QC alignment of each laser was verified with Cytometer Setup and Tracking Beads (Becton Dickinson) before sample acquisition. A forward scatter threshold of 15,000 was set to eliminate debris from list mode data, and a fixed number of events was collected. In some experiments mCherry fluorescence (excitation 561 nm, emission 610 nm) was also used to define sorting parameters. Drop delay determination and 96-well plate set-up setup was done using Accudrop beads (Becton Dickinson). Gating on forward scatter area versus height and side scatter area versus height was used to make the single cell determination. The specifications of the sort layout included single cell precision, 96-well collection device and target event of 1. After cultures reached 60-70% confluency, each well was split into two wells of a new 48- or 96-well plate, one for sequencing and the other to continue the cell line. Clones were screened based on the presence of an excision band using PCR (primers and expected band size from FIG. 6, Table 1). PCR was performed across each the 5′ and 3′ cut site (FIG. 6, Table 1), with one primer site located inside the excision region, to ensure absence of a band (for homozygous edits) or presence of the WT allele (for heterozygous edits). For all lines except C9-REX, the excision band (MCLAB) was Sanger sequenced. If the sequence was ambiguous (i.e., had overlapping nucleotide reads at the same mapped nucleotide position) the line was subcloned to achieve clone purity and clean sequencing. All lines were karyotyped (WiCell or Cell Line Genetics) after editing.

For all lines except C9-REx, editing efficiency was determined based on the PCR amplification of an excision band, and in the case of homozygous excisions, the absence of the WT band, in each clone (48 hand-picked clones or 96 single-cell FACS sorted clones). See FIG. 6, Table 1 for primers. For C9-REx this approach was not used since PCR could not amplify the large repeat expansion, and hence could not distinguish clones with excision of both the mutant and WT allele from clones with excision of the WT allele only. Therefore, single-molecule sequencing of clonal REx lines was used to determine the percentage of clones with an excision of the repeat expansion region (as described below).

PacBio single molecule sequencing to size the repeat expansion and determine repeat expansion excision (C9-REx) edits. Because polymerase amplification fails to accurately size the entirety of the C9orf72 GC rich repetitive region, single molecule sequencing⁵⁸ of a genomic region containing the repeat region was used. High molecular weight DNA was collected using Genomic Tip (Qiagen 10243) and confirmed absence of smearing by running the DNA on a 1.5% agarose gel. The Gladstone Genomics Core performed library preparation according to the “No Amp Targeted Sequencing” published protocol^{52,79} using 3-5 ug of DNA per sample as measured by Qubit. Briefly, we blocked the free ends of purified genomic DNA and then excised the gene region of interest using spCas9, a gRNA targeting 5′ to the repeat expansion (GGAAGAAAGAATTGCAATTA, SEQ ID NO: 720; SEQ ID NO: 748) and a gRNA targeting 3′ to the repeat expansion (TTGGTATTTAGAAAGGTGGT, SEQ ID NO: 721; SEQ ID NO: 749), as shown in Table 4 and Table 8. Excising the genomic region harboring the repeat expansion yields a 2639 bp fragment from the WT allele and a variable size of the mutant allele fragment depending on the size of the CCCCGG repeat. Adapters and barcodes were ligated to blunt free ends of DNA and sequenced 3-5 barcoded lines per SMRT Cell on either a Sequel I or Sequel II sequencer. A 3-pass filter was used such that each molecule of DNA had to be sequenced 3 times to be included in analysis. Repeat counts were compared from sequencing to Southern blot, performed by Celplor using 20 μg of input DNA and the previously published protocol

iPSC differentiation into motor neurons. The hNIL transgene cassette TET-on system in the CLYBL safe-harbor locus of a C9-patient line and WT line as used. Introduction of doxycycline for 3 days induced the expression of 3 human transcription factors NGN2, ISL1, LHX3. The previously published protocol^{49,78} was followed with notable exceptions, including higher concentrations of the growth factors BDNF, GDNF and NT-3.

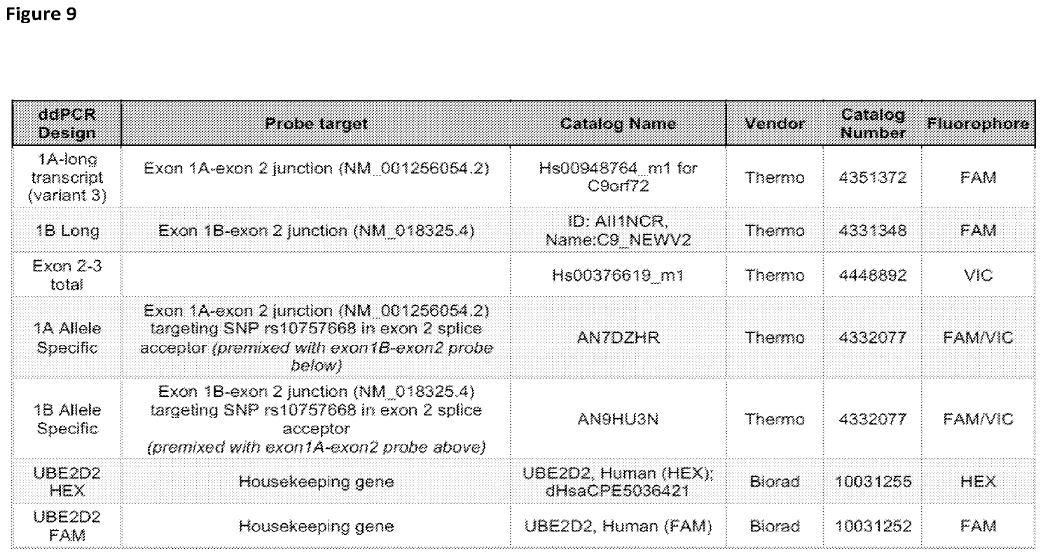

RNA quantification by ddPCR. 2-week old induced neurons were lysed with papain (Worthington LK003178) and RNA was isolated using Quick-RNA Microprep Kit (Zymo R1051). cDNA was synthesized using iScript™ Reverse Transcription Supermix (Biorad 1708841) and 500 ng of RNA. ddPCR was run with 3 technical replicates of each of 3 biologic replicates (independent wells of differentiated neurons) on the QX100 Droplet Reader (Bio-Rad 186-3002). Each ddPCR reaction consisted of 12.5 uL of 2× SuperMix for Probes (no dUTP) (Bio-Rad 186-3024), primer/probe (see FIG. 9), 5 ng of cDNA, and nuclease-free water up to 25 μL. Droplets were generated with QX 100 Droplet Generator (Bio-Rad 186-3001) and 20 μL of the reaction mixture with 70 μL of oil. The ddPCR reactions were run in a Deep Well C1000 Thermal Cycler (Bio-Rad 1851197) with the following cycling protocol: (1) 95° C. for 10 min; (2) 94° C. for 30 s; (3) 58° C. for 1 min; (4) steps 2; and 3 repeat 39 times; (5) 98° C. for 10 min; (6) hold at 4° C. Positive samples were thresholded as those with >10 positive droplets to avoid error due to noise. Positive droplets were quantified for each target and normalized the amount to our loading control (UBE2D2) (Bio-Rad QuantaSoft™ Analysis Pro Software). This housekeeping gene was chosen because its expression level remained stable across iPSCs and differentiated neurons⁸⁰.

For allele-specific expression of exon 1A- and 1B-containing transcripts, a coding SNP in the exon 2 splice acceptor (rs10757668) in our patient line was utilized. The ddPCR probe was centered on this SNP and used the same primers as above to amplify the exon 1A-exon 2 (Thermo, 4332077) and exon 1B-exon 2 junctions (Thermo, 4332077) (FIG. 9). Expression from each allele was quantified in a single reaction and reported as a ratio.

C9orf72 protein quantification by Simple Western. Protein quantification was performed by streptavidin-based Simple Western⁸¹ capillary reaction (WES; Bio-Techne) according the manufacturers protocol (Jess & Wes Separation Module SM1001 to SM1012⁸²), with the following specifications: protein was collected from cultured neurons 2-weeks post-induction in RIPA buffer with protease inhibitor and sonicated for 5 min, and denatured at 90 degrees C. for 10 min. 0.3 ug/ul protein from each sample was mixed with 1 ul 5× Master Mix and 0.1× Sample Buffer (EZ Standard Pack PS-ST01EZ-8) to a total volume of 5 ul. 3 ul of this mix was loaded per sample onto a 12-230 kDa plate (ProteinSimple SM-W004-1). Primary antibodies were mouse anti-C9orf72 (GeneTex, GTX634482, FIG. 11) at a 1:100 dilution and rabbit anti-GAPDH (AbCam, AB9485) at 1:1000 dilution (total volume 10 ul per lane). Duplexed secondaries included 9.5 ul of mouse (ProteinSimple, DM-002) and 0.5 ul of 20× anti-rabbit (ProteinSimple, 043-426) per lane. Reaction times: 25 min separation time at 375V, 5 min antibody dilutant time, 30 min primary antibody, 30 min secondary antibody; quantification at 4 seconds of detection (high dynamic range). Each antibody produced a single peak corresponding to each antibody under these optimized conditions: 57 kDa (C9orf72) and 42 kDa (GAPDH). Area under the curve was quantified for each peak and C9orf72 AUC was normalized to GAPDH AUC for each sample. Averages across 3 biological replicates (independent wells of neuronal differentiation) of neurons aged 14-days post-induction from each edited cell line were compared to the average protein expression of their respective unedited controls.

Dipeptide repeat quantification by Meso Scale Discovery (MDS) sandwich ELISA. 2 antibody combinations were found to be specific for detecting DPRs in 14-day-old iPSC-derived neurons harboring the C9orf72 repeat expansion (FIG. 12). We followed the manufacturer's protocol for the Small Spot Streptavidin Plate (L45SA, MSD). Poly-GA was detected using anti-GA antibody (MABN889, Millipore) at 1 mg/ml (capture) and 2 mg/ml (detect) final concentration and 18 μg total protein per sample (blocking buffer A, solution PBS). Poly-GP was detected using anti-GP antibody (affinity purified TALS828.179 from TargetALS, purification lot A-I 0757 and stock concentration 1.39 mg/ml). A-I 0757 anti-GP antibody was used at a final concentration of 2 mg/ml capture and 4 mg/ml detect with 18.5 μg total protein per sample (blocking buffer A, solution TBS). The plate was coated with capture antibody overnight at 4° C. with no agitation. The plate was blocked with 3% MSD Blocker A (R93BA, MSD) in 1×DPBS for 1 hour at 750 rpm, then incubated for 1 hour with protein lysate at 750 rpm at room temperature. Detection antibody was added after the lysate for 1 hour. Washes were performed between steps thrice with 1×DPBS+0.05% Tween-20. MSD Read Buffer A (R92TG, MSD) was added to the plate before being immediately placed in the MSD Model 1250 Sector Imager 2400 plate reader. Signal was calculated by comparing luminescence intensity for each control or edited patient line to background (i.e., C9-KO line), data was presented as a fold change above C9-KO baseline/background level.

TDP43 immunocytochemistry and quantification. 7-week-old neurons were fixed by adding 4% PFA directly to culture media for 30 min followed by 3 PBS washes of 10 min each. Cells were permeabilized by 1×DPBS 0.1% Triton-X in 3 washes of 10 min each at room temperature and blocked with 1×DPBS 0.1% Triton-X+5% BSA for 1 hour at room temperature. Primary antibodies: rabbit anti-TDP43 (10782-2-AP, Proteintech) at 1:500, beta-III-tubulin (480011, Invitrogen) at 1:250. Primary antibodies were incubated overnight at 4° C. Secondary antibodies included Goat anti-rabbit Alexa Fluor 488 nm and Goat anti-mouse Alexa Fluor 594 nm. Secondary antibodies were incubated at room temperature for 1 hour. DAPI (D1306, ThermoFisher Scientific) was added to the penultimate of five, 5 min PBS washes. After staining, cells were scanned on the ImageXpress Micro Confocal (Molecular Devices). TDP43 cells were quantified by hand-counting.

### Example 2: RNA Delivery Via AAV In Vivo

Synthesis of AAV for Cas9 and gRNA Delivery

The C9orf72 GC repeats expansion editing are conducted by CRISPR/Cas 9. The CRISPR system comprising Cas9 and gRNAs can be packaged in a single adeno-associated virus (AAV) particle to be delivered into the target cells. A promoter, for example, a single H1 promoter, can efficiently express both Cas9 and gRNAs. Due to this unique genetic element, an assembly composed of any Cas9 gene packaged in a single recombinant AAV, called AAV-H1-CRISPR, and a large number, for example 2 gRNAs, is possible. The ability to add gRNA allows AAV-H1-CRISPR to generate double-strand breaks unmatched site-specifically, thus minimizing the risk of known off-target mutagenesis.

1. Assembly of virus constructs. In order to facilitate the rapid assembly of viral vectors from modular components in the field of commercial laboratories, synthetic reusable modular cassettes can be synthesized for subsequent targets. The vector for C9orf72 repeat expansion gene editing are assembled to contain the specific H1-gRNA modules and the Cas9 endonuclease. The final assembly are verified by restriction mapping followed by complete sequencing. The final product is provided as an assembled viral construct of 1 mg of transfection grade C9orf72 ORF target locus specific AAV shuttle plasmid DNA without endotoxin and sequence errors.

2. Preparation and analysis of AAV-H1-CRISPR stock. Packaging of viral constructs, purification of viral particles and molecular survey of viral titer purity and infectivity are performed by cGMP certified core facilities according to industry standards. Production of viral stocks suitable qualitatively and quantitatively for preclinical studies. Prepared and purified by cGMP, with minimal infectivity of 0.9 IU/viral genome, minimal titer of viral genome 10¹²/ml and minimal yield of 10¹⁴ infectious units Endotoxin-free AAV stock.

3. Quantitative characterization of AAV-H1-CRISPR genome targets and off-target sites. The on target and predicted off target sites in the infected cell population are sequenced in depth. The modified/unmodified allele ratio provides a quantitative measure of efficiency; the on-target/off-target modification ratio becomes the final measure of specificity. The virus is tested in vivo using engineered mouse and human biopsy samples.

rAAV:Cas9/gRNA Administration

AAV CRISPR/Cas9 are injected via tail vein into mice at day 0 and day 5 with PBS for control animals. At day 5, one pair (AAV and PBS) animals are subjected to a retro orbital bleed for a blood sample, euthanized and tissues harvested. The second pair of animals receive a second tail vein injection of AAV-Cas9 or PBS and are euthanized for harvest of tissue 7 days later after retro orbital bleed. Tissues harvested are mouse frontal cortex, temporal cortex, parietal cortex, cerebellum and spinal cord tissues.

### Example 3: RNP Delivery Via RBP System In Vivo

### Binding of Conjugated Endosomolytic Polypeptide (ELP)-RNA Binding Protein Variants to Guide RNAs or RNP

In one instance, the RNA binding protein (RBP) is the N-terminal domain of a human UlA protein, also referred to herein as “U1A”. In some cases, the ELP is a synthetically modified ppTG21 peptide bearing a pyridyl disulfide leaving group to facilitate conjugation.

The purified Cas9 is mixed with a variant guide RNA (i.e., a guide RNA with or without one or more SL, as described above), as described in Rouet et al. (ibid). A prepared Cas9 solution is added to the prepared solution of sgRNA at the desired concentration. Guides or RNP are used to test their capacity to be bound by UlA variants and UlA-ppTG21 conjugates by fluorescence polarization binding assays using the experimental design described in Hochstrasser et al. Mol Cell. 2016 Sep. 1; 63(5):840-51. doi: 10.1016/j.molcel.2016.07.027.

Biolayer interferometry (BLI) is used to assess the capacity of Cas9 RNPs containing sgRNA variants to bind U1A (or U1A-ppTG21 conjugates) as well as to assess the persistence of any binding events. These assays approximate the experimental design described in Richardson et al. (Nat Biotechnol. 2016 March; 34 (3): 339-44. doi: 10.1038/nbt.3481). These experiments rely on a Cas9 protein covalently, site-specifically labeled with a biotin moiety, allowing loading onto a BLI sensor bearing streptavidin.

### Genome Editing Using U1A Variants and Modified Guide RNAs

Genome editing is performed as described in Rouet et al. (ibid), in particular the genome editing relying on the 1NLS Cas9 construct. Cas9 RNP is prepared and applied either alone, with addition of ppTG21, or with addition U1A-ppTG21 conjugates. Cas9 RNP mixtures are added to cells and returned to the incubator. Cells are harvested 44-48 h later, and genomic DNA are harvested. T7E1 analysis is performed as described in Rouet et al. (ibid).

An experiment is performed to assess the genome editing ability of different configurations of variant sgRNA bound by different U1A-ppTG21 conjugates (e.g. U1A (1), U1A (2), or U1A (3), which respectively represent U1A monoconjugated, bisconjugated, or trisconjugated with ppTG21). Another editing experiment is performed to assess the ability of the Cas9 RNP with adaptor-recruited ELPs (arELP) to perform ligand-enhanced (and thus, receptor-mediated) genome editing.

### RNP Administration

Various routes of administration suitable for use in a method of the present disclosure include various enteral and parenteral routes of administration, including, e.g., intratumoral, peritumoral, intramuscular, intratracheal, intracranial, subcutaneous, intradermal, topical application, intravenous, intraarterial, rectal, nasal, oral, and other enteral and parenteral routes of administration.

### Example 4: Treatment of the ALS/FTD Phenotype of FVB C9-500 Mice

### Mouse Models C9-500

The C9-500 BAC (Tg(C9orf72)500Lpwr) transgenic mouse line expresses a human C9orf72 gene with ˜500 hexanucleotide repeats (GGGGCC; or G4C2) in intron. The C9orf72 BAC transgenic line C9-500 was created by Dr. Laura P. W. Ranum (University of Florida). Briefly, a ˜98 kbp human bacterial artificial chromosome (BAC) 002: B7 subclone m5 30 (Chr9:27,527,137-27,625,470 [Human Genome, February 2009, GRCh37/hg19]) was microinjected into pronuclei of fertilized mouse eggs with an FVB/NJ background. The BAC has ˜52 kbp of transcriptionally-upstream (telomeric) and ˜19 kbp of transcriptionally-downstream (centromeric) flanking sequences that contain no other complete loci or confirmed genes (June 2016). Founder males were bred to FVB/NJ inbred females for germline transmission, establishing four C9-BAC founder lines. Founder line C9-500 (561KK) was identified with a single copy of the transgene harboring ˜500 GGGGCC repeats. The transgene analysis performed on the hemizygous mice suggested a single copy of the transgene has integrated on chromosome 6 (114,939,853-114,939,873 [mouse mm10]) and resulted in a 20 bp deletion of genomic region. It has further been confirmed that mice express the transgene and that dipeptide repeat (DPR) levels of polyGP (as measured by ELISA) are 100× higher than background at two months of age (See https://www.jax.org/strain/029099).

Other mouse models under development and manifesting the C9orf72 GC repeats expansion associated syndromes are also suitable targets for in vivo study.

### Mouse Embryofibroblasts (MEFs)

The C9-500 BAC (Tg(C9orf72)500Lpwr) transgenic mouse Mouse embryo fibroblasts (MEFs) are prepared from 17 day gestation embryos by mechanical and enzymatic dissociation and maintained in DMEM supplemented with 10% fetal bovine serum. MEF cells are prepared as previously described (Behringer et al., Manipulating the mouse embryo: A laboratory manual, Fourth edition. Cold Spring Harbor Laboratory Press, 2014) and genotyped by PCR using primers specific for the HIV transgene (Kopp J B, et al. Proc Natl Acad Sci USA 1992; 89:1577-1581; Dickie P, et al. Virology 1991; 185:109-119).

### DNA Analysis

Genomic DNA are isolated from cells/tissues using any standard protocol. Genomic DNA are analyzed by single molecule sequencing of a genomic region containing the repeat region.

Rna Analysis and Quantification by ddPCR

Total RNA is prepared from tissues using any standard protocol. Mouse frontal cortex, temporal cortex, parietal cortex, cerebellum and spinal cord tissues are lysed by papain (Worthington LK003178) and RNA is isolated using Quick-RNA Microprep Kit (Zymo R1051). cDNA is synthesized using iScript™ Reverse Transcription Supermix (Biorad 1708841) and 500 ng of RNA. ddPCR is run with 3 technical replicates of each of 3 biologic replicates (independent wells of differentiated neurons) on the QX100 Droplet Reader (Bio-Rad 186-3002). Each ddPCR reaction consists of 12.5 uL of 2× SuperMix for Probes (no dUTP) (Bio-Rad 186-3024), primer/probe, 5 ng of cDNA, and nuclease-free water up to 25 μL. Droplets are generated with QX 100 Droplet Generator (Bio-Rad 186-3001) and 20 μL of the reaction mixture with 70 μL of oil. The ddPCR reactions are run in a Deep Well C1000 Thermal Cycler (Bio-Rad 1851197) with the following cycling protocol: (1) 95° C. for 10 min; (2) 94° C. for 30 s; (3) 58° C. for 1 min; (4) steps 2; and 3 repeat 39 times; (5) 98° C. for 10 min; (6) hold at 4° C. Positive samples are thresholded as those with >10 positive droplets to avoid error due to noise. Positive droplets are quantified for each target and normalized the amount to our loading control (UBE2D2) (Bio-Rad QuantaSoft™ Analysis Pro Software).

For allele-specific expression of exon 1A- and 1B-containing transcripts, we are utilizing a coding SNP in the exon 2 splice acceptor (rs10757668) in our patient line. We center our ddPCR probe on this SNP and use the same primers as above to amplify the exon 1A-exon 2 (Thermo, 4332077) and exon 1B-exon 2 junctions (Thermo, 4332077). Expression from each allele is quantified in a single reaction and reported as a ratio.

### Protein Quantification by Simple Western

C9orf72 protein quantification is performed by streptavidin-based Simple Western capillary reaction (WES; Bio-Techne) according to the manufacturers protocol (Jess & Wes Separation Module SM1001 to SM101282), with the following specifications: protein is collected from mouse frontal cortex, temporal cortex, parietal cortex, cerebellum and spinal cord tissues in RIPA buffer with protease inhibitor and sonicated, and denatured. Protein from each sample is mixed with Sample Buffer (EZ Standard Pack PS-ST01EZ-8) and this mix is loaded per sample onto a 12-230 kDa plate (ProteinSimple SM-W004-1). Primary antibodies are mouse anti-C9orf72 (GeneTex, GTX634482, FIG. 11) at a 1:100 dilution and rabbit anti-GAPDH (AbCam, AB9485) at 1:1000 dilution (total volume 10 ul per lane). Duplexed secondaries include mouse (ProteinSimple, DM-002) and anti-rabbit (ProteinSimple, 043-426) per lane. Each antibody is produced a single peak corresponding to each antibody. Area under the curve is quantified for each peak and C9orf72 AUC is normalized to GAPDH AUC for each sample. Averages across 3 biological replicates (independent wells of neuronal differentiation) of mouse frontal cortex, temporal cortex, parietal cortex, cerebellum, and spinal cord tissues from each edited cell line is compared to the average protein expression of their respective unedited controls.

### Dipeptide Repeat Quantification by Meso Scale Discovery (MDS) Sandwich ELISA.

Poly-GA and poly-GP are quantified as follows: We follow the manufacturer's protocol for the Small Spot Streptavidin Plate (L45SA, MSD). Poly-GA is detected using anti-GA antibody (MABN889, Millipore) at 1 mg/ml (capture) and 2 mg/ml (detect) final concentration and 18 μg total protein per sample (blocking buffer A, solution PBS). Poly-GP is detected using anti-GP antibody (affinity purified TALS828.179 from TargetALS, purification lot A-I 0757 and stock concentration 1.39 mg/ml). A-I 0757 anti-GP antibody is used at a final concentration of 2 mg/ml capture and 4 mg/ml detect with 18.5 μg total protein per sample (blocking buffer A, solution TBS). The plate is coated with capture antibody overnight at 4° C. with no agitation. The plate is blocked with 3% MSD Blocker A (R93BA, MSD) in 1×DPBS for 1 hour at 750 rpm, then incubated for 1 hour with protein lysate at 750 rpm at room temperature. Detection antibody is added after the lysate for 1 hour. Washes are performed between steps thrice with 1×DPBS+0.05% Tween-20. MSD Read Buffer A (R92TG, MSD) is added to the plate before being immediately placed in the MSD Model 1250 Sector Imager 2400 plate reader. Signal is calculated by comparing luminescence intensity for each sample to background (i.e., C9-KO line), data is presented as a fold change above C9-KO baseline/background level.

TDP43 immunocytochemistry and quantification. Samples or tissues are fixed by directly adding 4% PFA for 30 min or after perfusion of mouse tissues followed by 3 PBS washes of 10 min each. Cells are permeabilized by 1×DPBS 0.1% Triton-X in 3 washes of 10 min each at room temperature and blocked with 1×DPBS 0.1% Triton-X+5% BSA for 1 hour at room temperature. Primary antibodies: rabbit anti-TDP43 (10782-2-AP, Proteintech) at 1:500, beta-III-tubulin (480011, Invitrogen) at 1:250. Primary antibodies are incubated overnight at 4° C. Secondary antibodies include Goat anti-rabbit Alexa Fluor 488 nm and Goat anti-mouse Alexa Fluor 594 nm. Secondary antibodies are incubated at room temperature for 1 hour. DAPI (D1306, ThermoFisher Scientific) is added to the penultimate of five, 5 min PBS washes. After staining, cells are scanned on the ImageXpress Micro Confocal (Molecular Devices). TDP43 cells are quantified by hand-counting.

### Treatment Regimen Study

Mice with ALS/FTD phenotype is employed to evaluate the efficacy of rAAV:Cas9/gRNA C9orf72. Prior to treatment study, mice are randomized into treatment groups based on body weight, gait analyses, grip strength, cage behavior, and open field testing or a combination of outcomes. Histological analyses include assessment of neuromuscular junctions in the tibialis and diaphragm muscles, quantification of motor neuron ventral roots, and immunohistochemistry of the brain and spinal cord using a variety of antibodies. Assessment of RNA foci and colocalization with neurons (Neu-N) in the brain or lower motor neurons (ChAT) in the spinal cord are performed.

Mice are assessed weekly for body weight change, gait analyses, grip strength, cage behavior, open field testing, assessment of neuromuscular junctions in the tibialis and diaphragm muscles, quantification of motor neuron ventral roots, immunohistochemistry of the brain and spinal cord using a variety of antibodies, and RNA foci and colocalization with neurons (Neu-N) in the brain or lower motor neurons (ChAT) in the spinal cord. The doses of test item to be administered is calculated daily in mg/kg based on the latest body weight of the mice.

For treatment, a first group of fifteen C9-500 BAC transgenic mice (after 14 weeks old) are iv injected at least one dose of rAAV:Cas9/gRNA C9orf72. A second group of fifteen mice are iv injected with the same dose of control rAAV. From week 14 to week 20, data of clinical relevancy is collected and compared across all groups.

The clinical endpoints are evaluated based on data of gait analyses, grip strength, cage behavior, open field testing, assessment of neuromuscular junctions in the tibialis and diaphragm muscles, quantification of motor neuron ventral roots, immunohistochemistry of the brain and spinal cord using a variety of antibodies, and RNA foci and colocalization with neurons (Neu-N) in the brain or lower motor neurons (ChAT) in the spinal cord from C9-500 BAC model

### Example 5: Screen of Allele-Specific gRNAs to Test the Efficiency of Excision of the Mutant c9-Orf72 Allele by CRISPR spCas9 gRNA Pairs in Human iPSCs Harboring a Mutant c9-Orf72 Repeat Expansion

A screen of pairs of gRNAs was performed to determine which gRNA pairs had maximal excision efficiency for excising the C9orf72 mutant allele (allele-specific excision). The ability of pairs of gRNAs to excise the mutant C9orf72 allele in a patient iPSC line harboring ˜200 repeats was measured. Each gRNA was designed to target a single nucleotide polymorphism (SNP) in cis with the C9orf72 repeat expansion so that pairs of gRNAs would selectively excise the mutant allele, leaving the normal allele intact.

An allele-specific gRNA 5′ was paired to the mutant repeat expansion with guide nucleic acid sequence as labeled by guide names 1-6 (corresponding to guide nucleic acids-DNA sequences in SEQ IDs 29, 570, 482, 700, 440, and 384; or guide nucleic acid-RNA sequences in SEQ IDs 739, 1266, 1178, 1398, 1136, and 1180) with an allele-specific gRNA 3′ to the repeat expansion with guide nucleic acid sequence as labeled by guide names A-D (corresponding to guide nucleic acids-DNA sequences in SEQ IDs 30, 290, 217, and 629; or guide nucleic acid-RNA sequences in SEQ IDs 740, 984, 911, and 1327) and tested their efficiency for excision of the mutant C9orf72 allele. The following allele-specific guide nucleic acids (see Table 6) were tested. Each 5′ guide nucleic acid was tested in combination with each 3′ guide nucleic acid.

TABLE 6
Allele-specific guide nucleic acid tested for efficiency of excision of the mutant
C9orf72 allele

| | Ch9 | | Guide | | | | |
|---|---|---|---|---|---|---|---|
| | position | | targeting | Guide nucleic | SEQ | Guide nucleic | SEQ |
| Guide | of | | strand | acids | ID | acids | ID |
| name | SNP | SNP ID | (+/−) | (DNA) | NO: | (RNA) | NO: |
| 1 | 27586164 | rs2453556 | minus | CTCTGTGAGAAGT | 29 | CUCUGUGAGAA | 739 |
| | | (A/G) | | TCTTATC | | GUUCUUAUC | |
| 2 | 27585699 | rs36062268 | positive | AACTCTGGGGGAA | 570 | AACUCUGGGGG | 1266 |
| | | (G/A) | | AAAAAGT | | AAAAAAAGU | |
| 3 | 27580676 | rs12350076 | minus | GACACAGCAGGAA | 482 | GACACAGCAGG | 1178 |
| | | (A/C) | | GGTTATC | | AAGGUUAUC | |
| 4 | 27578937 | rs2484322 | positive | GGAGGAACAGAG | 700 | GGAGGAACAGA | 1398 |
| | | (T/G) | | AGAGGGAA | | GAGAGGGAA | |
| 5 | 27578079 | rs12345062 | minus | GAAAGACTCATAG | 440 | GAAAGACUCAU | 1136 |
| | | (G/A) | | GACTAAT | | AGGACUAAU | |
| 6 | 27575465 | rs2453557 | positive | TCAGCTTCTGGGC | 384 | UCAGCUUCUGG | 1080 |
| | | (T/C) | | AGGCCAC | | GCAGGCCAC | |
| A | 27565107 | rs2492816 | positive | GACTTAAAAGAAA | 30 | GACUUAAAAGA | 740 |
| | | (G/A) | | TATTGTG | | AAUAUUGUG | |
| B | 27559723 | rs774356 | minus | AGTGCTACTTGAG | 290 | AGUGCUACUUG | 984 |
| | | (T/C) | | TTAGTTA | | AGUUAGUUA | |
| C | 27553878 | rs12349820 | positive | AACCCACCCCACT | 217 | AACCCACCCCAC | 911 |
| | | (T/C) | | ACAAACT | | UACAAACU | |
| D | 27551042 | rs2453565 | minus | TTTTATTTATTAAT | 629 | UUUUAUUUAUU | 1327 |
| | | (C/T) | | TGCCTA | | AAUUGCCUA | |

The gRNAs were ordered from Synthego. Cas9-gRNA RNP (spCas9 (40 μM), sgRNA (100 μM)) was delivered by nucleofection (Lonza AAF-1002B, Lonza AAF-1002X, Pulse Code=DS138) to 150,000 iPSCs suspended in 20 μl of P3 Buffer. The cells were recovered with mTesR plus supplemented with ROCK1 inhibitor (Selleckchem S1049) at 10 μM and Clone R (Stemcell 05888). Following a 48 hour recovery, we collected DNA from the pool of edited cells using Quick Extract (VMR 76081-768). Our novel excision reporter ddPCR assay which detected excision events was used to quantify excision efficiency.

A loss of a ddPCR probe centered on exon 2 to report on editing (excision of the mutant allele would lead to loss of probe binding site) was used. Normalizing probe binding to a housekeeping genomic loci (RPB30, Bio-Rad Assay ID dHsaCP2500350) allowed quantifying excision frequency to determine copy number or ratio. ddPCR was run with 3 technical replicates of each of 2 biologic replicates (independent electroporation events) on the QX100 Droplet Reader (Bio-Rad 186-3002). Each ddPCR reaction consisted of 12.5 uL of 2× SuperMix for Probes (no dUTP) (Bio-Rad 186-3024), primer/probe, 5 ng of DNA, and nuclease-free water up to 25 μL. Droplets were generated with QX 100 Droplet Generator (Bio-Rad 186-3001) and 20 μL of the reaction mixture with 70 μL of oil. The ddPCR reactions were run in a Deep Well C1000 Thermal Cycler (Bio-Rad 1851197) with the following cycling protocol: (1) 95° C. for 10 min; (2) 94° C. for 30 s; (3) 58° C. for 1 min; (4) steps 2; and 3 repeat 39 times; (5) 98° C. for 10 min; (6) hold at 4° C. We quantified positive droplets for each target and normalized the amount to the loading control (RPB30) (Bio-Rad QuantaSoft™ Analysis Pro Software).

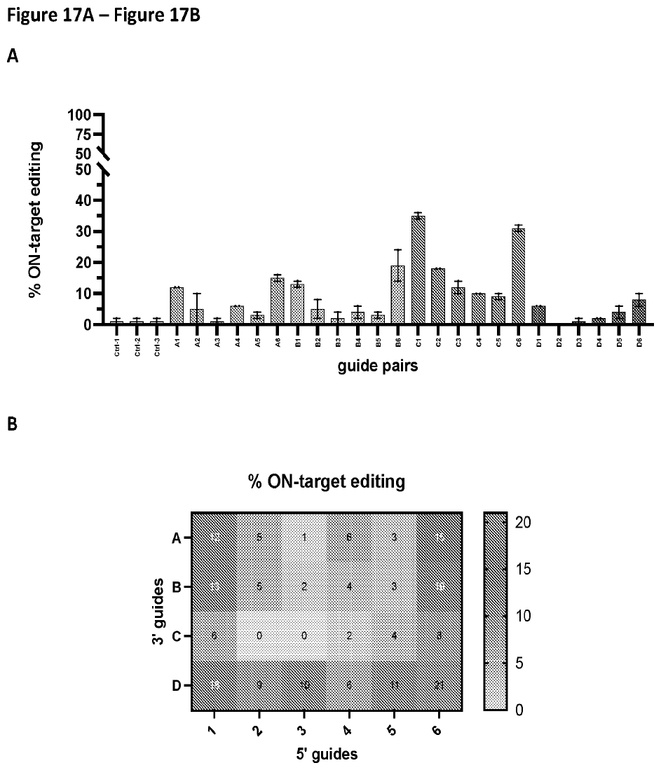

Some pairs of gRNAs were found to be more efficient than others (see FIG. 17A and FIG. 17B) and that while some individual gRNAs performed better overall, the pairs nonetheless had to be tested experimentally as an inefficient gRNA could decreased the overall efficacy (such as pair C6). In addition, there is no computational method to predict which gRNA pair is the most efficient, this must also be determined experimentally.

### Example 6: In Vivo Validation of Allele-Specific Excision and Repeat Expansion Excision Approaches in a Mouse Model of C9-ALS/FTD

The BAC-C9orf72 mouse (JAX 029099)^{83, 84} was chosen because it displays a number of features consistent with human C9-ALS, including: ˜500 repeats expressed at levels similar to those recorded in humans, motor neuron loss, motor deficits, pathologic hallmarks of human disease (RNA foci, dipeptide repeat expression, TDP-43 pathology) and early lethality (by 4-10 months). In addition, this mouse model contains a single insertion of the human mutant gene. Excising the human gene in this mouse is therefore a good model for the heterozygous, allele-specific excision we propose in human cells. We have sequenced the human transgene in this mouse and found that it contains our target SNPs.

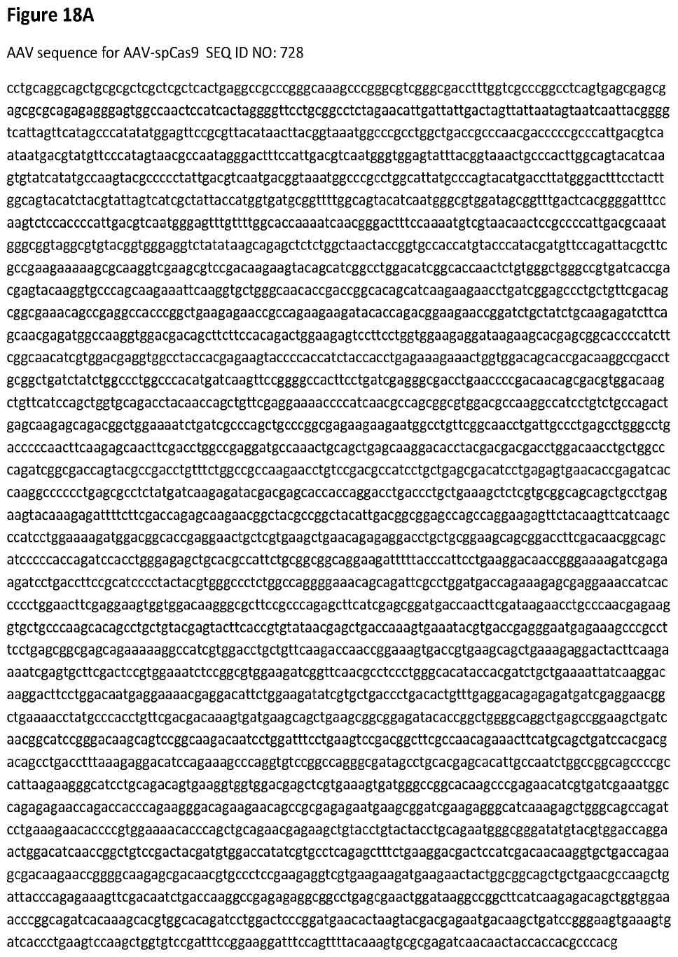

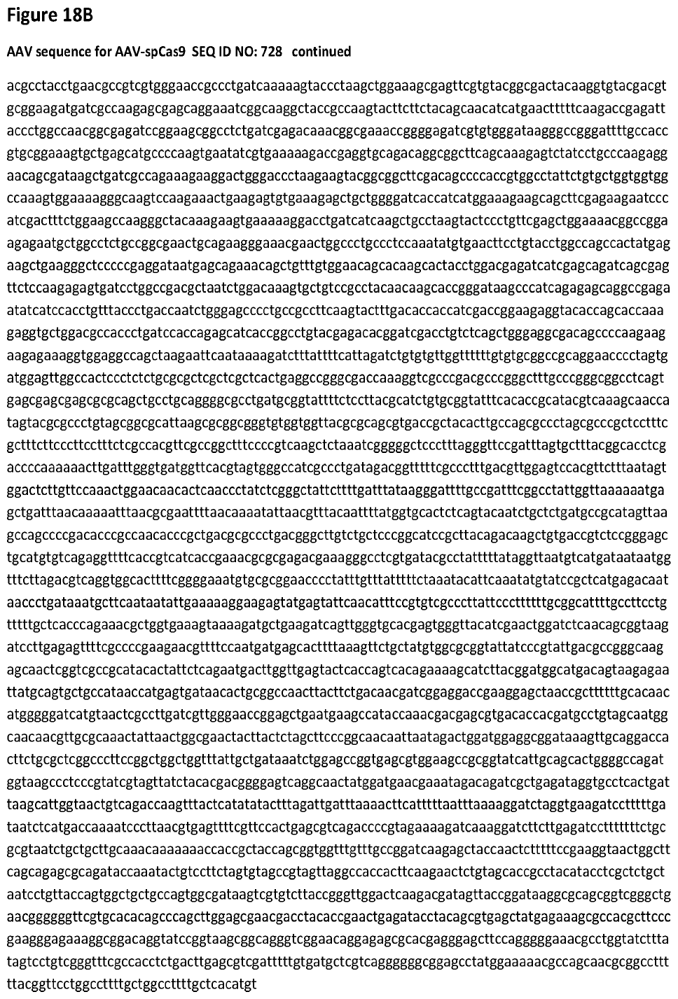

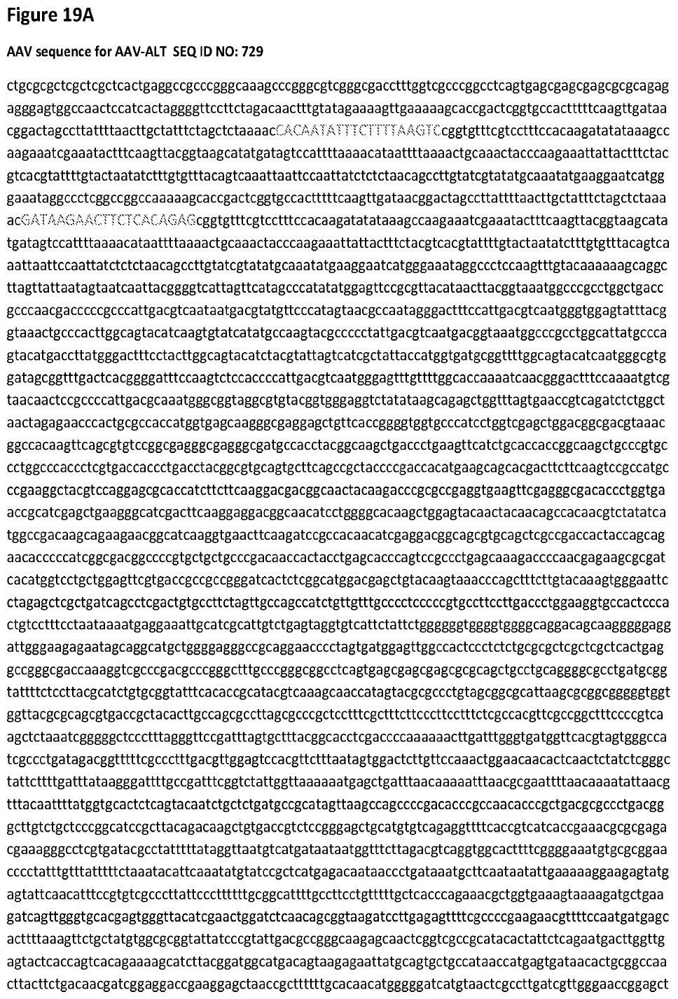

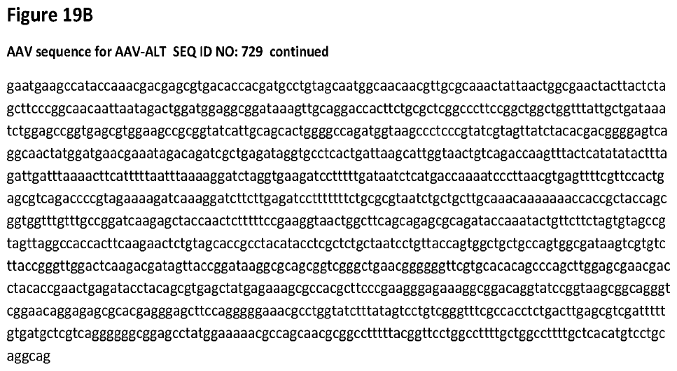

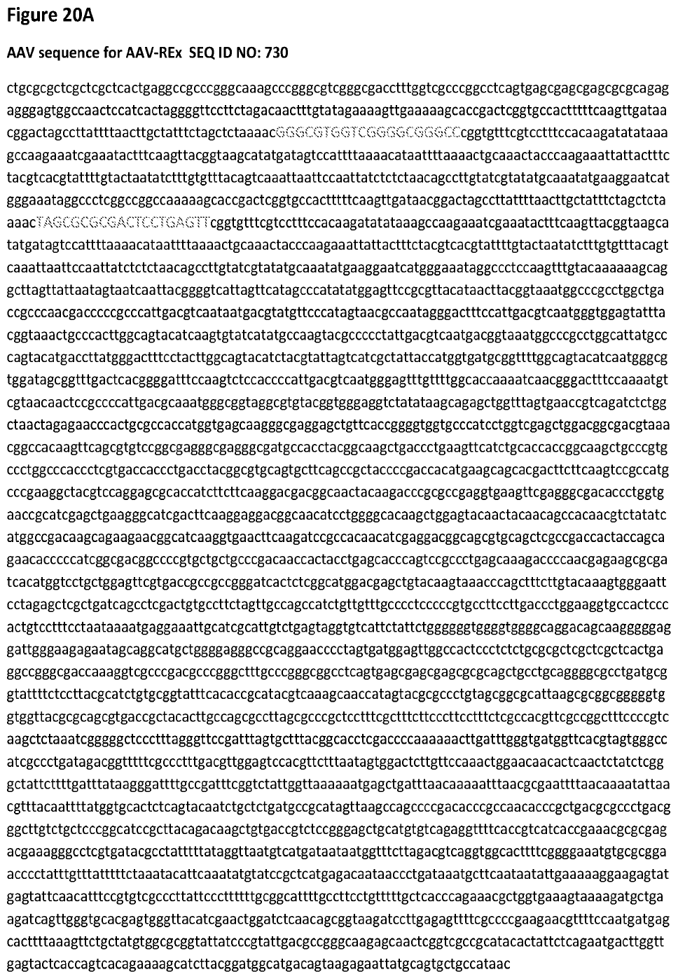

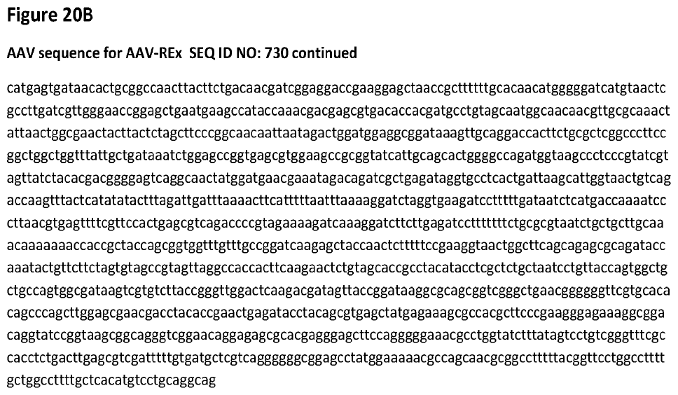

We generated three AAVs: One containing spCas9 (AAV-spCas9; FIG. 18A and FIG. 18B), a second containing the two ALT gRNAs (encoded by SEQ ID NO: 724 (CACAATATTTCTTTTAAGTC)) and by SEQ ID NO: 725 (GATAAGAACTTCTCACAGAG)) and a GFP reporter (AAV-ALT; FIG. 19A and FIG. 19B) and a third containing the REx gRNAs (encoded by SEQ ID NO: 726 (GGGCGTGGTCGGGGCGGGCC) and by SEQ ID NO: 727 (TAGCGCGCGACTCCTGAGTT)) and a GFP reporter (AAV-REx; FIG. 20A and FIG. 20B). Sequences encoding the gRNAs are in upper case in FIGS. 19 and 20. Three C9-mice were injected at 12 weeks of age with both the AAV-spCas9 and AAV-ALT. An additional three C9-mice at 12 weeks of age were injected with AAV-spCas9 and AAV-REx. An additional twelve mice were injected with PBS as sham-injected controls. Each mouse was injected with 4 ul total volume per hemisphere (2 ul of each vector) of 4e10-4e11 vg/ml virus into the striatum with the following coordinates (AP 0.2, ML 2.0, DV 2.5 mm from Bregma) using convection-enhanced delivery.

The mice are sacrificed at 6 weeks post injection and the effects of allele-specific vs repeat region excision vs sham injected control on C9orf72 pathology, including the percentage of cells demonstrating RNA foci and the level of C9orf72-dipeptide repeats polyGA and polyGP are measured. The editing efficiency using ddPCR assays and single-molecule sequencing are determined.

### Example 7: Allele Specific Excision of Mutant Alleles in ALS/FTD Patient Cells

An iPSC line from an ALS/FTD patient carrying a pathogenic C9orf72 repeat expansion for allele specific excision which excises the mutant allele alone, leaving the normal allele intact is used.

Construction of AAV for Cas9 and gRNA Delivery

The guide nucleic acids sequences that would excise a large segment of the C9orf72 locus which includes an allele-specific 5′ guide nucleic acid to the mutant repeat expansion are selected, e.g., SEQ IDs 29, 570, 482, 700, 440, 384, 739, 1266, 1178, 1398, 1136, or 1180 and an allele-specific 3′ guide nucleic acid to the repeat expansion e.g., SEQ IDs 30, 290, 217, 629, 740, 984, 911, and 1327. Through those guide nucleic acids pairs, at least 20 kb of the mutant allele (HET(Mut)x) starting at least 10 kb upstream of exon 1A and stretching all the way through exon 4 can be obtained. At least two types of AAV are generated for cell transfusion: a first one containing spCas9 (AAV-spCas9), a second one containing the nucleic acids sequences encoding a pair of allele-specific gRNAs (AAV-gRNA 5′&3′) and a reporter.

Transfusion iPSC Cultures of AAV Vectors

iPSC cultures from an ALS/FTD patient carrying a pathogenic C9orf72 are co-transduced with AAV-SpCas9 and AAV-gRNA 5′&3′. After 7 days, genomic DNA is harvested and analyzed for genome editing using ddPCR assays and single-molecule sequencing.

### Dipeptide Repeat Quantification by Meso Scale Discovery (MDS) Sandwich ELISA.

To evaluate the effects of allele specific excision, Poly-GA and poly-GP are quantified in the AAV vectors co-transduced C9orf72 ALS/FTD iPSC cell lines and untreated control cell line. The manufacturer's protocol for the Small Spot Streptavidin Plate (L45SA, MSD) is followed. Poly-GA is detected using anti-GA antibody (MABN889, Millipore) at 1 mg/ml (capture) and 2 mg/ml (detect) final concentration and 18 μg total protein per sample (blocking buffer A, solution PBS). Poly-GP is detected using anti-GP antibody (affinity purified TALS828.179 from TargetALS, purification lot A-I 0757 and stock concentration 1.39 mg/ml). A-I 0757 anti-GP antibody is used at a final concentration of 2 mg/ml capture and 4 mg/ml detect with 18.5 μg total protein per sample (blocking buffer A, solution TBS). The plate is coated with capture antibody overnight at 4° C. with no agitation. The plate is blocked with 3% MSD Blocker A (R93BA, MSD) in 1×DPBS for 1 hour at 750 rpm, then incubated for 1 hour with protein lysate at 750 rpm at room temperature. Detection antibody is added after the lysate for 1 hour. Washes are performed between steps thrice with 1×DPBS+0.05% Tween-20. MSD Read Buffer A (R92TG, MSD) is added to the plate before being immediately placed in the MSD Model 1250 Sector Imager 2400 plate reader. Signal is calculated by comparing luminescence intensity for each sample to background (i.e., C9-KO line), data is presented as a fold change above C9-KO baseline/background level.

TDP43 immunocytochemistry and quantification. TDP-43 accumulates are quantified in the AAV vectors co-transduced C9orf72 ALS/FTD iPSC cell lines and untreated control cell line. Cells are fixed by directly adding 4% PFA for 30 min or after perfusion of mouse tissues followed by 3 PBS washes of 10 min each. Cells are permeabilized by 1×DPBS 0.1% Triton-X in 3 washes of 10 min each at room temperature and blocked with 1×DPBS 0.1% Triton-X+5% BSA for 1 hour at room temperature. Primary antibodies: rabbit anti-TDP43 (10782-2-AP, Proteintech) at 1:500, beta-III-tubulin (480011, Invitrogen) at 1:250. Primary antibodies are incubated overnight at 4° C. Secondary antibodies include Goat anti-rabbit Alexa Fluor 488 nm and Goat anti-mouse Alexa Fluor 594 nm. Secondary antibodies are incubated at room temperature for 1 hour. DAPI (D1306, ThermoFisher Scientific) is added to the penultimate of five, 5 min PBS washes. After staining, cells are scanned on the ImageXpress Micro Confocal (Molecular Devices). TDP43 cells are quantified by hand-counting.

RNA foci visualization and quantification. The average percentage of the total number of cells containing RNA foci and the number of foci per 100 cells are calculated in the AAV vectors co-transduced C9orf72 ALS/FTD iPSC cell lines and untreated control cell line. Cells are imaged using a Widefield microscope. For quantification, at least 20 pictures are taken from randomly chosen microscopic fields, containing 100-300 cells for each treatment. The number of foci is counted.

All headings and section designations are used for clarity and reference purposes only and are not to be considered limiting in any way. For example, those of skill in the art will appreciate the usefulness of combining various embodiments from different headings and sections as appropriate according to the spirit and scope of the technology described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

All publications and patents cited in this specification are herein incorporated by reference for all purposes as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

### REFERENCES

1. Cantarero-Prieto, D., Leon, P. L., Blazquez-Fernandez, C., Juan, P. S. & Cobo, C. S. The economic cost of dementia: A systematic review. Dementia 19, 2637-2657 (2020).
2. Deb, A., Thornton, J. D., Sambamoorthi, U. & Innes, K. Direct and indirect cost of managing alzheimer's disease and related dementias in the United States. Expert Rev. Pharmacoecon. Outcomes Res. 17, 189-202 (2017).
3. Deb, A., Sambamoorthi, U., Thornton, J. D., Schreurs, B. & Innes, K. Direct medical expenditures associated with Alzheimer's and related dementias (ADRD) in a nationally representative sample of older adults—an excess cost approach. Aging Ment. Health 22, 619-624 (2018).
4. Jia, J. et al. The cost of Alzheimer's disease in China and re-estimation of costs worldwide. Alzheimers Dement 14, 483-491 (2018).
5. Gladman, M. & Zinman, L. The economic impact of amyotrophic lateral sclerosis: a systematic review. Expert Rev. Pharmacoecon. Outcomes Res. 15, 439-450 (2015).
6. GBD 2015 Neurological Disorders Collaborator Group. Global, regional, and national burden of neurological disorders during 1990-2015: a systematic analysis for the Global Burden of Disease Study 2015. Lancet Neurol. 16, 877-897 (2017).
7. DeJesus-Hernandez, M. et al. Expanded GGGGCC hexanucleotide repeat in noncoding region of C9ORF72 causes chromosome 9p-linked FTD and ALS. Neuron 72, 245-256 (2011).
8. Renton, A. E. et al. A hexanucleotide repeat expansion in C9ORF72 is the cause of chromosome 9p21-linked ALS-FTD. Neuron 72, 257-268 (2011).
9. Majounie, E. et al. Frequency of the C9orf72 hexanucleotide repeat expansion in patients with amyotrophic lateral sclerosis and frontotemporal dementia: a cross-sectional study. Lancet Neurol. 11, 323-330 (2012).
10. Jiang, J. et al. Gain of Toxicity from ALS/FTD-Linked Repeat Expansions in C9ORF72 Is Alleviated by Antisense Oligonucleotides Targeting GGGGCC-Containing RNAs. Neuron 90, 535-550 (2016).
11. Sareen, D. et al. Targeting RNA foci in iPSC-derived motor neurons from ALS patients with a C9ORF72 repeat expansion. Sci. Transl. Med. 5, 208ra149 (2013).
12. Donnelly, C. J. et al. RNA toxicity from the ALS/FTD C9ORF72 expansion is mitigated by antisense intervention. Neuron 80, 415-428 (2013).
13. Tran, H. et al. Suppression of mutant C9orf72 expression by a potent mixed backbone antisense oligonucleotide. Nat. Med. (2021) doi: 10.1038/s41591-021-01557-6.
14. Liu, Y. et al. Variant-selective stereopure oligonucleotides protect against pathologies associated with C9orf72-repeat expansion in preclinical models. Nat. Commun. 12, 847 (2021).
15. A Study to Assess the Safety, Tolerability, and Pharmacokinetics of BIIB078 in Adults With C9ORF72-Associated Amyotrophic Lateral Sclerosis-Full Text View-ClinicalTrials.gov. https://clinicaltrials.gov/ct2/show/NCT03626012.
16. Knott, G. J. & Doudna, J. A. CRISPR-Cas guides the future of genetic engineering. Science 361, 866-869 (2018).
17. Wang, S. et al. Nuclear export and translation of circular repeat-containing intronic RNA in C9ORF72-ALS/FTD. Nat. Commun. 12, 4908 (2021).
18. Lagier-Tourenne, C. et al. Targeted degradation of sense and antisense C9orf72 RNA foci as therapy for ALS and frontotemporal degeneration. Proc Natl Acad Sci USA 110, E4530-9 (2013).
19. Celona, B. et al. Suppression of C9orf72 RNA repeat-induced neurotoxicity by the ALS-associated RNA-binding protein Zfp106. eLife 6, (2017).
20. Zu, T. et al. RAN proteins and RNA foci from antisense transcripts in C9ORF72 ALS and frontotemporal dementia. Proc Natl Acad Sci USA 110, E4968-77 (2013).
21. Ash, P. E. A. et al. Unconventional translation of C9ORF72 GGGGCC expansion generates insoluble polypeptides specific to c9FTD/ALS. Neuron 77, 639-646 (2013).
22. Gendron, T. F. et al. Cerebellar c9RAN proteins associate with clinical and neuropathological characteristics of C9ORF72 repeat expansion carriers. Acta Neuropathol. 130, 559-573 (2015).
23. Mori, K. et al. The C9orf72 GGGGCC repeat is translated into aggregating dipeptide-repeat proteins in FTLD/ALS. Science 339, 1335-1338 (2013).
24. Mori, K. et al. Bidirectional transcripts of the expanded C9orf72 hexanucleotide repeat are translated into aggregating dipeptide repeat proteins. Acta Neuropathol. 126, 881-893 (2013).
25. Wen, X. et al. Antisense proline-arginine RAN dipeptides linked to C9ORF72-ALS/FTD form toxic nuclear aggregates that initiate in vitro and in vivo neuronal death. Neuron 84, 1213-1225 (2014).
26. Mackenzie, I. R. et al. Dipeptide repeat protein pathology in C9ORF72 mutation cases: clinico-pathological correlations. Acta Neuropathol. 126, 859-879 (2013).
27. Mann, D. M. A. et al. Dipeptide repeat proteins are present in the p62 positive inclusions in patients with frontotemporal lobar degeneration and motor neurone disease associated with expansions in C9ORF72. Acta Neuropathol. Commun. 1, 68 (2013).
28. Ryan, S., Rollinson, S., Hobbs, E. & Pickering-Brown, S. C9orf72 dipeptides disrupt the nucleocytoplasmic transport machinery and cause TDP-43 mislocalisation to the cytoplasm. Sci. Rep. 12, 4799 (2022).
29. Nanaura, H. et al. C9orf72-derived arginine-rich poly-dipeptides impede phase modifiers. Nat. Commun. 12, 5301 (2021).
30. Shi, Y. et al. Haploinsufficiency leads to neurodegeneration in C9ORF72 ALS/FTD human induced motor neurons. Nat. Med. 24, 313-325 (2018).
31. Ciura, S. et al. Loss of function of C9orf72 causes motor deficits in a zebrafish model of amyotrophic lateral sclerosis. Ann. Neurol. 74, 180-187 (2013).
32. Balendra, R. & Isaacs, A. M. C9orf72-mediated ALS and FTD: multiple pathways to disease. Nat. Rev. Neurol. 14, 544-558 (2018).
33. Shao, Q. et al. C9orf72 deficiency promotes motor deficits of a C9ALS/FTD mouse model in a dose-dependent manner. Acta Neuropathol. Commun. 7, 32 (2019).
34. gnomAD. https://gnomad.broadinstitute.org/gene/ENSG00000147894.
35. Harms, M. B. et al. Lack of C9ORF72 coding mutations supports a gain of function for repeat expansions in amyotrophic lateral sclerosis. Neurobiol. Aging 34, 2234.e13-9 (2013).
36. Burberry, A. et al. Loss-of-function mutations in the C9ORF72 mouse ortholog cause fatal autoimmune disease. Sci. Transl. Med. 8, 347ra93 (2016).
37. O'Rourke, J. G. et al. C9orf72 is required for proper macrophage and microglial function in mice. Science 351, 1324-1329 (2016).
38. Zhu, Q. et al. Reduced C9ORF72 function exacerbates gain of toxicity from ALS/FTD-causing repeat expansion in C9orf72. Nat. Neurosci. 23, 615-624 (2020).
39. Sudria-Lopez, E. et al. Full ablation of C9orf72 in mice causes immune system-related pathology and neoplastic events but no motor neuron defects. Acta Neuropathol. 132, 145-147 (2016).
40. Abo-Rady, M. et al. Knocking out C9ORF72 Exacerbates Axonal Trafficking Defects Associated with Hexanucleotide Repeat Expansion and Reduces Levels of Heat Shock Proteins. Stem Cell Reports 14, 390-405 (2020).
41. Pribadi, M. et al. CRISPR-Cas9 targeted deletion of the C9orf72 repeat expansion mutation corrects cellular phenotypes in patient-derived iPS cells. BioRxiv (2016) doi: 10.1101/051193.
42. Selvaraj, B. T. et al. C9ORF72 repeat expansion causes vulnerability of motor neurons to Ca2+-permeable AMPA receptor-mediated excitotoxicity. Nat. Commun. 9, 347 (2018).
43. Ababneh, N. A. et al. Correction of amyotrophic lateral sclerosis related phenotypes in induced pluripotent stem cell-derived motor neurons carrying a hexanucleotide expansion mutation in C9orf72 by CRISPR/Cas9 genome editing using homology-directed repair. Hum. Mol. Genet. 29, 2200-2217 (2020).
44. CRISPOR. http://crispor.tefor.net/.
45. Ferreira da Silva, J., Meyenberg, M. & Loizou, J. I. Tissue specificity of DNA repair: the CRISPR compass. Trends Genet. 37, 958-962 (2021).
46. Lee, S. H., Park, Y.-H., Jin, Y. B., Kim, S.-U. & Hur, J. K. CRISPR Diagnosis and Therapeutics with Single Base Pair Precision. Trends Mol. Med. (2019) doi: 10.1016/j.molmed.2019.09.008.
47. Watry, H. L. et al. Rapid, precise quantification of large DNA excisions and inversions by ddPCR. BioRxiv (2020) doi: 10.1101/2020.04.13.039297.
48. Shin, J. W. et al. Permanent inactivation of Huntington's disease mutation by personalized allele-specific CRISPR/Cas9. Hum. Mol. Genet. 25, 4566-4576 (2016).
49. Feliciano, C. M. et al. Allele-Specific Gene Editing Rescues Pathology in a Human Model of Charcot-Marie-Tooth Disease Type 2E. Front. Cell Dev. Biol. 9, 723023 (2021).
50. Monteys, A. M., Ebanks, S. A., Keiser, M. S. & Davidson, B. L. Crispr/cas9 editing of the mutant huntingtin allele in vitro and in vivo. Mol. Ther. 25, 12-23 (2017).
51. György, B. et al. Allele-specific gene editing prevents deafness in a model of dominant progressive hearing loss. Nat. Med. 25, 1123-1130 (2019).
52. Maule, G. et al. Allele specific repair of splicing mutations in cystic fibrosis through AsCas12a genome editing. Nat. Commun. 10, 3556 (2019).
53. Patrizi, C. et al. Allele-specific editing ameliorates dominant retinitis pigmentosa in a transgenic mouse model. Am. J. Hum. Genet. 108, 295-308 (2021).
54. CS52iALS-C9nxx-Cedars-Sinai Biomanufacturing Center. https://biomanufacturing.cedars-sinai.org/product/cs52ials-c9nxx/.
55. CS29iALS-C9nxx-Cedars-Sinai Biomanufacturing Center. https://biomanufacturing.cedars-sinai.org/product/cs29ials-c9nxx/.
56. Bram, E. et al. Comprehensive genotyping of the C9orf72 hexanucleotide repeat region in 2095 ALS samples from the NINDS collection using a two-mode, long-read PCR assay. Amyotroph. Lateral Scler. Frontotemporal Degener. 20, 107-114 (2019).
57. Buchman, V. L. et al. Simultaneous and independent detection of C9ORF72 alleles with low and high number of GGGGCC repeats using an optimised protocol of Southern blot hybridisation. Mol. Neurodegener. 8, 12 (2013).
58. Ebbert, M. T. W. et al. Long-read sequencing across the C9orf72 “GGGGCC” repeat expansion: implications for clinical use and genetic discovery efforts in human disease. Mol. Neurodegener. 13, 46 (2018).
59. Giesselmann, P. et al. Analysis of short tandem repeat expansions and their methylation state with nanopore sequencing. Nat. Biotechnol. 37, 1478-1481 (2019).
60. DeJesus-Hernandez, M. et al. Long-read targeted sequencing uncovers clinicopathological associations for C9orf72-linked diseases. Brain 144, 1082-1088 (2021).
61. Gillmore, J. D. et al. CRISPR-Cas9 In Vivo Gene Editing for Transthyretin Amyloidosis. N. Engl. J. Med. 385, 493-502 (2021).
62. Frangoul, H. et al. CRISPR-Cas9 Gene Editing for Sickle Cell Disease and β-Thalassemia. N. Engl. J. Med. 384, 252-260 (2021).
63. Stadtmauer, E. A. et al. CRISPR-engineered T cells in patients with refractory cancer. Science 367, (2020).
64. Haeussler, M. et al. Evaluation of off-target and on-target scoring algorithms and integration into the guide RNA selection tool CRISPOR. Genome Biol. 17, 148 (2016).
65. Keough, K. C. et al. AlleleAnalyzer: a tool for personalized and allele-specific sgRNA design. Genome Biol. 20, 167 (2019).
66. Sudmant, P. H. et al. An integrated map of structural variation in 2,504 human genomes. Nature 526, 75-81 (2015).
67. Ozcan, K. A., Ghaffari, L. T. & Haeusler, A. R. The effects of molecular crowding and CpG hypermethylation on DNA G-quadruplexes formed by the C9orf72 nucleotide repeat expansion. Sci. Rep. 11, 23213 (2021).
68. Cohen-Hadad, Y. et al. Marked Differences in C9orf72 Methylation Status and Isoform Expression between C9/ALS Human Embryonic and Induced Pluripotent Stem Cells. Stem Cell Reports 7, 927-940 (2016).
69. Malik, I., Kelley, C. P., Wang, E. T. & Todd, P. K. Molecular mechanisms underlying nucleotide repeat expansion disorders. Nat. Rev. Mol. Cell Biol. 22, 589-607 (2021).
70. van Blitterswijk, M. et al. Novel clinical associations with specific C9ORF72 transcripts in patients with repeat expansions in C9ORF72. Acta Neuropathol. 130, 863-876 (2015).
71. Rizzu, P. et al. C9orf72 is differentially expressed in the central nervous system and myeloid cells and consistently reduced in C9orf72, MAPT and GRN mutation carriers. Acta Neuropathol. Commun. 4, 37 (2016).
72. Vatsavayai, S. C., Nana, A. L., Yokoyama, J. S. & Seeley, W. W. C9orf72-FTD/ALS pathogenesis: evidence from human neuropathological studies. Acta Neuropathol. 137, 1-26 (2019).
73. Shen, C.-C. et al. Synthetic switch to minimize CRISPR off-target effects by self-restricting Cas9 transcription and translation. Nucleic Acids Res. 47, e13 (2019).
74. Kelkar, A. et al. Doxycycline-Dependent Self-Inactivation of CRISPR-Cas9 to Temporally Regulate On- and Off-Target Editing. Mol. Ther. 28, 29-41 (2020).
75. Hanlon, K. S. et al. High levels of AAV vector integration into CRISPR-induced DNA breaks. Nat. Commun. 10, 4439 (2019).
76. Nelson, C. E. et al. Long-term evaluation of AAV-CRISPR genome editing for Duchenne muscular dystrophy. Nat. Med. 25, 427-432 (2019).
77. Miyaoka, Y. et al. Isolation of single-base genome-edited human iPS cells without antibiotic selection. Nat. Methods 11, 291-293 (2014).
78. Fernandopulle, M. S. et al. Transcription Factor-Mediated Differentiation of Human iPSCs into Neurons. Curr. Protoc. Cell Biol. 79, e51 (2018).
79. No-Amp targeted sequencing—PacBio. https://www.pacb.com/products-and-services/applications/targeted-sequencing/no-amp-targeted-sequencing/.
80. iNeuron RNA-Seq|Kampmann Lab. https://kampmannlab.ucsf.edu/ineuron-rna-seq.
81. Nguyen, U., Squaglia, N., Boge, A. & Fung, P. A. The Simple Western™: a gel-free, blot-free, hands-free Western blotting reinvention. Nat. Methods 8, v-vi (2011).
82. Simple Western Technical Library::ProteinSimple. https://www.proteinsimple.com/technical_library.html?product=simplewestern&doctype=product_insert&def list=list.
83. Liu, Y. et al. C9orf72 BAC Mouse Model with Motor Deficits and Neurodegenerative Features of ALS/FTD. Neuron 90, 521-534 (2016).
84. Nguyen, L. et al. Survival and Motor Phenotypes in FVB C9-500 ALS/FTD BAC Transgenic Mice Reproduced by Multiple Labs. Neuron 108, 784-796.e3 (2020).

### Informal Sequence Listing

TABLE 1A
Repeat Expansion Excision guide nucleic
acids (gNAs) and primers

| | | | Seq |
|---|---|---|---|
| | | | ID |
| Edit Name | | REX | NO: |
| Edit Type | | Repeat Expansion | |
| | | Excision | |
| Excision | (WT | 7 bp if no RE, | |
| Size | allele) | 25 bp if 3 RE | |
| spCas9 Guides | 5′ gNA | AACTCAGGAGTCGCGCGCTA | 1 |
| Used | 3′ gNA | GGCCCGCCCCGACCACGCCC | 2 |
| Excision | F Primer | CCGCTAGGAAAGAGAGGTGCG | 3 |
| Primers | R Primer | GAGGAGAGCCCCCGCTTCTAC | 4 |
| 5′ Cut Site | F Primer | N/A | |
| Primers | R Primer | N/A | |
| 3′ Cut Site | F Primer | N/A | |
| Primers | R Primer | N/A | |

TABLE 1B
Exon 1A Excision guide nucleic acids
(gNAs) and primers

| | | | Seq |
|---|---|---|---|
| | | | ID |
| Edit Name | | 1AX | NO: |
| Edit Type | | Exon 1A Excision | |
| Excision | (WT | 227 bp | |
| Size | allele) | | |
| spCas9 Guides | 5′ gNA | TGCGATGACGTTTTCTCACG | 5 |
| Used | 3′ gNA | TACTGTGAGAGCAAGTAGTG | 6 |
| Excision | F Primer | GATCCAGCAGCCTCCCCTAT | 7 |
| Primers | R Primer | GCTACAGGCTGCGGTTGTTT | 8 |
| 5′ Cut Site | F Primer | TCCAGCAGCCTCCCCTATT | 9 |
| Primers | R Primer | TTTACGTGGGCGGAACTTGT | 10 |
| 3′ Cut Site | F Primer | AGAGAGGTGCGTCAAACAGC | 11 |
| Primers | R Primer | CTCCTGAGTTCCAGAGCTTG | 12 |
| | | C | |

Table 1C Exon 1B Excision guide nucleic acids (gNAs) and primers

TABLE 1C
Exon 1B Excision guide nucleic acids (gNAs)
and primers

| | | | Seq |
|---|---|---|---|
| Edit Name | | 1BX | ID NO: |
| Edit Type | | Exon 1B | |
| | | Excision | |
| Excision Size | (WT allele) | 124 bp | |
| spCas9 | 5′ gNA | CGTGGTCGGGG | 13 |
| Guides Used | | GGGGCCCGG | |
| | 3′ gNA | GCTGTTTGGGG | 14 |
| | | TTCGGCTGC | |
| Excision | F Primer | TAACCTACGGT | 15 |
| Primers | | GTCCCGCTA | |
| | R Primer | GGCAATTCCAC | 16 |
| | | CAGTCGCTA | |
| | F Primer | TACTCGCTGAG | 17 |
| | | GGTGAACAAG | |
| | R Primer | CAGTCGCTAGA | 18 |
| | | GGCGAAAGC | |
| 5′ Cut Site | F Primer | N/A | |
| Primers | | | |
| | R Primer | N/A | |
| 3′ Cut Site | F Primer | GTGGCTGTTT | 19 |
| Primers | | GGGGTTCGG | |
| | R Primer | CCAGTCGCTA | 20 |
| | | GAGGCGAAAG | |

TABLE 1D
Allele Specific Excision on REFERENCE allele
guide nucleic acids (gNAs) and primers

| | | | Seq |
|---|---|---|---|
| | | | ID |
| Edit Name | | HET(Ref)x | NO: |
| Edit Type | | Allele Specific | |
| | | Excision from | |
| | | upstream of | |
| | | Exon 1A to intron | |
| | | between Exon 3 and 4 | |
| | | on REFERENCE allele | |
| Excision | (WT | 21 kb | |
| Size | allele) | | |
| spCas9 Guides | 5′ gNA | CTCTGTGAGAAGTTTTTATC | 21 |
| Used | | | |
| Excision | 3′ gNA | GACTTAGAAGAAATATTGTG | 22 |
| Primers | F | | |
| | Primer | AGGAACCAAGCAGCCATGAA | 23 |
| | R | | |
| 5′ Cut Site | Primer | GGGAAGCCACACCCTTGTAA | 24 |
| Primers | F | | |
| | Primer | CTTTGGCACAGATAGGCCAC | 25 |
| | R | | |
| 3′ Cut Site | Primer | GGCAGGGTGACTGCTTTAAC | 26 |
| Primers | F | | |
| | Primer | TGCCCAGAATAAATTTTGGA | 27 |
| | R | TAACT | |
| | Primer | GGGAAGCCACACCCTTGTAA | 28 |

TABLE 1E
Allele Specific Excision on ALTERNATE allele
guide nucleic acids (gNAs) and primers

| | | | Seq |
|---|---|---|---|
| | | | ID |
| Edit Name | | HET(Alt)x | NO: |
| Edit Type | | Allele Specific | |
| | | Excision from | |
| | | upstream | |
| | | of Exon 1A to | |
| | | intron between | |
| | | Exon 3 | |
| | | and 4 on | |
| | | ALTERNATE allele | |
| Excision | (WT | | |
| Size | allele) | 21 kb | |
| spCas9 Guides | 5′ gNA | CTCTGTGAGAAGTTCTTATC | 29 |
| Used | 3′ gNA | GACTTAAAAGAAATATTGTG | 30 |
| Excision | F Primer | AGGAACCAAGCAGCCATGAA | 31 |
| Primers | R Primer | GGGAAGCCACACCCTTGTAA | 32 |
| 5′ Cut Site | F Primer | CTTTGGCACAGATAGGCCAC | 33 |
| Primers | R Primer | GGGAAGCCACACCCTTGTAA | 34 |
| 3′ Cut Site | F Primer | TGCCCAGAATAAATTTTGGA | 35 |
| | | TAACT | |
| Primers | R Primer | GGGAAGCCACACCCTTGTAA | 36 |

TABLE 1F
Excision from upstream of Exon1A to Exon 2
guide nucleic acids (gNAs) and primers

| | | | Seq |
|---|---|---|---|
| | | | ID |
| Edit Name | | KO (WT line) | NO: |
| Edit Type | | Excision from | |
| | | upstream of | |
| | | Exon1A to | |
| | | Exon 2 | |
| Excision | (WT | 7 kb | |
| Size | allele) | | |
| spCas9 Guides | 5′ gNA | TGTGCGAACCTTAATAGGGG | 37 |
| Used | 3′ gNA | AATGGGGATCGCAGCACATA | 38 |
| Excision | F Primer | GCAGACCAAAAGACGCAAGG | 39 |
| Primers | R Primer | ACCAGAAAATAAGCTTTCAA | 40 |
| | | CAGAT | |
| 5′ Cut Site | F Primer | GCAGACCAAAAGACGCAAGG | 41 |
| Primers | R Primer | CAGCGAGTACTGTGAGAGCA | 42 |
| 3′ Cut Site | F Primer | GGGTTAGGGGCCAAATCTCC | 43 |
| Primers | R Primer | ACCAGAAAATAAGCTTTCAA | 44 |
| | | CAGAT | |

TABLE 1G
Excision from upstream of Exon 1A to intron
between Exon 3 and 4 guide nucleic acids
(gNAs) and primers

| | | | Seq |
|---|---|---|---|
| | | | ID |
| Edit Name | | KO (Patient Line) | NO: |
| Edit Type | | Homozygous Excision | |
| | | from upstream of | |
| | | Exon 1A to intron | |
| | | between Exon 3 and | |
| | | 4 using 4 guides | |
| Excision | (WT | 21 kb | |
| Size | allele) | | |
| spCas9 Guides | 5′ gNA | CTCTGTGAGAAGTTTTTATC | 45 |
| Used | 5′ gNA | CTCTGTGAGAAGTTCTTATC | 46 |
| | 3′ gNA | GACTTAGAAGAAATATTGTG | 47 |
| | 3′ gNA | GACTTAAAAGAAATATTGTG | 48 |
| Excision | F Primer | AGGAACCAAGCAGCCATGAA | 49 |
| Primers | R Primer | GGGAAGCCACACCCTTGTAA | 50 |
| 5′ Cut Site | F Primer | CTTTGGCACAGATAGGCCAC | 51 |
| Primers | R Primer | GGCAGGGTGACTGCTTTAAC | 52 |
| 3′ Cut Site | F Primer | TGCCCAGAATAAATTTTGGA | 53 |
| Primers | | TAACT | |
| | R Primer | GGGAAGCCACACCCTTGTAA | 54 |

TABLE 2
all patients guide nucleic acids (gNAs)

| | | Seq | | Seq |
|---|---|---|---|---|
| | | ID | | ID |
| guide_id | gNA_ref | No | gNA_alt | No |
| SpCas9_0 | TTACAAAATGAACCTTGC | 55 | TTACAAAATGAACCTTGCAC | 56 |
| | AT | | | |
| SpCas9_1 | GAAGTTACTTAACCTATG | 57 | GAAGTTACTTAACCTGTGCA | 58 |
| | CA | | | |
| SpCas9_2 | AAACTTTCTGCTTAACTC | 59 | AAACTTTCTGCTTAACTCTC | 60 |
| | TC | | | |
| SpCas9_3 | CACATCTTTCTGATTCAT | 61 | CGCATCTTTCTGATTCATGA | 62 |
| | GA | | | |
| SpCas9_4 | ACATCTTTCTGATTCATG | 63 | GCATCTTTCTGATTCATGAT | 64 |
| | AT | | | |
| SpCas9_5 | GTTGTGTAAATTTGGTGG | 65 | ATTGTGTAAATTTGGTGGGT | 66 |
| | GT | | | |
| SpCas9_6 | GTCATTTTGTTGTGTAAA | 67 | GTCATTTTATTGTGTAAATT | 68 |
| | TT | | | |
| SpCas9_7 | ATTTTGTTGTGTAAATTT | 69 | ATTTTATTGTGTAAATTTGG | 70 |
| | GG | | | |
| SpCas9_8 | TTTTGTTGTGTAAATTTG | 71 | TTTTATTGTGTAAATTTGGT | 72 |
| | GT | | | |
| SpCas9_9 | TGTTGTGTAAATTTGGTG | 73 | TATTGTGTAAATTTGGTGGG | 74 |
| | GG | | | |
| SpCas9_10 | GTTGCAATGATTGCCAAA | 75 | TTTGCAATGATTGCCAAAGC | 76 |
| | GC | | | |
| SpCas9_11 | TCCATTATTTTTAGGCAG | 77 | TTTTTCCATTATTTTTAGGCAGA | 78 |
| | AA | | A | |
| SpCas9_16 | TTCCATTATTTTTAGGCA | 79 | TTTTTCCATTATTTTTAGGCAGA | 80 |
| | GA | | | |
| SpCas9_21 | TTTTTTTTTCCATTATTTT | 81 | TTTTTTTTTCCATTATTTTT | 82 |
| | T | | | |
| SpCas9_46 | TCCATTATTTTTAGGCAG | 83 | TCCATTATTTTTAGGCAGAA | 84 |
| | AA | | | |
| SpCas9_71 | TTCCATTATTTTTAGGCA | 85 | TTCCATTATTTTTAGGCAGA | 86 |
| | GA | | | |
| SpCas9_11 | TTTTTTTTTCCATTATTTT | 87 | TTTTTTTTTCCATTATTTTT | 88 |
| 1 | T | | | |
| SpCas9_13 | TCCATTATTTTTAGGCAG | 89 | TCCATTATTTTTAGGCAGAA | 90 |
| 6 | AA | | | |
| SpCas9_16 | TTCCATTATTTTTAGGCA | 91 | TTCCATTATTTTTAGGCAGA | 92 |
| 1 | GA | | | |
| SpCas9_20 | TTTTTTTTTCCATTATTTT | 93 | TTTTTTTTTCCATTATTTTT | 94 |
| 1 | T | | | |
| SpCas9_22 | TCCATTATTTTTAGGCAG | 95 | TTTTTTTTCCATTATTTTTAGGC | 96 |
| 6 | AA | | AGAA | |
| SpCas9_23 | TTCCATTATTTTTAGGCA | 97 | TTTTTTTTCCATTATTTTTAGGC | 98 |
| 1 | GA | | AGA | |
| SpCas9_23 | TTTTTTTTTCCATTATTTT | 99 | TTTTTTTTTCCATTATTTTT | 100 |
| 6 | T | | | |
| SpCas9_26 | TCCATTATTTTTAGGCAG | 101 | TTTCCATTATTTTTAGGCAGAA | 102 |
| 1 | AA | | | |
| SpCas9_26 | TTCCATTATTTTTAGGCA | 103 | TTTCCATTATTTTTAGGCAGA | 104 |
| 6 | GA | | | |
| SpCas9_27 | TTTTTTTTTCCATTATTTT | 105 | TTTTTTTTTCCATTATTTTT | 106 |
| 1 | T | | | |
| SpCas9_29 | AACAAAAAAACAATGTA | 107 | AACAAAAAACCAATGTACAA | 108 |
| 6 | CAA | | | |
| SpCas9_29 | ATTTTTTTTTTTTTTTGAG | 109 | ATATTTTTTTTTTTTTTTTTGAG | 110 |
| 7 | A | | A | |
| SpCas9_29 | ATTTTTTTTTTTTTTTGAG | 111 | ATTTTTTTTTTTTTTTGAGA | 112 |
| 9 | A | | | |
| SpCas9_30 | TCACTGCAACCTACACCT | 113 | TCACTGCAACCTCCACCTCC | 114 |
| 1 | CC | | | |
| SpCas9_30 | CACTGCAACCTACACCTC | 115 | CACTGCAACCTCCACCTCCT | 116 |
| 3 | CT | | | |
| SpCas9_30 | CGCTTGAACCCAGGAGGT | 117 | CGCTTGAACCCAGGAGGTGG | 118 |
| 5 | GT | | | |
| SpCas9_30 | CACTGCAACCTACACCTC | 119 | CACTGCAACCTACACCTCCG | 120 |
| 9 | CT | | | |
| SpCas9_31 | CGCTTGAACCCAGGAGGT | 121 | CGCTTGAACCCCGGAGGTGT | 122 |
| 3 | GT | | | |
| SpCas9_31 | GAGAATCGCTTGAACCCA | 123 | GAGAATCGCTTGAACCCCGG | 124 |
| 5 | GG | | | |
| SpCas9_31 | TCATATACCTGTACTTTT | 125 | TCATATACCCGTACTTTTGC | 126 |
| 6 | GC | | | |
| SpCas9_31 | CATATACCTGTACTTTTG | 127 | CATATACCCGTACTTTTGCA | 128 |
| 7 | CA | | | |
| SpCas9_31 | ATATACCTGTACTTTTGC | 129 | ATATACCCGTACTTTTGCAG | 130 |
| 8 | AG | | | |
| SpCas9_31 | TATACCTGTACTTTTGCA | 131 | TATACCCGTACTTTTGCAGG | 132 |
| 9 | GG | | | |
| SpCas9_32 | ACCTGTACTTTTGCAGGG | 133 | ACCCGTACTTTTGCAGGGGG | 134 |
| 0 | GG | | | |
| SpCas9_32 | TTTTTTTTTTTTTTGAGAA | 135 | TTTTTTTTTTTTTTGAGAAA | 136 |
| 1 | A | | | |
| SpCas9_32 | TTTTTTTTTTTTTTTGAGA | 137 | TTTTTTTTTTTTTTTGAGAA | 138 |
| 5 | A | | | |
| SpCas9_32 | TTTTTTTTTTTGAGAAAG | 139 | TTTTTTTTTTTGAGAAAGGG | 140 |
| 9 | GG | | | |
| SpCas9_33 | TTTTTTTTTTTTTTGAGAA | 141 | TTTTTTTTTTTTTTGAGAAA | 142 |
| 3 | A | | | |
| SpCas9_33 | TTTTTTTTTTTTTTTGAGA | 143 | TTTTTTTTTTTTTTTGAGAA | 144 |
| 7 | A | | | |
| SpCas9_34 | TTTTTTTTTTTGAGAAAG | 145 | TTTTTTTTTTTGAGAAAGGG | 146 |
| 1 | GG | | | |
| SpCas9_34 | TATACTCTACATAACATA | 147 | TATATTCTACATAACATAAT | 148 |
| 5 | AT | | | |
| SpCas9_34 | ATACTCTACATAACATAA | 149 | ATATTCTACATAACATAATA | 150 |
| 6 | TA | | | |
| SpCas9_34 | ATATATAGAACAGGCATT | 151 | ATATATAGAACAGGCACTGT | 152 |
| 7 | GT | | | |
| SpCas9_34 | TAAAAAAAGTAGAGACC | 153 | TAAAAAAAGTAGAGACCTTT | 154 |
| 8 | CTT | | | |
| SpCas9_34 | TTTTTTGAGATGGCAAGA | 155 | GTTTTTGAGATGGCAAGAAT | 156 |
| 9 | AT | | | |
| SpCas9_35 | TTTTTTGTTTTTTTTTGAG | 157 | TTTTTGTTTTGTTTTTGAGA | 158 |
| 0 | A | | | |
| SpCas9_35 | CGTTTTGCGGCTATTGTG | 159 | CGCTTTGCGGCTATTGTGAA | 160 |
| 1 | AA | | | |
| SpCas9_35 | GTTTTGCGGCTATTGTGA | 161 | GCTTTGCGGCTATTGTGAAT | 162 |
| 2 | AT | | | |
| SpCas9_35 | TAGGTATTTTATTCGTTTT | 163 | TAGGTATTTTATTCGCTTTG | 164 |
| 3 | G | | | |
| SpCas9_35 | GCAAAACGAATAAAATA | 165 | GCAAAGCGAATAAAATACCT | 166 |
| 4 | CCT | | | |
| SpCas9_35 | CTGAAAAGGAGTGGTGA | 167 | CTGAAAAGGAGCGGTGAGAG | 168 |
| 6 | GAG | | | |
| SpCas9_35 | ACTCCTTTTCAGCATAGT | 169 | GCTCCTTTTCAGCATAGTTC | 170 |
| 7 | TC | | | |
| SpCas9_35 | TGATTTTTCTTTTTTGAGA | 171 | TGATTTTTCTTTTTTGAGAC | 172 |
| 8 | C | | | |
| SpCas9_35 | GATTTTTCTTTTTTGAGAC | 173 | GATTTTTCTTTTTTGAGACA | 174 |
| 9 | A | | | |
| SpCas9_36 | ACCTCAACCTCCTGAATA | 175 | ATCTCAACCTCCTGAATAGC | 176 |
| 0 | GC | | | |
| SpCas9_36 | CCTCAACCTCCTGAATAG | 177 | TCTCAACCTCCTGAATAGCT | 178 |
| 1 | CT | | | |
| SpCas9_36 | ATTCAGGAGGTTGAGGTG | 179 | ATTCAGGAGGTTGAGATGGG | 180 |
| 2 | GG | | | |
| SpCas9_36 | GTGGGAGGATTGTTTGAG | 181 | ATGGGAGGATTGTTTGAGCC | 182 |
| 3 | CC | | | |
| SpCas9_36 | GCTATTCAGGAGGTTGAG | 183 | GCTATTCAGGAGGTTGAGAT | 184 |
| 4 | GT | | | |
| SpCas9_36 | AGCTATTCAGGAGGTTGA | 185 | AGCTATTCAGGAGGTTGAGA | 186 |
| 5 | GG | | | |
| SpCas9_36 | TCCTGAATAGCTGGGACT | 187 | TCCTGAATAGCTGGGACTAC | 188 |
| 6 | AT | | | |
| SpCas9_36 | ACCTATAGTCCCAGCTAT | 189 | ACCTGTAGTCCCAGCTATTC | 190 |
| 7 | TC | | | |
| SpCas9_36 | TATAGTCCCAGCTATTCA | 191 | TGTAGTCCCAGCTATTCAGG | 192 |
| 8 | GG | | | |
| SpCas9_36 | AAAAAAAAATTAGCTTG | 193 | AAAAAAAAATTAGCTTGGTA | 194 |
| 9 | GTA | | | |
| SpCas9_38 | AAAAAAAAAAAAAATTA | 195 | AAAAAAAAAAAAAATTAGCT | 196 |
| 5 | GCT | | | |
| SpCas9_40 | AAAAAAAAATTAGCTTG | 197 | AAAAAAAAATTAGCTTGGTA | 198 |
| 1 | GTA | | | |
| SpCas9_41 | AAAAAAAAAAAAAATTA | 199 | AAAAAAAAAAAAAATTAGCT | 200 |
| 7 | GCT | | | |
| SpCas9_43 | AAAAAAAAATTAGCTTG | 201 | AAAAAAAAATTAGCTTGGTA | 202 |
| 3 | GTA | | | |
| SpCas9_44 | AAAAAAAAAAAAAATTA | 203 | AAAAAAAAAAAAAATTAGCT | 204 |
| 9 | GCT | | | |
| SpCas9_46 | AAAAAAAAATTAGCTTG | 205 | AAAAAAAAATTAGCTTGGTA | 206 |
| 5 | GTA | | | |
| SpCas9_48 | AAAAAAAAAAAAAATTA | 207 | AAAAAAAAAAAAAATTAGCT | 208 |
| 1 | GCT | | | |
| SpCas9_49 | CACCTGTAATCCCAGCAC | 209 | CATCTGTAATCCCAGCACTT | 210 |
| 7 | TT | | | |
| SpCas9_49 | ACCTGTAATCCCAGCACT | 211 | ATCTGTAATCCCAGCACTTT | 212 |
| 8 | TT | | | |
| SpCas9_49 | CTTGGCTCTCTATTCTCCA | 213 | CTTGGCTCTGTATTCTCCAT | 214 |
| 9 | T | | | |
| SpCas9_50 | CCCCACTACAAACTGGGC | 215 | CCCCACTACAAACCGGGCAA | 216 |
| 0 | AA | | | |
| SpCas9_50 | AACCCACCCCACTACAAA | 217 | AACCCACCCCACTACAAACC | 218 |
| 5 | CT | | | |
| SpCas9_50 | TGCCCAGTTTGTAGTGGG | 219 | TGCCCGGTTTGTAGTGGGGT | 220 |
| 7 | GT | | | |
| SpCas9_50 | TTGCCCAGTTTGTAGTGG | 221 | TTGCCCGGTTTGTAGTGGGG | 222 |
| 8 | GG | | | |
| SpCas9_50 | CCTTTGCCCAGTTTGTAG | 223 | CCTTTGCCCGGTTTGTAGTG | 224 |
| 9 | TG | | | |
| SpCas9_51 | TCCTTTGCCCAGTTTGTA | 225 | TCCTTTGCCCGGTTTGTAGT | 226 |
| 0 | GT | | | |
| SpCas9_51 | GTCCTTTGCCCAGTTTGT | 227 | GTCCTTTGCCCGGTTTGTAG | 228 |
| 1 | AG | | | |
| SpCas9_51 | AGAAAAATTTGTATTCCT | 229 | AGAACAATTTGTATTCCTTT | 230 |
| 2 | TT | | | |
| SpCas9_51 | AGAATGGGTTTGCAACCA | 231 | AGAATGGGTTTGCAACCGTT | 232 |
| 4 | TT | | | |
| SpCas9_51 | GGAAAAAAGAAAATGTT | 233 | GGAAAAAAGAAAATATTTCA | 234 |
| 5 | TCA | | | |
| SpCas9_51 | AAAATGCTTGCAATAATG | 235 | AAAATGCTTGCAATAATGCC | 236 |
| 6 | CC | | | |
| SpCas9_52 | AAAATGCTTGCAATAATG | 237 | AAAATGCTTGCAATAATGCC | 238 |
| 5 | CC | | | |
| SpCas9_54 | TCCCAAAGCTCTGTTCTT | 239 | ACCCAAAGCTCTGTTCTTAC | 240 |
| 3 | AC | | | |
| SpCas9_54 | GAGCTTTGGGAGTGCAAG | 241 | GAGCTTTGGGTGTGCAAGGC | 242 |
| 5 | GC | | | |
| SpCas9_54 | AACAGAGCTTTGGGAGTG | 243 | AACAGAGCTTTGGGTGTGCA | 244 |
| 6 | CA | | | |
| SpCas9_54 | CTTTGGGAGTGCAAGGCA | 245 | CTTTGGGTGTGCAAGGCAGG | 246 |
| 7 | GG | | | |
| SpCas9_54 | TCCCAAAGCTCTGTTCTT | 247 | TCCCAAAGCTCTGTTCTTAT | 248 |
| 8 | AC | | | |
| SpCas9_55 | GCCTGTAAGAACAGAGCT | 249 | GCCTATAAGAACAGAGCTTT | 250 |
| 0 | TT | | | |
| SpCas9_55 | TGCCTGTAAGAACAGAGC | 251 | TGCCTATAAGAACAGAGCTT | 252 |
| 1 | TT | | | |
| SpCas9_55 | TCCTTTTGAATTTACTGTT | 253 | TCCTTTTGAATTTACTGTAC | 254 |
| 2 | C | | | |
| SpCas9_55 | GCCTGAACAGTAAATTCA | 255 | GCCTGTACAGTAAATTCAAA | 256 |
| 3 | AA | | | |
| SpCas9_55 | GAAGCTTTGTGCTGCCTT | 257 | GAAGTTTTGTGCTGCCTTTC | 258 |
| 4 | TC | | | |
| SpCas9_55 | ACTGGCTACCCCAACTGA | 259 | ACTGCCTACCCCAACTGATT | 260 |
| 5 | TT | | | |
| SpCas9_55 | TCATCTTTGTATAATGTC | 261 | TCATCTTCGTATAATGTCAA | 262 |
| 6 | AA | | | |
| SpCas9_55 | ATAGTTTTGAAATTTCCT | 263 | ACAGTTTTGAAATTTCCTCT | 264 |
| 7 | CT | | | |
| SpCas9_55 | TTTTTTATTTCATTTTGCC | 265 | TTTTTTATTTCATTTTGCTG | 266 |
| 8 | G | | | |
| SpCas9_55 | AATTATTTATAATACTTA | 267 | AAATATTTATAATACTTAAA | 268 |
| 9 | AA | | | |
| SpCas9_56 | CAATTATTTATAATACTT | 269 | CAAATATTTATAATACTTAA | 270 |
| 0 | AA | | | |
| SpCas9_56 | TTAAAAAAAAAAAAGTC | 271 | GTTAAAAAAAAAAAAAGTCCT | 272 |
| 1 | CTG | | G | |
| SpCas9_56 | TTTTAACACATGCTTAAA | 273 | TTTTAACACATGCTTAAATC | 274 |
| 3 | TC | | | |
| SpCas9_56 | AGTCCTGAGGCTAGACAT | 275 | TGTCCTGAGGCTAGACATGT | 276 |
| 4 | GT | | | |
| SpCas9_56 | TTAAAAAAAAAAAAGTC | 277 | TTAAAAAAAAAAATGTCCTG | 278 |
| 5 | CTG | | | |
| SpCas9_56 | CAGAAGCTATAAAAGTTG | 279 | CAGAAGCTATAAAAGTTCCA | 280 |
| 7 | CA | | | |
| SpCas9_56 | AAAAAAAAACACTCCAA | 281 | AAACAAAAAAACACTCCAACT | 282 |
| 8 | CTA | | A | |
| SpCas9_56 | TTAGTTTATTAGCTAATG | 283 | TTGGTTTATTAGCTAATGCT | 284 |
| 9 | CT | | | |
| SpCas9_57 | AGCATTAGCTAATAAACT | 285 | AGCATTAGCTAATAAACCAA | 286 |
| 0 | AA | | | |
| SpCas9_57 | ATTAACTCAAGTAGCACT | 287 | ACTAACTCAAGTAGCACTGA | 288 |
| 1 | GA | | | |
| SpCas9_57 | AGTGCTACTTGAGTTAAT | 289 | AGTGCTACTTGAGTTAGTTA | 290 |
| 3 | TA | | | |
| SpCas9_57 | CTCAAGTAGCACTGAAGG | 291 | CTCAAGTAACACTGAAGGAA | 292 |
| 5 | AA | | | |
| SpCas9_57 | TCAAGTAGCACTGAAGG | 293 | TCAAGTAACACTGAAGGAAA | 294 |
| 6 | AAA | | | |
| SpCas9_57 | ATTAACTCAAGTAGCACT | 295 | ATTAACTCAAGTAACACTGA | 296 |
| 7 | GA | | | |
| SpCas9_57 | AGTGCTACTTGAGTTAAT | 297 | AGTGTTACTTGAGTTAATTA | 298 |
| 9 | TA | | | |
| SpCas9_58 | CATTTATACACTACTGAC | 299 | CATTTATACACAACTGACAC | 300 |
| 1 | AC | | | |
| SpCas9_58 | CAGTGTCAGTAGTGTATA | 301 | CAGTGTCAGTTGTGTATAAA | 302 |
| 2 | AA | | | |
| SpCas9_58 | TTATAACAACCCTACACA | 303 | TTATAACAACCCCACACATT | 304 |
| 3 | TT | | | |
| SpCas9_58 | GTAATAGTACCTAATGTG | 305 | GTAATAGTACCTAATGTGTG | 306 |
| 4 | TA | | | |
| SpCas9_58 | GTGCACAGAGATTGTTTT | 307 | GTACACAGAGATTGTTTTTT | 308 |
| 5 | TT | | | |
| SpCas9_58 | AGTGCACAGAGATTGTTT | 309 | AGTACACAGAGATTGTTTTT | 310 |
| 6 | TT | | | |
| SpCas9_58 | TGCACAGAGATTGTTTTT | 311 | TACACAGAGATTGTTTTTTG | 312 |
| 7 | TG | | | |
| SpCas9_58 | TGTGTGCTTATTTGCTAA | 313 | TGTGTGCCTATTTGCTAAAG | 314 |
| 8 | AG | | | |
| SpCas9_58 | AATAAGCACACACAAAA | 315 | AATAGGCACACACAAAATCC | 316 |
| 9 | TCC | | | |
| SpCas9_59 | ATATTCTAATTTTAATAG | 317 | ATATTCTAATTTTTATAGTA | 318 |
| 0 | TA | | | |
| SpCas9_59 | TTCTTTTTTTTTTTTTGAG | 319 | TTTTCTTTTTTTTTTTTTTTGAGA | 320 |
| 1 | A | | | |
| SpCas9_59 | TTCTTTTTTTTTTTTTGAG | 321 | TTCTTTTTTTTTTTTTGAGA | 322 |
| 3 | A | | | |
| SpCas9_59 | TTTCGCCATGTTGGCCAG | 323 | TTTCGCCATGTTGGCCAGGC | 324 |
| 5 | GT | | | |
| SpCas9_59 | TCAGAAATTTGACACCAA | 325 | TCAGAAATTTGACACCAGCC | 326 |
| 6 | CC | | | |
| SpCas9_59 | TGACACCAACCTGGCCAA | 327 | TGACACCAGCCTGGCCAACA | 328 |
| 7 | CA | | | |
| SpCas9_59 | TTTGCAAGAACTCAATCA | 329 | TTTGCAAGAACTCAGTCAAA | 330 |
| 8 | AA | | | |
| SpCas9_59 | GGTGTTGTTGAGCTTCAG | 331 | GGTGTTGTTGAGCTTCAGTG | 332 |
| 9 | TG | | | |
| SpCas9_60 | GGTGTTGTTGAGCTTCAG | 333 | TTGGTGTTGTTGAGCTTCAGTG | 334 |
| 3 | TG | | | |
| SpCas9_60 | GTTCAAGTTTGGGAAGTA | 335 | GTTCGAGTTTGGGAAGTACA | 336 |
| 5 | CA | | | |
| SpCas9_60 | CTGCTGAGAAGTTCAAGT | 337 | CTGCTGAGAAGTTCGAGTTT | 338 |
| 6 | TT | | | |
| SpCas9_60 | ACTGCTGAGAAGTTCAAG | 339 | ACTGCTGAGAAGTTCGAGTT | 340 |
| 7 | TT | | | |
| SpCas9_60 | CAAGTTTGGGAAGTACAA | 341 | CGAGTTTGGGAAGTACAAGG | 342 |
| 8 | GG | | | |
| SpCas9_60 | TCAACTTAAGTAAAGGTT | 343 | TCAACTTAAGCAAAGGTTTC | 344 |
| 9 | TC | | | |
| SpCas9_61 | ATAGTAGTCAACTTAAGT | 345 | ATAGTAGTCAACTTAAGCAA | 346 |
| 0 | AA | | | |
| SpCas9_61 | AGTAAACTTATCGCAAGT | 347 | AGTAAACTTACCGCAAGTTG | 348 |
| 1 | TG | | | |
| SpCas9_61 | GACTTAGAAGAAATATTG | 22 | GACTTAAAAGAAATATTGTG | 46 |
| 2 | TG | | | |
| SpCas9_61 | GAAGAAATATTGTGTGGA | 349 | AAAGAAATATTGTGTGGACC | 350 |
| 3 | CC | | | |
| SpCas9_61 | TGTCTCTGATATGTTCTTT | 351 | TGTCTCTGATATGTTCTTTA | 352 |
| 4 | A | | | |
| SpCas9_61 | CTGGGCAAACAGAAAAA | 353 | CTGGGCAAACAGATAAAAAA | 354 |
| 5 | AAA | | | |
| SpCas9_61 | TGGGCAAACAGAAAAAA | 355 | TGGGCAAACAGATAAAAAAA | 356 |
| 6 | AAA | | | |
| SpCas9_61 | TAGGGAAAAAATGGGGG | 357 | TAGGGAAACAATGGGGGCAG | 358 |
| 7 | CAG | | | |
| SpCas9_61 | GAAAAAATGGGGGCAGG | 359 | GAAACAATGGGGGCAGGGGT | 360 |
| 8 | GGT | | | |
| SpCas9_61 | TTTCTACTTTAGGGAAAA | 361 | TTTCTACTTTAGGGAAACAA | 362 |
| 9 | AA | | | |
| SpCas9_62 | TTCTACTTTAGGGAAAAA | 363 | TTCTACTTTAGGGAAACAAT | 364 |
| 0 | AT | | | |
| SpCas9_62 | TCTACTTTAGGGAAAAAA | 365 | TCTACTTTAGGGAAACAATG | 366 |
| 1 | TG | | | |
| SpCas9_62 | CTACTTTAGGGAAAAAAT | 367 | CTACTTTAGGGAAACAATGG | 368 |
| 2 | GG | | | |
| SpCas9_62 | TTTAGGGAAAAAATGGG | 369 | TTTAGGGAAACAATGGGGGC | 370 |
| 3 | GGC | | | |
| SpCas9_62 | TTAGGGAAAAAATGGGG | 371 | TTAGGGAAACAATGGGGGCA | 372 |
| 4 | GCA | | | |
| SpCas9_62 | GTTTCATAATGTGAGAAA | 373 | GTTTTATAATGTGAGAAAAA | 374 |
| 5 | AA | | | |
| SpCas9_62 | ATTATGAAACTCTTAAAA | 375 | ATTATAAAACTCTTAAAATC | 376 |
| 6 | TC | | | |
| SpCas9_62 | AGCATTATATATTCAACT | 377 | ACCATTATATATTCAACTAC | 378 |
| 7 | AC | | | |
| SpCas9_62 | CAACGGGCAGTTGCAGC | 379 | CAACGGGCAGTTACAGCAGC | 380 |
| 8 | AGC | | | |
| SpCas9_62 | GCAGTTGCAGCAGCTGGA | 381 | GCAGTTACAGCAGCTGGACT | 382 |
| 9 | CT | | | |
| SpCas9_63 | TCAGCTTCTGGGCAGGCC | 383 | TCAGCTTCTGGGCAGGCCAC | 384 |
| 0 | AT | | | |
| SpCas9_63 | CAGCTTCTGGGCAGGCCA | 385 | CAGCTTCTGGGCAGGCCACA | 386 |
| 1 | TA | | | |
| SpCas9_63 | TAGGGAACTTAAAATCAT | 387 | CAGGGAACTTAAAATCATGA | 388 |
| 2 | GA | | | |
| SpCas9_63 | TCATGATTTTAAGTTCCC | 389 | TCATGATTTTAAGTTCCCTG | 390 |
| 3 | TA | | | |
| SpCas9_63 | TTTTGATTTTACAGGTTC | 391 | TTTTGATTTTACAGGTTCAC | 392 |
| 4 | AT | | | |
| SpCas9_63 | TTACAGGTTCATAGGTAG | 393 | TTACAGGTTCACAGGTAGAA | 394 |
| 5 | AA | | | |
| SpCas9_63 | TTTACAGGTTCATAGGTA | 395 | TTTACAGGTTCACAGGTAGA | 396 |
| 6 | GA | | | |
| SpCas9_63 | TGAGTGGGCTCCCAAAAC | 397 | TGAGTGGGCTCCGAAAACCT | 398 |
| 7 | CT | | | |
| SpCas9_63 | GAGTGGGCTCCCAAAACC | 399 | GAGTGGGCTCCGAAAACCTT | 400 |
| 8 | TT | | | |
| SpCas9_63 | TGTCTTGTCATAGCTCTG | 401 | TCTCTTGTCATAGCTCTGCT | 402 |
| 9 | CT | | | |
| SpCas9_64 | ATCACTGGGTATATACCC | 403 | ATCCCTGGGTATATACCCAA | 404 |
| 0 | AA | | | |
| SpCas9_64 | TGACCCAGCCATCCCATC | 405 | TGACCCAGCCATCCCATCCC | 406 |
| 1 | AC | | | |
| SpCas9_64 | GACCCAGCCATCCCATCA | 407 | GACCCAGCCATCCCATCCCT | 408 |
| 2 | CT | | | |
| SpCas9_64 | TTTGGGTATATACCCAGT | 409 | TTTGGGTATATACCCAGGGA | 410 |
| 3 | GA | | | |
| SpCas9_64 | TACCCAGTGATGGGATGG | 411 | TACCCAGGGATGGGATGGCT | 412 |
| 4 | CT | | | |
| SpCas9_64 | ATACCCAGTGATGGGATG | 413 | ATACCCAGGGATGGGATGGC | 414 |
| 5 | GC | | | |
| SpCas9_64 | GTATATACCCAGTGATGG | 415 | GTATATACCCAGGGATGGGA | 416 |
| 6 | GA | | | |
| SpCas9_64 | TTGGGTATATACCCAGTG | 417 | TTGGGTATATACCCAGGGAT | 418 |
| 7 | AT | | | |
| SpCas9_64 | TATTCACAATAGCAAAGA | 419 | TGTTCACAATAGCAAAGACT | 420 |
| 8 | CT | | | |
| SpCas9_64 | TCACACTCTGGGGACTGT | 421 | TCACACTCCGGGGACTGTTG | 422 |
| 9 | TG | | | |
| SpCas9_65 | CACACTCTGGGGACTGTT | 423 | CACACTCCGGGGACTGTTGT | 424 |
| 0 | GT | | | |
| SpCas9_65 | ACACTCTGGGGACTGTTG | 425 | ACACTCCGGGGACTGTTGTG | 426 |
| 1 | TG | | | |
| SpCas9_65 | CTCTGGGGACTGTTGTGG | 427 | CTCCGGGGACTGTTGTGGGG | 428 |
| 2 | GG | | | |
| SpCas9_65 | TCTGGGGACTGTTGTGGG | 429 | TCCGGGGACTGTTGTGGGGT | 430 |
| 3 | GT | | | |
| SpCas9_65 | CTGGGGACTGTTGTGGGG | 431 | CCGGGGACTGTTGTGGGGTG | 432 |
| 4 | TG | | | |
| SpCas9_65 | TGGGGACTGTTGTGGGGT | 433 | CGGGGACTGTTGTGGGGTGG | 434 |
| 5 | GG | | | |
| SpCas9_65 | GAAGGGGAACATCACAC | 435 | GAAGGGGAACATCACACTCC | 436 |
| 7 | TCT | | | |
| SpCas9_65 | AAGGGGAACATCACACT | 437 | AAGGGGAACATCACACTCCG | 438 |
| 9 | CTG | | | |
| SpCas9_66 | GAAAGACTCATAGGACT | 439 | GAAAGACTCATAGGACTAAT | 440 |
| 0 | AAC | | | |
| SpCas9_66 | GTGCTCATGCCTATGAGC | 441 | GTGCTCATGCCTATGAACAT | 442 |
| 1 | AT | | | |
| SpCas9_66 | TTCTAATAGCCTATGCTC | 443 | TTCTAATAGCCTATGTTCAT | 444 |
| 2 | AT | | | |
| SpCas9_66 | TTCACAGATCCCTAGAAC | 445 | TTCACAGATACCTAGAACAG | 446 |
| 3 | AG | | | |
| SpCas9_66 | TGACTTTGGAACCGGCTA | 447 | TGACTTTGGAACCTGCTAAC | 448 |
| 5 | AC | | | |
| SpCas9_66 | GGAACAGAGAGAGGGAA | 449 | GGAACAGAGAGAGGGAAAGG | 450 |
| 7 | AGT | | | |
| SpCas9_66 | GAACAGAGAGAGGGAAA | 451 | GAACAGAGAGAGGGAAAGGG | 452 |
| 9 | GTG | | | |
| SpCas9_67 | AACAGAGAGAGGGAAAG | 453 | AACAGAGAGAGGGAAAGGGG | 454 |
| 0 | TGG | | | |
| SpCas9_67 | AGAGAGAGGGAAAGTGG | 455 | AGAGAGAGGGAAAGGGGGGG | 456 |
| 1 | GGG | | | |
| SpCas9_67 | AGGCTCCATCTCCAACGT | 457 | AGGCTCCATCTCCAACGCTG | 458 |
| 2 | TG | | | |
| SpCas9_67 | CCAGGCTCCATCTCCAAC | 459 | CCAGGCTCCATCTCCAACGC | 460 |
| 3 | GT | | | |
| SpCas9_67 | CAGGCTCCATCTCCAACG | 461 | CAGGCTCCATCTCCAACGCT | 462 |
| 4 | TT | | | |
| SpCas9_67 | TCAGTTGCAATCCCCAAC | 463 | TCAGTTGCAATCCCCAGCGT | 464 |
| 5 | GT | | | |
| SpCas9_67 | GCAATCCCCAACGTTGGA | 465 | GCAATCCCCAGCGTTGGAGA | 466 |
| 6 | GA | | | |
| SpCas9_67 | CCAACGTTGGAGATGGA | 467 | CCAGCGTTGGAGATGGAGCC | 468 |
| 7 | GCC | | | |
| SpCas9_67 | AATCTTATCACATACATA | 469 | AATCTTATCGCATACATAAA | 470 |
| 8 | AA | | | |
| SpCas9_67 | TTTAGAATAAGACTGGAA | 471 | TTTAGAGTAAGACTGGAACT | 472 |
| 9 | CT | | | |
| SpCas9_68 | TCCCCAGTTTAGAATAAG | 473 | TCCCCAGTTTAGAGTAAGAC | 474 |
| 0 | AC | | | |
| SpCas9_68 | TCCAGTCTTATTCTAAAC | 475 | TCCAGTCTTACTCTAAACTG | 476 |
| 1 | TG | | | |
| SpCas9_68 | TTCCAGTCTTATTCTAAA | 477 | TTCCAGTCTTACTCTAAACT | 478 |
| 2 | CT | | | |
| SpCas9_68 | GTTCCAGTCTTATTCTAA | 479 | GTTCCAGTCTTACTCTAAAC | 480 |
| 3 | AC | | | |
| SpCas9_68 | GACACAGCAGGAAGTTT | 481 | GACACAGCAGGAAGGTTATC | 482 |
| 4 | ATC | | | |
| SpCas9_68 | TTCACTCTTGTTGTCCAG | 483 | TTCACTCTTGTTGTCCAAGC | 484 |
| 5 | GC | | | |
| SpCas9_68 | TTGTCCAGGCTGGAGTGC | 485 | TTGTCCAAGCTGGAGTGCAA | 486 |
| 6 | AA | | | |
| SpCas9_68 | CGTGCCATTGCACTCCAG | 487 | CGTGCCATTGCACTCCAGCT | 488 |
| 7 | CC | | | |
| SpCas9_68 | TCCCGAAGTGCTGAGATT | 489 | TCCCAAAGTGCTGAGATTAC | 490 |
| 8 | AC | | | |
| SpCas9_68 | CTCAGCACTTCGGGAGGC | 491 | CTCAGCACTTTGGGAGGCCA | 492 |
| 9 | CA | | | |
| SpCas9_69 | TGTAATCTCAGCACTTCG | 493 | TGTAATCTCAGCACTTTGGG | 494 |
| 0 | GG | | | |
| SpCas9_69 | ACCTGTAATCTCAGCACT | 495 | ACCTGTAATCTCAGCACTTT | 496 |
| 1 | TC | | | |
| SpCas9_69 | GCACTTCGGGAGGCCAA | 497 | GCACTTTGGGAGGCCAAGGA | 498 |
| 2 | GGA | | | |
| SpCas9_69 | AGCACTTCGGGAGGCCA | 499 | AGCACTTTGGGAGGCCAAGG | 500 |
| 3 | AGG | | | |
| SpCas9_69 | TGCCTTAATCATTACGGA | 501 | TGCCTCAATCATTACGGACT | 502 |
| 4 | CT | | | |
| SpCas9_69 | TGTGGTTGCCTTAATCAT | 503 | TGTGGTTGCCTCAATCATTA | 504 |
| 6 | TA | | | |
| SpCas9_69 | CTTAATCATTACGGACTA | 505 | CTCAATCATTACGGACTAGG | 506 |
| 7 | GG | | | |
| SpCas9_69 | CGCCTAGTCCGTAATGAT | 507 | CGCCTAGTCCGTAATGATTG | 508 |
| 9 | TA | | | |
| SpCas9_70 | TGCCTTAATCATTACGGA | 509 | TGCCTTAATCATTACAGACT | 510 |
| 1 | CT | | | |
| SpCas9_70 | CTTAATCATTACGGACTA | 511 | CTTAATCATTACAGACTAGG | 512 |
| 3 | GG | | | |
| SpCas9_70 | CGCCTAGTCCGTAATGAT | 513 | CGCCTAGTCTGTAATGATTA | 514 |
| 5 | TA | | | |
| SpCas9_70 | GGGGTTTCACCATGTTAG | 515 | GGAGTTTCACCATGTTAGTC | 516 |
| 7 | TC | | | |
| SpCas9_70 | TTGTATTTTTAGTAGAGA | 517 | TTGTATTTTTAGTAGAGATG | 518 |
| 8 | TG | | | |
| SpCas9_71 | TTTGTATTTTTAGTAGAG | 519 | TTTGTATTTTTAGTAGAGAT | 520 |
| 0 | AT | | | |
| SpCas9_71 | TTTTGTATTTTTAGTAGA | 521 | TTTTGTATTTTTAGTAGAGA | 522 |
| 2 | GA | | | |
| SpCas9_71 | GAATACAGAGTTCTGGGG | 523 | GAATACAGAGTTCTTGGGAG | 524 |
| 3 | AG | | | |
| SpCas9_71 | GCCTGGAATACAGAGTTC | 525 | GCCTGGAATACAGAGTTCTT | 526 |
| 5 | TG | | | |
| SpCas9_71 | TCCCCAGAACTCTGTATT | 527 | TCCCAAGAACTCTGTATTCC | 528 |
| 7 | CC | | | |
| SpCas9_71 | AAAGCTAGGCCAGTTGCT | 529 | AAAGCTAGGCCGGTTGCTCA | 530 |
| 9 | CA | | | |
| SpCas9_72 | TAAAGCTAGGCCAGTTGC | 531 | TAAAGCTAGGCCGGTTGCTC | 532 |
| 0 | TC | | | |
| SpCas9_72 | AGAGAAGCTTGAAAGTC | 533 | AGAGAAGCTTCAAAGTCAAG | 534 |
| 1 | AAG | | | |
| SpCas9_72 | TGGAGAGGTGAGGACAT | 535 | TGGAGAGGTGGGGACATTTC | 536 |
| 2 | TTC | | | |
| SpCas9_72 | CAATTTCCTCCTCTTGAA | 537 | CAAATTCCTCCTCTTGAAAA | 538 |
| 3 | AA | | | |
| SpCas9_72 | TTTTCAAGAGGAGGAAAT | 539 | TTTTCAAGAGGAGGAATTTG | 540 |
| 4 | TG | | | |
| SpCas9_72 | AAAAAAAAAAAGCTCTT | 541 | AAAAAAAAAAAGCTCTTACC | 542 |
| 5 | ACC | | | |
| SpCas9_72 | AAAAGCTCTTACCTGGTT | 543 | AAAAGCTCTTACCTGGTTGC | 544 |
| 9 | GC | | | |
| SpCas9_73 | AAAAAAAAAAAGCTCTT | 545 | AAAAAAAAAAAGCTCTTACC | 546 |
| 3 | ACC | | | |
| SpCas9_73 | AAAAGCTCTTACCTGGTT | 547 | AAAAGCTCTTACCTGGTTGC | 548 |
| 7 | GC | | | |
| SpCas9_74 | TTTTTTTTTAAATCAACAT | 549 | TTTTTTTTAAAATCAACATA | 550 |
| 1 | A | | | |
| SpCas9_74 | AAACAACTGATAGTCGTC | 551 | AAACAACTGATAATCGTCAG | 552 |
| 2 | AG | | | |
| SpCas9_74 | ATGCCTAAATAATGTCAT | 553 | ATGCCTAAATAATGTCATCG | 554 |
| 3 | TG | | | |
| SpCas9_74 | TTTCCACAATGACATTAT | 555 | TTTCCACGATGACATTATTT | 556 |
| 4 | TT | | | |
| SpCas9_74 | GCAGAAAGTGACAGTGA | 557 | GCAGAAAGTGACTGTGAGAC | 558 |
| 5 | GAC | | | |
| SpCas9_74 | AGTGACAGTGAGACTGG | 559 | AGTGACTGTGAGACTGGAAA | 560 |
| 6 | AAA | | | |
| SpCas9_74 | AAGTGACAGTGAGACTG | 561 | AAGTGACTGTGAGACTGGAA | 562 |
| 7 | GAA | | | |
| SpCas9_74 | CAGTGAGACTGGAAAGG | 563 | CTGTGAGACTGGAAAGGGAT | 564 |
| 8 | GAT | | | |
| SpCas9_74 | TCACTCTCACTCTGTTGC | 565 | TCATTCTCACTCTGTTGCCC | 566 |
| 9 | CC | | | |
| SpCas9_75 | GTGAGAGTGAGACTCTTT | 567 | GTGAGAATGAGACTCTTTCT | 568 |
| 0 | CT | | | |
| SpCas9_75 | AACTCTGGGGGAAAAGA | 569 | AACTCTGGGGGAAAAAAAGT | 570 |
| 1 | AGT | | | |
| SpCas9_75 | CTTCTTTTCCCCCAGAGTT | 571 | CTTTTTTTCCCCCAGAGTTC | 572 |
| 2 | C | | | |
| SpCas9_75 | GATGAATGAATCTTGTTT | 573 | GATGAATGAATTTTGTTTAC | 574 |
| 3 | AC | | | |
| SpCas9_75 | ATGAATGAATCTTGTTTA | 575 | ATGAATGAATTTTGTTTACA | 576 |
| 4 | CA | | | |
| SpCas9_75 | AGATTCATTCATCTCAAA | 577 | AAATTCATTCATCTCAAATT | 578 |
| 5 | TT | | | |
| SpCas9_75 | AAGATTCATTCATCTCAA | 579 | AAAATTCATTCATCTCAAAT | 580 |
| 6 | AT | | | |
| SpCas9_75 | GAGAAGTTTTTATCTGGT | 581 | GAGAAGTTCTTATCTGGTGA | 582 |
| 7 | GA | | | |
| SpCas9_75 | CTCTGTGAGAAGTTTTTA | 21 | CTCTGTGAGAAGTTCTTATC | 29 |
| 8 | TC | | | |
| SpCas9_75 | AGAAGAAATGTCTATGA | 583 | AGAAAAAATGTCTATGAAAG | 584 |
| 9 | AAG | | | |
| SpCas9_76 | GACATTTCTTCTGCATGG | 585 | GACATTTTTTCTGCATGGGT | 586 |
| 1 | GT | | | |
| SpCas9_76 | CATAGACATTTCTTCTGC | 587 | CATAGACATTTTTTCTGCAT | 588 |
| 2 | AT | | | |
| SpCas9_76 | TCATAGACATTTCTTCTG | 589 | TCATAGACATTTTTTCTGCA | 590 |
| 4 | CA | | | |
| SpCas9_76 | TGTCTATGAAAGCGGTCA | 591 | TGTCTGTGAAAGCGGTCAAA | 592 |
| 6 | AA | | | |
| SpCas9_76 | ATGTCTATGAAAGCGGTC | 593 | ATGTCTGTGAAAGCGGTCAA | 594 |
| 7 | AA | | | |
| SpCas9_76 | AGAAGAAATGTCTATGA | 595 | AGAAGAAATGTCTGTGAAAG | 596 |
| 8 | AAG | | | |
| SpCas9_77 | CATAGACATTTCTTCTGC | 597 | CACAGACATTTCTTCTGCAT | 598 |
| 0 | AT | | | |
| SpCas9_77 | TCATAGACATTTCTTCTG | 599 | TCACAGACATTTCTTCTGCA | 600 |
| 2 | CA | | | |
| SpCas9_77 | AATGAGTAGCTCTGCAAA | 601 | AATGAGTAGCTTTGCAAAAG | 602 |
| 4 | AG | | | |
| SpCas9_77 | TCTGCAAAAGTGGATTTT | 603 | TTTGCAAAAGTGGATTTTCC | 604 |
| 5 | CC | | | |
| SpCas9_77 | CTACTCATTTTTTGATAAT | 605 | CTACTCATTTTTCGATAATT | 606 |
| 6 | T | | | |
| SpCas9_77 | GCTACTCATTTTTTGATA | 607 | GCTACTCATTTTTCGATAAT | 608 |
| 7 | AT | | | |
| SpCas9_77 | AAGACATACAAATGGCA | 609 | AAGACGTACAAATGGCAAAC | 610 |
| 8 | AAC | | | |
| SpCas9_78 | TTCAAGAGAAGACATAC | 611 | TTCAAGAGAAGACGTACAAA | 612 |
| 0 | AAA | | | |
| SpCas9_78 | AAGACATACAAATGGCA | 613 | AAGACATACAAGTGGCAAAC | 614 |
| 2 | AAC | | | |
| SpCas9_78 | TTCAAGAGAAGACATAC | 615 | TTCAAGAGAAGACATACAAG | 616 |
| 4 | AAA | | | |
| SpCas9_78 | TATGTGTTCTGCCTCCCCT | 617 | TACGTGTTCTGCCTCCCCTA | 618 |
| 6 | A | | | |
| SpCas9_78 | CTCTTTTTTTTTTTTTG | 619 | CCTCCTTTTTTTTTTGA | 620 |
| 7 | A | | | |
| SpCas9_79 | CTCTTTTTTTTTTTTTTTG | 621 | CCTCTTTTTTTTTTTTTTTTGA | 622 |
| 1 | A | | | |
| SpCas9_79 | CGTCAAAAAAAAAAAAA | 623 | GTCAAAAAAAAAAAAAAG | 624 |
| 5 | AAG | | | |
| SpCas9_79 | CTCTTTTTTTTTTTTTTTG | 625 | TCCTCTTTTTTTTTTGA | 626 |
| 8 | A | | | |
| SpCas9_80 | CGTCAAAAAAAAAAAAA | 627 | TCAAAAAAAAAAAAAAAAAG | 628 |
| 2 | AAG | | | |
| SpCas9_83 | TTTTATTTATTAATTGCCT | 629 | CCCCCCCCCCCCCCCCCCCC | 630 |
| 0 | A | | | |
| SpCas9_84 | GGTTTCTTTCTTCTGTAGT | 631 | CCCCCCCCCCCCCCCCCCCC | 632 |
| 0 | G | | | |
| SpCas9_85 | CCCAGCTATTCAGGAGGT | 633 | CCCCCCCCCCCCCCCCCCCC | 634 |
| 0 | TG | | | |
| SpCas9_87 | CTCCCAAAGTGCTGGGAT | 635 | GGGGGGGGGGGGGGGGGGGG | 636 |
| 0 | TA | | | |
| SpCas9_87 | CTTTCTTGAGGATGTAGC | 637 | CCCCCCCCCCCCCCCCCCCC | 638 |
| 7 | CA | | | |
| SpCas9_88 | AAATGTGAAAGCTGGTCA | 639 | GGGGGGGGGGGGGGGGGGGG | 640 |
| 7 | AG | | | |
| SpCas9_89 | TCTGGGCAAACAGAAAA | 641 | GGGGGGGGGGGGGGGGGGGG | 642 |
| 4 | AAA | | | |
| SpCas9_90 | GGTGGTTCTGCCCAAGGT | 643 | CCCCCCCCCCCCCCCCCCCC | 644 |
| 1 | TT | | | |
| SpCas9_91 | TGCATTGTTCCTCTGTTCT | 645 | CCCCCCCCCCCCCCCCCCCC | 646 |
| 1 | A | | | |
| SpCas9_92 | TGTAGAAGTGACTTTGGA | 647 | CCCCCCCCCCCCCCCCCCCC | 648 |
| 1 | AC | | | |
| SpCas9_93 | AGAGTTTCACTCTTGTTG | 649 | GGGGGGGGGGGGGGGGGGGG | 650 |
| 1 | TC | | | |
| SpCas9_93 | ATGTGGTTGCCTTAATCA | 651 | GGGGGGGGGGGGGGGGGGGG | 652 |
| 8 | TT | | | |
| SpCas9_94 | TTGTATTTTTAGTAGAGA | 653 | CCCCCCCCCCCCCCCCCCCC | 654 |
| 5 | TG | | | |
| SpCas9_96 | TTTGTATTTTTAGTAGAG | 655 | CCCCCCCCCCCCCCCCCCCC | 656 |
| 5 | AT | | | |
| SpCas9_98 | AAGGCCTGGAATACAGA | 657 | GGGGGGGGGGGGGGGGGGGG | 658 |
| 5 | GTT | | | |
| SpCas9_99 | TTACATTTTGGGGGCAAA | 659 | GGGGGGGGGGGGGGGGGGGG | 660 |
| 2 | TA | | | |
| SpCas9_99 | AGGGACTGGTTTCATGAC | 661 | CCCCCCCCCCCCCCCCCCCC | 662 |
| 9 | TA | | | |
| SpCas9_10 | GGGGGGGGGGGGGGGGG | 663 | TATATTAATATTATAATAAT | 664 |
| 09 | GGG | | | |
| SpCas9_10 | GGGGGGGGGGGGGGGGG | 665 | ATGTACAAAGGTTTTTCTTC | 666 |
| 34 | GGG | | | |
| SpCas9_10 | GGGGGGGGGGGGGGGGG | 667 | CCTCCTGGGTTCAAGCGATT | 668 |
| 59 | GGG | | | |
| SpCas9_10 | GGGGGGGGGGGGGGGGG | 669 | CTCACTGCAACCTACACCTC | 670 |
| 84 | GGG | | | |
| SpCas9_11 | GGGGGGGGGGGGGGGGG | 671 | TACTTTTGCAGGGGGTGGAG | 672 |
| 09 | GGG | | | |
| SpCas9_11 | GGGGGGGGGGGGGGGGG | 673 | GGCAAAGGACATGAAAATTT | 674 |
| 34 | GGG | | | |
| SpCas9_11 | GGGGGGGGGGGGGGGGG | 675 | CTTTCCTAATCAGTTGGGGT | 676 |
| 59 | GGG | | | |
| SpCas9_11 | GGGGGGGGGGGGGGGGG | 677 | TTCTCAGACTTATTTGACCA | 678 |
| 84 | GGG | | | |
| SpCas9_12 | GGGGGGGGGGGGGGGGG | 679 | AAGAAAAGTGCAAAATCCTT | 680 |
| 09 | GGG | | | |
| SpCas9_12 | GGGGGGGGGGGGGGGGG | 681 | CACATTAGGTACTATTACTA | 682 |
| 34 | GGG | | | |
| SpCas9_12 | GGGGGGGGGGGGGGGGG | 683 | GCACACAGTTTTGTTTCAAA | 684 |
| 59 | GGG | | | |
| SpCas9_12 | GGGGGGGGGGGGGGGGG | 685 | ATTTGCTAAAGTGGCTAATA | 686 |
| 84 | GGG | | | |
| SpCas9_13 | GGGGGGGGGGGGGGGGG | 687 | ATCCTTTAGTAACCTCCACA | 688 |
| 09 | GGG | | | |
| SpCas9_13 | GGGGGGGGGGGGGGGGG | 689 | TTCTAAGTTTCCTCAACTTG | 690 |
| 34 | GGG | | | |
| SpCas9_13 | GGGGGGGGGGGGGGGGG | 691 | TTATATATTCAACTACAGGA | 692 |
| 59 | GGG | | | |
| SpCas9_13 | GGGGGGGGGGGGGGGGG | 693 | TGGGTATATACCCAAAGGAC | 694 |
| 84 | GGG | | | |
| SpCas9_14 | GGGGGGGGGGGGGGGGG | 695 | GGGTATATACCCAAAGGACT | 696 |
| 09 | GGG | | | |
| SpCas9_14 | GGGGGGGGGGGGGGGGG | 697 | GGGACTGTTGTGGGGTGGGG | 698 |
| 34 | GGG | | | |
| SpCas9_14 | GGGGGGGGGGGGGGGGG | 699 | GGAGGAACAGAGAGAGGGAA | 700 |
| 59 | GGG | | | |
| SpCas9_14 | GGGGGGGGGGGGGGGGG | 701 | GAGGAACAGAGAGAGGGAAA | 702 |
| 84 | GGG | | | |
| SpCas9_15 | GGGGGGGGGGGGGGGGG | 703 | TCCTGCTGTGTCAAAAATGC | 704 |
| 09 | GGG | | | |
| SpCas9_15 | GGGGGGGGGGGGGGGGG | 705 | GCCTAGCTTTACCCCTCTAC | 706 |
| 34 | GGG | | | |
| SpCas9_15 | GGGGGGGGGGGGGGGGG | 707 | ACCTCTCCATTTTGTGTGAG | 708 |
| 59 | GGG | | | |
| SpCas9_15 | GGGGGGGGGGGGGGGGG | 709 | CCTCTCCATTTTGTGTGAGC | 710 |
| 84 | GGG | | | |
| SpCas9_16 | GGGGGGGGGGGGGGGGG | 711 | TACATTATAAATCAGGAGAC | 712 |
| 09 | GGG | | | |

TABLE 3
guide nucleic acids (gNAs) and primers
for cell line generation

| | | Seq |
|---|---|---|
| | | ID |
| Name | Sequence | No |
| CLYBL safe-harbor | ATGTTGGAAGG | 713 |
| locus gNA | ATGAGGAAA | |
| CLYBL safe-harbor | CAGACAAGTCA | 714 |
| locus homology | GTAGGGCCA | |
| arm junction primer 1 | | |
| CLYBL safe-harbor | AGAAGACTTCC | 715 |
| locus homology | TCTGCCCTC | |
| arm junction primer 2 | | |
| CLYBL wild-type primer 1 | TGACTAAACAC | 716 |
| | TGTGCCCCA | |
| CLYBL wild-type primer 2 | AGGCAGGATGA | 717 |
| | ATTGGTGGA | |
| Nemomycin primer 1 | CATGGCTGATG | 718 |
| | CAATGCG | |
| Nemomycin primer 2 | TCGCTTGGTGG | 719 |
| | TCGAATG | |

TABLE 4
guide nucleic acids (gNAs) for excising
the genomic region with repeat expansion
for single molecule sequencing

| Name | Sequence | Seq ID NO. |
|---|---|---|
| 5′ gNA | GGAAGAAAGAATTGCAATTA | 720 |
| 3′ gNA | TTGGTATTTAGAAAGGTGGT | 721 |

TABLE 5
guide nucleic acids (gNAs) binding sequence

| | | Seq |
|---|---|---|
| | | ID |
| Name | Sequence | NO. |
| protein- | rGrUrU rUrUrA rGrArG | 722 |
| binding | rCrUrA rUrGrC rU | |
| sequence in | | |
| crRNA | | |
| protein- | rG rUrUrU rUrArG | 723 |
| binding | rArGrC rUrArG rArArA | |
| sequence in | rUrArG rCrArA rGrUrU | |
| sgRNA | rArArA rArUrA rArGrG | |
| | rCrUrA rGrUrC rCrGrU | |
| | rUrArU rCrArA rCrUrU | |
| | rGrArA rArArA rGrUrG | |
| | rGrCrA rCrCrG rArGrU | |
| | rCrGrG rUrGrC | |
| | mU*mU*mU* rU | |

TABLE 7
DNA sequences encoding gRNAs for in vivo
validation of allele-specific excision

| Encoded gRNA | Sequence | Seq ID NO. |
|---|---|---|
| ALT 3′gRNAs | CACAATATTTCTTTTAAGTC | 724 |
| ALT 5′gRNAs | GATAAGAACTTCTCACAGAG | 725 |
| REx 3′gRNAs | GGGCGTGGTCGGGGGGGGCC | 726 |
| REx 5′gRNAs | TAGCGCGCGACTCCTGAGTT | 727 |

TABLE 8
gRNA sequences

| Edit Name | Sequence | Seq ID NO: |
|---|---|---|
| Repeat Expansion Excision-5′ sgRNA | AACUCAGGAGUCGCGCGCUA | 731 |
| Repeat Expansion Excision-3′ sgRNA | GGCCCGCCCCGACCACGCCC | 732 |
| Exon 1A Excision-5′ sgRNA | UGCGAUGACGUUUUCUCACG | 733 |
| Exon 1A Excision-3′ sgRNA | UACUGUGAGAGCAAGUAGUG | 734 |
| Exon 1B Excision-5′ sgRNA | CGUGGUCGGGGCGGGCCCGG | 735 |
| Exon 1B Excision-3′ sgRNA | GCUGUUUGGGGUUCGGCUGC | 736 |
| HET(Ref)x-5′ sgRNA | CUCUGUGAGAAGUUUUUAUC | 737 |
| HET(Ref)x-3′ sgRNA | GACUUAGAAGAAAUAUUGUG | 738 |
| HET(Alt)x-5′ sgRNA | CUCUGUGAGAAGUUCUUAUC | 739 |
| HET(Alt)x-3′ sgRNA | GACUUAAAAGAAAUAUUGUG | 740 |
| KO (WT line)- 5′ sgRNA | UGUGCGAACCUUAAUAGGGG | 741 |
| KO (WT line)- 3′ sgRNA | AAUGGGGAUCGCAGCACAUA | 742 |
| KO (Patient Line)-5′ sgRNA | CUCUGUGAGAAGUUUUUAUC | 743 |
| KO (Patient Line)-3′ sgRNA | CUCUGUGAGAAGUUCUUAUC | 744 |
| KO (Patient Line)-5′ sgRNA | GACUUAGAAGAAAUAUUGUG | 745 |
| KO (Patient Line)-3′ sgRNA | GACUUAAAAGAAAUAUUGUG | 746 |
| CLYBL safe-harbor locus gRNA | AUGUUGGAAGGAUGAGGAAA | 747 |
| gRNAs for excising the genomic region | GGAAGAAAGAAUUGCAAUUA | 748 |
| with repeat expansion for single molecule | | |
| sequencing-5′ gRNA | | |
| gRNAs for excising the genomic region | UUGGUAUUUAGAAAGGUGGU | 749 |
| with repeat expansion for single molecule | | |
| sequencing-3′ gRNA | | |

TABLE 9
gRNA sequences for allele specific excision

| | | Seq ID | | Seq ID |
|---|---|---|---|---|
| guide_id | gRNA_ref | No | gRNA_alt | No |
| SpCas9_0 | UUACAAAAUGAACCU | 749 | UUACAAAAUGAACCUUGCAC | 750 |
| | UGCAU | | | |
| SpCas9_1 | GAAGUUACUUAACCU | 751 | GAAGUUACUUAACCUGUGCA | 752 |
| | AUGCA | | | |
| SpCas9_2 | AAACUUUCUGCUUAA | 753 | AAACUUUCUGCUUAACUCUC | 754 |
| | CUCUC | | | |
| SpCas9_3 | CACAUCUUUCUGAUU | 755 | CGCAUCUUUCUGAUUCAUGA | 756 |
| | CAUGA | | | |
| SpCas9_4 | ACAUCUUUCUGAUUC | 757 | GCAUCUUUCUGAUUCAUGAU | 758 |
| | AUGAU | | | |
| SpCas9_5 | GUUGUGUAAAUUUG | 759 | AUUGUGUAAAUUUGGUGGGU | 760 |
| | GUGGGU | | | |
| SpCas9_6 | GUCAUUUUGUUGUGU | 761 | GUCAUUUUAUUGUGUAAAUU | 762 |
| | AAAUU | | | |
| SpCas9_7 | AUUUUGUUGUGUAA | 763 | AUUUUAUUGUGUAAAUUUGG | 764 |
| | AUUUGG | | | |
| SpCas9_8 | UUUUGUUGUGUAAA | 765 | UUUUAUUGUGUAAAUUUGGU | 766 |
| | UUUGGU | | | |
| SpCas9_9 | UGUUGUGUAAAUUU | 767 | UAUUGUGUAAAUUUGGUGGG | 768 |
| | GGUGGG | | | |
| SpCas9_1 | GUUGCAAUGAUUGCC | 769 | UUUGCAAUGAUUGCCAAAGC | 770 |
| 0 | AAAGC | | | |
| SpCas9_1 | UCCAUUAUUUUUAGG | 771 | UUUUUCCAUUAUUUUUAGGC | 772 |
| 1 | CAGAA | | AGAA | |
| SpCas9_1 | UUCCAUUAUUUUUAG | 773 | UUUUUCCAUUAUUUUUAGGC | 774 |
| 6 | GCAGA | | AGA | |
| SpCas9_2 | UUUUUUUUUCCAUUA | 775 | UUUUUUUUUCCAUUAUUUUU | 776 |
| 1 | UUUUU | | | |
| SpCas9_4 | UCCAUUAUUUUUAGG | 777 | UCCAUUAUUUUUAGGCAGAA | 778 |
| 6 | CAGAA | | | |
| SpCas9_7 | UUCCAUUAUUUUUAG | 779 | UUCCAUUAUUUUUAGGCAGA | 780 |
| 1 | GCAGA | | | |
| SpCas9_1 | UUUUUUUUUCCAUUA | 781 | UUUUUUUUUCCAUUAUUUUU | 782 |
| 11 | UUUUU | | | |
| SpCas9_1 | UCCAUUAUUUUUAGG | 783 | UCCAUUAUUUUUAGGCAGAA | 784 |
| 36 | CAGAA | | | |
| SpCas9_1 | UUCCAUUAUUUUUAG | 785 | UUCCAUUAUUUUUAGGCAGA | 786 |
| 61 | GCAGA | | | |
| SpCas9_2 | UUUUUUUUUCCAUUA | 787 | UUUUUUUUUCCAUUAUUUUU | 788 |
| 01 | UUUUU | | | |
| SpCas9_2 | UCCAUUAUUUUUAGG | 789 | UUUUUUUUCCAUUAUUUUUA | 790 |
| 26 | CAGAA | | GGCAGAA | |
| SpCas9_2 | UUCCAUUAUUUUUAG | 791 | UUUUUUUUCCAUUAUUUUUA | 792 |
| 31 | GCAGA | | GGCAGA | |
| SpCas9_2 | UUUUUUUUUCCAUUA | 793 | UUUUUUUUUCCAUUAUUUUU | 794 |
| 36 | UUUUU | | | |
| SpCas9_2 | UCCAUUAUUUUUAGG | 795 | UUUCCAUUAUUUUUAGGCAG | 796 |
| 61 | CAGAA | | AA | |
| SpCas9_2 | UUCCAUUAUUUUUAG | 797 | UUUCCAUUAUUUUUAGGCAG | 798 |
| 66 | GCAGA | | A | |
| SpCas9_2 | UUUUUUUUUCCAUUA | 799 | UUUUUUUUUCCAUUAUUUUU | 800 |
| 71 | UUUUU | | | |
| SpCas9_2 | AACAAAAAAACAAUG | 801 | AACAAAAAACCAAUGUACAA | 802 |
| 96 | UACAA | | | |
| SpCas9_2 | AUUUUUUUUUUUUU | 803 | AUAUUUUUUUUUUUUUUUUU | 804 |
| 97 | UUGAGA | | GAGA | |
| SpCas9_2 | AUUUUUUUUUUUUU | 805 | AUUUUUUUUUUUUUUUGAGA | 806 |
| 99 | UUGAGA | | | |
| SpCas9_3 | UCACUGCAACCUACA | 807 | UCACUGCAACCUCCACCUCC | 808 |
| 01 | CCUCC | | | |
| SpCas9_3 | CACUGCAACCUACAC | 809 | CACUGCAACCUCCACCUCCU | 810 |
| 03 | CUCCU | | | |
| SpCas9_3 | CGCUUGAACCCAGGA | 811 | CGCUUGAACCCAGGAGGUGG | 812 |
| 05 | GGUGU | | | |
| SpCas9_3 | CACUGCAACCUACAC | 813 | CACUGCAACCUACACCUCCG | 814 |
| 09 | CUCCU | | | |
| SpCas9_3 | CGCUUGAACCCAGGA | 815 | CGCUUGAACCCCGGAGGUGU | 816 |
| 13 | GGUGU | | | |
| SpCas9_3 | GAGAAUCGCUUGAAC | 817 | GAGAAUCGCUUGAACCCCGG | 818 |
| 15 | CCAGG | | | |
| SpCas9_3 | UCAUAUACCUGUACU | 819 | UCAUAUACCCGUACUUUUGC | 820 |
| 16 | UUUGC | | | |
| SpCas9_3 | CAUAUACCUGUACUU | 821 | CAUAUACCCGUACUUUUGCA | 822 |
| 17 | UUGCA | | | |
| SpCas9_3 | AUAUACCUGUACUUU | 823 | AUAUACCCGUACUUUUGCAG | 824 |
| 18 | UGCAG | | | |
| SpCas9_3 | UAUACCUGUACUUUU | 825 | UAUACCCGUACUUUUGCAGG | 826 |
| 19 | GCAGG | | | |
| SpCas9_3 | ACCUGUACUUUUGCA | 827 | ACCCGUACUUUUGCAGGGGG | 828 |
| 20 | GGGGG | | | |
| SpCas9_3 | UUUUUUUUUUUUUU | 829 | UUUUUUUUUUUUUUGAGAAA | 830 |
| 21 | GAGAAA | | | |
| SpCas9_3 | UUUUUUUUUUUUUU | 831 | UUUUUUUUUUUUUUUGAGAA | 832 |
| 25 | UGAGAA | | | |
| SpCas9_3 | UUUUUUUUUUUGAG | 833 | UUUUUUUUUUUGAGAAAGGG | 834 |
| 29 | AAAGGG | | | |
| SpCas9_3 | UUUUUUUUUUUUUU | 835 | UUUUUUUUUUUUUUGAGAAA | 836 |
| 33 | GAGAAA | | | |
| SpCas9_3 | UUUUUUUUUUUUUU | 837 | UUUUUUUUUUUUUUUGAGAA | 838 |
| 37 | UGAGAA | | | |
| SpCas9_3 | UUUUUUUUUUUGAG | 839 | UUUUUUUUUUUGAGAAAGGG | 840 |
| 41 | AAAGGG | | | |
| SpCas9_3 | UAUACUCUACAUAAC | 841 | UAUAUUCUACAUAACAUAAU | 842 |
| 45 | AUAAU | | | |
| SpCas9_3 | AUACUCUACAUAACA | 843 | AUAUUCUACAUAACAUAAUA | 844 |
| 46 | UAAUA | | | |
| SpCas9_3 | AUAUAUAGAACAGGC | 845 | AUAUAUAGAACAGGCACUGU | 846 |
| 47 | AUUGU | | | |
| SpCas9_3 | UAAAAAAAGUAGAG | 847 | UAAAAAAAGUAGAGACCUUU | 848 |
| 48 | ACCCUU | | | |
| SpCas9_3 | UUUUUUGAGAUGGCA | 849 | GUUUUUGAGAUGGCAAGAAU | 850 |
| 49 | AGAAU | | | |
| SpCas9_3 | UUUUUUGUUUUUUU | 851 | UUUUUGUUUUGUUUUUGAGA | 852 |
| 50 | UUGAGA | | | |
| SpCas9_3 | CGUUUUGCGGCUAUU | 853 | CGCUUUGCGGCUAUUGUGAA | 854 |
| 51 | GUGAA | | | |
| SpCas9_3 | GUUUUGCGGCUAUUG | 855 | GCUUUGCGGCUAUUGUGAAU | 856 |
| 52 | UGAAU | | | |
| SpCas9_3 | UAGGUAUUUUAUUCG | 857 | UAGGUAUUUUAUUCGCUUUG | 858 |
| 53 | UUUUG | | | |
| SpCas9_3 | GCAAAACGAAUAAAA | 859 | GCAAAGCGAAUAAAAUACCU | 860 |
| 54 | UACCU | | | |
| SpCas9_3 | CUGAAAAGGAGUGGU | 861 | CUGAAAAGGAGCGGUGAGAG | 862 |
| 56 | GAGAG | | | |
| SpCas9_3 | ACUCCUUUUCAGCAU | 863 | GCUCCUUUUCAGCAUAGUUC | 864 |
| 57 | AGUUC | | | |
| SpCas9_3 | UGAUUUUUCUUUUUU | 865 | UGAUUUUUCUUUUUUGAGAC | 866 |
| 58 | GAGAC | | | |
| SpCas9_3 | GAUUUUUCUUUUUUG | 867 | GAUUUUUCUUUUUUGAGACA | 868 |
| 59 | AGACA | | | |
| SpCas9_3 | ACCUCAACCUCCUGA | 869 | AUCUCAACCUCCUGAAUAGC | 870 |
| 60 | AUAGC | | | |
| SpCas9_3 | CCUCAACCUCCUGAA | 871 | UCUCAACCUCCUGAAUAGCU | 872 |
| 61 | UAGCU | | | |
| SpCas9_3 | AUUCAGGAGGUUGAG | 873 | AUUCAGGAGGUUGAGAUGGG | 874 |
| 62 | GUGGG | | | |
| SpCas9_3 | GUGGGAGGAUUGUU | 875 | AUGGGAGGAUUGUUUGAGCC | 876 |
| 63 | UGAGCC | | | |
| SpCas9_3 | GCUAUUCAGGAGGUU | 877 | GCUAUUCAGGAGGUUGAGAU | 878 |
| 64 | GAGGU | | | |
| SpCas9_3 | AGCUAUUCAGGAGGU | 879 | AGCUAUUCAGGAGGUUGAGA | 880 |
| 65 | UGAGG | | | |
| SpCas9_3 | UCCUGAAUAGCUGGG | 881 | UCCUGAAUAGCUGGGACUAC | 882 |
| 66 | ACUAU | | | |
| SpCas9_3 | ACCUAUAGUCCCAGC | 883 | ACCUGUAGUCCCAGCUAUUC | 884 |
| 67 | UAUUC | | | |
| SpCas9_3 | UAUAGUCCCAGCUAU | 885 | UGUAGUCCCAGCUAUUCAGG | 886 |
| 68 | UCAGG | | | |
| SpCas9_3 | AAAAAAAAAUUAGCU | 887 | AAAAAAAAAUUAGCUUGGUA | 888 |
| 69 | UGGUA | | | |
| SpCas9_3 | AAAAAAAAAAAAAA | 889 | AAAAAAAAAAAAAAUUAGCU | 890 |
| 85 | UUAGCU | | | |
| SpCas9_4 | AAAAAAAAAUUAGCU | 891 | AAAAAAAAAUUAGCUUGGUA | 892 |
| 01 | UGGUA | | | |
| SpCas9_4 | AAAAAAAAAAAAAA | 893 | AAAAAAAAAAAAAAUUAGCU | 894 |
| 17 | UUAGCU | | | |
| SpCas9_4 | AAAAAAAAAUUAGCU | 895 | AAAAAAAAAUUAGCUUGGUA | 896 |
| 33 | UGGUA | | | |
| SpCas9_4 | AAAAAAAAAAAAAA | 897 | AAAAAAAAAAAAAAUUAGCU | 898 |
| 49 | UUAGCU | | | |
| SpCas9_4 | AAAAAAAAAUUAGCU | 899 | AAAAAAAAAUUAGCUUGGUA | 900 |
| 65 | UGGUA | | | |
| SpCas9_4 | AAAAAAAAAAAAAA | 901 | AAAAAAAAAAAAAAUUAGCU | 902 |
| 81 | UUAGCU | | | |
| SpCas9_4 | CACCUGUAAUCCCAG | 903 | CAUCUGUAAUCCCAGCACUU | 904 |
| 97 | CACUU | | | |
| SpCas9_4 | ACCUGUAAUCCCAGC | 905 | AUCUGUAAUCCCAGCACUUU | 906 |
| 98 | ACUUU | | | |
| SpCas9_4 | CUUGGCUCUCUAUUC | 907 | CUUGGCUCUGUAUUCUCCAU | 908 |
| 99 | UCCAU | | | |
| SpCas9_5 | CCCCACUACAAACUG | 909 | CCCCACUACAAACCGGGCAA | 910 |
| 00 | GGCAA | | | |
| SpCas9_5 | AACCCACCCCACUAC | 911 | AACCCACCCCACUACAAACC | 912 |
| 05 | AAACU | | | |
| SpCas9_5 | UGCCCAGUUUGUAGU | 913 | UGCCCGGUUUGUAGUGGGGU | 914 |
| 07 | GGGGU | | | |
| SpCas9_5 | UUGCCCAGUUUGUAG | 915 | UUGCCCGGUUUGUAGUGGGG | 916 |
| 08 | UGGGG | | | |
| SpCas9_5 | CCUUUGCCCAGUUUG | 917 | CCUUUGCCCGGUUUGUAGUG | 918 |
| 09 | UAGUG | | | |
| SpCas9_5 | UCCUUUGCCCAGUUU | 919 | UCCUUUGCCCGGUUUGUAGU | 920 |
| 10 | GUAGU | | | |
| SpCas9_5 | GUCCUUUGCCCAGUU | 921 | GUCCUUUGCCCGGUUUGUAG | 922 |
| 11 | UGUAG | | | |
| SpCas9_5 | AGAAAAAUUUGUAU | 923 | AGAACAAUUUGUAUUCCUUU | 924 |
| 12 | UCCUUU | | | |
| SpCas9_5 | AGAAUGGGUUUGCAA | 925 | AGAAUGGGUUUGCAACCGUU | 926 |
| 14 | CCAUU | | | |
| SpCas9_5 | GGAAAAAAGAAAAU | 927 | GGAAAAAAGAAAAUAUUUCA | 928 |
| 15 | GUUUCA | | | |
| SpCas9_5 | AAAAUGCUUGCAAUA | 929 | AAAAUGCUUGCAAUAAUGCC | 930 |
| 16 | AUGCC | | | |
| SpCas9_5 | AAAAUGCUUGCAAUA | 931 | AAAAUGCUUGCAAUAAUGCC | 932 |
| 25 | AUGCC | | | |
| SpCas9_5 | UCCCAAAGCUCUGUU | 933 | ACCCAAAGCUCUGUUCUUAC | 934 |
| 43 | CUUAC | | | |
| SpCas9_5 | GAGCUUUGGGAGUGC | 935 | GAGCUUUGGGUGUGCAAGGC | 936 |
| 45 | AAGGC | | | |
| SpCas9_5 | AACAGAGCUUUGGGA | 937 | AACAGAGCUUUGGGUGUGCA | 938 |
| 46 | GUGCA | | | |
| SpCas9_5 | CUUUGGGAGUGCAAG | 939 | CUUUGGGUGUGCAAGGCAGG | 940 |
| 47 | GCAGG | | | |
| SpCas9_5 | UCCCAAAGCUCUGUU | 941 | UCCCAAAGCUCUGUUCUUAU | 942 |
| 48 | CUUAC | | | |
| SpCas9_5 | GCCUGUAAGAACAGA | 943 | GCCUAUAAGAACAGAGCUUU | 944 |
| 50 | GCUUU | | | |
| SpCas9_5 | UGCCUGUAAGAACAG | 945 | UGCCUAUAAGAACAGAGCUU | 946 |
| 51 | AGCUU | | | |
| SpCas9_5 | UCCUUUUGAAUUUAC | 947 | UCCUUUUGAAUUUACUGUAC | 948 |
| 52 | UGUUC | | | |
| SpCas9_5 | GCCUGAACAGUAAAU | 949 | GCCUGUACAGUAAAUUCAAA | 950 |
| 53 | UCAAA | | | |
| SpCas9_5 | GAAGCUUUGUGCUGC | 951 | GAAGUUUUGUGCUGCCUUUC | 952 |
| 54 | CUUUC | | | |
| SpCas9_5 | ACUGGCUACCCCAAC | 953 | ACUGCCUACCCCAACUGAUU | 954 |
| 55 | UGAUU | | | |
| SpCas9_5 | UCAUCUUUGUAUAAU | 955 | UCAUCUUCGUAUAAUGUCAA | 956 |
| 56 | GUCAA | | | |
| SpCas9_5 | AUAGUUUUGAAAUU | 957 | ACAGUUUUGAAAUUUCCUCU | 958 |
| 57 | UCCUCU | | | |
| SpCas9_5 | UUUUUUAUUUCAUUU | 959 | UUUUUUAUUUCAUUUUGCUG | 960 |
| 58 | UGCCG | | | |
| SpCas9_5 | AAUUAUUUAUAAUAC | 961 | AAAUAUUUAUAAUACUUAAA | 962 |
| 59 | UUAAA | | | |
| SpCas9_5 | CAAUUAUUUAUAAUA | 963 | CAAAUAUUUAUAAUACUUAA | 964 |
| 60 | CUUAA | | | |
| SpCas9_5 | UUAAAAAAAAAAAA | 965 | GUUAAAAAAAAAAAAAGUCC | 966 |
| 61 | GUCCUG | | UG | |
| SpCas9_5 | UUUUAACACAUGCUU | 967 | UUUUAACACAUGCUUAAAUC | 968 |
| 63 | AAAUC | | | |
| SpCas9_5 | AGUCCUGAGGCUAGA | 969 | UGUCCUGAGGCUAGACAUGU | 970 |
| 64 | CAUGU | | | |
| SpCas9_5 | UUAAAAAAAAAAAA | 971 | UUAAAAAAAAAAAUGUCCUG | 972 |
| 65 | GUCCUG | | | |
| SpCas9_5 | CAGAAGCUAUAAAAG | 973 | CAGAAGCUAUAAAAGUUCCA | 974 |
| 67 | UUGCA | | | |
| SpCas9_5 | AAAAAAAAACACUCC | 975 | AAACAAAAAAACACUCCAAC | 976 |
| 68 | AACUA | | UA | |
| SpCas9_5 | UUAGUUUAUUAGCUA | 977 | UUGGUUUAUUAGCUAAUGCU | 978 |
| 69 | AUGCU | | | |
| SpCas9_5 | AGCAUUAGCUAAUAA | 979 | AGCAUUAGCUAAUAAACCAA | 980 |
| 70 | ACUAA | | | |
| SpCas9_5 | AUUAACUCAAGUAGC | 981 | ACUAACUCAAGUAGCACUGA | 982 |
| 71 | ACUGA | | | |
| SpCas9_5 | AGUGCUACUUGAGUU | 983 | AGUGCUACUUGAGUUAGUUA | 984 |
| 73 | AAUUA | | | |
| SpCas9_5 | CUCAAGUAGCACUGA | 985 | CUCAAGUAACACUGAAGGAA | 986 |
| 75 | AGGAA | | | |
| SpCas9_5 | UCAAGUAGCACUGAA | 987 | UCAAGUAACACUGAAGGAAA | 988 |
| 76 | GGAAA | | | |
| SpCas9_5 | AUUAACUCAAGUAGC | 989 | AUUAACUCAAGUAACACUGA | 990 |
| 77 | ACUGA | | | |
| SpCas9_5 | AGUGCUACUUGAGUU | 991 | AGUGUUACUUGAGUUAAUUA | 992 |
| 79 | AAUUA | | | |
| SpCas9_5 | CAUUUAUACACUACU | 993 | CAUUUAUACACAACUGACAC | 994 |
| 81 | GACAC | | | |
| SpCas9_5 | CAGUGUCAGUAGUGU | 995 | CAGUGUCAGUUGUGUAUAAA | 996 |
| 82 | AUAAA | | | |
| SpCas9_5 | UUAUAACAACCCUAC | 997 | UUAUAACAACCCCACACAUU | 998 |
| 83 | ACAUU | | | |
| SpCas9_5 | GUAAUAGUACCUAAU | 999 | GUAAUAGUACCUAAUGUGUG | 1000 |
| 84 | GUGUA | | | |
| SpCas9_5 | GUGCACAGAGAUUGU | 1001 | GUACACAGAGAUUGUUUUUU | 1002 |
| 85 | UUUUU | | | |
| SpCas9_5 | AGUGCACAGAGAUUG | 1003 | AGUACACAGAGAUUGUUUUU | 1004 |
| 86 | UUUUU | | | |
| SpCas9_5 | UGCACAGAGAUUGUU | 1005 | UACACAGAGAUUGUUUUUUG | 1006 |
| 87 | UUUUG | | | |
| SpCas9_5 | UGUGUGCUUAUUUGC | 1007 | UGUGUGCCUAUUUGCUAAAG | 1008 |
| 88 | UAAAG | | | |
| SpCas9_5 | AAUAAGCACACACAA | 1009 | AAUAGGCACACACAAAAUCC | 1010 |
| 89 | AAUCC | | | |
| SpCas9_5 | AUAUUCUAAUUUUAA | 1011 | AUAUUCUAAUUUUUAUAGUA | 1012 |
| 90 | UAGUA | | | |
| SpCas9_5 | UUCUUUUUUUUUUUU | 1013 | UUUUCUUUUUUUUUUUUUUU | 1014 |
| 91 | UGAGA | | GAGA | |
| SpCas9_5 | UUCUUUUUUUUUUUU | 1015 | UUCUUUUUUUUUUUUUGAGA | 1016 |
| 93 | UGAGA | | | |
| SpCas9_5 | UUUCGCCAUGUUGGC | 1017 | UUUCGCCAUGUUGGCCAGGC | 1018 |
| 95 | CAGGU | | | |
| SpCas9_5 | UCAGAAAUUUGACAC | 1019 | UCAGAAAUUUGACACCAGCC | 1020 |
| 96 | CAACC | | | |
| SpCas9_5 | UGACACCAACCUGGC | 1021 | UGACACCAGCCUGGCCAACA | 1022 |
| 97 | CAACA | | | |
| SpCas9_5 | UUUGCAAGAACUCAA | 1023 | UUUGCAAGAACUCAGUCAAA | 1024 |
| 98 | UCAAA | | | |
| SpCas9_5 | GGUGUUGUUGAGCUU | 1025 | GGUGUUGUUGAGCUUCAGUG | 1026 |
| 99 | CAGUG | | | |
| SpCas9_6 | GGUGUUGUUGAGCUU | 1027 | UUGGUGUUGUUGAGCUUCAG | 1028 |
| 03 | CAGUG | | UG | |
| SpCas9_6 | GUUCAAGUUUGGGAA | 1029 | GUUCGAGUUUGGGAAGUACA | 1030 |
| 05 | GUACA | | | |
| SpCas9_6 | CUGCUGAGAAGUUCA | 1031 | CUGCUGAGAAGUUCGAGUUU | 1032 |
| 06 | AGUUU | | | |
| SpCas9_6 | ACUGCUGAGAAGUUC | 1033 | ACUGCUGAGAAGUUCGAGUU | 1034 |
| 07 | AAGUU | | | |
| SpCas9_6 | CAAGUUUGGGAAGUA | 1035 | CGAGUUUGGGAAGUACAAGG | 1036 |
| 08 | CAAGG | | | |
| SpCas9_6 | UCAACUUAAGUAAAG | 1037 | UCAACUUAAGCAAAGGUUUC | 1038 |
| 09 | GUUUC | | | |
| SpCas9_6 | AUAGUAGUCAACUUA | 1039 | AUAGUAGUCAACUUAAGCAA | 1040 |
| 10 | AGUAA | | | |
| SpCas9_6 | AGUAAACUUAUCGCA | 1041 | AGUAAACUUACCGCAAGUUG | 1042 |
| 11 | AGUUG | | | |
| SpCas9_6 | GACUUAGAAGAAAUA | 1043 | GACUUAAAAGAAAUAUUGUG | 1044 |
| 12 | UUGUG | | | |
| SpCas9_6 | GAAGAAAUAUUGUG | 1045 | AAAGAAAUAUUGUGUGGACC | 1046 |
| 13 | UGGACC | | | |
| SpCas9_6 | UGUCUCUGAUAUGUU | 1047 | UGUCUCUGAUAUGUUCUUUA | 1048 |
| 14 | CUUUA | | | |
| SpCas9_6 | CUGGGCAAACAGAAA | 1049 | CUGGGCAAACAGAUAAAAAA | 1050 |
| 15 | AAAAA | | | |
| SpCas9_6 | UGGGCAAACAGAAAA | 1051 | UGGGCAAACAGAUAAAAAAA | 1052 |
| 16 | AAAAA | | | |
| SpCas9_6 | UAGGGAAAAAAUGG | 1053 | UAGGGAAACAAUGGGGGCAG | 1054 |
| 17 | GGGCAG | | | |
| SpCas9_6 | GAAAAAAUGGGGGCA | 1055 | GAAACAAUGGGGGCAGGGGU | 1056 |
| 18 | GGGGU | | | |
| SpCas9_6 | UUUCUACUUUAGGGA | 1057 | UUUCUACUUUAGGGAAACAA | 1058 |
| 19 | AAAAA | | | |
| SpCas9_6 | UUCUACUUUAGGGAA | 1059 | UUCUACUUUAGGGAAACAAU | 1060 |
| 20 | AAAAU | | | |
| SpCas9_6 | UCUACUUUAGGGAAA | 1061 | UCUACUUUAGGGAAACAAUG | 1062 |
| 21 | AAAUG | | | |
| SpCas9_6 | CUACUUUAGGGAAAA | 1063 | CUACUUUAGGGAAACAAUGG | 1064 |
| 22 | AAUGG | | | |
| SpCas9_6 | UUUAGGGAAAAAAU | 1065 | UUUAGGGAAACAAUGGGGGC | 1066 |
| 23 | GGGGGC | | | |
| SpCas9_6 | UUAGGGAAAAAAUG | 1067 | UUAGGGAAACAAUGGGGGCA | 1068 |
| 24 | GGGGCA | | | |
| SpCas9_6 | GUUUCAUAAUGUGAG | 1069 | GUUUUAUAAUGUGAGAAAAA | 1070 |
| 25 | AAAAA | | | |
| SpCas9_6 | AUUAUGAAACUCUUA | 1071 | AUUAUAAAACUCUUAAAAUC | 1072 |
| 26 | AAAUC | | | |
| SpCas9_6 | AGCAUUAUAUAUUCA | 1073 | ACCAUUAUAUAUUCAACUAC | 1074 |
| 27 | ACUAC | | | |
| SpCas9_6 | CAACGGGCAGUUGCA | 1075 | CAACGGGCAGUUACAGCAGC | 1076 |
| 28 | GCAGC | | | |
| SpCas9_6 | GCAGUUGCAGCAGCU | 1077 | GCAGUUACAGCAGCUGGACU | 1078 |
| 29 | GGACU | | | |
| SpCas9_6 | UCAGCUUCUGGGCAG | 1079 | UCAGCUUCUGGGCAGGCCAC | 1080 |
| 30 | GCCAU | | | |
| SpCas9_6 | CAGCUUCUGGGCAGG | 1081 | CAGCUUCUGGGCAGGCCACA | 1082 |
| 31 | CCAUA | | | |
| SpCas9_6 | UAGGGAACUUAAAAU | 1083 | CAGGGAACUUAAAAUCAUGA | 1084 |
| 32 | CAUGA | | | |
| SpCas9_6 | UCAUGAUUUUAAGUU | 1085 | UCAUGAUUUUAAGUUCCCUG | 1086 |
| 33 | CCCUA | | | |
| SpCas9_6 | UUUUGAUUUUACAGG | 1087 | UUUUGAUUUUACAGGUUCAC | 1088 |
| 34 | UUCAU | | | |
| SpCas9_6 | UUACAGGUUCAUAGG | 1089 | UUACAGGUUCACAGGUAGAA | 1090 |
| 35 | UAGAA | | | |
| SpCas9_6 | UUUACAGGUUCAUAG | 1091 | UUUACAGGUUCACAGGUAGA | 1092 |
| 36 | GUAGA | | | |
| SpCas9_6 | UGAGUGGGCUCCCAA | 1093 | UGAGUGGGCUCCGAAAACCU | 1094 |
| 37 | AACCU | | | |
| SpCas9_6 | GAGUGGGCUCCCAAA | 1095 | GAGUGGGCUCCGAAAACCUU | 1096 |
| 38 | ACCUU | | | |
| SpCas9_6 | UGUCUUGUCAUAGCU | 1097 | UCUCUUGUCAUAGCUCUGCU | 1098 |
| 39 | CUGCU | | | |
| SpCas9_6 | AUCACUGGGUAUAUA | 1099 | AUCCCUGGGUAUAUACCCAA | 1100 |
| 40 | CCCAA | | | |
| SpCas9_6 | UGACCCAGCCAUCCC | 1101 | UGACCCAGCCAUCCCAUCCC | 1102 |
| 41 | AUCAC | | | |
| SpCas9_6 | GACCCAGCCAUCCCA | 1103 | GACCCAGCCAUCCCAUCCCU | 1104 |
| 42 | UCACU | | | |
| SpCas9_6 | UUUGGGUAUAUACCC | 1105 | UUUGGGUAUAUACCCAGGGA | 1106 |
| 43 | AGUGA | | | |
| SpCas9_6 | UACCCAGUGAUGGGA | 1107 | UACCCAGGGAUGGGAUGGCU | 1108 |
| 44 | UGGCU | | | |
| SpCas9_6 | AUACCCAGUGAUGGG | 1109 | AUACCCAGGGAUGGGAUGGC | 1110 |
| 45 | AUGGC | | | |
| SpCas9_6 | GUAUAUACCCAGUGA | 1111 | GUAUAUACCCAGGGAUGGGA | 1112 |
| 46 | UGGGA | | | |
| SpCas9_6 | UUGGGUAUAUACCCA | 1113 | UUGGGUAUAUACCCAGGGAU | 1114 |
| 47 | GUGAU | | | |
| SpCas9_6 | UAUUCACAAUAGCAA | 1115 | UGUUCACAAUAGCAAAGACU | 1116 |
| 48 | AGACU | | | |
| SpCas9_6 | UCACACUCUGGGGAC | 1117 | UCACACUCCGGGGACUGUUG | 1118 |
| 49 | UGUUG | | | |
| SpCas9_6 | CACACUCUGGGGACU | 1119 | CACACUCCGGGGACUGUUGU | 1120 |
| 50 | GUUGU | | | |
| SpCas9_6 | ACACUCUGGGGACUG | 1121 | ACACUCCGGGGACUGUUGUG | 1122 |
| 51 | UUGUG | | | |
| SpCas9_6 | CUCUGGGGACUGUUG | 1123 | CUCCGGGGACUGUUGUGGGG | 1124 |
| 52 | UGGGG | | | |
| SpCas9_6 | UCUGGGGACUGUUGU | 1125 | UCCGGGGACUGUUGUGGGGU | 1126 |
| 53 | GGGGU | | | |
| SpCas9_6 | CUGGGGACUGUUGUG | 1127 | CCGGGGACUGUUGUGGGGUG | 1128 |
| 54 | GGGUG | | | |
| SpCas9_6 | UGGGGACUGUUGUGG | 1129 | CGGGGACUGUUGUGGGGUGG | 1130 |
| 55 | GGUGG | | | |
| SpCas9_6 | GAAGGGGAACAUCAC | 1131 | GAAGGGGAACAUCACACUCC | 1132 |
| 57 | ACUCU | | | |
| SpCas9_6 | AAGGGGAACAUCACA | 1133 | AAGGGGAACAUCACACUCCG | 1134 |
| 59 | CUCUG | | | |
| SpCas9_6 | GAAAGACUCAUAGGA | 1135 | GAAAGACUCAUAGGACUAAU | 1136 |
| 60 | CUAAC | | | |
| SpCas9_6 | GUGCUCAUGCCUAUG | 1137 | GUGCUCAUGCCUAUGAACAU | 1138 |
| 61 | AGCAU | | | |
| SpCas9_6 | UUCUAAUAGCCUAUG | 1139 | UUCUAAUAGCCUAUGUUCAU | 1140 |
| 62 | CUCAU | | | |
| SpCas9_6 | UUCACAGAUCCCUAG | 1141 | UUCACAGAUACCUAGAACAG | 1142 |
| 63 | AACAG | | | |
| SpCas9_6 | UGACUUUGGAACCGG | 1143 | UGACUUUGGAACCUGCUAAC | 1144 |
| 65 | CUAAC | | | |
| SpCas9_6 | GGAACAGAGAGAGGG | 1145 | GGAACAGAGAGAGGGAAAGG | 1146 |
| 67 | AAAGU | | | |
| SpCas9_6 | GAACAGAGAGAGGGA | 1147 | GAACAGAGAGAGGGAAAGGG | 1148 |
| 69 | AAGUG | | | |
| SpCas9_6 | AACAGAGAGAGGGAA | 1149 | AACAGAGAGAGGGAAAGGGG | 1150 |
| 70 | AGUGG | | | |
| SpCas9_6 | AGAGAGAGGGAAAG | 1151 | AGAGAGAGGGAAAGGGGGGG | 1152 |
| 71 | UGGGGG | | | |
| SpCas9_6 | AGGCUCCAUCUCCAA | 1153 | AGGCUCCAUCUCCAACGCUG | 1154 |
| 72 | CGUUG | | | |
| SpCas9_6 | CCAGGCUCCAUCUCC | 1155 | CCAGGCUCCAUCUCCAACGC | 1156 |
| 73 | AACGU | | | |
| SpCas9_6 | CAGGCUCCAUCUCCA | 1157 | CAGGCUCCAUCUCCAACGCU | 1158 |
| 74 | ACGUU | | | |
| SpCas9_6 | UCAGUUGCAAUCCCC | 1159 | UCAGUUGCAAUCCCCAGCGU | 1160 |
| 75 | AACGU | | | |
| SpCas9_6 | GCAAUCCCCAACGUU | 1161 | GCAAUCCCCAGCGUUGGAGA | 1162 |
| 76 | GGAGA | | | |
| SpCas9_6 | CCAACGUUGGAGAUG | 1163 | CCAGCGUUGGAGAUGGAGCC | 1164 |
| 77 | GAGCC | | | |
| SpCas9_6 | AAUCUUAUCACAUAC | 1165 | AAUCUUAUCGCAUACAUAAA | 1166 |
| 78 | AUAAA | | | |
| SpCas9_6 | UUUAGAAUAAGACUG | 1167 | UUUAGAGUAAGACUGGAACU | 1168 |
| 79 | GAACU | | | |
| SpCas9_6 | UCCCCAGUUUAGAAU | 1169 | UCCCCAGUUUAGAGUAAGAC | 1170 |
| 80 | AAGAC | | | |
| SpCas9_6 | UCCAGUCUUAUUCUA | 117 | UCCAGUCUUACUCUAAACUG | 1172 |
| 81 | AACUG | | | |
| SpCas9_6 | UUCCAGUCUUAUUCU | 1173 | UUCCAGUCUUACUCUAAACU | 1174 |
| 82 | AAACU | | | |
| SpCas9_6 | GUUCCAGUCUUAUUC | 1175 | GUUCCAGUCUUACUCUAAAC | 1176 |
| 83 | UAAAC | | | |
| SpCas9_6 | GACACAGCAGGAAGU | 1177 | GACACAGCAGGAAGGUUAUC | 1178 |
| 84 | UUAUC | | | |
| SpCas9_6 | UUCACUCUUGUUGUC | 1179 | UUCACUCUUGUUGUCCAAGC | 1180 |
| 85 | CAGGC | | | |
| SpCas9_6 | UUGUCCAGGCUGGAG | 1181 | UUGUCCAAGCUGGAGUGCAA | 1182 |
| 86 | UGCAA | | | |
| SpCas9_6 | CGUGCCAUUGCACUC | 1183 | CGUGCCAUUGCACUCCAGCU | 1184 |
| 87 | CAGCC | | | |
| SpCas9_6 | UCCCGAAGUGCUGAG | 1185 | UCCCAAAGUGCUGAGAUUAC | 1186 |
| 88 | AUUAC | | | |
| SpCas9_6 | CUCAGCACUUCGGGA | 1187 | CUCAGCACUUUGGGAGGCCA | 1188 |
| 89 | GGCCA | | | |
| SpCas9_6 | UGUAAUCUCAGCACU | 1189 | UGUAAUCUCAGCACUUUGGG | 1190 |
| 90 | UCGGG | | | |
| SpCas9_6 | ACCUGUAAUCUCAGC | 1191 | ACCUGUAAUCUCAGCACUUU | 1192 |
| 91 | ACUUC | | | |
| SpCas9_6 | GCACUUCGGGAGGCC | 1193 | GCACUUUGGGAGGCCAAGGA | 1194 |
| 92 | AAGGA | | | |
| SpCas9_6 | AGCACUUCGGGAGGC | 1195 | AGCACUUUGGGAGGCCAAGG | 1196 |
| 93 | CAAGG | | | |
| SpCas9_6 | UGCCUUAAUCAUUAC | 1197 | UGCCUCAAUCAUUACGGACU | 1198 |
| 94 | GGACU | | | |
| SpCas9_6 | UGUGGUUGCCUUAAU | 1199 | UGUGGUUGCCUCAAUCAUUA | 1200 |
| 96 | CAUUA | | | |
| SpCas9_6 | CUUAAUCAUUACGGA | 1201 | CUCAAUCAUUACGGACUAGG | 1202 |
| 97 | CUAGG | | | |
| SpCas9_6 | CGCCUAGUCCGUAAU | 1203 | CGCCUAGUCCGUAAUGAUUG | 1204 |
| 99 | GAUUA | | | |
| SpCas9_7 | UGCCUUAAUCAUUAC | 1205 | UGCCUUAAUCAUUACAGACU | 1206 |
| 01 | GGACU | | | |
| SpCas9_7 | CUUAAUCAUUACGGA | 1207 | CUUAAUCAUUACAGACUAGG | 1208 |
| 03 | CUAGG | | | |
| SpCas9_7 | CGCCUAGUCCGUAAU | 1209 | CGCCUAGUCUGUAAUGAUUA | 1210 |
| 05 | GAUUA | | | |
| SpCas9_7 | GGGGUUUCACCAUGU | 1211 | GGAGUUUCACCAUGUUAGUC | 1212 |
| 07 | UAGUC | | | |
| SpCas9_7 | UUGUAUUUUUAGUA | 1213 | UUGUAUUUUUAGUAGAGAUG | 1214 |
| 08 | GAGAUG | | | |
| SpCas9_7 | UUUGUAUUUUUAGU | 1215 | UUUGUAUUUUUAGUAGAGAU | 1216 |
| 10 | AGAGAU | | | |
| SpCas9_7 | UUUUGUAUUUUUAG | 1217 | UUUUGUAUUUUUAGUAGAGA | 1218 |
| 12 | UAGAGA | | | |
| SpCas9_7 | GAAUACAGAGUUCUG | 1219 | GAAUACAGAGUUCUUGGGAG | 1220 |
| 13 | GGGAG | | | |
| SpCas9_7 | GCCUGGAAUACAGAG | 1221 | GCCUGGAAUACAGAGUUCUU | 1222 |
| 15 | UUCUG | | | |
| SpCas9_7 | UCCCCAGAACUCUGU | 1223 | UCCCAAGAACUCUGUAUUCC | 1224 |
| 17 | AUUCC | | | |
| SpCas9_7 | AAAGCUAGGCCAGUU | 1225 | AAAGCUAGGCCGGUUGCUCA | 1226 |
| 19 | GCUCA | | | |
| SpCas9_7 | UAAAGCUAGGCCAGU | 1227 | UAAAGCUAGGCCGGUUGCUC | 1228 |
| 20 | UGCUC | | | |
| SpCas9_7 | AGAGAAGCUUGAAAG | 1229 | AGAGAAGCUUCAAAGUCAAG | 1230 |
| 21 | UCAAG | | | |
| SpCas9_7 | UGGAGAGGUGAGGAC | 1231 | UGGAGAGGUGGGGACAUUUC | 1232 |
| 22 | AUUUC | | | |
| SpCas9_7 | CAAUUUCCUCCUCUU | 1233 | CAAAUUCCUCCUCUUGAAAA | 1234 |
| 23 | GAAAA | | | |
| SpCas9_7 | UUUUCAAGAGGAGGA | 1235 | UUUUCAAGAGGAGGAAUUUG | 1236 |
| 24 | AAUUG | | | |
| SpCas9_7 | AAAAAAAAAAAGCUC | 1237 | AAAAAAAAAAAGCUCUUACC | 1238 |
| 25 | UUACC | | | |
| SpCas9_7 | AAAAGCUCUUACCUG | 1239 | AAAAGCUCUUACCUGGUUGC | 1240 |
| 29 | GUUGC | | | |
| SpCas9_7 | AAAAAAAAAAAGCUC | 1241 | AAAAAAAAAAAGCUCUUACC | 1242 |
| 33 | UUACC | | | |
| SpCas9_7 | AAAAGCUCUUACCUG | 1243 | AAAAGCUCUUACCUGGUUGC | 1244 |
| 37 | GUUGC | | | |
| SpCas9_7 | UUUUUUUUUAAAUCA | 1245 | UUUUUUUUAAAAUCAACAUA | 1246 |
| 41 | ACAUA | | | |
| SpCas9_7 | AAACAACUGAUAGUC | 1247 | AAACAACUGAUAAUCGUCAG | 1248 |
| 42 | GUCAG | | | |
| SpCas9_7 | AUGCCUAAAUAAUGU | 1249 | AUGCCUAAAUAAUGUCAUCG | 1250 |
| 43 | CAUUG | | | |
| SpCas9_7 | UUUCCACAAUGACAU | 1251 | UUUCCACGAUGACAUUAUUU | 1252 |
| 44 | UAUUU | | | |
| SpCas9_7 | GCAGAAAGUGACAGU | 1253 | GCAGAAAGUGACUGUGAGAC | 1254 |
| 45 | GAGAC | | | |
| SpCas9_7 | AGUGACAGUGAGACU | 1255 | AGUGACUGUGAGACUGGAAA | 1256 |
| 46 | GGAAA | | | |
| SpCas9_7 | AAGUGACAGUGAGAC | 1257 | AAGUGACUGUGAGACUGGAA | 1258 |
| 47 | UGGAA | | | |
| SpCas9_7 | CAGUGAGACUGGAAA | 1259 | CUGUGAGACUGGAAAGGGAU | 1260 |
| 48 | GGGAU | | | |
| SpCas9_7 | UCACUCUCACUCUGU | 1261 | UCAUUCUCACUCUGUUGCCC | 1262 |
| 49 | UGCCC | | | |
| SpCas9_7 | GUGAGAGUGAGACUC | 1263 | GUGAGAAUGAGACUCUUUCU | 1264 |
| 50 | UUUCU | | | |
| SpCas9_7 | AACUCUGGGGGAAAA | 1265 | AACUCUGGGGGAAAAAAAGU | 1266 |
| 51 | GAAGU | | | |
| SpCas9_7 | CUUCUUUUCCCCCAG | 1267 | CUUUUUUUCCCCCAGAGUUC | 1268 |
| 52 | AGUUC | | | |
| SpCas9_7 | GAUGAAUGAAUCUUG | 1269 | GAUGAAUGAAUUUUGUUUAC | 1270 |
| 53 | UUUAC | | | |
| SpCas9_7 | AUGAAUGAAUCUUGU | 1271 | AUGAAUGAAUUUUGUUUACA | 1272 |
| 54 | UUACA | | | |
| SpCas9_7 | AGAUUCAUUCAUCUC | 1273 | AAAUUCAUUCAUCUCAAAUU | 1274 |
| 55 | AAAUU | | | |
| SpCas9_7 | AAGAUUCAUUCAUCU | 1275 | AAAAUUCAUUCAUCUCAAAU | 1276 |
| 56 | CAAAU | | | |
| SpCas9_7 | GAGAAGUUUUUAUCU | 1277 | GAGAAGUUCUUAUCUGGUGA | 1278 |
| 57 | GGUGA | | | |
| SpCas9_7 | CUCUGUGAGAAGUUU | 1279 | CUCUGUGAGAAGUUCUUAUC | 1280 |
| 58 | UUAUC | | | |
| SpCas9_7 | AGAAGAAAUGUCUAU | 1281 | AGAAAAAAUGUCUAUGAAAG | 1282 |
| 59 | GAAAG | | | |
| SpCas9_7 | GACAUUUCUUCUGCA | 1283 | GACAUUUUUUCUGCAUGGGU | 1284 |
| 61 | UGGGU | | | |
| SpCas9_7 | CAUAGACAUUUCUUC | 1285 | CAUAGACAUUUUUUCUGCAU | 1286 |
| 62 | UGCAU | | | |
| SpCas9_7 | UCAUAGACAUUUCUU | 1287 | UCAUAGACAUUUUUUCUGCA | 1288 |
| 64 | CUGCA | | | |
| SpCas9_7 | UGUCUAUGAAAGCGG | 1289 | UGUCUGUGAAAGCGGUCAAA | 1290 |
| 66 | UCAAA | | | |
| SpCas9_7 | AUGUCUAUGAAAGCG | 1291 | AUGUCUGUGAAAGCGGUCAA | 1292 |
| 67 | GUCAA | | | |
| SpCas9_7 | AGAAGAAAUGUCUAU | 1293 | AGAAGAAAUGUCUGUGAAAG | 1294 |
| 68 | GAAAG | | | |
| SpCas9_7 | CAUAGACAUUUCUUC | 1295 | CACAGACAUUUCUUCUGCAU | 1296 |
| 70 | UGCAU | | | |
| SpCas9_7 | UCAUAGACAUUUCUU | 1297 | UCACAGACAUUUCUUCUGCA | 1298 |
| 72 | CUGCA | | | |
| SpCas9_7 | AAUGAGUAGCUCUGC | 1299 | AAUGAGUAGCUUUGCAAAAG | 1300 |
| 74 | AAAAG | | | |
| SpCas9_7 | UCUGCAAAAGUGGAU | 1301 | UUUGCAAAAGUGGAUUUUCC | 1302 |
| 75 | UUUCC | | | |
| SpCas9_7 | CUACUCAUUUUUUGA | 1303 | CUACUCAUUUUUCGAUAAUU | 1304 |
| 76 | UAAUU | | | |
| SpCas9_7 | GCUACUCAUUUUUUG | 1305 | GCUACUCAUUUUUCGAUAAU | 1306 |
| 77 | AUAAU | | | |
| SpCas9_7 | AAGACAUACAAAUGG | 1307 | AAGACGUACAAAUGGCAAAC | 1308 |
| 78 | CAAAC | | | |
| SpCas9_7 | UUCAAGAGAAGACAU | 1309 | UUCAAGAGAAGACGUACAAA | 1310 |
| 80 | ACAAA | | | |
| SpCas9_7 | AAGACAUACAAAUGG | 1311 | AAGACAUACAAGUGGCAAAC | 1312 |
| 82 | CAAAC | | | |
| SpCas9_7 | UUCAAGAGAAGACAU | 1313 | UUCAAGAGAAGACAUACAAG | 1314 |
| 84 | ACAAA | | | |
| SpCas9_7 | UAUGUGUUCUGCCUC | 1315 | UACGUGUUCUGCCUCCCCUA | 1316 |
| 86 | CCCUA | | | |
| SpCas9_7 | CUCUUUUUUUUUUUU | 1317 | CCUCCUUUUUUUUUUUUUUU | 1318 |
| 87 | UUUGA | | GA | |
| SpCas9_7 | CUCUUUUUUUUUUUU | 1319 | CCUCUUUUUUUUUUUUUUUU | 1320 |
| 91 | UUUGA | | GA | |
| SpCas9_7 | CGUCAAAAAAAAAAA | 1321 | GUCAAAAAAAAAAAAAAAAG | 1322 |
| 95 | AAAAG | | | |
| SpCas9_7 | CUCUUUUUUUUUUUU | 1323 | UCCUCUUUUUUUUUUUUUUU | 1324 |
| 98 | UUUGA | | UUGA | |
| SpCas9_8 | CGUCAAAAAAAAAAA | 1325 | UCAAAAAAAAAAAAAAAAAG | 1326 |
| 02 | AAAAG | | | |
| SpCas9_8 | UUUUAUUUAUUAAU | 1327 | CCCCCCCCCCCCCCCCCCCC | 1328 |
| 30 | UGCCUA | | | |
| SpCas9_8 | GGUUUCUUUCUUCUG | 1329 | CCCCCCCCCCCCCCCCCCCC | 1330 |
| 40 | UAGUG | | | |
| SpCas9_8 | CCCAGCUAUUCAGGA | 1331 | CCCCCCCCCCCCCCCCCCCC | 1332 |
| 50 | GGUUG | | | |
| SpCas9_8 | CUCCCAAAGUGCUGG | 1333 | GGGGGGGGGGGGGGGGGGGG | 1334 |
| 70 | GAUUA | | | |
| SpCas9_8 | CUUUCUUGAGGAUGU | 1335 | CCCCCCCCCCCCCCCCCCCC | 1336 |
| 77 | AGCCA | | | |
| SpCas9_8 | AAAUGUGAAAGCUGG | 1337 | GGGGGGGGGGGGGGGGGGGG | 1338 |
| 87 | UCAAG | | | |
| SpCas9_8 | UCUGGGCAAACAGAA | 1339 | GGGGGGGGGGGGGGGGGGGG | 1340 |
| 94 | AAAAA | | | |
| SpCas9_9 | GGUGGUUCUGCCCAA | 1341 | CCCCCCCCCCCCCCCCCCCC | 1342 |
| 01 | GGUUU | | | |
| SpCas9_9 | UGCAUUGUUCCUCUG | 1343 | CCCCCCCCCCCCCCCCCCCC | 1344 |
| 11 | UUCUA | | | |
| SpCas9_9 | UGUAGAAGUGACUUU | 1345 | CCCCCCCCCCCCCCCCCCCC | 1346 |
| 21 | GGAAC | | | |
| SpCas9_9 | AGAGUUUCACUCUUG | 1347 | GGGGGGGGGGGGGGGGGGGG | 1348 |
| 31 | UUGUC | | | |
| SpCas9_9 | AUGUGGUUGCCUUAA | 1349 | GGGGGGGGGGGGGGGGGGGG | 1350 |
| 38 | UCAUU | | | |
| SpCas9_9 | UUGUAUUUUUAGUA | 1351 | CCCCCCCCCCCCCCCCCCCC | 1352 |
| 45 | GAGAUG | | | |
| SpCas9_9 | UUUGUAUUUUUAGU | 1353 | CCCCCCCCCCCCCCCCCCCC | 1354 |
| 65 | AGAGAU | | | |
| SpCas9_9 | AAGGCCUGGAAUACA | 1355 | GGGGGGGGGGGGGGGGGGGG | 1356 |
| 85 | GAGUU | | | |
| SpCas9_9 | UUACAUUUUGGGGGC | 1357 | GGGGGGGGGGGGGGGGGGGG | 1358 |
| 92 | AAAUA | | | |
| SpCas9_9 | AGGGACUGGUUUCAU | 1359 | CCCCCCCCCCCCCCCCCCCC | 1360 |
| 99 | GACUA | | | |
| SpCas9_1 | GGGGGGGGGGGGGG | 1361 | UAUAUUAAUAUUAUAAUAAU | 1362 |
| 009 | GGGGGG | | | |
| SpCas9_1 | GGGGGGGGGGGGGG | 1363 | AUGUACAAAGGUUUUUCUUC | 1364 |
| 034 | GGGGGG | | | |
| SpCas9_1 | GGGGGGGGGGGGGG | 1365 | CCUCCUGGGUUCAAGCGAUU | 1366 |
| 059 | GGGGGG | | | |
| SpCas9_1 | GGGGGGGGGGGGGG | 1367 | CUCACUGCAACCUACACCUC | 1368 |
| 084 | GGGGGG | | | |
| SpCas9_1 | GGGGGGGGGGGGGG | 1369 | UACUUUUGCAGGGGGUGGAG | 1370 |
| 109 | GGGGGG | | | |
| SpCas9_1 | GGGGGGGGGGGGGG | 1371 | GGCAAAGGACAUGAAAAUUU | 1372 |
| 134 | GGGGGG | | | |
| SpCas9_1 | GGGGGGGGGGGGGG | 1373 | CUUUCCUAAUCAGUUGGGGU | 1374 |
| 159 | GGGGGG | | | |
| SpCas9_1 | GGGGGGGGGGGGGG | 1375 | UUCUCAGACUUAUUUGACCA | 1376 |
| 184 | GGGGGG | | | |
| SpCas9_1 | GGGGGGGGGGGGGG | 1377 | AAGAAAAGUGCAAAAUCCUU | 1378 |
| 209 | GGGGGG | | | |
| SpCas9_1 | GGGGGGGGGGGGGG | 1379 | CACAUUAGGUACUAUUACUA | 1380 |
| 234 | GGGGGG | | | |
| SpCas9_1 | GGGGGGGGGGGGGG | 1381 | GCACACAGUUUUGUUUCAAA | 1382 |
| 259 | GGGGGG | | | |
| SpCas9_1 | GGGGGGGGGGGGGG | 1383 | AUUUGCUAAAGUGGCUAAUA | 1384 |
| 284 | GGGGGG | | | |
| SpCas9_1 | GGGGGGGGGGGGGG | 1385 | AUCCUUUAGUAACCUCCACA | 1386 |
| 309 | GGGGGG | | | |
| SpCas9_1 | GGGGGGGGGGGGGG | 1387 | UUCUAAGUUUCCUCAACUUG | 1388 |
| 334 | GGGGGG | | | |
| SpCas9_1 | GGGGGGGGGGGGGG | 1389 | UUAUAUAUUCAACUACAGGA | 1390 |
| 359 | GGGGGG | | | |
| SpCas9_1 | GGGGGGGGGGGGGG | 1391 | UGGGUAUAUACCCAAAGGAC | 1392 |
| 384 | GGGGGG | | | |
| SpCas9_1 | GGGGGGGGGGGGGG | 1393 | GGGUAUAUACCCAAAGGACU | 1394 |
| 409 | GGGGGG | | | |
| SpCas9_1 | GGGGGGGGGGGGGG | 1395 | GGGACUGUUGUGGGGUGGGG | 1396 |
| 434 | GGGGGG | | | |
| SpCas9_1 | GGGGGGGGGGGGGG | 1397 | GGAGGAACAGAGAGAGGGAA | 1398 |
| 459 | GGGGGG | | | |
| SpCas9_1 | GGGGGGGGGGGGGG | 1399 | GAGGAACAGAGAGAGGGAAA | 1400 |
| 484 | GGGGGG | | | |
| SpCas9_1 | GGGGGGGGGGGGGG | 1401 | UCCUGCUGUGUCAAAAAUGC | 1402 |
| 509 | GGGGGG | | | |
| SpCas9_1 | GGGGGGGGGGGGGG | 1403 | GCCUAGCUUUACCCCUCUAC | 1404 |
| 534 | GGGGGG | | | |
| SpCas9_1 | GGGGGGGGGGGGGG | 1405 | ACCUCUCCAUUUUGUGUGAG | 1406 |
| 559 | GGGGGG | | | |
| SpCas9_1 | GGGGGGGGGGGGGG | 1407 | CCUCUCCAUUUUGUGUGAGC | 1408 |
| 584 | GGGGGG | | | |
| SpCas9_1 | GGGGGGGGGGGGGG | 1409 | UACAUUAUAAAUCAGGAGAC | 1410 |
| 609 | GGGGGG | | | |

What is claimed is:

1. A composition for correcting a C9orf72 GC repeat expansion mutation comprising a guide nucleic acid sequence complementary to a target site in cis with the 2 mutation, wherein the guide nucleic acid sequence is at least 90% identical to a sequence set out in Table 1, Table 2, Table 8, or Table 9.

2. A composition for correcting a C9orf72 GC repeat expansion mutation comprising a guide nucleic acid sequence complementary to a target site in cis with the 2 mutation, wherein the target site is located in a region between 25 kbp upstream and 28 kbp downstream of a transcription start site of the C9orf72 gene.

3. A nucleic acid encoding CRISPR-Cas ribonucleoprotein (RNP) complex for correcting a C9orf72 GC repeat expansion mutation comprising a sequence of a guide nucleic acid having a sequence set out in Table 1, Table 2, Table 8, or Table 9, wherein the nucleic acid is delivered to a target site through a functional carrier.

4. The nucleic acid of claim 3, wherein the functional carrier is selected from the group consisting of viral vectors, a modified RNA binding protein and a compound disclosed in U.S. Pat. No. 10,085,1367.

5. A method of correcting a C9orf72 GC repeat expansion mutation in a host cell comprising administering to the host cell an endonuclease and two or more guide nucleic acids having a sequence set out in Table 1, Table 2, Table 8, or Table 9.

6. The method of claim 5, wherein a first guide nucleic acid is targeting a sequence upstream of the GC repeat expansion region, wherein a second guide nucleic acid is targeting a sequence downstream of the GC repeat expansion region, wherein the first guide nucleic acid sequence comprising SEQ ID NO. 1, or SEQ ID NO. 731, wherein the second guide nucleic acid sequence comprising SEQ ID NO: 2, or SEQ ID NO. 732, and further comprising the steps of:
a. excising a region that contains a GC repeats expansion in the mutant allele by cleaving one or both strands of DNA at a first target nucleic acid sequence and at a second target nucleic acid sequence with the endonuclease; and
b. excising a region in the normal allele by cleaving one or both strands of DNA at a first target nucleic acid sequence and at a second target nucleic acid sequence with the endonuclease.

7. The method of claim 5, wherein a first guide nucleic acid is targeting a sequence upstream of the exon 1A at the C9orf72 locus, wherein a second guide nucleic acid is targeting a sequence downstream of the exon 1B at the C9orf72 locus, wherein the first guide nucleic acid sequence comprising SEQ ID NO. 21, or SEQ ID NO. 737, wherein the second guide nucleic acid sequence comprising SEQ ID NO: 22, or SEQ ID NO. 738, and further comprising excising a region that contains exon 1A, exon 1B and at least a portion of GC repeats expansion in the mutant allele by cleaving one or both strands of DNA at a first target nucleic acid sequence and at a second target nucleic acid sequence with the endonuclease.

8. The method of claim 5, wherein a first guide nucleic acid is targeting a sequence upstream of a transcriptional start site at the C9orf72 locus, wherein a second guide nucleic acid is targeting a sequence downstream of the transcriptional start site at the C9orf72 locus, wherein the first guide nucleic acid sequence comprising SEQ ID NO. 5, or SEQ ID NO. 733, wherein the second guide nucleic acid sequence comprising SEQ ID NO: 6, or SEQ ID NO. 734, and further comprising the steps of:
a. excising a region that contains the transcriptional start site in the mutant allele by cleaving one or both strands of DNA at a first target nucleic acid sequence and at a second target nucleic acid sequence with the endonuclease;
b. excising a region that contains the transcriptional start site in the normal allele by cleaving one or both strands of DNA at a first target nucleic acid sequence and a second target nucleic acid sequence with the endonuclease; and
c. changing expression level of the C9orf72 gene in the host cells.

9. A population of engineered cells modified by the method of any of the claims 5-8, wherein a C9orf72 GC repeat expansion mutation in the cells have been corrected.

10. A method of treating C9orf72 GC repeat expansion mutation associated diseases in a subject, comprising administering a population of engineered cells, wherein the C9orf72 GC repeat expansion mutation have been corrected by the method of any of the claims 5-8.


SEQUENCE LISTING (1411 sequences)

SEQ ID NO: 1 - DNA, 20 bp, synthetic construct
  source 1..20: mol_type = other DNA
aactcaggagtcgcgcgcta

SEQ ID NO: 2 - DNA, 20 bp, synthetic construct
  source 1..20: mol_type = other DNA
ggcccgccccgaccacgccc

SEQ ID NO: 3 - DNA, 21 bp, synthetic construct
  source 1..21: mol_type = other DNA
ccgctaggaaagagaggtgcg

SEQ ID NO: 4 - DNA, 21 bp, synthetic construct
  source 1..21: mol_type = other DNA
gaggagagcccccgcttctac

SEQ ID NO: 5 - DNA, 20 bp, synthetic construct
  source 1..20: mol_type = other DNA
tgcgatgacgttttctcacg

SEQ ID NO: 6 - DNA, 20 bp, synthetic construct
  source 1..20: mol_type = other DNA
tactgtgagagcaagtagtg

SEQ ID NO: 7 - DNA, 20 bp, synthetic construct
  source 1..20: mol_type = other DNA
gatccagcagcctcccctat

SEQ ID NO: 8 - DNA, 20 bp, synthetic construct
  source 1..20: mol_type = other DNA
gctacaggctgcggttgttt

SEQ ID NO: 9 - DNA, 19 bp, synthetic construct
  source 1..19: mol_type = other DNA
tccagcagcctcccctatt

SEQ ID NO: 10 - DNA, 20 bp, synthetic construct
  source 1..20: mol_type = other DNA
tttacgtgggcggaacttgt

SEQ ID NO: 11 - DNA, 20 bp, synthetic construct
  source 1..20: mol_type = other DNA
agagaggtgcgtcaaacagc

SEQ ID NO: 12 - DNA, 21 bp, synthetic construct
  source 1..21: mol_type = other DNA
ctcctgagttccagagcttgc

SEQ ID NO: 13 - DNA, 20 bp, synthetic construct
  source 1..20: mol_type = other DNA
cgtggtcggggcgggcccgg

SEQ ID NO: 14 - DNA, 20 bp, synthetic construct
  source 1..20: mol_type = other DNA
gctgtttggggttcggctgc

SEQ ID NO: 15 - DNA, 20 bp, synthetic construct
  source 1..20: mol_type = other DNA
taacctacggtgtcccgcta

SEQ ID NO: 16 - DNA, 20 bp, synthetic construct
  source 1..20: mol_type = other DNA
ggcaattccaccagtcgcta

SEQ ID NO: 17 - DNA, 21 bp, synthetic construct
  source 1..21: mol_type = other DNA
tactcgctgagggtgaacaag

SEQ ID NO: 18 - DNA, 20 bp, synthetic construct
  source 1..20: mol_type = other DNA
cagtcgctagaggcgaaagc

SEQ ID NO: 19 - DNA, 19 bp, synthetic construct
  source 1..19: mol_type = other DNA
gtggctgtttggggttcgg

SEQ ID NO: 20 - DNA, 20 bp, synthetic construct
  source 1..20: mol_type = other DNA
ccagtcgctagaggcgaaag

SEQ ID NO: 21 - DNA, 20 bp, synthetic construct
  source 1..20: mol_type = other DNA
ctctgtgagaagtttttatc

SEQ ID NO: 22 - DNA, 20 bp, synthetic construct
  source 1..20: mol_type = other DNA
gacttagaagaaatattgtg

SEQ ID NO: 23 - DNA, 20 bp, synthetic construct
  source 1..20: mol_type = other DNA
aggaaccaagcagccatgaa

SEQ ID NO: 24 - DNA, 20 bp, synthetic construct
  source 1..20: mol_type = other DNA
gggaagccacacccttgtaa

SEQ ID NO: 25 - DNA, 20 bp, synthetic construct
  source 1..20: mol_type = other DNA
ctttggcacagataggccac

SEQ ID NO: 26 - DNA, 20 bp, synthetic construct
  source 1..20: mol_type = other DNA
ggcagggtgactgctttaac

SEQ ID NO: 27 - DNA, 25 bp, synthetic construct
  source 1..25: mol_type = other DNA
tgcccagaataaattttggataact

SEQ ID NO: 28 - DNA, 20 bp, synthetic construct
  source 1..20: mol_type = other DNA
gggaagccacacccttgtaa

SEQ ID NO: 29 - DNA, 20 bp, synthetic construct
  source 1..20: mol_type = other DNA
ctctgtgagaagttcttatc

SEQ ID NO: 30 - DNA, 20 bp, synthetic construct
  source 1..20: mol_type = other DNA
gacttaaaagaaatattgtg

SEQ ID NO: 31 - DNA, 20 bp, synthetic construct
  source 1..20: mol_type = other DNA
aggaaccaagcagccatgaa

SEQ ID NO: 32 - DNA, 20 bp, synthetic construct
  source 1..20: mol_type = other DNA
gggaagccacacccttgtaa

SEQ ID NO: 33 - DNA, 20 bp, synthetic construct
  source 1..20: mol_type = other DNA
ctttggcacagataggccac

SEQ ID NO: 34 - DNA, 20 bp, synthetic construct
  source 1..20: mol_type = other DNA
gggaagccacacccttgtaa

SEQ ID NO: 35 - DNA, 25 bp, synthetic construct
  source 1..25: mol_type = other DNA
tgcccagaataaattttggataact

SEQ ID NO: 36 - DNA, 20 bp, synthetic construct
  source 1..20: mol_type = other DNA
gggaagccacacccttgtaa

SEQ ID NO: 37 - DNA, 20 bp, synthetic construct
  source 1..20: mol_type = other DNA
tgtgcgaaccttaatagggg

SEQ ID NO: 38 - DNA, 20 bp, synthetic construct
  source 1..20: mol_type = other DNA
aatggggatcgcagcacata

SEQ ID NO: 39 - DNA, 20 bp, synthetic construct
  source 1..20: mol_type = other DNA
gcagaccaaaagacgcaagg

SEQ ID NO: 40 - DNA, 25 bp, synthetic construct
  source 1..25: mol_type = other DNA
accagaaaataagctttcaacagat

SEQ ID NO: 41 - DNA, 20 bp, synthetic construct
  source 1..20: mol_type = other DNA
gcagaccaaaagacgcaagg

SEQ ID NO: 42 - DNA, 20 bp, synthetic construct
  source 1..20: mol_type = other DNA
cagcgagtactgtgagagca

SEQ ID NO: 43 - DNA, 20 bp, synthetic construct
  source 1..20: mol_type = other DNA
gggttaggggccaaatctcc

SEQ ID NO: 44 - DNA, 25 bp, synthetic construct
  source 1..25: mol_type = other DNA
accagaaaataagctttcaacagat

SEQ ID NO: 45 - DNA, 20 bp, synthetic construct
  source 1..20: mol_type = other DNA
ctctgtgagaagtttttatc

SEQ ID NO: 46 - DNA, 20 bp, synthetic construct
  source 1..20: mol_type = other DNA
ctctgtgagaagttcttatc

SEQ ID NO: 47 - DNA, 20 bp, synthetic construct
  source 1..20: mol_type = other DNA
gacttagaagaaatattgtg

SEQ ID NO: 48 - DNA, 20 bp, synthetic construct
  source 1..20: mol_type = other DNA
gacttaaaagaaatattgtg

SEQ ID NO: 49 - DNA, 20 bp, synthetic construct
  source 1..20: mol_type = other DNA
aggaaccaagcagccatgaa

SEQ ID NO: 50 - DNA, 20 bp, synthetic construct
  source 1..20: mol_type = other DNA
gggaagccacacccttgtaa

SEQ ID NO: 51 - DNA, 20 bp, synthetic construct
  source 1..20: mol_type = other DNA
ctttggcacagataggccac

SEQ ID NO: 52 - DNA, 20 bp, synthetic construct
  source 1..20: mol_type = other DNA
ggcagggtgactgctttaac

SEQ ID NO: 53 - DNA, 25 bp, synthetic construct
  source 1..25: mol_type = other DNA
tgcccagaataaattttggataact

SEQ ID NO: 54 - DNA, 20 bp, synthetic construct
  source 1..20: mol_type = other DNA
gggaagccacacccttgtaa

SEQ ID NO: 55 - DNA, 20 bp, synthetic construct
  source 1..20: mol_type = other DNA
ttacaaaatgaaccttgcat

SEQ ID NO: 56 - DNA, 20 bp, synthetic construct
  source 1..20: mol_type = other DNA
ttacaaaatgaaccttgcac

SEQ ID NO: 57 - DNA, 20 bp, synthetic construct
  source 1..20: mol_type = other DNA
gaagttacttaacctatgca

SEQ ID NO: 58 - DNA, 20 bp, synthetic construct
  source 1..20: mol_type = other DNA
gaagttacttaacctgtgca

SEQ ID NO: 59 - DNA, 20 bp, synthetic construct
  source 1..20: mol_type = other DNA
aaactttctgcttaactctc

SEQ ID NO: 60 - DNA, 20 bp, synthetic construct
  source 1..20: mol_type = other DNA
aaactttctgcttaactctc

SEQ ID NO: 61 - DNA, 20 bp, synthetic construct
  source 1..20: mol_type = other DNA
cacatctttctgattcatga

SEQ ID NO: 62 - DNA, 20 bp, synthetic construct
  source 1..20: mol_type = other DNA
cgcatctttctgattcatga

SEQ ID NO: 63 - DNA, 20 bp, synthetic construct
  source 1..20: mol_type = other DNA
acatctttctgattcatgat

SEQ ID NO: 64 - DNA, 20 bp, synthetic construct
  source 1..20: mol_type = other DNA
gcatctttctgattcatgat

SEQ ID NO: 65 - DNA, 20 bp, synthetic construct
  source 1..20: mol_type = other DNA
gttgtgtaaatttggtgggt

SEQ ID NO: 66 - DNA, 20 bp, synthetic construct
  source 1..20: mol_type = other DNA
attgtgtaaatttggtgggt

SEQ ID NO: 67 - DNA, 20 bp, synthetic construct
  source 1..20: mol_type = other DNA
gtcattttgttgtgtaaatt

SEQ ID NO: 68 - DNA, 20 bp, synthetic construct
  source 1..20: mol_type = other DNA
gtcattttattgtgtaaatt

SEQ ID NO: 69 - DNA, 20 bp, synthetic construct
  source 1..20: mol_type = other DNA
attttgttgtgtaaatttgg

SEQ ID NO: 70 - DNA, 20 bp, synthetic construct
  source 1..20: mol_type = other DNA
attttattgtgtaaatttgg

SEQ ID NO: 71 - DNA, 20 bp, synthetic construct
  source 1..20: mol_type = other DNA
ttttgttgtgtaaatttggt

SEQ ID NO: 72 - DNA, 20 bp, synthetic construct
  source 1..20: mol_type = other DNA
ttttattgtgtaaatttggt

SEQ ID NO: 73 - DNA, 20 bp, synthetic construct
  source 1..20: mol_type = other DNA
tgttgtgtaaatttggtggg

SEQ ID NO: 74 - DNA, 20 bp, synthetic construct
  source 1..20: mol_type = other DNA
tattgtgtaaatttggtggg

SEQ ID NO: 75 - DNA, 20 bp, synthetic construct
  source 1..20: mol_type = other DNA
gttgcaatgattgccaaagc

SEQ ID NO: 76 - DNA, 20 bp, synthetic construct
  source 1..20: mol_type = other DNA
tttgcaatgattgccaaagc

SEQ ID NO: 77 - DNA, 20 bp, synthetic construct
  source 1..20: mol_type = other DNA
tccattatttttaggcagaa

SEQ ID NO: 78 - DNA, 24 bp, synthetic construct
  source 1..24: mol_type = other DNA
tttttccattatttttaggcagaa

SEQ ID NO: 79 - DNA, 20 bp, synthetic construct
  source 1..20: mol_type = other DNA
ttccattatttttaggcaga

SEQ ID NO: 80 - DNA, 23 bp, synthetic construct
  source 1..23: mol_type = other DNA
tttttccattatttttaggcaga

SEQ ID NO: 81 - DNA, 20 bp, synthetic construct
  source 1..20: mol_type = other DNA
tttttttttccattattttt

SEQ ID NO: 82 - DNA, 20 bp, synthetic construct
  source 1..20: mol_type = other DNA
tttttttttccattattttt

SEQ ID NO: 83 - DNA, 20 bp, synthetic construct
  source 1..20: mol_type = other DNA
tccattatttttaggcagaa

SEQ ID NO: 84 - DNA, 20 bp, synthetic construct
  source 1..20: mol_type = other DNA
tccattatttttaggcagaa

SEQ ID NO: 85 - DNA, 20 bp, synthetic construct
  source 1..20: mol_type = other DNA
ttccattatttttaggcaga

SEQ ID NO: 86 - DNA, 20 bp, synthetic construct
  source 1..20: mol_type = other DNA
ttccattatttttaggcaga

SEQ ID NO: 87 - DNA, 20 bp, synthetic construct
  source 1..20: mol_type = other DNA
tttttttttccattattttt

SEQ ID NO: 88 - DNA, 20 bp, synthetic construct
  source 1..20: mol_type = other DNA
tttttttttccattattttt

SEQ ID NO: 89 - DNA, 20 bp, synthetic construct
  source 1..20: mol_type = other DNA
tccattatttttaggcagaa

SEQ ID NO: 90 - DNA, 20 bp, synthetic construct
  source 1..20: mol_type = other DNA
tccattatttttaggcagaa

SEQ ID NO: 91 - DNA, 20 bp, synthetic construct
  source 1..20: mol_type = other DNA
ttccattatttttaggcaga

SEQ ID NO: 92 - DNA, 20 bp, synthetic construct
  source 1..20: mol_type = other DNA
ttccattatttttaggcaga

SEQ ID NO: 93 - DNA, 20 bp, synthetic construct
  source 1..20: mol_type = other DNA
tttttttttccattattttt

SEQ ID NO: 94 - DNA, 20 bp, synthetic construct
  source 1..20: mol_type = other DNA
tttttttttccattattttt

SEQ ID NO: 95 - DNA, 20 bp, synthetic construct
  source 1..20: mol_type = other DNA
tccattatttttaggcagaa

SEQ ID NO: 96 - DNA, 27 bp, synthetic construct
  source 1..27: mol_type = other DNA
ttttttttccattatttttaggcagaa

SEQ ID NO: 97 - DNA, 20 bp, synthetic construct
  source 1..20: mol_type = other DNA
ttccattatttttaggcaga

SEQ ID NO: 98 - DNA, 26 bp, synthetic construct
  source 1..26: mol_type = other DNA
ttttttttccattatttttaggcaga

SEQ ID NO: 99 - DNA, 20 bp, synthetic construct
  source 1..20: mol_type = other DNA
tttttttttccattattttt

SEQ ID NO: 100 - DNA, 20 bp, synthetic construct
  source 1..20: mol_type = other DNA
tttttttttccattattttt

SEQ ID NO: 101 - DNA, 20 bp, synthetic construct
  source 1..20: mol_type = other DNA
tccattatttttaggcagaa

SEQ ID NO: 102 - DNA, 22 bp, synthetic construct
  source 1..22: mol_type = other DNA
tttccattatttttaggcagaa

SEQ ID NO: 103 - DNA, 20 bp, synthetic construct
  source 1..20: mol_type = other DNA
ttccattatttttaggcaga

SEQ ID NO: 104 - DNA, 21 bp, synthetic construct
  source 1..21: mol_type = other DNA
tttccattatttttaggcaga

SEQ ID NO: 105 - DNA, 20 bp, synthetic construct
  source 1..20: mol_type = other DNA
tttttttttccattattttt

SEQ ID NO: 106 - DNA, 20 bp, synthetic construct
  source 1..20: mol_type = other DNA
tttttttttccattattttt

SEQ ID NO: 107 - DNA, 20 bp, synthetic construct
  source 1..20: mol_type = other DNA
aacaaaaaaacaatgtacaa

SEQ ID NO: 108 - DNA, 20 bp, synthetic construct
  source 1..20: mol_type = other DNA
aacaaaaaaccaatgtacaa

SEQ ID NO: 109 - DNA, 20 bp, synthetic construct
  source 1..20: mol_type = other DNA
atttttttttttttttgaga

SEQ ID NO: 110 - DNA, 24 bp, synthetic construct
  source 1..24: mol_type = other DNA
atatttttttttttttttttgaga

SEQ ID NO: 111 - DNA, 20 bp, synthetic construct
  source 1..20: mol_type = other DNA
atttttttttttttttgaga

SEQ ID NO: 112 - DNA, 20 bp, synthetic construct
  source 1..20: mol_type = other DNA
atttttttttttttttgaga

SEQ ID NO: 113 - DNA, 20 bp, synthetic construct
  source 1..20: mol_type = other DNA
tcactgcaacctacacctcc

SEQ ID NO: 114 - DNA, 20 bp, synthetic construct
  source 1..20: mol_type = other DNA
tcactgcaacctccacctcc

SEQ ID NO: 115 - DNA, 20 bp, synthetic construct
  source 1..20: mol_type = other DNA
cactgcaacctacacctcct

SEQ ID NO: 116 - DNA, 20 bp, synthetic construct
  source 1..20: mol_type = other DNA
cactgcaacctccacctcct

SEQ ID NO: 117 - DNA, 20 bp, synthetic construct
  source 1..20: mol_type = other DNA
cgcttgaacccaggaggtgt

SEQ ID NO: 118 - DNA, 20 bp, synthetic construct
  source 1..20: mol_type = other DNA
cgcttgaacccaggaggtgg

SEQ ID NO: 119 - DNA, 20 bp, synthetic construct
  source 1..20: mol_type = other DNA
cactgcaacctacacctcct

SEQ ID NO: 120 - DNA, 20 bp, synthetic construct
  source 1..20: mol_type = other DNA
cactgcaacctacacctccg

SEQ ID NO: 121 - DNA, 20 bp, synthetic construct
  source 1..20: mol_type = other DNA
cgcttgaacccaggaggtgt

SEQ ID NO: 122 - DNA, 20 bp, synthetic construct
  source 1..20: mol_type = other DNA
cgcttgaaccccggaggtgt

SEQ ID NO: 123 - DNA, 20 bp, synthetic construct
  source 1..20: mol_type = other DNA
gagaatcgcttgaacccagg

SEQ ID NO: 124 - DNA, 20 bp, synthetic construct
  source 1..20: mol_type = other DNA
gagaatcgcttgaaccccgg

SEQ ID NO: 125 - DNA, 20 bp, synthetic construct
  source 1..20: mol_type = other DNA
tcatatacctgtacttttgc

SEQ ID NO: 126 - DNA, 20 bp, synthetic construct
  source 1..20: mol_type = other DNA
tcatatacccgtacttttgc

SEQ ID NO: 127 - DNA, 20 bp, synthetic construct
  source 1..20: mol_type = other DNA
catatacctgtacttttgca

SEQ ID NO: 128 - DNA, 20 bp, synthetic construct
  source 1..20: mol_type = other DNA
catatacccgtacttttgca

SEQ ID NO: 129 - DNA, 20 bp, synthetic construct
  source 1..20: mol_type = other DNA
atatacctgtacttttgcag

SEQ ID NO: 130 - DNA, 20 bp, synthetic construct
  source 1..20: mol_type = other DNA
atatacccgtacttttgcag

SEQ ID NO: 131 - DNA, 20 bp, synthetic construct
  source 1..20: mol_type = other DNA
tatacctgtacttttgcagg

SEQ ID NO: 132 - DNA, 20 bp, synthetic construct
  source 1..20: mol_type = other DNA
tatacccgtacttttgcagg

SEQ ID NO: 133 - DNA, 20 bp, synthetic construct
  source 1..20: mol_type = other DNA
acctgtacttttgcaggggg

SEQ ID NO: 134 - DNA, 20 bp, synthetic construct
  source 1..20: mol_type = other DNA
acccgtacttttgcaggggg

SEQ ID NO: 135 - DNA, 20 bp, synthetic construct
  source 1..20: mol_type = other DNA
ttttttttttttttgagaaa

SEQ ID NO: 136 - DNA, 20 bp, synthetic construct
  source 1..20: mol_type = other DNA
ttttttttttttttgagaaa

SEQ ID NO: 137 - DNA, 20 bp, synthetic construct
  source 1..20: mol_type = other DNA
tttttttttttttttgagaa

SEQ ID NO: 138 - DNA, 20 bp, synthetic construct
  source 1..20: mol_type = other DNA
tttttttttttttttgagaa

SEQ ID NO: 139 - DNA, 20 bp, synthetic construct
  source 1..20: mol_type = other DNA
tttttttttttgagaaaggg

SEQ ID NO: 140 - DNA, 20 bp, synthetic construct
  source 1..20: mol_type = other DNA
tttttttttttgagaaaggg

SEQ ID NO: 141 - DNA, 20 bp, synthetic construct
  source 1..20: mol_type = other DNA
ttttttttttttttgagaaa

SEQ ID NO: 142 - DNA, 20 bp, synthetic construct
  source 1..20: mol_type = other DNA
ttttttttttttttgagaaa

SEQ ID NO: 143 - DNA, 20 bp, synthetic construct
  source 1..20: mol_type = other DNA
tttttttttttttttgagaa

SEQ ID NO: 144 - DNA, 20 bp, synthetic construct
  source 1..20: mol_type = other DNA
tttttttttttttttgagaa

SEQ ID NO: 145 - DNA, 20 bp, synthetic construct
  source 1..20: mol_type = other DNA
tttttttttttgagaaaggg

SEQ ID NO: 146 - DNA, 20 bp, synthetic construct
  source 1..20: mol_type = other DNA
tttttttttttgagaaaggg

SEQ ID NO: 147 - DNA, 20 bp, synthetic construct
  source 1..20: mol_type = other DNA
tatactctacataacataat

SEQ ID NO: 148 - DNA, 20 bp, synthetic construct
  source 1..20: mol_type = other DNA
tatattctacataacataat

SEQ ID NO: 149 - DNA, 20 bp, synthetic construct
  source 1..20: mol_type = other DNA
atactctacataacataata

SEQ ID NO: 150 - DNA, 20 bp, synthetic construct
  source 1..20: mol_type = other DNA
atattctacataacataata

SEQ ID NO: 151 - DNA, 20 bp, synthetic construct
  source 1..20: mol_type = other DNA
atatatagaacaggcattgt

SEQ ID NO: 152 - DNA, 20 bp, synthetic construct
  source 1..20: mol_type = other DNA
atatatagaacaggcactgt

SEQ ID NO: 153 - DNA, 20 bp, synthetic construct
  source 1..20: mol_type = other DNA
taaaaaaagtagagaccctt

SEQ ID NO: 154 - DNA, 20 bp, synthetic construct
  source 1..20: mol_type = other DNA
taaaaaaagtagagaccttt

SEQ ID NO: 155 - DNA, 20 bp, synthetic construct
  source 1..20: mol_type = other DNA
ttttttgagatggcaagaat

SEQ ID NO: 156 - DNA, 20 bp, synthetic construct
  source 1..20: mol_type = other DNA
gtttttgagatggcaagaat

SEQ ID NO: 157 - DNA, 20 bp, synthetic construct
  source 1..20: mol_type = other DNA
ttttttgtttttttttgaga

SEQ ID NO: 158 - DNA, 20 bp, synthetic construct
  source 1..20: mol_type = other DNA
tttttgttttgtttttgaga

SEQ ID NO: 159 - DNA, 20 bp, synthetic construct
  source 1..20: mol_type = other DNA
cgttttgcggctattgtgaa

SEQ ID NO: 160 - DNA, 20 bp, synthetic construct
  source 1..20: mol_type = other DNA
cgctttgcggctattgtgaa

SEQ ID NO: 161 - DNA, 20 bp, synthetic construct
  source 1..20: mol_type = other DNA
gttttgcggctattgtgaat

SEQ ID NO: 162 - DNA, 20 bp, synthetic construct
  source 1..20: mol_type = other DNA
gctttgcggctattgtgaat

SEQ ID NO: 163 - DNA, 20 bp, synthetic construct
  source 1..20: mol_type = other DNA
taggtattttattcgttttg

SEQ ID NO: 164 - DNA, 20 bp, synthetic construct
  source 1..20: mol_type = other DNA
taggtattttattcgctttg

SEQ ID NO: 165 - DNA, 20 bp, synthetic construct
  source 1..20: mol_type = other DNA
gcaaaacgaataaaatacct

SEQ ID NO: 166 - DNA, 20 bp, synthetic construct
  source 1..20: mol_type = other DNA
gcaaagcgaataaaatacct

SEQ ID NO: 167 - DNA, 20 bp, synthetic construct
  source 1..20: mol_type = other DNA
ctgaaaaggagtggtgagag

SEQ ID NO: 168 - DNA, 20 bp, synthetic construct
  source 1..20: mol_type = other DNA
ctgaaaaggagcggtgagag

SEQ ID NO: 169 - DNA, 20 bp, synthetic construct
  source 1..20: mol_type = other DNA
actccttttcagcatagttc

SEQ ID NO: 170 - DNA, 20 bp, synthetic construct
  source 1..20: mol_type = other DNA
gctccttttcagcatagttc

SEQ ID NO: 171 - DNA, 20 bp, synthetic construct
  source 1..20: mol_type = other DNA
tgatttttcttttttgagac

SEQ ID NO: 172 - DNA, 20 bp, synthetic construct
  source 1..20: mol_type = other DNA
tgatttttcttttttgagac

SEQ ID NO: 173 - DNA, 20 bp, synthetic construct
  source 1..20: mol_type = other DNA
gatttttcttttttgagaca

SEQ ID NO: 174 - DNA, 20 bp, synthetic construct
  source 1..20: mol_type = other DNA
gatttttcttttttgagaca

SEQ ID NO: 175 - DNA, 20 bp, synthetic construct
  source 1..20: mol_type = other DNA
acctcaacctcctgaatagc

SEQ ID NO: 176 - DNA, 20 bp, synthetic construct
  source 1..20: mol_type = other DNA
atctcaacctcctgaatagc

SEQ ID NO: 177 - DNA, 20 bp, synthetic construct
  source 1..20: mol_type = other DNA
cctcaacctcctgaatagct

SEQ ID NO: 178 - DNA, 20 bp, synthetic construct
  source 1..20: mol_type = other DNA
tctcaacctcctgaatagct

SEQ ID NO: 179 - DNA, 20 bp, synthetic construct
  source 1..20: mol_type = other DNA
attcaggaggttgaggtggg

SEQ ID NO: 180 - DNA, 20 bp, synthetic construct
  source 1..20: mol_type = other DNA
attcaggaggttgagatggg

SEQ ID NO: 181 - DNA, 20 bp, synthetic construct
  source 1..20: mol_type = other DNA
gtgggaggattgtttgagcc

SEQ ID NO: 182 - DNA, 20 bp, synthetic construct
  source 1..20: mol_type = other DNA
atgggaggattgtttgagcc

SEQ ID NO: 183 - DNA, 20 bp, synthetic construct
  source 1..20: mol_type = other DNA
gctattcaggaggttgaggt

SEQ ID NO: 184 - DNA, 20 bp, synthetic construct
  source 1..20: mol_type = other DNA
gctattcaggaggttgagat

SEQ ID NO: 185 - DNA, 20 bp, synthetic construct
  source 1..20: mol_type = other DNA
agctattcaggaggttgagg

SEQ ID NO: 186 - DNA, 20 bp, synthetic construct
  source 1..20: mol_type = other DNA
agctattcaggaggttgaga

SEQ ID NO: 187 - DNA, 20 bp, synthetic construct
  source 1..20: mol_type = other DNA
tcctgaatagctgggactat

SEQ ID NO: 188 - DNA, 20 bp, synthetic construct
  source 1..20: mol_type = other DNA
tcctgaatagctgggactac

SEQ ID NO: 189 - DNA, 20 bp, synthetic construct
  source 1..20: mol_type = other DNA
acctatagtcccagctattc

SEQ ID NO: 190 - DNA, 20 bp, synthetic construct
  source 1..20: mol_type = other DNA
acctgtagtcccagctattc

SEQ ID NO: 191 - DNA, 20 bp, synthetic construct
  source 1..20: mol_type = other DNA
tatagtcccagctattcagg

SEQ ID NO: 192 - DNA, 20 bp, synthetic construct
  source 1..20: mol_type = other DNA
tgtagtcccagctattcagg

SEQ ID NO: 193 - DNA, 20 bp, synthetic construct
  source 1..20: mol_type = other DNA
aaaaaaaaattagcttggta

SEQ ID NO: 194 - DNA, 20 bp, synthetic construct
  source 1..20: mol_type = other DNA
aaaaaaaaattagcttggta

SEQ ID NO: 195 - DNA, 20 bp, synthetic construct
  source 1..20: mol_type = other DNA
aaaaaaaaaaaaaattagct

SEQ ID NO: 196 - DNA, 20 bp, synthetic construct
  source 1..20: mol_type = other DNA
aaaaaaaaaaaaaattagct

SEQ ID NO: 197 - DNA, 20 bp, synthetic construct
  source 1..20: mol_type = other DNA
aaaaaaaaattagcttggta

SEQ ID NO: 198 - DNA, 20 bp, synthetic construct
  source 1..20: mol_type = other DNA
aaaaaaaaattagcttggta

SEQ ID NO: 199 - DNA, 20 bp, synthetic construct
  source 1..20: mol_type = other DNA
aaaaaaaaaaaaaattagct

SEQ ID NO: 200 - DNA, 20 bp, synthetic construct
  source 1..20: mol_type = other DNA
aaaaaaaaaaaaaattagct

SEQ ID NO: 201 - DNA, 20 bp, synthetic construct
  source 1..20: mol_type = other DNA
aaaaaaaaattagcttggta

SEQ ID NO: 202 - DNA, 20 bp, synthetic construct
  source 1..20: mol_type = other DNA
aaaaaaaaattagcttggta

SEQ ID NO: 203 - DNA, 20 bp, synthetic construct
  source 1..20: mol_type = other DNA
aaaaaaaaaaaaaattagct

SEQ ID NO: 204 - DNA, 20 bp, synthetic construct
  source 1..20: mol_type = other DNA
aaaaaaaaaaaaaattagct

SEQ ID NO: 205 - DNA, 20 bp, synthetic construct
  source 1..20: mol_type = other DNA
aaaaaaaaattagcttggta

SEQ ID NO: 206 - DNA, 20 bp, synthetic construct
  source 1..20: mol_type = other DNA
aaaaaaaaattagcttggta

SEQ ID NO: 207 - DNA, 20 bp, synthetic construct
  source 1..20: mol_type = other DNA
aaaaaaaaaaaaaattagct

SEQ ID NO: 208 - DNA, 20 bp, synthetic construct
  source 1..20: mol_type = other DNA
aaaaaaaaaaaaaattagct

SEQ ID NO: 209 - DNA, 20 bp, synthetic construct
  source 1..20: mol_type = other DNA
cacctgtaatcccagcactt

SEQ ID NO: 210 - DNA, 20 bp, synthetic construct
  source 1..20: mol_type = other DNA
catctgtaatcccagcactt

SEQ ID NO: 211 - DNA, 20 bp, synthetic construct
  source 1..20: mol_type = other DNA
acctgtaatcccagcacttt

SEQ ID NO: 212 - DNA, 20 bp, synthetic construct
  source 1..20: mol_type = other DNA
atctgtaatcccagcacttt

SEQ ID NO: 213 - DNA, 20 bp, synthetic construct
  source 1..20: mol_type = other DNA
cttggctctctattctccat

SEQ ID NO: 214 - DNA, 20 bp, synthetic construct
  source 1..20: mol_type = other DNA
cttggctctgtattctccat

SEQ ID NO: 215 - DNA, 20 bp, synthetic construct
  source 1..20: mol_type = other DNA
ccccactacaaactgggcaa

SEQ ID NO: 216 - DNA, 20 bp, synthetic construct
  source 1..20: mol_type = other DNA
ccccactacaaaccgggcaa

SEQ ID NO: 217 - DNA, 20 bp, synthetic construct
  source 1..20: mol_type = other DNA
aacccaccccactacaaact

SEQ ID NO: 218 - DNA, 20 bp, synthetic construct
  source 1..20: mol_type = other DNA
aacccaccccactacaaacc

SEQ ID NO: 219 - DNA, 20 bp, synthetic construct
  source 1..20: mol_type = other DNA
tgcccagtttgtagtggggt

SEQ ID NO: 220 - DNA, 20 bp, synthetic construct
  source 1..20: mol_type = other DNA
tgcccggtttgtagtggggt

SEQ ID NO: 221 - DNA, 20 bp, synthetic construct
  source 1..20: mol_type = other DNA
ttgcccagtttgtagtgggg

SEQ ID NO: 222 - DNA, 20 bp, synthetic construct
  source 1..20: mol_type = other DNA
ttgcccggtttgtagtgggg

SEQ ID NO: 223 - DNA, 20 bp, synthetic construct
  source 1..20: mol_type = other DNA
cctttgcccagtttgtagtg

SEQ ID NO: 224 - DNA, 20 bp, synthetic construct
  source 1..20: mol_type = other DNA
cctttgcccggtttgtagtg

SEQ ID NO: 225 - DNA, 20 bp, synthetic construct
  source 1..20: mol_type = other DNA
tcctttgcccagtttgtagt

SEQ ID NO: 226 - DNA, 20 bp, synthetic construct
  source 1..20: mol_type = other DNA
tcctttgcccggtttgtagt

SEQ ID NO: 227 - DNA, 20 bp, synthetic construct
  source 1..20: mol_type = other DNA
gtcctttgcccagtttgtag

SEQ ID NO: 228 - DNA, 20 bp, synthetic construct
  source 1..20: mol_type = other DNA
gtcctttgcccggtttgtag

SEQ ID NO: 229 - DNA, 20 bp, synthetic construct
  source 1..20: mol_type = other DNA
agaaaaatttgtattccttt

SEQ ID NO: 230 - DNA, 20 bp, synthetic construct
  source 1..20: mol_type = other DNA
agaacaatttgtattccttt

SEQ ID NO: 231 - DNA, 20 bp, synthetic construct
  source 1..20: mol_type = other DNA
agaatgggtttgcaaccatt

SEQ ID NO: 232 - DNA, 20 bp, synthetic construct
  source 1..20: mol_type = other DNA
agaatgggtttgcaaccgtt

SEQ ID NO: 233 - DNA, 20 bp, synthetic construct
  source 1..20: mol_type = other DNA
ggaaaaaagaaaatgtttca

SEQ ID NO: 234 - DNA, 20 bp, synthetic construct
  source 1..20: mol_type = other DNA
ggaaaaaagaaaatatttca

SEQ ID NO: 235 - DNA, 20 bp, synthetic construct
  source 1..20: mol_type = other DNA
aaaatgcttgcaataatgcc

SEQ ID NO: 236 - DNA, 20 bp, synthetic construct
  source 1..20: mol_type = other DNA
aaaatgcttgcaataatgcc

SEQ ID NO: 237 - DNA, 20 bp, synthetic construct
  source 1..20: mol_type = other DNA
aaaatgcttgcaataatgcc

SEQ ID NO: 238 - DNA, 20 bp, synthetic construct
  source 1..20: mol_type = other DNA
aaaatgcttgcaataatgcc

SEQ ID NO: 239 - DNA, 20 bp, synthetic construct
  source 1..20: mol_type = other DNA
tcccaaagctctgttcttac

SEQ ID NO: 240 - DNA, 20 bp, synthetic construct
  source 1..20: mol_type = other DNA
acccaaagctctgttcttac

SEQ ID NO: 241 - DNA, 20 bp, synthetic construct
  source 1..20: mol_type = other DNA
gagctttgggagtgcaaggc

SEQ ID NO: 242 - DNA, 20 bp, synthetic construct
  source 1..20: mol_type = other DNA
gagctttgggtgtgcaaggc

SEQ ID NO: 243 - DNA, 20 bp, synthetic construct
  source 1..20: mol_type = other DNA
aacagagctttgggagtgca

SEQ ID NO: 244 - DNA, 20 bp, synthetic construct
  source 1..20: mol_type = other DNA
aacagagctttgggtgtgca

SEQ ID NO: 245 - DNA, 20 bp, synthetic construct
  source 1..20: mol_type = other DNA
ctttgggagtgcaaggcagg

SEQ ID NO: 246 - DNA, 20 bp, synthetic construct
  source 1..20: mol_type = other DNA
ctttgggtgtgcaaggcagg

SEQ ID NO: 247 - DNA, 20 bp, synthetic construct
  source 1..20: mol_type = other DNA
tcccaaagctctgttcttac

SEQ ID NO: 248 - DNA, 20 bp, synthetic construct
  source 1..20: mol_type = other DNA
tcccaaagctctgttcttat

SEQ ID NO: 249 - DNA, 20 bp, synthetic construct
  source 1..20: mol_type = other DNA
gcctgtaagaacagagcttt

SEQ ID NO: 250 - DNA, 20 bp, synthetic construct
  source 1..20: mol_type = other DNA
gcctataagaacagagcttt

SEQ ID NO: 251 - DNA, 20 bp, synthetic construct
  source 1..20: mol_type = other DNA
tgcctgtaagaacagagctt

SEQ ID NO: 252 - DNA, 20 bp, synthetic construct
  source 1..20: mol_type = other DNA
tgcctataagaacagagctt

SEQ ID NO: 253 - DNA, 20 bp, synthetic construct
  source 1..20: mol_type = other DNA
tccttttgaatttactgttc

SEQ ID NO: 254 - DNA, 20 bp, synthetic construct
  source 1..20: mol_type = other DNA
tccttttgaatttactgtac

SEQ ID NO: 255 - DNA, 20 bp, synthetic construct
  source 1..20: mol_type = other DNA
gcctgaacagtaaattcaaa

SEQ ID NO: 256 - DNA, 20 bp, synthetic construct
  source 1..20: mol_type = other DNA
gcctgtacagtaaattcaaa

SEQ ID NO: 257 - DNA, 20 bp, synthetic construct
  source 1..20: mol_type = other DNA
gaagctttgtgctgcctttc

SEQ ID NO: 258 - DNA, 20 bp, synthetic construct
  source 1..20: mol_type = other DNA
gaagttttgtgctgcctttc

SEQ ID NO: 259 - DNA, 20 bp, synthetic construct
  source 1..20: mol_type = other DNA
actggctaccccaactgatt

SEQ ID NO: 260 - DNA, 20 bp, synthetic construct
  source 1..20: mol_type = other DNA
actgcctaccccaactgatt

SEQ ID NO: 261 - DNA, 20 bp, synthetic construct
  source 1..20: mol_type = other DNA
tcatctttgtataatgtcaa

SEQ ID NO: 262 - DNA, 20 bp, synthetic construct
  source 1..20: mol_type = other DNA
tcatcttcgtataatgtcaa

SEQ ID NO: 263 - DNA, 20 bp, synthetic construct
  source 1..20: mol_type = other DNA
atagttttgaaatttcctct

SEQ ID NO: 264 - DNA, 20 bp, synthetic construct
  source 1..20: mol_type = other DNA
acagttttgaaatttcctct

SEQ ID NO: 265 - DNA, 20 bp, synthetic construct
  source 1..20: mol_type = other DNA
ttttttatttcattttgccg

SEQ ID NO: 266 - DNA, 20 bp, synthetic construct
  source 1..20: mol_type = other DNA
ttttttatttcattttgctg

SEQ ID NO: 267 - DNA, 20 bp, synthetic construct
  source 1..20: mol_type = other DNA
aattatttataatacttaaa

SEQ ID NO: 268 - DNA, 20 bp, synthetic construct
  source 1..20: mol_type = other DNA
aaatatttataatacttaaa

SEQ ID NO: 269 - DNA, 20 bp, synthetic construct
  source 1..20: mol_type = other DNA
caattatttataatacttaa

SEQ ID NO: 270 - DNA, 20 bp, synthetic construct
  source 1..20: mol_type = other DNA
caaatatttataatacttaa

SEQ ID NO: 271 - DNA, 20 bp, synthetic construct
  source 1..20: mol_type = other DNA
ttaaaaaaaaaaaagtcctg

SEQ ID NO: 272 - DNA, 22 bp, synthetic construct
  source 1..22: mol_type = other DNA
gttaaaaaaaaaaaaagtcctg

SEQ ID NO: 273 - DNA, 20 bp, synthetic construct
  source 1..20: mol_type = other DNA
ttttaacacatgcttaaatc

SEQ ID NO: 274 - DNA, 20 bp, synthetic construct
  source 1..20: mol_type = other DNA
ttttaacacatgcttaaatc

SEQ ID NO: 275 - DNA, 20 bp, synthetic construct
  source 1..20: mol_type = other DNA
agtcctgaggctagacatgt

SEQ ID NO: 276 - DNA, 20 bp, synthetic construct
  source 1..20: mol_type = other DNA
tgtcctgaggctagacatgt

SEQ ID NO: 277 - DNA, 20 bp, synthetic construct
  source 1..20: mol_type = other DNA
ttaaaaaaaaaaaagtcctg

SEQ ID NO: 278 - DNA, 20 bp, synthetic construct
  source 1..20: mol_type = other DNA
ttaaaaaaaaaaatgtcctg

SEQ ID NO: 279 - DNA, 20 bp, synthetic construct
  source 1..20: mol_type = other DNA
cagaagctataaaagttgca

SEQ ID NO: 280 - DNA, 20 bp, synthetic construct
  source 1..20: mol_type = other DNA
cagaagctataaaagttcca

SEQ ID NO: 281 - DNA, 20 bp, synthetic construct
  source 1..20: mol_type = other DNA
aaaaaaaaacactccaacta

SEQ ID NO: 282 - DNA, 22 bp, synthetic construct
  source 1..22: mol_type = other DNA
aaacaaaaaaacactccaacta

SEQ ID NO: 283 - DNA, 20 bp, synthetic construct
  source 1..20: mol_type = other DNA
ttagtttattagctaatgct

SEQ ID NO: 284 - DNA, 20 bp, synthetic construct
  source 1..20: mol_type = other DNA
ttggtttattagctaatgct

SEQ ID NO: 285 - DNA, 20 bp, synthetic construct
  source 1..20: mol_type = other DNA
agcattagctaataaactaa

SEQ ID NO: 286 - DNA, 20 bp, synthetic construct
  source 1..20: mol_type = other DNA
agcattagctaataaaccaa

SEQ ID NO: 287 - DNA, 20 bp, synthetic construct
  source 1..20: mol_type = other DNA
attaactcaagtagcactga

SEQ ID NO: 288 - DNA, 20 bp, synthetic construct
  source 1..20: mol_type = other DNA
actaactcaagtagcactga

SEQ ID NO: 289 - DNA, 20 bp, synthetic construct
  source 1..20: mol_type = other DNA
agtgctacttgagttaatta

SEQ ID NO: 290 - DNA, 20 bp, synthetic construct
  source 1..20: mol_type = other DNA
agtgctacttgagttagtta

SEQ ID NO: 291 - DNA, 20 bp, synthetic construct
  source 1..20: mol_type = other DNA
ctcaagtagcactgaaggaa

SEQ ID NO: 292 - DNA, 20 bp, synthetic construct
  source 1..20: mol_type = other DNA
ctcaagtaacactgaaggaa

SEQ ID NO: 293 - DNA, 20 bp, synthetic construct
  source 1..20: mol_type = other DNA
tcaagtagcactgaaggaaa

SEQ ID NO: 294 - DNA, 20 bp, synthetic construct
  source 1..20: mol_type = other DNA
tcaagtaacactgaaggaaa

SEQ ID NO: 295 - DNA, 20 bp, synthetic construct
  source 1..20: mol_type = other DNA
attaactcaagtagcactga

SEQ ID NO: 296 - DNA, 20 bp, synthetic construct
  source 1..20: mol_type = other DNA
attaactcaagtaacactga

SEQ ID NO: 297 - DNA, 20 bp, synthetic construct
  source 1..20: mol_type = other DNA
agtgctacttgagttaatta

SEQ ID NO: 298 - DNA, 20 bp, synthetic construct
  source 1..20: mol_type = other DNA
agtgttacttgagttaatta

SEQ ID NO: 299 - DNA, 20 bp, synthetic construct
  source 1..20: mol_type = other DNA
catttatacactactgacac

SEQ ID NO: 300 - DNA, 20 bp, synthetic construct
  source 1..20: mol_type = other DNA
catttatacacaactgacac

SEQ ID NO: 301 - DNA, 20 bp, synthetic construct
  source 1..20: mol_type = other DNA
cagtgtcagtagtgtataaa

SEQ ID NO: 302 - DNA, 20 bp, synthetic construct
  source 1..20: mol_type = other DNA
cagtgtcagttgtgtataaa

SEQ ID NO: 303 - DNA, 20 bp, synthetic construct
  source 1..20: mol_type = other DNA
ttataacaaccctacacatt

SEQ ID NO: 304 - DNA, 20 bp, synthetic construct
  source 1..20: mol_type = other DNA
ttataacaaccccacacatt

SEQ ID NO: 305 - DNA, 20 bp, synthetic construct
  source 1..20: mol_type = other DNA
gtaatagtacctaatgtgta

SEQ ID NO: 306 - DNA, 20 bp, synthetic construct
  source 1..20: mol_type = other DNA
gtaatagtacctaatgtgtg

SEQ ID NO: 307 - DNA, 20 bp, synthetic construct
  source 1..20: mol_type = other DNA
gtgcacagagattgtttttt

SEQ ID NO: 308 - DNA, 20 bp, synthetic construct
  source 1..20: mol_type = other DNA
gtacacagagattgtttttt

SEQ ID NO: 309 - DNA, 20 bp, synthetic construct
  source 1..20: mol_type = other DNA
agtgcacagagattgttttt

SEQ ID NO: 310 - DNA, 20 bp, synthetic construct
  source 1..20: mol_type = other DNA
agtacacagagattgttttt

SEQ ID NO: 311 - DNA, 20 bp, synthetic construct
  source 1..20: mol_type = other DNA
tgcacagagattgttttttg

SEQ ID NO: 312 - DNA, 20 bp, synthetic construct
  source 1..20: mol_type = other DNA
tacacagagattgttttttg

SEQ ID NO: 313 - DNA, 20 bp, synthetic construct
  source 1..20: mol_type = other DNA
tgtgtgcttatttgctaaag

SEQ ID NO: 314 - DNA, 20 bp, synthetic construct
  source 1..20: mol_type = other DNA
tgtgtgcctatttgctaaag

SEQ ID NO: 315 - DNA, 20 bp, synthetic construct
  source 1..20: mol_type = other DNA
aataagcacacacaaaatcc

SEQ ID NO: 316 - DNA, 20 bp, synthetic construct
  source 1..20: mol_type = other DNA
aataggcacacacaaaatcc

SEQ ID NO: 317 - DNA, 20 bp, synthetic construct
  source 1..20: mol_type = other DNA
atattctaattttaatagta

SEQ ID NO: 318 - DNA, 20 bp, synthetic construct
  source 1..20: mol_type = other DNA
atattctaatttttatagta

SEQ ID NO: 319 - DNA, 20 bp, synthetic construct
  source 1..20: mol_type = other DNA
ttctttttttttttttgaga

SEQ ID NO: 320 - DNA, 24 bp, synthetic construct
  source 1..24: mol_type = other DNA
ttttctttttttttttttttgaga

SEQ ID NO: 321 - DNA, 20 bp, synthetic construct
  source 1..20: mol_type = other DNA
ttctttttttttttttgaga

SEQ ID NO: 322 - DNA, 20 bp, synthetic construct
  source 1..20: mol_type = other DNA
ttctttttttttttttgaga

SEQ ID NO: 323 - DNA, 20 bp, synthetic construct
  source 1..20: mol_type = other DNA
tttcgccatgttggccaggt

SEQ ID NO: 324 - DNA, 20 bp, synthetic construct
  source 1..20: mol_type = other DNA
tttcgccatgttggccaggc

SEQ ID NO: 325 - DNA, 20 bp, synthetic construct
  source 1..20: mol_type = other DNA
tcagaaatttgacaccaacc

SEQ ID NO: 326 - DNA, 20 bp, synthetic construct
  source 1..20: mol_type = other DNA
tcagaaatttgacaccagcc

SEQ ID NO: 327 - DNA, 20 bp, synthetic construct
  source 1..20: mol_type = other DNA
tgacaccaacctggccaaca

SEQ ID NO: 328 - DNA, 20 bp, synthetic construct
  source 1..20: mol_type = other DNA
tgacaccagcctggccaaca

SEQ ID NO: 329 - DNA, 20 bp, synthetic construct
  source 1..20: mol_type = other DNA
tttgcaagaactcaatcaaa

SEQ ID NO: 330 - DNA, 20 bp, synthetic construct
  source 1..20: mol_type = other DNA
tttgcaagaactcagtcaaa

SEQ ID NO: 331 - DNA, 20 bp, synthetic construct
  source 1..20: mol_type = other DNA
ggtgttgttgagcttcagtg

SEQ ID NO: 332 - DNA, 20 bp, synthetic construct
  source 1..20: mol_type = other DNA
ggtgttgttgagcttcagtg

SEQ ID NO: 333 - DNA, 20 bp, synthetic construct
  source 1..20: mol_type = other DNA
ggtgttgttgagcttcagtg

SEQ ID NO: 334 - DNA, 22 bp, synthetic construct
  source 1..22: mol_type = other DNA
ttggtgttgttgagcttcagtg

SEQ ID NO: 335 - DNA, 20 bp, synthetic construct
  source 1..20: mol_type = other DNA
gttcaagtttgggaagtaca

SEQ ID NO: 336 - DNA, 20 bp, synthetic construct
  source 1..20: mol_type = other DNA
gttcgagtttgggaagtaca

SEQ ID NO: 337 - DNA, 20 bp, synthetic construct
  source 1..20: mol_type = other DNA
ctgctgagaagttcaagttt

SEQ ID NO: 338 - DNA, 20 bp, synthetic construct
  source 1..20: mol_type = other DNA
ctgctgagaagttcgagttt

SEQ ID NO: 339 - DNA, 20 bp, synthetic construct
  source 1..20: mol_type = other DNA
actgctgagaagttcaagtt

SEQ ID NO: 340 - DNA, 20 bp, synthetic construct
  source 1..20: mol_type = other DNA
actgctgagaagttcgagtt

SEQ ID NO: 341 - DNA, 20 bp, synthetic construct
  source 1..20: mol_type = other DNA
caagtttgggaagtacaagg

SEQ ID NO: 342 - DNA, 20 bp, synthetic construct
  source 1..20: mol_type = other DNA
cgagtttgggaagtacaagg

SEQ ID NO: 343 - DNA, 20 bp, synthetic construct
  source 1..20: mol_type = other DNA
tcaacttaagtaaaggtttc

SEQ ID NO: 344 - DNA, 20 bp, synthetic construct
  source 1..20: mol_type = other DNA
tcaacttaagcaaaggtttc

SEQ ID NO: 345 - DNA, 20 bp, synthetic construct
  source 1..20: mol_type = other DNA
atagtagtcaacttaagtaa

SEQ ID NO: 346 - DNA, 20 bp, synthetic construct
  source 1..20: mol_type = other DNA
atagtagtcaacttaagcaa

SEQ ID NO: 347 - DNA, 20 bp, synthetic construct
  source 1..20: mol_type = other DNA
agtaaacttatcgcaagttg

SEQ ID NO: 348 - DNA, 20 bp, synthetic construct
  source 1..20: mol_type = other DNA
agtaaacttaccgcaagttg

SEQ ID NO: 349 - DNA, 20 bp, synthetic construct
  source 1..20: mol_type = other DNA
gaagaaatattgtgtggacc

SEQ ID NO: 350 - DNA, 20 bp, synthetic construct
  source 1..20: mol_type = other DNA
aaagaaatattgtgtggacc

SEQ ID NO: 351 - DNA, 20 bp, synthetic construct
  source 1..20: mol_type = other DNA
tgtctctgatatgttcttta

SEQ ID NO: 352 - DNA, 20 bp, synthetic construct
  source 1..20: mol_type = other DNA
tgtctctgatatgttcttta

SEQ ID NO: 353 - DNA, 20 bp, synthetic construct
  source 1..20: mol_type = other DNA
ctgggcaaacagaaaaaaaa

SEQ ID NO: 354 - DNA, 20 bp, synthetic construct
  source 1..20: mol_type = other DNA
ctgggcaaacagataaaaaa

SEQ ID NO: 355 - DNA, 20 bp, synthetic construct
  source 1..20: mol_type = other DNA
tgggcaaacagaaaaaaaaa

SEQ ID NO: 356 - DNA, 20 bp, synthetic construct
  source 1..20: mol_type = other DNA
tgggcaaacagataaaaaaa

SEQ ID NO: 357 - DNA, 20 bp, synthetic construct
  source 1..20: mol_type = other DNA
tagggaaaaaatgggggcag

SEQ ID NO: 358 - DNA, 20 bp, synthetic construct
  source 1..20: mol_type = other DNA
tagggaaacaatgggggcag

SEQ ID NO: 359 - DNA, 20 bp, synthetic construct
  source 1..20: mol_type = other DNA
gaaaaaatgggggcaggggt

SEQ ID NO: 360 - DNA, 20 bp, synthetic construct
  source 1..20: mol_type = other DNA
gaaacaatgggggcaggggt

SEQ ID NO: 361 - DNA, 20 bp, synthetic construct
  source 1..20: mol_type = other DNA
tttctactttagggaaaaaa

SEQ ID NO: 362 - DNA, 20 bp, synthetic construct
  source 1..20: mol_type = other DNA
tttctactttagggaaacaa

SEQ ID NO: 363 - DNA, 20 bp, synthetic construct
  source 1..20: mol_type = other DNA
ttctactttagggaaaaaat

SEQ ID NO: 364 - DNA, 20 bp, synthetic construct
  source 1..20: mol_type = other DNA
ttctactttagggaaacaat

SEQ ID NO: 365 - DNA, 20 bp, synthetic construct
  source 1..20: mol_type = other DNA
tctactttagggaaaaaatg

SEQ ID NO: 366 - DNA, 20 bp, synthetic construct
  source 1..20: mol_type = other DNA
tctactttagggaaacaatg

SEQ ID NO: 367 - DNA, 20 bp, synthetic construct
  source 1..20: mol_type = other DNA
ctactttagggaaaaaatgg

SEQ ID NO: 368 - DNA, 20 bp, synthetic construct
  source 1..20: mol_type = other DNA
ctactttagggaaacaatgg

SEQ ID NO: 369 - DNA, 20 bp, synthetic construct
  source 1..20: mol_type = other DNA
tttagggaaaaaatgggggc

SEQ ID NO: 370 - DNA, 20 bp, synthetic construct
  source 1..20: mol_type = other DNA
tttagggaaacaatgggggc

SEQ ID NO: 371 - DNA, 20 bp, synthetic construct
  source 1..20: mol_type = other DNA
ttagggaaaaaatgggggca

SEQ ID NO: 372 - DNA, 20 bp, synthetic construct
  source 1..20: mol_type = other DNA
ttagggaaacaatgggggca

SEQ ID NO: 373 - DNA, 20 bp, synthetic construct
  source 1..20: mol_type = other DNA
gtttcataatgtgagaaaaa

SEQ ID NO: 374 - DNA, 20 bp, synthetic construct
  source 1..20: mol_type = other DNA
gttttataatgtgagaaaaa

SEQ ID NO: 375 - DNA, 20 bp, synthetic construct
  source 1..20: mol_type = other DNA
attatgaaactcttaaaatc

SEQ ID NO: 376 - DNA, 20 bp, synthetic construct
  source 1..20: mol_type = other DNA
attataaaactcttaaaatc

SEQ ID NO: 377 - DNA, 20 bp, synthetic construct
  source 1..20: mol_type = other DNA
agcattatatattcaactac

SEQ ID NO: 378 - DNA, 20 bp, synthetic construct
  source 1..20: mol_type = other DNA
accattatatattcaactac

SEQ ID NO: 379 - DNA, 20 bp, synthetic construct
  source 1..20: mol_type = other DNA
caacgggcagttgcagcagc

SEQ ID NO: 380 - DNA, 20 bp, synthetic construct
  source 1..20: mol_type = other DNA
caacgggcagttacagcagc

SEQ ID NO: 381 - DNA, 20 bp, synthetic construct
  source 1..20: mol_type = other DNA
gcagttgcagcagctggact

SEQ ID NO: 382 - DNA, 20 bp, synthetic construct
  source 1..20: mol_type = other DNA
gcagttacagcagctggact

SEQ ID NO: 383 - DNA, 20 bp, synthetic construct
  source 1..20: mol_type = other DNA
tcagcttctgggcaggccat

SEQ ID NO: 384 - DNA, 20 bp, synthetic construct
  source 1..20: mol_type = other DNA
tcagcttctgggcaggccac

SEQ ID NO: 385 - DNA, 20 bp, synthetic construct
  source 1..20: mol_type = other DNA
cagcttctgggcaggccata

SEQ ID NO: 386 - DNA, 20 bp, synthetic construct
  source 1..20: mol_type = other DNA
cagcttctgggcaggccaca

SEQ ID NO: 387 - DNA, 20 bp, synthetic construct
  source 1..20: mol_type = other DNA
tagggaacttaaaatcatga

SEQ ID NO: 388 - DNA, 20 bp, synthetic construct
  source 1..20: mol_type = other DNA
cagggaacttaaaatcatga

SEQ ID NO: 389 - DNA, 20 bp, synthetic construct
  source 1..20: mol_type = other DNA
tcatgattttaagttcccta

SEQ ID NO: 390 - DNA, 20 bp, synthetic construct
  source 1..20: mol_type = other DNA
tcatgattttaagttccctg

SEQ ID NO: 391 - DNA, 20 bp, synthetic construct
  source 1..20: mol_type = other DNA
ttttgattttacaggttcat

SEQ ID NO: 392 - DNA, 20 bp, synthetic construct
  source 1..20: mol_type = other DNA
ttttgattttacaggttcac

SEQ ID NO: 393 - DNA, 20 bp, synthetic construct
  source 1..20: mol_type = other DNA
ttacaggttcataggtagaa

SEQ ID NO: 394 - DNA, 20 bp, synthetic construct
  source 1..20: mol_type = other DNA
ttacaggttcacaggtagaa

SEQ ID NO: 395 - DNA, 20 bp, synthetic construct
  source 1..20: mol_type = other DNA
tttacaggttcataggtaga

SEQ ID NO: 396 - DNA, 20 bp, synthetic construct
  source 1..20: mol_type = other DNA
tttacaggttcacaggtaga

SEQ ID NO: 397 - DNA, 20 bp, synthetic construct
  source 1..20: mol_type = other DNA
tgagtgggctcccaaaacct

SEQ ID NO: 398 - DNA, 20 bp, synthetic construct
  source 1..20: mol_type = other DNA
tgagtgggctccgaaaacct

SEQ ID NO: 399 - DNA, 20 bp, synthetic construct
  source 1..20: mol_type = other DNA
gagtgggctcccaaaacctt

SEQ ID NO: 400 - DNA, 20 bp, synthetic construct
  source 1..20: mol_type = other DNA
gagtgggctccgaaaacctt

SEQ ID NO: 401 - DNA, 20 bp, synthetic construct
  source 1..20: mol_type = other DNA
tgtcttgtcatagctctgct

SEQ ID NO: 402 - DNA, 20 bp, synthetic construct
  source 1..20: mol_type = other DNA
tctcttgtcatagctctgct

SEQ ID NO: 403 - DNA, 20 bp, synthetic construct
  source 1..20: mol_type = other DNA
atcactgggtatatacccaa

SEQ ID NO: 404 - DNA, 20 bp, synthetic construct
  source 1..20: mol_type = other DNA
atccctgggtatatacccaa

SEQ ID NO: 405 - DNA, 20 bp, synthetic construct
  source 1..20: mol_type = other DNA
tgacccagccatcccatcac

SEQ ID NO: 406 - DNA, 20 bp, synthetic construct
  source 1..20: mol_type = other DNA
tgacccagccatcccatccc

SEQ ID NO: 407 - DNA, 20 bp, synthetic construct
  source 1..20: mol_type = other DNA
gacccagccatcccatcact

SEQ ID NO: 408 - DNA, 20 bp, synthetic construct
  source 1..20: mol_type = other DNA
gacccagccatcccatccct

SEQ ID NO: 409 - DNA, 20 bp, synthetic construct
  source 1..20: mol_type = other DNA
tttgggtatatacccagtga

SEQ ID NO: 410 - DNA, 20 bp, synthetic construct
  source 1..20: mol_type = other DNA
tttgggtatatacccaggga

SEQ ID NO: 411 - DNA, 20 bp, synthetic construct
  source 1..20: mol_type = other DNA
tacccagtgatgggatggct

SEQ ID NO: 412 - DNA, 20 bp, synthetic construct
  source 1..20: mol_type = other DNA
tacccagggatgggatggct

SEQ ID NO: 413 - DNA, 20 bp, synthetic construct
  source 1..20: mol_type = other DNA
atacccagtgatgggatggc

SEQ ID NO: 414 - DNA, 20 bp, synthetic construct
  source 1..20: mol_type = other DNA
atacccagggatgggatggc

SEQ ID NO: 415 - DNA, 20 bp, synthetic construct
  source 1..20: mol_type = other DNA
gtatatacccagtgatggga

SEQ ID NO: 416 - DNA, 20 bp, synthetic construct
  source 1..20: mol_type = other DNA
gtatatacccagggatggga

SEQ ID NO: 417 - DNA, 20 bp, synthetic construct
  source 1..20: mol_type = other DNA
ttgggtatatacccagtgat

SEQ ID NO: 418 - DNA, 20 bp, synthetic construct
  source 1..20: mol_type = other DNA
ttgggtatatacccagggat

SEQ ID NO: 419 - DNA, 20 bp, synthetic construct
  source 1..20: mol_type = other DNA
tattcacaatagcaaagact

SEQ ID NO: 420 - DNA, 20 bp, synthetic construct
  source 1..20: mol_type = other DNA
tgttcacaatagcaaagact

SEQ ID NO: 421 - DNA, 20 bp, synthetic construct
  source 1..20: mol_type = other DNA
tcacactctggggactgttg

SEQ ID NO: 422 - DNA, 20 bp, synthetic construct
  source 1..20: mol_type = other DNA
tcacactccggggactgttg

SEQ ID NO: 423 - DNA, 20 bp, synthetic construct
  source 1..20: mol_type = other DNA
cacactctggggactgttgt

SEQ ID NO: 424 - DNA, 20 bp, synthetic construct
  source 1..20: mol_type = other DNA
cacactccggggactgttgt

SEQ ID NO: 425 - DNA, 20 bp, synthetic construct
  source 1..20: mol_type = other DNA
acactctggggactgttgtg

SEQ ID NO: 426 - DNA, 20 bp, synthetic construct
  source 1..20: mol_type = other DNA
acactccggggactgttgtg

SEQ ID NO: 427 - DNA, 20 bp, synthetic construct
  source 1..20: mol_type = other DNA
ctctggggactgttgtgggg

SEQ ID NO: 428 - DNA, 20 bp, synthetic construct
  source 1..20: mol_type = other DNA
ctccggggactgttgtgggg

SEQ ID NO: 429 - DNA, 20 bp, synthetic construct
  source 1..20: mol_type = other DNA
tctggggactgttgtggggt

SEQ ID NO: 430 - DNA, 20 bp, synthetic construct
  source 1..20: mol_type = other DNA
tccggggactgttgtggggt

SEQ ID NO: 431 - DNA, 20 bp, synthetic construct
  source 1..20: mol_type = other DNA
ctggggactgttgtggggtg

SEQ ID NO: 432 - DNA, 20 bp, synthetic construct
  source 1..20: mol_type = other DNA
ccggggactgttgtggggtg

SEQ ID NO: 433 - DNA, 20 bp, synthetic construct
  source 1..20: mol_type = other DNA
tggggactgttgtggggtgg

SEQ ID NO: 434 - DNA, 20 bp, synthetic construct
  source 1..20: mol_type = other DNA
cggggactgttgtggggtgg

SEQ ID NO: 435 - DNA, 20 bp, synthetic construct
  source 1..20: mol_type = other DNA
gaaggggaacatcacactct

SEQ ID NO: 436 - DNA, 20 bp, synthetic construct
  source 1..20: mol_type = other DNA
gaaggggaacatcacactcc

SEQ ID NO: 437 - DNA, 20 bp, synthetic construct
  source 1..20: mol_type = other DNA
aaggggaacatcacactctg

SEQ ID NO: 438 - DNA, 20 bp, synthetic construct
  source 1..20: mol_type = other DNA
aaggggaacatcacactccg

SEQ ID NO: 439 - DNA, 20 bp, synthetic construct
  source 1..20: mol_type = other DNA
gaaagactcataggactaac

SEQ ID NO: 440 - DNA, 20 bp, synthetic construct
  source 1..20: mol_type = other DNA
gaaagactcataggactaat

SEQ ID NO: 441 - DNA, 20 bp, synthetic construct
  source 1..20: mol_type = other DNA
gtgctcatgcctatgagcat

SEQ ID NO: 442 - DNA, 20 bp, synthetic construct
  source 1..20: mol_type = other DNA
gtgctcatgcctatgaacat

SEQ ID NO: 443 - DNA, 20 bp, synthetic construct
  source 1..20: mol_type = other DNA
ttctaatagcctatgctcat

SEQ ID NO: 444 - DNA, 20 bp, synthetic construct
  source 1..20: mol_type = other DNA
ttctaatagcctatgttcat

SEQ ID NO: 445 - DNA, 20 bp, synthetic construct
  source 1..20: mol_type = other DNA
ttcacagatccctagaacag

SEQ ID NO: 446 - DNA, 20 bp, synthetic construct
  source 1..20: mol_type = other DNA
ttcacagatacctagaacag

SEQ ID NO: 447 - DNA, 20 bp, synthetic construct
  source 1..20: mol_type = other DNA
tgactttggaaccggctaac

SEQ ID NO: 448 - DNA, 20 bp, synthetic construct
  source 1..20: mol_type = other DNA
tgactttggaacctgctaac

SEQ ID NO: 449 - DNA, 20 bp, synthetic construct
  source 1..20: mol_type = other DNA
ggaacagagagagggaaagt

SEQ ID NO: 450 - DNA, 20 bp, synthetic construct
  source 1..20: mol_type = other DNA
ggaacagagagagggaaagg

SEQ ID NO: 451 - DNA, 20 bp, synthetic construct
  source 1..20: mol_type = other DNA
gaacagagagagggaaagtg

SEQ ID NO: 452 - DNA, 20 bp, synthetic construct
  source 1..20: mol_type = other DNA
gaacagagagagggaaaggg

SEQ ID NO: 453 - DNA, 20 bp, synthetic construct
  source 1..20: mol_type = other DNA
aacagagagagggaaagtgg

SEQ ID NO: 454 - DNA, 20 bp, synthetic construct
  source 1..20: mol_type = other DNA
aacagagagagggaaagggg

SEQ ID NO: 455 - DNA, 20 bp, synthetic construct
  source 1..20: mol_type = other DNA
agagagagggaaagtggggg

SEQ ID NO: 456 - DNA, 20 bp, synthetic construct
  source 1..20: mol_type = other DNA
agagagagggaaaggggggg

SEQ ID NO: 457 - DNA, 20 bp, synthetic construct
  source 1..20: mol_type = other DNA
aggctccatctccaacgttg

SEQ ID NO: 458 - DNA, 20 bp, synthetic construct
  source 1..20: mol_type = other DNA
aggctccatctccaacgctg

SEQ ID NO: 459 - DNA, 20 bp, synthetic construct
  source 1..20: mol_type = other DNA
ccaggctccatctccaacgt

SEQ ID NO: 460 - DNA, 20 bp, synthetic construct
  source 1..20: mol_type = other DNA
ccaggctccatctccaacgc

SEQ ID NO: 461 - DNA, 20 bp, synthetic construct
  source 1..20: mol_type = other DNA
caggctccatctccaacgtt

SEQ ID NO: 462 - DNA, 20 bp, synthetic construct
  source 1..20: mol_type = other DNA
caggctccatctccaacgct

SEQ ID NO: 463 - DNA, 20 bp, synthetic construct
  source 1..20: mol_type = other DNA
tcagttgcaatccccaacgt

SEQ ID NO: 464 - DNA, 20 bp, synthetic construct
  source 1..20: mol_type = other DNA
tcagttgcaatccccagcgt

SEQ ID NO: 465 - DNA, 20 bp, synthetic construct
  source 1..20: mol_type = other DNA
gcaatccccaacgttggaga

SEQ ID NO: 466 - DNA, 20 bp, synthetic construct
  source 1..20: mol_type = other DNA
gcaatccccagcgttggaga

SEQ ID NO: 467 - DNA, 20 bp, synthetic construct
  source 1..20: mol_type = other DNA
ccaacgttggagatggagcc

SEQ ID NO: 468 - DNA, 20 bp, synthetic construct
  source 1..20: mol_type = other DNA
ccagcgttggagatggagcc

SEQ ID NO: 469 - DNA, 20 bp, synthetic construct
  source 1..20: mol_type = other DNA
aatcttatcacatacataaa

SEQ ID NO: 470 - DNA, 20 bp, synthetic construct
  source 1..20: mol_type = other DNA
aatcttatcgcatacataaa

SEQ ID NO: 471 - DNA, 20 bp, synthetic construct
  source 1..20: mol_type = other DNA
tttagaataagactggaact

SEQ ID NO: 472 - DNA, 20 bp, synthetic construct
  source 1..20: mol_type = other DNA
tttagagtaagactggaact

SEQ ID NO: 473 - DNA, 20 bp, synthetic construct
  source 1..20: mol_type = other DNA
tccccagtttagaataagac

SEQ ID NO: 474 - DNA, 20 bp, synthetic construct
  source 1..20: mol_type = other DNA
tccccagtttagagtaagac

SEQ ID NO: 475 - DNA, 20 bp, synthetic construct
  source 1..20: mol_type = other DNA
tccagtcttattctaaactg

SEQ ID NO: 476 - DNA, 20 bp, synthetic construct
  source 1..20: mol_type = other DNA
tccagtcttactctaaactg

SEQ ID NO: 477 - DNA, 20 bp, synthetic construct
  source 1..20: mol_type = other DNA
ttccagtcttattctaaact

SEQ ID NO: 478 - DNA, 20 bp, synthetic construct
  source 1..20: mol_type = other DNA
ttccagtcttactctaaact

SEQ ID NO: 479 - DNA, 20 bp, synthetic construct
  source 1..20: mol_type = other DNA
gttccagtcttattctaaac

SEQ ID NO: 480 - DNA, 20 bp, synthetic construct
  source 1..20: mol_type = other DNA
gttccagtcttactctaaac

SEQ ID NO: 481 - DNA, 20 bp, synthetic construct
  source 1..20: mol_type = other DNA
gacacagcaggaagtttatc

SEQ ID NO: 482 - DNA, 20 bp, synthetic construct
  source 1..20: mol_type = other DNA
gacacagcaggaaggttatc

SEQ ID NO: 483 - DNA, 20 bp, synthetic construct
  source 1..20: mol_type = other DNA
ttcactcttgttgtccaggc

SEQ ID NO: 484 - DNA, 20 bp, synthetic construct
  source 1..20: mol_type = other DNA
ttcactcttgttgtccaagc

SEQ ID NO: 485 - DNA, 20 bp, synthetic construct
  source 1..20: mol_type = other DNA
ttgtccaggctggagtgcaa

SEQ ID NO: 486 - DNA, 20 bp, synthetic construct
  source 1..20: mol_type = other DNA
ttgtccaagctggagtgcaa

SEQ ID NO: 487 - DNA, 20 bp, synthetic construct
  source 1..20: mol_type = other DNA
cgtgccattgcactccagcc

SEQ ID NO: 488 - DNA, 20 bp, synthetic construct
  source 1..20: mol_type = other DNA
cgtgccattgcactccagct

SEQ ID NO: 489 - DNA, 20 bp, synthetic construct
  source 1..20: mol_type = other DNA
tcccgaagtgctgagattac

SEQ ID NO: 490 - DNA, 20 bp, synthetic construct
  source 1..20: mol_type = other DNA
tcccaaagtgctgagattac

SEQ ID NO: 491 - DNA, 20 bp, synthetic construct
  source 1..20: mol_type = other DNA
ctcagcacttcgggaggcca

SEQ ID NO: 492 - DNA, 20 bp, synthetic construct
  source 1..20: mol_type = other DNA
ctcagcactttgggaggcca

SEQ ID NO: 493 - DNA, 20 bp, synthetic construct
  source 1..20: mol_type = other DNA
tgtaatctcagcacttcggg

SEQ ID NO: 494 - DNA, 20 bp, synthetic construct
  source 1..20: mol_type = other DNA
tgtaatctcagcactttggg

SEQ ID NO: 495 - DNA, 20 bp, synthetic construct
  source 1..20: mol_type = other DNA
acctgtaatctcagcacttc

SEQ ID NO: 496 - DNA, 20 bp, synthetic construct
  source 1..20: mol_type = other DNA
acctgtaatctcagcacttt

SEQ ID NO: 497 - DNA, 20 bp, synthetic construct
  source 1..20: mol_type = other DNA
gcacttcgggaggccaagga

SEQ ID NO: 498 - DNA, 20 bp, synthetic construct
  source 1..20: mol_type = other DNA
gcactttgggaggccaagga

SEQ ID NO: 499 - DNA, 20 bp, synthetic construct
  source 1..20: mol_type = other DNA
agcacttcgggaggccaagg

SEQ ID NO: 500 - DNA, 20 bp, synthetic construct
  source 1..20: mol_type = other DNA
agcactttgggaggccaagg

SEQ ID NO: 501 - DNA, 20 bp, synthetic construct
  source 1..20: mol_type = other DNA
tgccttaatcattacggact

SEQ ID NO: 502 - DNA, 20 bp, synthetic construct
  source 1..20: mol_type = other DNA
tgcctcaatcattacggact

SEQ ID NO: 503 - DNA, 20 bp, synthetic construct
  source 1..20: mol_type = other DNA
tgtggttgccttaatcatta

SEQ ID NO: 504 - DNA, 20 bp, synthetic construct
  source 1..20: mol_type = other DNA
tgtggttgcctcaatcatta

SEQ ID NO: 505 - DNA, 20 bp, synthetic construct
  source 1..20: mol_type = other DNA
cttaatcattacggactagg

SEQ ID NO: 506 - DNA, 20 bp, synthetic construct
  source 1..20: mol_type = other DNA
ctcaatcattacggactagg

SEQ ID NO: 507 - DNA, 20 bp, synthetic construct
  source 1..20: mol_type = other DNA
cgcctagtccgtaatgatta

SEQ ID NO: 508 - DNA, 20 bp, synthetic construct
  source 1..20: mol_type = other DNA
cgcctagtccgtaatgattg

SEQ ID NO: 509 - DNA, 20 bp, synthetic construct
  source 1..20: mol_type = other DNA
tgccttaatcattacggact

SEQ ID NO: 510 - DNA, 20 bp, synthetic construct
  source 1..20: mol_type = other DNA
tgccttaatcattacagact

SEQ ID NO: 511 - DNA, 20 bp, synthetic construct
  source 1..20: mol_type = other DNA
cttaatcattacggactagg

SEQ ID NO: 512 - DNA, 20 bp, synthetic construct
  source 1..20: mol_type = other DNA
cttaatcattacagactagg

SEQ ID NO: 513 - DNA, 20 bp, synthetic construct
  source 1..20: mol_type = other DNA
cgcctagtccgtaatgatta

SEQ ID NO: 514 - DNA, 20 bp, synthetic construct
  source 1..20: mol_type = other DNA
cgcctagtctgtaatgatta

SEQ ID NO: 515 - DNA, 20 bp, synthetic construct
  source 1..20: mol_type = other DNA
ggggtttcaccatgttagtc

SEQ ID NO: 516 - DNA, 20 bp, synthetic construct
  source 1..20: mol_type = other DNA
ggagtttcaccatgttagtc

SEQ ID NO: 517 - DNA, 20 bp, synthetic construct
  source 1..20: mol_type = other DNA
ttgtatttttagtagagatg

SEQ ID NO: 518 - DNA, 20 bp, synthetic construct
  source 1..20: mol_type = other DNA
ttgtatttttagtagagatg

SEQ ID NO: 519 - DNA, 20 bp, synthetic construct
  source 1..20: mol_type = other DNA
tttgtatttttagtagagat

SEQ ID NO: 520 - DNA, 20 bp, synthetic construct
  source 1..20: mol_type = other DNA
tttgtatttttagtagagat

SEQ ID NO: 521 - DNA, 20 bp, synthetic construct
  source 1..20: mol_type = other DNA
ttttgtatttttagtagaga

SEQ ID NO: 522 - DNA, 20 bp, synthetic construct
  source 1..20: mol_type = other DNA
ttttgtatttttagtagaga

SEQ ID NO: 523 - DNA, 20 bp, synthetic construct
  source 1..20: mol_type = other DNA
gaatacagagttctggggag

SEQ ID NO: 524 - DNA, 20 bp, synthetic construct
  source 1..20: mol_type = other DNA
gaatacagagttcttgggag

SEQ ID NO: 525 - DNA, 20 bp, synthetic construct
  source 1..20: mol_type = other DNA
gcctggaatacagagttctg

SEQ ID NO: 526 - DNA, 20 bp, synthetic construct
  source 1..20: mol_type = other DNA
gcctggaatacagagttctt

SEQ ID NO: 527 - DNA, 20 bp, synthetic construct
  source 1..20: mol_type = other DNA
tccccagaactctgtattcc

SEQ ID NO: 528 - DNA, 20 bp, synthetic construct
  source 1..20: mol_type = other DNA
tcccaagaactctgtattcc

SEQ ID NO: 529 - DNA, 20 bp, synthetic construct
  source 1..20: mol_type = other DNA
aaagctaggccagttgctca

SEQ ID NO: 530 - DNA, 20 bp, synthetic construct
  source 1..20: mol_type = other DNA
aaagctaggccggttgctca

SEQ ID NO: 531 - DNA, 20 bp, synthetic construct
  source 1..20: mol_type = other DNA
taaagctaggccagttgctc

SEQ ID NO: 532 - DNA, 20 bp, synthetic construct
  source 1..20: mol_type = other DNA
taaagctaggccggttgctc

SEQ ID NO: 533 - DNA, 20 bp, synthetic construct
  source 1..20: mol_type = other DNA
agagaagcttgaaagtcaag

SEQ ID NO: 534 - DNA, 20 bp, synthetic construct
  source 1..20: mol_type = other DNA
agagaagcttcaaagtcaag

SEQ ID NO: 535 - DNA, 20 bp, synthetic construct
  source 1..20: mol_type = other DNA
tggagaggtgaggacatttc

SEQ ID NO: 536 - DNA, 20 bp, synthetic construct
  source 1..20: mol_type = other DNA
tggagaggtggggacatttc

SEQ ID NO: 537 - DNA, 20 bp, synthetic construct
  source 1..20: mol_type = other DNA
caatttcctcctcttgaaaa

SEQ ID NO: 538 - DNA, 20 bp, synthetic construct
  source 1..20: mol_type = other DNA
caaattcctcctcttgaaaa

SEQ ID NO: 539 - DNA, 20 bp, synthetic construct
  source 1..20: mol_type = other DNA
ttttcaagaggaggaaattg

SEQ ID NO: 540 - DNA, 20 bp, synthetic construct
  source 1..20: mol_type = other DNA
ttttcaagaggaggaatttg

SEQ ID NO: 541 - DNA, 20 bp, synthetic construct
  source 1..20: mol_type = other DNA
aaaaaaaaaaagctcttacc

SEQ ID NO: 542 - DNA, 20 bp, synthetic construct
  source 1..20: mol_type = other DNA
aaaaaaaaaaagctcttacc

SEQ ID NO: 543 - DNA, 20 bp, synthetic construct
  source 1..20: mol_type = other DNA
aaaagctcttacctggttgc

SEQ ID NO: 544 - DNA, 20 bp, synthetic construct
  source 1..20: mol_type = other DNA
aaaagctcttacctggttgc

SEQ ID NO: 545 - DNA, 20 bp, synthetic construct
  source 1..20: mol_type = other DNA
aaaaaaaaaaagctcttacc

SEQ ID NO: 546 - DNA, 20 bp, synthetic construct
  source 1..20: mol_type = other DNA
aaaaaaaaaaagctcttacc

SEQ ID NO: 547 - DNA, 20 bp, synthetic construct
  source 1..20: mol_type = other DNA
aaaagctcttacctggttgc

SEQ ID NO: 548 - DNA, 20 bp, synthetic construct
  source 1..20: mol_type = other DNA
aaaagctcttacctggttgc

SEQ ID NO: 549 - DNA, 20 bp, synthetic construct
  source 1..20: mol_type = other DNA
tttttttttaaatcaacata

SEQ ID NO: 550 - DNA, 20 bp, synthetic construct
  source 1..20: mol_type = other DNA
ttttttttaaaatcaacata

SEQ ID NO: 551 - DNA, 20 bp, synthetic construct
  source 1..20: mol_type = other DNA
aaacaactgatagtcgtcag

SEQ ID NO: 552 - DNA, 20 bp, synthetic construct
  source 1..20: mol_type = other DNA
aaacaactgataatcgtcag

SEQ ID NO: 553 - DNA, 20 bp, synthetic construct
  source 1..20: mol_type = other DNA
atgcctaaataatgtcattg

SEQ ID NO: 554 - DNA, 20 bp, synthetic construct
  source 1..20: mol_type = other DNA
atgcctaaataatgtcatcg

SEQ ID NO: 555 - DNA, 20 bp, synthetic construct
  source 1..20: mol_type = other DNA
tttccacaatgacattattt

SEQ ID NO: 556 - DNA, 20 bp, synthetic construct
  source 1..20: mol_type = other DNA
tttccacgatgacattattt

SEQ ID NO: 557 - DNA, 20 bp, synthetic construct
  source 1..20: mol_type = other DNA
gcagaaagtgacagtgagac

SEQ ID NO: 558 - DNA, 20 bp, synthetic construct
  source 1..20: mol_type = other DNA
gcagaaagtgactgtgagac

SEQ ID NO: 559 - DNA, 20 bp, synthetic construct
  source 1..20: mol_type = other DNA
agtgacagtgagactggaaa

SEQ ID NO: 560 - DNA, 20 bp, synthetic construct
  source 1..20: mol_type = other DNA
agtgactgtgagactggaaa

SEQ ID NO: 561 - DNA, 20 bp, synthetic construct
  source 1..20: mol_type = other DNA
aagtgacagtgagactggaa

SEQ ID NO: 562 - DNA, 20 bp, synthetic construct
  source 1..20: mol_type = other DNA
aagtgactgtgagactggaa

SEQ ID NO: 563 - DNA, 20 bp, synthetic construct
  source 1..20: mol_type = other DNA
cagtgagactggaaagggat

SEQ ID NO: 564 - DNA, 20 bp, synthetic construct
  source 1..20: mol_type = other DNA
ctgtgagactggaaagggat

SEQ ID NO: 565 - DNA, 20 bp, synthetic construct
  source 1..20: mol_type = other DNA
tcactctcactctgttgccc

SEQ ID NO: 566 - DNA, 20 bp, synthetic construct
  source 1..20: mol_type = other DNA
tcattctcactctgttgccc

SEQ ID NO: 567 - DNA, 20 bp, synthetic construct
  source 1..20: mol_type = other DNA
gtgagagtgagactctttct

SEQ ID NO: 568 - DNA, 20 bp, synthetic construct
  source 1..20: mol_type = other DNA
gtgagaatgagactctttct

SEQ ID NO: 569 - DNA, 20 bp, synthetic construct
  source 1..20: mol_type = other DNA
aactctgggggaaaagaagt

SEQ ID NO: 570 - DNA, 20 bp, synthetic construct
  source 1..20: mol_type = other DNA
aactctgggggaaaaaaagt

SEQ ID NO: 571 - DNA, 20 bp, synthetic construct
  source 1..20: mol_type = other DNA
cttcttttcccccagagttc

SEQ ID NO: 572 - DNA, 20 bp, synthetic construct
  source 1..20: mol_type = other DNA
ctttttttcccccagagttc

SEQ ID NO: 573 - DNA, 20 bp, synthetic construct
  source 1..20: mol_type = other DNA
gatgaatgaatcttgtttac

SEQ ID NO: 574 - DNA, 20 bp, synthetic construct
  source 1..20: mol_type = other DNA
gatgaatgaattttgtttac

SEQ ID NO: 575 - DNA, 20 bp, synthetic construct
  source 1..20: mol_type = other DNA
atgaatgaatcttgtttaca

SEQ ID NO: 576 - DNA, 20 bp, synthetic construct
  source 1..20: mol_type = other DNA
atgaatgaattttgtttaca

SEQ ID NO: 577 - DNA, 20 bp, synthetic construct
  source 1..20: mol_type = other DNA
agattcattcatctcaaatt

SEQ ID NO: 578 - DNA, 20 bp, synthetic construct
  source 1..20: mol_type = other DNA
aaattcattcatctcaaatt

SEQ ID NO: 579 - DNA, 20 bp, synthetic construct
  source 1..20: mol_type = other DNA
aagattcattcatctcaaat

SEQ ID NO: 580 - DNA, 20 bp, synthetic construct
  source 1..20: mol_type = other DNA
aaaattcattcatctcaaat

SEQ ID NO: 581 - DNA, 20 bp, synthetic construct
  source 1..20: mol_type = other DNA
gagaagtttttatctggtga

SEQ ID NO: 582 - DNA, 20 bp, synthetic construct
  source 1..20: mol_type = other DNA
gagaagttcttatctggtga

SEQ ID NO: 583 - DNA, 20 bp, synthetic construct
  source 1..20: mol_type = other DNA
agaagaaatgtctatgaaag

SEQ ID NO: 584 - DNA, 20 bp, synthetic construct
  source 1..20: mol_type = other DNA
agaaaaaatgtctatgaaag

SEQ ID NO: 585 - DNA, 20 bp, synthetic construct
  source 1..20: mol_type = other DNA
gacatttcttctgcatgggt

SEQ ID NO: 586 - DNA, 20 bp, synthetic construct
  source 1..20: mol_type = other DNA
gacattttttctgcatgggt

SEQ ID NO: 587 - DNA, 20 bp, synthetic construct
  source 1..20: mol_type = other DNA
catagacatttcttctgcat

SEQ ID NO: 588 - DNA, 20 bp, synthetic construct
  source 1..20: mol_type = other DNA
catagacattttttctgcat

SEQ ID NO: 589 - DNA, 20 bp, synthetic construct
  source 1..20: mol_type = other DNA
tcatagacatttcttctgca

SEQ ID NO: 590 - DNA, 20 bp, synthetic construct
  source 1..20: mol_type = other DNA
tcatagacattttttctgca

SEQ ID NO: 591 - DNA, 20 bp, synthetic construct
  source 1..20: mol_type = other DNA
tgtctatgaaagcggtcaaa

SEQ ID NO: 592 - DNA, 20 bp, synthetic construct
  source 1..20: mol_type = other DNA
tgtctgtgaaagcggtcaaa

SEQ ID NO: 593 - DNA, 20 bp, synthetic construct
  source 1..20: mol_type = other DNA
atgtctatgaaagcggtcaa

SEQ ID NO: 594 - DNA, 20 bp, synthetic construct
  source 1..20: mol_type = other DNA
atgtctgtgaaagcggtcaa

SEQ ID NO: 595 - DNA, 20 bp, synthetic construct
  source 1..20: mol_type = other DNA
agaagaaatgtctatgaaag

SEQ ID NO: 596 - DNA, 20 bp, synthetic construct
  source 1..20: mol_type = other DNA
agaagaaatgtctgtgaaag

SEQ ID NO: 597 - DNA, 20 bp, synthetic construct
  source 1..20: mol_type = other DNA
catagacatttcttctgcat

SEQ ID NO: 598 - DNA, 20 bp, synthetic construct
  source 1..20: mol_type = other DNA
cacagacatttcttctgcat

SEQ ID NO: 599 - DNA, 20 bp, synthetic construct
  source 1..20: mol_type = other DNA
tcatagacatttcttctgca

SEQ ID NO: 600 - DNA, 20 bp, synthetic construct
  source 1..20: mol_type = other DNA
tcacagacatttcttctgca

SEQ ID NO: 601 - DNA, 20 bp, synthetic construct
  source 1..20: mol_type = other DNA
aatgagtagctctgcaaaag

SEQ ID NO: 602 - DNA, 20 bp, synthetic construct
  source 1..20: mol_type = other DNA
aatgagtagctttgcaaaag

SEQ ID NO: 603 - DNA, 20 bp, synthetic construct
  source 1..20: mol_type = other DNA
tctgcaaaagtggattttcc

SEQ ID NO: 604 - DNA, 20 bp, synthetic construct
  source 1..20: mol_type = other DNA
tttgcaaaagtggattttcc

SEQ ID NO: 605 - DNA, 20 bp, synthetic construct
  source 1..20: mol_type = other DNA
ctactcattttttgataatt

SEQ ID NO: 606 - DNA, 20 bp, synthetic construct
  source 1..20: mol_type = other DNA
ctactcatttttcgataatt

SEQ ID NO: 607 - DNA, 20 bp, synthetic construct
  source 1..20: mol_type = other DNA
gctactcattttttgataat

SEQ ID NO: 608 - DNA, 20 bp, synthetic construct
  source 1..20: mol_type = other DNA
gctactcatttttcgataat

SEQ ID NO: 609 - DNA, 20 bp, synthetic construct
  source 1..20: mol_type = other DNA
aagacatacaaatggcaaac

SEQ ID NO: 610 - DNA, 20 bp, synthetic construct
  source 1..20: mol_type = other DNA
aagacgtacaaatggcaaac

SEQ ID NO: 611 - DNA, 20 bp, synthetic construct
  source 1..20: mol_type = other DNA
ttcaagagaagacatacaaa

SEQ ID NO: 612 - DNA, 20 bp, synthetic construct
  source 1..20: mol_type = other DNA
ttcaagagaagacgtacaaa

SEQ ID NO: 613 - DNA, 20 bp, synthetic construct
  source 1..20: mol_type = other DNA
aagacatacaaatggcaaac

SEQ ID NO: 614 - DNA, 20 bp, synthetic construct
  source 1..20: mol_type = other DNA
aagacatacaagtggcaaac

SEQ ID NO: 615 - DNA, 20 bp, synthetic construct
  source 1..20: mol_type = other DNA
ttcaagagaagacatacaaa

SEQ ID NO: 616 - DNA, 20 bp, synthetic construct
  source 1..20: mol_type = other DNA
ttcaagagaagacatacaag

SEQ ID NO: 617 - DNA, 20 bp, synthetic construct
  source 1..20: mol_type = other DNA
tatgtgttctgcctccccta

SEQ ID NO: 618 - DNA, 20 bp, synthetic construct
  source 1..20: mol_type = other DNA
tacgtgttctgcctccccta

SEQ ID NO: 619 - DNA, 20 bp, synthetic construct
  source 1..20: mol_type = other DNA
ctctttttttttttttttga

SEQ ID NO: 620 - DNA, 22 bp, synthetic construct
  source 1..22: mol_type = other DNA
cctcctttttttttttttttga

SEQ ID NO: 621 - DNA, 20 bp, synthetic construct
  source 1..20: mol_type = other DNA
ctctttttttttttttttga

SEQ ID NO: 622 - DNA, 22 bp, synthetic construct
  source 1..22: mol_type = other DNA
cctcttttttttttttttttga

SEQ ID NO: 623 - DNA, 20 bp, synthetic construct
  source 1..20: mol_type = other DNA
cgtcaaaaaaaaaaaaaaag

SEQ ID NO: 624 - DNA, 20 bp, synthetic construct
  source 1..20: mol_type = other DNA
gtcaaaaaaaaaaaaaaaag

SEQ ID NO: 625 - DNA, 20 bp, synthetic construct
  source 1..20: mol_type = other DNA
ctctttttttttttttttga

SEQ ID NO: 626 - DNA, 24 bp, synthetic construct
  source 1..24: mol_type = other DNA
tcctctttttttttttttttttga

SEQ ID NO: 627 - DNA, 20 bp, synthetic construct
  source 1..20: mol_type = other DNA
cgtcaaaaaaaaaaaaaaag

SEQ ID NO: 628 - DNA, 20 bp, synthetic construct
  source 1..20: mol_type = other DNA
tcaaaaaaaaaaaaaaaaag

SEQ ID NO: 629 - DNA, 20 bp, synthetic construct
  source 1..20: mol_type = other DNA
ttttatttattaattgccta

SEQ ID NO: 630 - DNA, 20 bp, synthetic construct
  source 1..20: mol_type = other DNA
cccccccccccccccccccc

SEQ ID NO: 631 - DNA, 20 bp, synthetic construct
  source 1..20: mol_type = other DNA
ggtttctttcttctgtagtg

SEQ ID NO: 632 - DNA, 20 bp, synthetic construct
  source 1..20: mol_type = other DNA
cccccccccccccccccccc

SEQ ID NO: 633 - DNA, 20 bp, synthetic construct
  source 1..20: mol_type = other DNA
cccagctattcaggaggttg

SEQ ID NO: 634 - DNA, 20 bp, synthetic construct
  source 1..20: mol_type = other DNA
cccccccccccccccccccc

SEQ ID NO: 635 - DNA, 20 bp, synthetic construct
  source 1..20: mol_type = other DNA
ctcccaaagtgctgggatta

SEQ ID NO: 636 - DNA, 20 bp, synthetic construct
  source 1..20: mol_type = other DNA
gggggggggggggggggggg

SEQ ID NO: 637 - DNA, 20 bp, synthetic construct
  source 1..20: mol_type = other DNA
ctttcttgaggatgtagcca

SEQ ID NO: 638 - DNA, 20 bp, synthetic construct
  source 1..20: mol_type = other DNA
cccccccccccccccccccc

SEQ ID NO: 639 - DNA, 20 bp, synthetic construct
  source 1..20: mol_type = other DNA
aaatgtgaaagctggtcaag

SEQ ID NO: 640 - DNA, 20 bp, synthetic construct
  source 1..20: mol_type = other DNA
gggggggggggggggggggg

SEQ ID NO: 641 - DNA, 20 bp, synthetic construct
  source 1..20: mol_type = other DNA
tctgggcaaacagaaaaaaa

SEQ ID NO: 642 - DNA, 20 bp, synthetic construct
  source 1..20: mol_type = other DNA
gggggggggggggggggggg

SEQ ID NO: 643 - DNA, 20 bp, synthetic construct
  source 1..20: mol_type = other DNA
ggtggttctgcccaaggttt

SEQ ID NO: 644 - DNA, 20 bp, synthetic construct
  source 1..20: mol_type = other DNA
cccccccccccccccccccc

SEQ ID NO: 645 - DNA, 20 bp, synthetic construct
  source 1..20: mol_type = other DNA
tgcattgttcctctgttcta

SEQ ID NO: 646 - DNA, 20 bp, synthetic construct
  source 1..20: mol_type = other DNA
cccccccccccccccccccc

SEQ ID NO: 647 - DNA, 20 bp, synthetic construct
  source 1..20: mol_type = other DNA
tgtagaagtgactttggaac

SEQ ID NO: 648 - DNA, 20 bp, synthetic construct
  source 1..20: mol_type = other DNA
cccccccccccccccccccc

SEQ ID NO: 649 - DNA, 20 bp, synthetic construct
  source 1..20: mol_type = other DNA
agagtttcactcttgttgtc

SEQ ID NO: 650 - DNA, 20 bp, synthetic construct
  source 1..20: mol_type = other DNA
gggggggggggggggggggg

SEQ ID NO: 651 - DNA, 20 bp, synthetic construct
  source 1..20: mol_type = other DNA
atgtggttgccttaatcatt

SEQ ID NO: 652 - DNA, 20 bp, synthetic construct
  source 1..20: mol_type = other DNA
gggggggggggggggggggg

SEQ ID NO: 653 - DNA, 20 bp, synthetic construct
  source 1..20: mol_type = other DNA
ttgtatttttagtagagatg

SEQ ID NO: 654 - DNA, 20 bp, synthetic construct
  source 1..20: mol_type = other DNA
cccccccccccccccccccc

SEQ ID NO: 655 - DNA, 20 bp, synthetic construct
  source 1..20: mol_type = other DNA
tttgtatttttagtagagat

SEQ ID NO: 656 - DNA, 20 bp, synthetic construct
  source 1..20: mol_type = other DNA
cccccccccccccccccccc

SEQ ID NO: 657 - DNA, 20 bp, synthetic construct
  source 1..20: mol_type = other DNA
aaggcctggaatacagagtt

SEQ ID NO: 658 - DNA, 20 bp, synthetic construct
  source 1..20: mol_type = other DNA
gggggggggggggggggggg

SEQ ID NO: 659 - DNA, 20 bp, synthetic construct
  source 1..20: mol_type = other DNA
ttacattttgggggcaaata

SEQ ID NO: 660 - DNA, 20 bp, synthetic construct
  source 1..20: mol_type = other DNA
gggggggggggggggggggg

SEQ ID NO: 661 - DNA, 20 bp, synthetic construct
  source 1..20: mol_type = other DNA
agggactggtttcatgacta

SEQ ID NO: 662 - DNA, 20 bp, synthetic construct
  source 1..20: mol_type = other DNA
cccccccccccccccccccc

SEQ ID NO: 663 - DNA, 20 bp, synthetic construct
  source 1..20: mol_type = other DNA
gggggggggggggggggggg

SEQ ID NO: 664 - DNA, 20 bp, synthetic construct
  source 1..20: mol_type = other DNA
tatattaatattataataat

SEQ ID NO: 665 - DNA, 20 bp, synthetic construct
  source 1..20: mol_type = other DNA
gggggggggggggggggggg

SEQ ID NO: 666 - DNA, 20 bp, synthetic construct
  source 1..20: mol_type = other DNA
atgtacaaaggtttttcttc

SEQ ID NO: 667 - DNA, 20 bp, synthetic construct
  source 1..20: mol_type = other DNA
gggggggggggggggggggg

SEQ ID NO: 668 - DNA, 20 bp, synthetic construct
  source 1..20: mol_type = other DNA
cctcctgggttcaagcgatt

SEQ ID NO: 669 - DNA, 20 bp, synthetic construct
  source 1..20: mol_type = other DNA
gggggggggggggggggggg

SEQ ID NO: 670 - DNA, 20 bp, synthetic construct
  source 1..20: mol_type = other DNA
ctcactgcaacctacacctc

SEQ ID NO: 671 - DNA, 20 bp, synthetic construct
  source 1..20: mol_type = other DNA
gggggggggggggggggggg

SEQ ID NO: 672 - DNA, 20 bp, synthetic construct
  source 1..20: mol_type = other DNA
tacttttgcagggggtggag

SEQ ID NO: 673 - DNA, 20 bp, synthetic construct
  source 1..20: mol_type = other DNA
gggggggggggggggggggg

SEQ ID NO: 674 - DNA, 20 bp, synthetic construct
  source 1..20: mol_type = other DNA
ggcaaaggacatgaaaattt

SEQ ID NO: 675 - DNA, 20 bp, synthetic construct
  source 1..20: mol_type = other DNA
gggggggggggggggggggg

SEQ ID NO: 676 - DNA, 20 bp, synthetic construct
  source 1..20: mol_type = other DNA
ctttcctaatcagttggggt

SEQ ID NO: 677 - DNA, 20 bp, synthetic construct
  source 1..20: mol_type = other DNA
gggggggggggggggggggg

SEQ ID NO: 678 - DNA, 20 bp, synthetic construct
  source 1..20: mol_type = other DNA
ttctcagacttatttgacca

SEQ ID NO: 679 - DNA, 20 bp, synthetic construct
  source 1..20: mol_type = other DNA
gggggggggggggggggggg

SEQ ID NO: 680 - DNA, 20 bp, synthetic construct
  source 1..20: mol_type = other DNA
aagaaaagtgcaaaatcctt

SEQ ID NO: 681 - DNA, 20 bp, synthetic construct
  source 1..20: mol_type = other DNA
gggggggggggggggggggg

SEQ ID NO: 682 - DNA, 20 bp, synthetic construct
  source 1..20: mol_type = other DNA
cacattaggtactattacta

SEQ ID NO: 683 - DNA, 20 bp, synthetic construct
  source 1..20: mol_type = other DNA
gggggggggggggggggggg

SEQ ID NO: 684 - DNA, 20 bp, synthetic construct
  source 1..20: mol_type = other DNA
gcacacagttttgtttcaaa

SEQ ID NO: 685 - DNA, 20 bp, synthetic construct
  source 1..20: mol_type = other DNA
gggggggggggggggggggg

SEQ ID NO: 686 - DNA, 20 bp, synthetic construct
  source 1..20: mol_type = other DNA
atttgctaaagtggctaata

SEQ ID NO: 687 - DNA, 20 bp, synthetic construct
  source 1..20: mol_type = other DNA
gggggggggggggggggggg

SEQ ID NO: 688 - DNA, 20 bp, synthetic construct
  source 1..20: mol_type = other DNA
atcctttagtaacctccaca

SEQ ID NO: 689 - DNA, 20 bp, synthetic construct
  source 1..20: mol_type = other DNA
gggggggggggggggggggg

SEQ ID NO: 690 - DNA, 20 bp, synthetic construct
  source 1..20: mol_type = other DNA
ttctaagtttcctcaacttg

SEQ ID NO: 691 - DNA, 20 bp, synthetic construct
  source 1..20: mol_type = other DNA
gggggggggggggggggggg

SEQ ID NO: 692 - DNA, 20 bp, synthetic construct
  source 1..20: mol_type = other DNA
ttatatattcaactacagga

SEQ ID NO: 693 - DNA, 20 bp, synthetic construct
  source 1..20: mol_type = other DNA
gggggggggggggggggggg

SEQ ID NO: 694 - DNA, 20 bp, synthetic construct
  source 1..20: mol_type = other DNA
tgggtatatacccaaaggac

SEQ ID NO: 695 - DNA, 20 bp, synthetic construct
  source 1..20: mol_type = other DNA
gggggggggggggggggggg

SEQ ID NO: 696 - DNA, 20 bp, synthetic construct
  source 1..20: mol_type = other DNA
gggtatatacccaaaggact

SEQ ID NO: 697 - DNA, 20 bp, synthetic construct
  source 1..20: mol_type = other DNA
gggggggggggggggggggg

SEQ ID NO: 698 - DNA, 20 bp, synthetic construct
  source 1..20: mol_type = other DNA
gggactgttgtggggtgggg

SEQ ID NO: 699 - DNA, 20 bp, synthetic construct
  source 1..20: mol_type = other DNA
gggggggggggggggggggg

SEQ ID NO: 700 - DNA, 20 bp, synthetic construct
  source 1..20: mol_type = other DNA
ggaggaacagagagagggaa

SEQ ID NO: 701 - DNA, 20 bp, synthetic construct
  source 1..20: mol_type = other DNA
gggggggggggggggggggg

SEQ ID NO: 702 - DNA, 20 bp, synthetic construct
  source 1..20: mol_type = other DNA
gaggaacagagagagggaaa

SEQ ID NO: 703 - DNA, 20 bp, synthetic construct
  source 1..20: mol_type = other DNA
gggggggggggggggggggg

SEQ ID NO: 704 - DNA, 20 bp, synthetic construct
  source 1..20: mol_type = other DNA
tcctgctgtgtcaaaaatgc

SEQ ID NO: 705 - DNA, 20 bp, synthetic construct
  source 1..20: mol_type = other DNA
gggggggggggggggggggg

SEQ ID NO: 706 - DNA, 20 bp, synthetic construct
  source 1..20: mol_type = other DNA
gcctagctttacccctctac

SEQ ID NO: 707 - DNA, 20 bp, synthetic construct
  source 1..20: mol_type = other DNA
gggggggggggggggggggg

SEQ ID NO: 708 - DNA, 20 bp, synthetic construct
  source 1..20: mol_type = other DNA
acctctccattttgtgtgag

SEQ ID NO: 709 - DNA, 20 bp, synthetic construct
  source 1..20: mol_type = other DNA
gggggggggggggggggggg

SEQ ID NO: 710 - DNA, 20 bp, synthetic construct
  source 1..20: mol_type = other DNA
cctctccattttgtgtgagc

SEQ ID NO: 711 - DNA, 20 bp, synthetic construct
  source 1..20: mol_type = other DNA
gggggggggggggggggggg

SEQ ID NO: 712 - DNA, 20 bp, synthetic construct
  source 1..20: mol_type = other DNA
tacattataaatcaggagac

SEQ ID NO: 713 - DNA, 20 bp, synthetic construct
  source 1..20: mol_type = other DNA
atgttggaaggatgaggaaa

SEQ ID NO: 714 - DNA, 20 bp, synthetic construct
  source 1..20: mol_type = other DNA
cagacaagtcagtagggcca

SEQ ID NO: 715 - DNA, 20 bp, synthetic construct
  source 1..20: mol_type = other DNA
agaagacttcctctgccctc

SEQ ID NO: 716 - DNA, 20 bp, synthetic construct
  source 1..20: mol_type = other DNA
tgactaaacactgtgcccca

SEQ ID NO: 717 - DNA, 20 bp, synthetic construct
  source 1..20: mol_type = other DNA
aggcaggatgaattggtgga

SEQ ID NO: 718 - DNA, 18 bp, synthetic construct
  source 1..18: mol_type = other DNA
catggctgatgcaatgcg

SEQ ID NO: 719 - DNA, 18 bp, synthetic construct
  source 1..18: mol_type = other DNA
tcgcttggtggtcgaatg

SEQ ID NO: 720 - DNA, 20 bp, synthetic construct
  source 1..20: mol_type = other DNA
ggaagaaagaattgcaatta

SEQ ID NO: 721 - DNA, 20 bp, synthetic construct
  source 1..20: mol_type = other DNA
ttggtatttagaaaggtggt

SEQ ID NO: 722 - RNA, 16 nt, synthetic construct
  source 1..16: mol_type = other RNA
gttttagagctatgct

SEQ ID NO: 723 - RNA, 80 nt, synthetic construct
  source 1..80: mol_type = other RNA
  modified_base 77: mod_base = OTHER; note = 2'-O-methyl modified nucleotide with phosphorothioate linkage
  modified_base 78: mod_base = OTHER; note = 2'-O-methyl modified nucleotide with phosphorothioate linkage
  modified_base 79: mod_base = OTHER; note = 2'-O-methyl modified nucleotide with phosphorothioate linkage
gttttagagctagaaatagcaagttaaaataaggctagtccgttatcaacttgaaaaagtggcaccgagtcggtgctttt

SEQ ID NO: 724 - DNA, 20 bp, synthetic construct
  source 1..20: mol_type = other DNA
cacaatatttcttttaagtc

SEQ ID NO: 725 - DNA, 20 bp, synthetic construct
  source 1..20: mol_type = other DNA
gataagaacttctcacagag

SEQ ID NO: 726 - DNA, 20 bp, synthetic construct
  source 1..20: mol_type = other DNA
gggcgtggtcggggcgggcc

SEQ ID NO: 727 - DNA, 20 bp, synthetic construct
  source 1..20: mol_type = other DNA
tagcgcgcgactcctgagtt

SEQ ID NO: 728 - DNA, 7760 bp, Adeno-associated virus
  source 1..7760: mol_type = genomic DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagaacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgtacccatacgatgttccagattacgcttcgccgaagaaaaagcgcaaggtcgaagcgtccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcaataaaagatctttattttcattagatctgtgtgttggttttttgtgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 729 - DNA, 5249 bp, Adeno-associated virus
  source 1..5249: mol_type = genomic DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgaaaaagcaccgactcggtgccactttttcaagttgataacggactagccttattttaacttgctatttctagctctaaaaccacaatatttcttttaagtccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcggccggccaaaaagcaccgactcggtgccactttttcaagttgataacggactagccttattttaacttgctatttctagctctaaaacgataagaacttctcacagagcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggcttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatctctggctaactagagaacccactgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 730 - DNA, 5249 bp, Adeno-associated virus
  source 1..5249: mol_type = genomic DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgaaaaagcaccgactcggtgccactttttcaagttgataacggactagccttattttaacttgctatttctagctctaaaacgggcgtggtcggggcgggcccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcggccggccaaaaagcaccgactcggtgccactttttcaagttgataacggactagccttattttaacttgctatttctagctctaaaactagcgcgcgactcctgagttcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggcttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatctctggctaactagagaacccactgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 731 - RNA, 20 nt, synthetic construct
  source 1..20: mol_type = other RNA
aactcaggagtcgcgcgcta

SEQ ID NO: 732 - RNA, 20 nt, synthetic construct
  source 1..20: mol_type = other RNA
ggcccgccccgaccacgccc

SEQ ID NO: 733 - RNA, 20 nt, synthetic construct
  source 1..20: mol_type = other RNA
tgcgatgacgttttctcacg

SEQ ID NO: 734 - RNA, 20 nt, synthetic construct
  source 1..20: mol_type = other RNA
tactgtgagagcaagtagtg

SEQ ID NO: 735 - RNA, 20 nt, synthetic construct
  source 1..20: mol_type = other RNA
cgtggtcggggcgggcccgg

SEQ ID NO: 736 - RNA, 20 nt, synthetic construct
  source 1..20: mol_type = other RNA
gctgtttggggttcggctgc

SEQ ID NO: 737 - RNA, 20 nt, synthetic construct
  source 1..20: mol_type = other RNA
ctctgtgagaagtttttatc

SEQ ID NO: 738 - RNA, 20 nt, synthetic construct
  source 1..20: mol_type = other RNA
gacttagaagaaatattgtg

SEQ ID NO: 739 - RNA, 20 nt, synthetic construct
  source 1..20: mol_type = other RNA
ctctgtgagaagttcttatc

SEQ ID NO: 740 - RNA, 20 nt, synthetic construct
  source 1..20: mol_type = other RNA
gacttaaaagaaatattgtg

SEQ ID NO: 741 - RNA, 20 nt, synthetic construct
  source 1..20: mol_type = other RNA
tgtgcgaaccttaatagggg

SEQ ID NO: 742 - RNA, 20 nt, synthetic construct
  source 1..20: mol_type = other RNA
aatggggatcgcagcacata

SEQ ID NO: 743 - RNA, 20 nt, synthetic construct
  source 1..20: mol_type = other RNA
ctctgtgagaagtttttatc

SEQ ID NO: 744 - RNA, 20 nt, synthetic construct
  source 1..20: mol_type = other RNA
ctctgtgagaagttcttatc

SEQ ID NO: 745 - RNA, 20 nt, synthetic construct
  source 1..20: mol_type = other RNA
gacttagaagaaatattgtg

SEQ ID NO: 746 - RNA, 20 nt, synthetic construct
  source 1..20: mol_type = other RNA
gacttaaaagaaatattgtg

SEQ ID NO: 747 - RNA, 20 nt, synthetic construct
  source 1..20: mol_type = other RNA
atgttggaaggatgaggaaa

SEQ ID NO: 748 - RNA, 20 nt, synthetic construct
  source 1..20: mol_type = other RNA
ggaagaaagaattgcaatta

SEQ ID NO: 749 - RNA, 20 nt, synthetic construct
  source 1..20: mol_type = other RNA
ttacaaaatgaaccttgcat

SEQ ID NO: 750 - RNA, 20 nt, synthetic construct
  source 1..20: mol_type = other RNA
ttacaaaatgaaccttgcac

SEQ ID NO: 751 - RNA, 20 nt, synthetic construct
  source 1..20: mol_type = other RNA
gaagttacttaacctatgca

SEQ ID NO: 752 - RNA, 20 nt, synthetic construct
  source 1..20: mol_type = other RNA
gaagttacttaacctgtgca

SEQ ID NO: 753 - RNA, 20 nt, synthetic construct
  source 1..20: mol_type = other RNA
aaactttctgcttaactctc

SEQ ID NO: 754 - RNA, 20 nt, synthetic construct
  source 1..20: mol_type = other RNA
aaactttctgcttaactctc

SEQ ID NO: 755 - RNA, 20 nt, synthetic construct
  source 1..20: mol_type = other RNA
cacatctttctgattcatga

SEQ ID NO: 756 - RNA, 20 nt, synthetic construct
  source 1..20: mol_type = other RNA
cgcatctttctgattcatga

SEQ ID NO: 757 - RNA, 20 nt, synthetic construct
  source 1..20: mol_type = other RNA
acatctttctgattcatgat

SEQ ID NO: 758 - RNA, 20 nt, synthetic construct
  source 1..20: mol_type = other RNA
gcatctttctgattcatgat

SEQ ID NO: 759 - RNA, 20 nt, synthetic construct
  source 1..20: mol_type = other RNA
gttgtgtaaatttggtgggt

SEQ ID NO: 760 - RNA, 20 nt, synthetic construct
  source 1..20: mol_type = other RNA
attgtgtaaatttggtgggt

SEQ ID NO: 761 - RNA, 20 nt, synthetic construct
  source 1..20: mol_type = other RNA
gtcattttgttgtgtaaatt

SEQ ID NO: 762 - RNA, 20 nt, synthetic construct
  source 1..20: mol_type = other RNA
gtcattttattgtgtaaatt

SEQ ID NO: 763 - RNA, 20 nt, synthetic construct
  source 1..20: mol_type = other RNA
attttgttgtgtaaatttgg

SEQ ID NO: 764 - RNA, 20 nt, synthetic construct
  source 1..20: mol_type = other RNA
attttattgtgtaaatttgg

SEQ ID NO: 765 - RNA, 20 nt, synthetic construct
  source 1..20: mol_type = other RNA
ttttgttgtgtaaatttggt

SEQ ID NO: 766 - RNA, 20 nt, synthetic construct
  source 1..20: mol_type = other RNA
ttttattgtgtaaatttggt

SEQ ID NO: 767 - RNA, 20 nt, synthetic construct
  source 1..20: mol_type = other RNA
tgttgtgtaaatttggtggg

SEQ ID NO: 768 - RNA, 20 nt, synthetic construct
  source 1..20: mol_type = other RNA
tattgtgtaaatttggtggg

SEQ ID NO: 769 - RNA, 20 nt, synthetic construct
  source 1..20: mol_type = other RNA
gttgcaatgattgccaaagc

SEQ ID NO: 770 - RNA, 20 nt, synthetic construct
  source 1..20: mol_type = other RNA
tttgcaatgattgccaaagc

SEQ ID NO: 771 - RNA, 20 nt, synthetic construct
  source 1..20: mol_type = other RNA
tccattatttttaggcagaa

SEQ ID NO: 772 - RNA, 24 nt, synthetic construct
  source 1..24: mol_type = other RNA
tttttccattatttttaggcagaa

SEQ ID NO: 773 - RNA, 20 nt, synthetic construct
  source 1..20: mol_type = other RNA
ttccattatttttaggcaga

SEQ ID NO: 774 - RNA, 23 nt, synthetic construct
  source 1..23: mol_type = other RNA
tttttccattatttttaggcaga

SEQ ID NO: 775 - RNA, 20 nt, synthetic construct
  source 1..20: mol_type = other RNA
tttttttttccattattttt

SEQ ID NO: 776 - RNA, 20 nt, synthetic construct
  source 1..20: mol_type = other RNA
tttttttttccattattttt

SEQ ID NO: 777 - RNA, 20 nt, synthetic construct
  source 1..20: mol_type = other RNA
tccattatttttaggcagaa

SEQ ID NO: 778 - RNA, 20 nt, synthetic construct
  source 1..20: mol_type = other RNA
tccattatttttaggcagaa

SEQ ID NO: 779 - RNA, 20 nt, synthetic construct
  source 1..20: mol_type = other RNA
ttccattatttttaggcaga

SEQ ID NO: 780 - RNA, 20 nt, synthetic construct
  source 1..20: mol_type = other RNA
ttccattatttttaggcaga

SEQ ID NO: 781 - RNA, 20 nt, synthetic construct
  source 1..20: mol_type = other RNA
tttttttttccattattttt

SEQ ID NO: 782 - RNA, 20 nt, synthetic construct
  source 1..20: mol_type = other RNA
tttttttttccattattttt

SEQ ID NO: 783 - RNA, 20 nt, synthetic construct
  source 1..20: mol_type = other RNA
tccattatttttaggcagaa

SEQ ID NO: 784 - RNA, 20 nt, synthetic construct
  source 1..20: mol_type = other RNA
tccattatttttaggcagaa

SEQ ID NO: 785 - RNA, 20 nt, synthetic construct
  source 1..20: mol_type = other RNA
ttccattatttttaggcaga

SEQ ID NO: 786 - RNA, 20 nt, synthetic construct
  source 1..20: mol_type = other RNA
ttccattatttttaggcaga

SEQ ID NO: 787 - RNA, 20 nt, synthetic construct
  source 1..20: mol_type = other RNA
tttttttttccattattttt

SEQ ID NO: 788 - RNA, 20 nt, synthetic construct
  source 1..20: mol_type = other RNA
tttttttttccattattttt

SEQ ID NO: 789 - RNA, 20 nt, synthetic construct
  source 1..20: mol_type = other RNA
tccattatttttaggcagaa

SEQ ID NO: 790 - RNA, 27 nt, synthetic construct
  source 1..27: mol_type = other RNA
ttttttttccattatttttaggcagaa

SEQ ID NO: 791 - RNA, 20 nt, synthetic construct
  source 1..20: mol_type = other RNA
ttccattatttttaggcaga

SEQ ID NO: 792 - RNA, 26 nt, synthetic construct
  source 1..26: mol_type = other RNA
ttttttttccattatttttaggcaga

SEQ ID NO: 793 - RNA, 20 nt, synthetic construct
  source 1..20: mol_type = other RNA
tttttttttccattattttt

SEQ ID NO: 794 - RNA, 20 nt, synthetic construct
  source 1..20: mol_type = other RNA
tttttttttccattattttt

SEQ ID NO: 795 - RNA, 20 nt, synthetic construct
  source 1..20: mol_type = other RNA
tccattatttttaggcagaa

SEQ ID NO: 796 - RNA, 22 nt, synthetic construct
  source 1..22: mol_type = other RNA
tttccattatttttaggcagaa

SEQ ID NO: 797 - RNA, 20 nt, synthetic construct
  source 1..20: mol_type = other RNA
ttccattatttttaggcaga

SEQ ID NO: 798 - RNA, 21 nt, synthetic construct
  source 1..21: mol_type = other RNA
tttccattatttttaggcaga

SEQ ID NO: 799 - RNA, 20 nt, synthetic construct
  source 1..20: mol_type = other RNA
tttttttttccattattttt

SEQ ID NO: 800 - RNA, 20 nt, synthetic construct
  source 1..20: mol_type = other RNA
tttttttttccattattttt

SEQ ID NO: 801 - RNA, 20 nt, synthetic construct
  source 1..20: mol_type = other RNA
aacaaaaaaacaatgtacaa

SEQ ID NO: 802 - RNA, 20 nt, synthetic construct
  source 1..20: mol_type = other RNA
aacaaaaaaccaatgtacaa

SEQ ID NO: 803 - RNA, 20 nt, synthetic construct
  source 1..20: mol_type = other RNA
atttttttttttttttgaga

SEQ ID NO: 804 - RNA, 24 nt, synthetic construct
  source 1..24: mol_type = other RNA
atatttttttttttttttttgaga

SEQ ID NO: 805 - RNA, 20 nt, synthetic construct
  source 1..20: mol_type = other RNA
atttttttttttttttgaga

SEQ ID NO: 806 - RNA, 20 nt, synthetic construct
  source 1..20: mol_type = other RNA
atttttttttttttttgaga

SEQ ID NO: 807 - RNA, 20 nt, synthetic construct
  source 1..20: mol_type = other RNA
tcactgcaacctacacctcc

SEQ ID NO: 808 - RNA, 20 nt, synthetic construct
  source 1..20: mol_type = other RNA
tcactgcaacctccacctcc

SEQ ID NO: 809 - RNA, 20 nt, synthetic construct
  source 1..20: mol_type = other RNA
cactgcaacctacacctcct

SEQ ID NO: 810 - RNA, 20 nt, synthetic construct
  source 1..20: mol_type = other RNA
cactgcaacctccacctcct

SEQ ID NO: 811 - RNA, 20 nt, synthetic construct
  source 1..20: mol_type = other RNA
cgcttgaacccaggaggtgt

SEQ ID NO: 812 - RNA, 20 nt, synthetic construct
  source 1..20: mol_type = other RNA
cgcttgaacccaggaggtgg

SEQ ID NO: 813 - RNA, 20 nt, synthetic construct
  source 1..20: mol_type = other RNA
cactgcaacctacacctcct

SEQ ID NO: 814 - RNA, 20 nt, synthetic construct
  source 1..20: mol_type = other RNA
cactgcaacctacacctccg

SEQ ID NO: 815 - RNA, 20 nt, synthetic construct
  source 1..20: mol_type = other RNA
cgcttgaacccaggaggtgt

SEQ ID NO: 816 - RNA, 20 nt, synthetic construct
  source 1..20: mol_type = other RNA
cgcttgaaccccggaggtgt

SEQ ID NO: 817 - RNA, 20 nt, synthetic construct
  source 1..20: mol_type = other RNA
gagaatcgcttgaacccagg

SEQ ID NO: 818 - RNA, 20 nt, synthetic construct
  source 1..20: mol_type = other RNA
gagaatcgcttgaaccccgg

SEQ ID NO: 819 - RNA, 20 nt, synthetic construct
  source 1..20: mol_type = other RNA
tcatatacctgtacttttgc

SEQ ID NO: 820 - RNA, 20 nt, synthetic construct
  source 1..20: mol_type = other RNA
tcatatacccgtacttttgc

SEQ ID NO: 821 - RNA, 20 nt, synthetic construct
  source 1..20: mol_type = other RNA
catatacctgtacttttgca

SEQ ID NO: 822 - RNA, 20 nt, synthetic construct
  source 1..20: mol_type = other RNA
catatacccgtacttttgca

SEQ ID NO: 823 - RNA, 20 nt, synthetic construct
  source 1..20: mol_type = other RNA
atatacctgtacttttgcag

SEQ ID NO: 824 - RNA, 20 nt, synthetic construct
  source 1..20: mol_type = other RNA
atatacccgtacttttgcag

SEQ ID NO: 825 - RNA, 20 nt, synthetic construct
  source 1..20: mol_type = other RNA
tatacctgtacttttgcagg

SEQ ID NO: 826 - RNA, 20 nt, synthetic construct
  source 1..20: mol_type = other RNA
tatacccgtacttttgcagg

SEQ ID NO: 827 - RNA, 20 nt, synthetic construct
  source 1..20: mol_type = other RNA
acctgtacttttgcaggggg

SEQ ID NO: 828 - RNA, 20 nt, synthetic construct
  source 1..20: mol_type = other RNA
acccgtacttttgcaggggg

SEQ ID NO: 829 - RNA, 20 nt, synthetic construct
  source 1..20: mol_type = other RNA
ttttttttttttttgagaaa

SEQ ID NO: 830 - RNA, 20 nt, synthetic construct
  source 1..20: mol_type = other RNA
ttttttttttttttgagaaa

SEQ ID NO: 831 - RNA, 20 nt, synthetic construct
  source 1..20: mol_type = other RNA
tttttttttttttttgagaa

SEQ ID NO: 832 - RNA, 20 nt, synthetic construct
  source 1..20: mol_type = other RNA
tttttttttttttttgagaa

SEQ ID NO: 833 - RNA, 20 nt, synthetic construct
  source 1..20: mol_type = other RNA
tttttttttttgagaaaggg

SEQ ID NO: 834 - RNA, 20 nt, synthetic construct
  source 1..20: mol_type = other RNA
tttttttttttgagaaaggg

SEQ ID NO: 835 - RNA, 20 nt, synthetic construct
  source 1..20: mol_type = other RNA
ttttttttttttttgagaaa

SEQ ID NO: 836 - RNA, 20 nt, synthetic construct
  source 1..20: mol_type = other RNA
ttttttttttttttgagaaa

SEQ ID NO: 837 - RNA, 20 nt, synthetic construct
  source 1..20: mol_type = other RNA
tttttttttttttttgagaa

SEQ ID NO: 838 - RNA, 20 nt, synthetic construct
  source 1..20: mol_type = other RNA
tttttttttttttttgagaa

SEQ ID NO: 839 - RNA, 20 nt, synthetic construct
  source 1..20: mol_type = other RNA
tttttttttttgagaaaggg

SEQ ID NO: 840 - RNA, 20 nt, synthetic construct
  source 1..20: mol_type = other RNA
tttttttttttgagaaaggg

SEQ ID NO: 841 - RNA, 20 nt, synthetic construct
  source 1..20: mol_type = other RNA
tatactctacataacataat

SEQ ID NO: 842 - RNA, 20 nt, synthetic construct
  source 1..20: mol_type = other RNA
tatattctacataacataat

SEQ ID NO: 843 - RNA, 20 nt, synthetic construct
  source 1..20: mol_type = other RNA
atactctacataacataata

SEQ ID NO: 844 - RNA, 20 nt, synthetic construct
  source 1..20: mol_type = other RNA
atattctacataacataata

SEQ ID NO: 845 - RNA, 20 nt, synthetic construct
  source 1..20: mol_type = other RNA
atatatagaacaggcattgt

SEQ ID NO: 846 - RNA, 20 nt, synthetic construct
  source 1..20: mol_type = other RNA
atatatagaacaggcactgt

SEQ ID NO: 847 - RNA, 20 nt, synthetic construct
  source 1..20: mol_type = other RNA
taaaaaaagtagagaccctt

SEQ ID NO: 848 - RNA, 20 nt, synthetic construct
  source 1..20: mol_type = other RNA
taaaaaaagtagagaccttt

SEQ ID NO: 849 - RNA, 20 nt, synthetic construct
  source 1..20: mol_type = other RNA
ttttttgagatggcaagaat

SEQ ID NO: 850 - RNA, 20 nt, synthetic construct
  source 1..20: mol_type = other RNA
gtttttgagatggcaagaat

SEQ ID NO: 851 - RNA, 20 nt, synthetic construct
  source 1..20: mol_type = other RNA
ttttttgtttttttttgaga

SEQ ID NO: 852 - RNA, 20 nt, synthetic construct
  source 1..20: mol_type = other RNA
tttttgttttgtttttgaga

SEQ ID NO: 853 - RNA, 20 nt, synthetic construct
  source 1..20: mol_type = other RNA
cgttttgcggctattgtgaa

SEQ ID NO: 854 - RNA, 20 nt, synthetic construct
  source 1..20: mol_type = other RNA
cgctttgcggctattgtgaa

SEQ ID NO: 855 - RNA, 20 nt, synthetic construct
  source 1..20: mol_type = other RNA
gttttgcggctattgtgaat

SEQ ID NO: 856 - RNA, 20 nt, synthetic construct
  source 1..20: mol_type = other RNA
gctttgcggctattgtgaat

SEQ ID NO: 857 - RNA, 20 nt, synthetic construct
  source 1..20: mol_type = other RNA
taggtattttattcgttttg

SEQ ID NO: 858 - RNA, 20 nt, synthetic construct
  source 1..20: mol_type = other RNA
taggtattttattcgctttg

SEQ ID NO: 859 - RNA, 20 nt, synthetic construct
  source 1..20: mol_type = other RNA
gcaaaacgaataaaatacct

SEQ ID NO: 860 - RNA, 20 nt, synthetic construct
  source 1..20: mol_type = other RNA
gcaaagcgaataaaatacct

SEQ ID NO: 861 - RNA, 20 nt, synthetic construct
  source 1..20: mol_type = other RNA
ctgaaaaggagtggtgagag

SEQ ID NO: 862 - RNA, 20 nt, synthetic construct
  source 1..20: mol_type = other RNA
ctgaaaaggagcggtgagag

SEQ ID NO: 863 - RNA, 20 nt, synthetic construct
  source 1..20: mol_type = other RNA
actccttttcagcatagttc

SEQ ID NO: 864 - RNA, 20 nt, synthetic construct
  source 1..20: mol_type = other RNA
gctccttttcagcatagttc

SEQ ID NO: 865 - RNA, 20 nt, synthetic construct
  source 1..20: mol_type = other RNA
tgatttttcttttttgagac

SEQ ID NO: 866 - RNA, 20 nt, synthetic construct
  source 1..20: mol_type = other RNA
tgatttttcttttttgagac

SEQ ID NO: 867 - RNA, 20 nt, synthetic construct
  source 1..20: mol_type = other RNA
gatttttcttttttgagaca

SEQ ID NO: 868 - RNA, 20 nt, synthetic construct
  source 1..20: mol_type = other RNA
gatttttcttttttgagaca

SEQ ID NO: 869 - RNA, 20 nt, synthetic construct
  source 1..20: mol_type = other RNA
acctcaacctcctgaatagc

SEQ ID NO: 870 - RNA, 20 nt, synthetic construct
  source 1..20: mol_type = other RNA
atctcaacctcctgaatagc

SEQ ID NO: 871 - RNA, 20 nt, synthetic construct
  source 1..20: mol_type = other RNA
cctcaacctcctgaatagct

SEQ ID NO: 872 - RNA, 20 nt, synthetic construct
  source 1..20: mol_type = other RNA
tctcaacctcctgaatagct

SEQ ID NO: 873 - RNA, 20 nt, synthetic construct
  source 1..20: mol_type = other RNA
attcaggaggttgaggtggg

SEQ ID NO: 874 - RNA, 20 nt, synthetic construct
  source 1..20: mol_type = other RNA
attcaggaggttgagatggg

SEQ ID NO: 875 - RNA, 20 nt, synthetic construct
  source 1..20: mol_type = other RNA
gtgggaggattgtttgagcc

SEQ ID NO: 876 - RNA, 20 nt, synthetic construct
  source 1..20: mol_type = other RNA
atgggaggattgtttgagcc

SEQ ID NO: 877 - RNA, 20 nt, synthetic construct
  source 1..20: mol_type = other RNA
gctattcaggaggttgaggt

SEQ ID NO: 878 - RNA, 20 nt, synthetic construct
  source 1..20: mol_type = other RNA
gctattcaggaggttgagat

SEQ ID NO: 879 - RNA, 20 nt, synthetic construct
  source 1..20: mol_type = other RNA
agctattcaggaggttgagg

SEQ ID NO: 880 - RNA, 20 nt, synthetic construct
  source 1..20: mol_type = other RNA
agctattcaggaggttgaga

SEQ ID NO: 881 - RNA, 20 nt, synthetic construct
  source 1..20: mol_type = other RNA
tcctgaatagctgggactat

SEQ ID NO: 882 - RNA, 20 nt, synthetic construct
  source 1..20: mol_type = other RNA
tcctgaatagctgggactac

SEQ ID NO: 883 - RNA, 20 nt, synthetic construct
  source 1..20: mol_type = other RNA
acctatagtcccagctattc

SEQ ID NO: 884 - RNA, 20 nt, synthetic construct
  source 1..20: mol_type = other RNA
acctgtagtcccagctattc

SEQ ID NO: 885 - RNA, 20 nt, synthetic construct
  source 1..20: mol_type = other RNA
tatagtcccagctattcagg

SEQ ID NO: 886 - RNA, 20 nt, synthetic construct
  source 1..20: mol_type = other RNA
tgtagtcccagctattcagg

SEQ ID NO: 887 - RNA, 20 nt, synthetic construct
  source 1..20: mol_type = other RNA
aaaaaaaaattagcttggta

SEQ ID NO: 888 - RNA, 20 nt, synthetic construct
  source 1..20: mol_type = other RNA
aaaaaaaaattagcttggta

SEQ ID NO: 889 - RNA, 20 nt, synthetic construct
  source 1..20: mol_type = other RNA
aaaaaaaaaaaaaattagct

SEQ ID NO: 890 - RNA, 20 nt, synthetic construct
  source 1..20: mol_type = other RNA
aaaaaaaaaaaaaattagct

SEQ ID NO: 891 - RNA, 20 nt, synthetic construct
  source 1..20: mol_type = other RNA
aaaaaaaaattagcttggta

SEQ ID NO: 892 - RNA, 20 nt, synthetic construct
  source 1..20: mol_type = other RNA
aaaaaaaaattagcttggta

SEQ ID NO: 893 - RNA, 20 nt, synthetic construct
  source 1..20: mol_type = other RNA
aaaaaaaaaaaaaattagct

SEQ ID NO: 894 - RNA, 20 nt, synthetic construct
  source 1..20: mol_type = other RNA
aaaaaaaaaaaaaattagct

SEQ ID NO: 895 - RNA, 20 nt, synthetic construct
  source 1..20: mol_type = other RNA
aaaaaaaaattagcttggta

SEQ ID NO: 896 - RNA, 20 nt, synthetic construct
  source 1..20: mol_type = other RNA
aaaaaaaaattagcttggta

SEQ ID NO: 897 - RNA, 20 nt, synthetic construct
  source 1..20: mol_type = other RNA
aaaaaaaaaaaaaattagct

SEQ ID NO: 898 - RNA, 20 nt, synthetic construct
  source 1..20: mol_type = other RNA
aaaaaaaaaaaaaattagct

SEQ ID NO: 899 - RNA, 20 nt, synthetic construct
  source 1..20: mol_type = other RNA
aaaaaaaaattagcttggta

SEQ ID NO: 900 - RNA, 20 nt, synthetic construct
  source 1..20: mol_type = other RNA
aaaaaaaaattagcttggta

SEQ ID NO: 901 - RNA, 20 nt, synthetic construct
  source 1..20: mol_type = other RNA
aaaaaaaaaaaaaattagct

SEQ ID NO: 902 - RNA, 20 nt, synthetic construct
  source 1..20: mol_type = other RNA
aaaaaaaaaaaaaattagct

SEQ ID NO: 903 - RNA, 20 nt, synthetic construct
  source 1..20: mol_type = other RNA
cacctgtaatcccagcactt

SEQ ID NO: 904 - RNA, 20 nt, synthetic construct
  source 1..20: mol_type = other RNA
catctgtaatcccagcactt

SEQ ID NO: 905 - RNA, 20 nt, synthetic construct
  source 1..20: mol_type = other RNA
acctgtaatcccagcacttt

SEQ ID NO: 906 - RNA, 20 nt, synthetic construct
  source 1..20: mol_type = other RNA
atctgtaatcccagcacttt

SEQ ID NO: 907 - RNA, 20 nt, synthetic construct
  source 1..20: mol_type = other RNA
cttggctctctattctccat

SEQ ID NO: 908 - RNA, 20 nt, synthetic construct
  source 1..20: mol_type = other RNA
cttggctctgtattctccat

SEQ ID NO: 909 - RNA, 20 nt, synthetic construct
  source 1..20: mol_type = other RNA
ccccactacaaactgggcaa

SEQ ID NO: 910 - RNA, 20 nt, synthetic construct
  source 1..20: mol_type = other RNA
ccccactacaaaccgggcaa

SEQ ID NO: 911 - RNA, 20 nt, synthetic construct
  source 1..20: mol_type = other RNA
aacccaccccactacaaact

SEQ ID NO: 912 - RNA, 20 nt, synthetic construct
  source 1..20: mol_type = other RNA
aacccaccccactacaaacc

SEQ ID NO: 913 - RNA, 20 nt, synthetic construct
  source 1..20: mol_type = other RNA
tgcccagtttgtagtggggt

SEQ ID NO: 914 - RNA, 20 nt, synthetic construct
  source 1..20: mol_type = other RNA
tgcccggtttgtagtggggt

SEQ ID NO: 915 - RNA, 20 nt, synthetic construct
  source 1..20: mol_type = other RNA
ttgcccagtttgtagtgggg

SEQ ID NO: 916 - RNA, 20 nt, synthetic construct
  source 1..20: mol_type = other RNA
ttgcccggtttgtagtgggg

SEQ ID NO: 917 - RNA, 20 nt, synthetic construct
  source 1..20: mol_type = other RNA
cctttgcccagtttgtagtg

SEQ ID NO: 918 - RNA, 20 nt, synthetic construct
  source 1..20: mol_type = other RNA
cctttgcccggtttgtagtg

SEQ ID NO: 919 - RNA, 20 nt, synthetic construct
  source 1..20: mol_type = other RNA
tcctttgcccagtttgtagt

SEQ ID NO: 920 - RNA, 20 nt, synthetic construct
  source 1..20: mol_type = other RNA
tcctttgcccggtttgtagt

SEQ ID NO: 921 - RNA, 20 nt, synthetic construct
  source 1..20: mol_type = other RNA
gtcctttgcccagtttgtag

SEQ ID NO: 922 - RNA, 20 nt, synthetic construct
  source 1..20: mol_type = other RNA
gtcctttgcccggtttgtag

SEQ ID NO: 923 - RNA, 20 nt, synthetic construct
  source 1..20: mol_type = other RNA
agaaaaatttgtattccttt

SEQ ID NO: 924 - RNA, 20 nt, synthetic construct
  source 1..20: mol_type = other RNA
agaacaatttgtattccttt

SEQ ID NO: 925 - RNA, 20 nt, synthetic construct
  source 1..20: mol_type = other RNA
agaatgggtttgcaaccatt

SEQ ID NO: 926 - RNA, 20 nt, synthetic construct
  source 1..20: mol_type = other RNA
agaatgggtttgcaaccgtt

SEQ ID NO: 927 - RNA, 20 nt, synthetic construct
  source 1..20: mol_type = other RNA
ggaaaaaagaaaatgtttca

SEQ ID NO: 928 - RNA, 20 nt, synthetic construct
  source 1..20: mol_type = other RNA
ggaaaaaagaaaatatttca

SEQ ID NO: 929 - RNA, 20 nt, synthetic construct
  source 1..20: mol_type = other RNA
aaaatgcttgcaataatgcc

SEQ ID NO: 930 - RNA, 20 nt, synthetic construct
  source 1..20: mol_type = other RNA
aaaatgcttgcaataatgcc

SEQ ID NO: 931 - RNA, 20 nt, synthetic construct
  source 1..20: mol_type = other RNA
aaaatgcttgcaataatgcc

SEQ ID NO: 932 - RNA, 20 nt, synthetic construct
  source 1..20: mol_type = other RNA
aaaatgcttgcaataatgcc

SEQ ID NO: 933 - RNA, 20 nt, synthetic construct
  source 1..20: mol_type = other RNA
tcccaaagctctgttcttac

SEQ ID NO: 934 - RNA, 20 nt, synthetic construct
  source 1..20: mol_type = other RNA
acccaaagctctgttcttac

SEQ ID NO: 935 - RNA, 20 nt, synthetic construct
  source 1..20: mol_type = other RNA
gagctttgggagtgcaaggc

SEQ ID NO: 936 - RNA, 20 nt, synthetic construct
  source 1..20: mol_type = other RNA
gagctttgggtgtgcaaggc

SEQ ID NO: 937 - RNA, 20 nt, synthetic construct
  source 1..20: mol_type = other RNA
aacagagctttgggagtgca

SEQ ID NO: 938 - RNA, 20 nt, synthetic construct
  source 1..20: mol_type = other RNA
aacagagctttgggtgtgca

SEQ ID NO: 939 - RNA, 20 nt, synthetic construct
  source 1..20: mol_type = other RNA
ctttgggagtgcaaggcagg

SEQ ID NO: 940 - RNA, 20 nt, synthetic construct
  source 1..20: mol_type = other RNA
ctttgggtgtgcaaggcagg

SEQ ID NO: 941 - RNA, 20 nt, synthetic construct
  source 1..20: mol_type = other RNA
tcccaaagctctgttcttac

SEQ ID NO: 942 - RNA, 20 nt, synthetic construct
  source 1..20: mol_type = other RNA
tcccaaagctctgttcttat

SEQ ID NO: 943 - RNA, 20 nt, synthetic construct
  source 1..20: mol_type = other RNA
gcctgtaagaacagagcttt

SEQ ID NO: 944 - RNA, 20 nt, synthetic construct
  source 1..20: mol_type = other RNA
gcctataagaacagagcttt

SEQ ID NO: 945 - RNA, 20 nt, synthetic construct
  source 1..20: mol_type = other RNA
tgcctgtaagaacagagctt

SEQ ID NO: 946 - RNA, 20 nt, synthetic construct
  source 1..20: mol_type = other RNA
tgcctataagaacagagctt

SEQ ID NO: 947 - RNA, 20 nt, synthetic construct
  source 1..20: mol_type = other RNA
tccttttgaatttactgttc

SEQ ID NO: 948 - RNA, 20 nt, synthetic construct
  source 1..20: mol_type = other RNA
tccttttgaatttactgtac

SEQ ID NO: 949 - RNA, 20 nt, synthetic construct
  source 1..20: mol_type = other RNA
gcctgaacagtaaattcaaa

SEQ ID NO: 950 - RNA, 20 nt, synthetic construct
  source 1..20: mol_type = other RNA
gcctgtacagtaaattcaaa

SEQ ID NO: 951 - RNA, 20 nt, synthetic construct
  source 1..20: mol_type = other RNA
gaagctttgtgctgcctttc

SEQ ID NO: 952 - RNA, 20 nt, synthetic construct
  source 1..20: mol_type = other RNA
gaagttttgtgctgcctttc

SEQ ID NO: 953 - RNA, 20 nt, synthetic construct
  source 1..20: mol_type = other RNA
actggctaccccaactgatt

SEQ ID NO: 954 - RNA, 20 nt, synthetic construct
  source 1..20: mol_type = other RNA
actgcctaccccaactgatt

SEQ ID NO: 955 - RNA, 20 nt, synthetic construct
  source 1..20: mol_type = other RNA
tcatctttgtataatgtcaa

SEQ ID NO: 956 - RNA, 20 nt, synthetic construct
  source 1..20: mol_type = other RNA
tcatcttcgtataatgtcaa

SEQ ID NO: 957 - RNA, 20 nt, synthetic construct
  source 1..20: mol_type = other RNA
atagttttgaaatttcctct

SEQ ID NO: 958 - RNA, 20 nt, synthetic construct
  source 1..20: mol_type = other RNA
acagttttgaaatttcctct

SEQ ID NO: 959 - RNA, 20 nt, synthetic construct
  source 1..20: mol_type = other RNA
ttttttatttcattttgccg

SEQ ID NO: 960 - RNA, 20 nt, synthetic construct
  source 1..20: mol_type = other RNA
ttttttatttcattttgctg

SEQ ID NO: 961 - RNA, 20 nt, synthetic construct
  source 1..20: mol_type = other RNA
aattatttataatacttaaa

SEQ ID NO: 962 - RNA, 20 nt, synthetic construct
  source 1..20: mol_type = other RNA
aaatatttataatacttaaa

SEQ ID NO: 963 - RNA, 20 nt, synthetic construct
  source 1..20: mol_type = other RNA
caattatttataatacttaa

SEQ ID NO: 964 - RNA, 20 nt, synthetic construct
  source 1..20: mol_type = other RNA
caaatatttataatacttaa

SEQ ID NO: 965 - RNA, 20 nt, synthetic construct
  source 1..20: mol_type = other RNA
ttaaaaaaaaaaaagtcctg

SEQ ID NO: 966 - RNA, 22 nt, synthetic construct
  source 1..22: mol_type = other RNA
gttaaaaaaaaaaaaagtcctg

SEQ ID NO: 967 - RNA, 20 nt, synthetic construct
  source 1..20: mol_type = other RNA
ttttaacacatgcttaaatc

SEQ ID NO: 968 - RNA, 20 nt, synthetic construct
  source 1..20: mol_type = other RNA
ttttaacacatgcttaaatc

SEQ ID NO: 969 - RNA, 20 nt, synthetic construct
  source 1..20: mol_type = other RNA
agtcctgaggctagacatgt

SEQ ID NO: 970 - RNA, 20 nt, synthetic construct
  source 1..20: mol_type = other RNA
tgtcctgaggctagacatgt

SEQ ID NO: 971 - RNA, 20 nt, synthetic construct
  source 1..20: mol_type = other RNA
ttaaaaaaaaaaaagtcctg

SEQ ID NO: 972 - RNA, 20 nt, synthetic construct
  source 1..20: mol_type = other RNA
ttaaaaaaaaaaatgtcctg

SEQ ID NO: 973 - RNA, 20 nt, synthetic construct
  source 1..20: mol_type = other RNA
cagaagctataaaagttgca

SEQ ID NO: 974 - RNA, 20 nt, synthetic construct
  source 1..20: mol_type = other RNA
cagaagctataaaagttcca

SEQ ID NO: 975 - RNA, 20 nt, synthetic construct
  source 1..20: mol_type = other RNA
aaaaaaaaacactccaacta

SEQ ID NO: 976 - RNA, 22 nt, synthetic construct
  source 1..22: mol_type = other RNA
aaacaaaaaaacactccaacta

SEQ ID NO: 977 - RNA, 20 nt, synthetic construct
  source 1..20: mol_type = other RNA
ttagtttattagctaatgct

SEQ ID NO: 978 - RNA, 20 nt, synthetic construct
  source 1..20: mol_type = other RNA
ttggtttattagctaatgct

SEQ ID NO: 979 - RNA, 20 nt, synthetic construct
  source 1..20: mol_type = other RNA
agcattagctaataaactaa

SEQ ID NO: 980 - RNA, 20 nt, synthetic construct
  source 1..20: mol_type = other RNA
agcattagctaataaaccaa

SEQ ID NO: 981 - RNA, 20 nt, synthetic construct
  source 1..20: mol_type = other RNA
attaactcaagtagcactga

SEQ ID NO: 982 - RNA, 20 nt, synthetic construct
  source 1..20: mol_type = other RNA
actaactcaagtagcactga

SEQ ID NO: 983 - RNA, 20 nt, synthetic construct
  source 1..20: mol_type = other RNA
agtgctacttgagttaatta

SEQ ID NO: 984 - RNA, 20 nt, synthetic construct
  source 1..20: mol_type = other RNA
agtgctacttgagttagtta

SEQ ID NO: 985 - RNA, 20 nt, synthetic construct
  source 1..20: mol_type = other RNA
ctcaagtagcactgaaggaa

SEQ ID NO: 986 - RNA, 20 nt, synthetic construct
  source 1..20: mol_type = other RNA
ctcaagtaacactgaaggaa

SEQ ID NO: 987 - RNA, 20 nt, synthetic construct
  source 1..20: mol_type = other RNA
tcaagtagcactgaaggaaa

SEQ ID NO: 988 - RNA, 20 nt, synthetic construct
  source 1..20: mol_type = other RNA
tcaagtaacactgaaggaaa

SEQ ID NO: 989 - RNA, 20 nt, synthetic construct
  source 1..20: mol_type = other RNA
attaactcaagtagcactga

SEQ ID NO: 990 - RNA, 20 nt, synthetic construct
  source 1..20: mol_type = other RNA
attaactcaagtaacactga

SEQ ID NO: 991 - RNA, 20 nt, synthetic construct
  source 1..20: mol_type = other RNA
agtgctacttgagttaatta

SEQ ID NO: 992 - RNA, 20 nt, synthetic construct
  source 1..20: mol_type = other RNA
agtgttacttgagttaatta

SEQ ID NO: 993 - RNA, 20 nt, synthetic construct
  source 1..20: mol_type = other RNA
catttatacactactgacac

SEQ ID NO: 994 - RNA, 20 nt, synthetic construct
  source 1..20: mol_type = other RNA
catttatacacaactgacac

SEQ ID NO: 995 - RNA, 20 nt, synthetic construct
  source 1..20: mol_type = other RNA
cagtgtcagtagtgtataaa

SEQ ID NO: 996 - RNA, 20 nt, synthetic construct
  source 1..20: mol_type = other RNA
cagtgtcagttgtgtataaa

SEQ ID NO: 997 - RNA, 20 nt, synthetic construct
  source 1..20: mol_type = other RNA
ttataacaaccctacacatt

SEQ ID NO: 998 - RNA, 20 nt, synthetic construct
  source 1..20: mol_type = other RNA
ttataacaaccccacacatt

SEQ ID NO: 999 - RNA, 20 nt, synthetic construct
  source 1..20: mol_type = other RNA
gtaatagtacctaatgtgta

SEQ ID NO: 1000 - RNA, 20 nt, synthetic construct
  source 1..20: mol_type = other RNA
gtaatagtacctaatgtgtg

SEQ ID NO: 1001 - RNA, 20 nt, synthetic construct
  source 1..20: mol_type = other RNA
gtgcacagagattgtttttt

SEQ ID NO: 1002 - RNA, 20 nt, synthetic construct
  source 1..20: mol_type = other RNA
gtacacagagattgtttttt

SEQ ID NO: 1003 - RNA, 20 nt, synthetic construct
  source 1..20: mol_type = other RNA
agtgcacagagattgttttt

SEQ ID NO: 1004 - RNA, 20 nt, synthetic construct
  source 1..20: mol_type = other RNA
agtacacagagattgttttt

SEQ ID NO: 1005 - RNA, 20 nt, synthetic construct
  source 1..20: mol_type = other RNA
tgcacagagattgttttttg

SEQ ID NO: 1006 - RNA, 20 nt, synthetic construct
  source 1..20: mol_type = other RNA
tacacagagattgttttttg

SEQ ID NO: 1007 - RNA, 20 nt, synthetic construct
  source 1..20: mol_type = other RNA
tgtgtgcttatttgctaaag

SEQ ID NO: 1008 - RNA, 20 nt, synthetic construct
  source 1..20: mol_type = other RNA
tgtgtgcctatttgctaaag

SEQ ID NO: 1009 - RNA, 20 nt, synthetic construct
  source 1..20: mol_type = other RNA
aataagcacacacaaaatcc

SEQ ID NO: 1010 - RNA, 20 nt, synthetic construct
  source 1..20: mol_type = other RNA
aataggcacacacaaaatcc

SEQ ID NO: 1011 - RNA, 20 nt, synthetic construct
  source 1..20: mol_type = other RNA
atattctaattttaatagta

SEQ ID NO: 1012 - RNA, 20 nt, synthetic construct
  source 1..20: mol_type = other RNA
atattctaatttttatagta

SEQ ID NO: 1013 - RNA, 20 nt, synthetic construct
  source 1..20: mol_type = other RNA
ttctttttttttttttgaga

SEQ ID NO: 1014 - RNA, 24 nt, synthetic construct
  source 1..24: mol_type = other RNA
ttttctttttttttttttttgaga

SEQ ID NO: 1015 - RNA, 20 nt, synthetic construct
  source 1..20: mol_type = other RNA
ttctttttttttttttgaga

SEQ ID NO: 1016 - RNA, 20 nt, synthetic construct
  source 1..20: mol_type = other RNA
ttctttttttttttttgaga

SEQ ID NO: 1017 - RNA, 20 nt, synthetic construct
  source 1..20: mol_type = other RNA
tttcgccatgttggccaggt

SEQ ID NO: 1018 - RNA, 20 nt, synthetic construct
  source 1..20: mol_type = other RNA
tttcgccatgttggccaggc

SEQ ID NO: 1019 - RNA, 20 nt, synthetic construct
  source 1..20: mol_type = other RNA
tcagaaatttgacaccaacc

SEQ ID NO: 1020 - RNA, 20 nt, synthetic construct
  source 1..20: mol_type = other RNA
tcagaaatttgacaccagcc

SEQ ID NO: 1021 - RNA, 20 nt, synthetic construct
  source 1..20: mol_type = other RNA
tgacaccaacctggccaaca

SEQ ID NO: 1022 - RNA, 20 nt, synthetic construct
  source 1..20: mol_type = other RNA
tgacaccagcctggccaaca

SEQ ID NO: 1023 - RNA, 20 nt, synthetic construct
  source 1..20: mol_type = other RNA
tttgcaagaactcaatcaaa

SEQ ID NO: 1024 - RNA, 20 nt, synthetic construct
  source 1..20: mol_type = other RNA
tttgcaagaactcagtcaaa

SEQ ID NO: 1025 - RNA, 20 nt, synthetic construct
  source 1..20: mol_type = other RNA
ggtgttgttgagcttcagtg

SEQ ID NO: 1026 - RNA, 20 nt, synthetic construct
  source 1..20: mol_type = other RNA
ggtgttgttgagcttcagtg

SEQ ID NO: 1027 - RNA, 20 nt, synthetic construct
  source 1..20: mol_type = other RNA
ggtgttgttgagcttcagtg

SEQ ID NO: 1028 - RNA, 22 nt, synthetic construct
  source 1..22: mol_type = other RNA
ttggtgttgttgagcttcagtg

SEQ ID NO: 1029 - RNA, 20 nt, synthetic construct
  source 1..20: mol_type = other RNA
gttcaagtttgggaagtaca

SEQ ID NO: 1030 - RNA, 20 nt, synthetic construct
  source 1..20: mol_type = other RNA
gttcgagtttgggaagtaca

SEQ ID NO: 1031 - RNA, 20 nt, synthetic construct
  source 1..20: mol_type = other RNA
ctgctgagaagttcaagttt

SEQ ID NO: 1032 - RNA, 20 nt, synthetic construct
  source 1..20: mol_type = other RNA
ctgctgagaagttcgagttt

SEQ ID NO: 1033 - RNA, 20 nt, synthetic construct
  source 1..20: mol_type = other RNA
actgctgagaagttcaagtt

SEQ ID NO: 1034 - RNA, 20 nt, synthetic construct
  source 1..20: mol_type = other RNA
actgctgagaagttcgagtt

SEQ ID NO: 1035 - RNA, 20 nt, synthetic construct
  source 1..20: mol_type = other RNA
caagtttgggaagtacaagg

SEQ ID NO: 1036 - RNA, 20 nt, synthetic construct
  source 1..20: mol_type = other RNA
cgagtttgggaagtacaagg

SEQ ID NO: 1037 - RNA, 20 nt, synthetic construct
  source 1..20: mol_type = other RNA
tcaacttaagtaaaggtttc

SEQ ID NO: 1038 - RNA, 20 nt, synthetic construct
  source 1..20: mol_type = other RNA
tcaacttaagcaaaggtttc

SEQ ID NO: 1039 - RNA, 20 nt, synthetic construct
  source 1..20: mol_type = other RNA
atagtagtcaacttaagtaa

SEQ ID NO: 1040 - RNA, 20 nt, synthetic construct
  source 1..20: mol_type = other RNA
atagtagtcaacttaagcaa

SEQ ID NO: 1041 - RNA, 20 nt, synthetic construct
  source 1..20: mol_type = other RNA
agtaaacttatcgcaagttg

SEQ ID NO: 1042 - RNA, 20 nt, synthetic construct
  source 1..20: mol_type = other RNA
agtaaacttaccgcaagttg

SEQ ID NO: 1043 - RNA, 20 nt, synthetic construct
  source 1..20: mol_type = other RNA
gacttagaagaaatattgtg

SEQ ID NO: 1044 - RNA, 20 nt, synthetic construct
  source 1..20: mol_type = other RNA
gacttaaaagaaatattgtg

SEQ ID NO: 1045 - RNA, 20 nt, synthetic construct
  source 1..20: mol_type = other RNA
gaagaaatattgtgtggacc

SEQ ID NO: 1046 - RNA, 20 nt, synthetic construct
  source 1..20: mol_type = other RNA
aaagaaatattgtgtggacc

SEQ ID NO: 1047 - RNA, 20 nt, synthetic construct
  source 1..20: mol_type = other RNA
tgtctctgatatgttcttta

SEQ ID NO: 1048 - RNA, 20 nt, synthetic construct
  source 1..20: mol_type = other RNA
tgtctctgatatgttcttta

SEQ ID NO: 1049 - RNA, 20 nt, synthetic construct
  source 1..20: mol_type = other RNA
ctgggcaaacagaaaaaaaa

SEQ ID NO: 1050 - RNA, 20 nt, synthetic construct
  source 1..20: mol_type = other RNA
ctgggcaaacagataaaaaa

SEQ ID NO: 1051 - RNA, 20 nt, synthetic construct
  source 1..20: mol_type = other RNA
tgggcaaacagaaaaaaaaa

SEQ ID NO: 1052 - RNA, 20 nt, synthetic construct
  source 1..20: mol_type = other RNA
tgggcaaacagataaaaaaa

SEQ ID NO: 1053 - RNA, 20 nt, synthetic construct
  source 1..20: mol_type = other RNA
tagggaaaaaatgggggcag

SEQ ID NO: 1054 - RNA, 20 nt, synthetic construct
  source 1..20: mol_type = other RNA
tagggaaacaatgggggcag

SEQ ID NO: 1055 - RNA, 20 nt, synthetic construct
  source 1..20: mol_type = other RNA
gaaaaaatgggggcaggggt

SEQ ID NO: 1056 - RNA, 20 nt, synthetic construct
  source 1..20: mol_type = other RNA
gaaacaatgggggcaggggt

SEQ ID NO: 1057 - RNA, 20 nt, synthetic construct
  source 1..20: mol_type = other RNA
tttctactttagggaaaaaa

SEQ ID NO: 1058 - RNA, 20 nt, synthetic construct
  source 1..20: mol_type = other RNA
tttctactttagggaaacaa

SEQ ID NO: 1059 - RNA, 20 nt, synthetic construct
  source 1..20: mol_type = other RNA
ttctactttagggaaaaaat

SEQ ID NO: 1060 - RNA, 20 nt, synthetic construct
  source 1..20: mol_type = other RNA
ttctactttagggaaacaat

SEQ ID NO: 1061 - RNA, 20 nt, synthetic construct
  source 1..20: mol_type = other RNA
tctactttagggaaaaaatg

SEQ ID NO: 1062 - RNA, 20 nt, synthetic construct
  source 1..20: mol_type = other RNA
tctactttagggaaacaatg

SEQ ID NO: 1063 - RNA, 20 nt, synthetic construct
  source 1..20: mol_type = other RNA
ctactttagggaaaaaatgg

SEQ ID NO: 1064 - RNA, 20 nt, synthetic construct
  source 1..20: mol_type = other RNA
ctactttagggaaacaatgg

SEQ ID NO: 1065 - RNA, 20 nt, synthetic construct
  source 1..20: mol_type = other RNA
tttagggaaaaaatgggggc

SEQ ID NO: 1066 - RNA, 20 nt, synthetic construct
  source 1..20: mol_type = other RNA
tttagggaaacaatgggggc

SEQ ID NO: 1067 - RNA, 20 nt, synthetic construct
  source 1..20: mol_type = other RNA
ttagggaaaaaatgggggca

SEQ ID NO: 1068 - RNA, 20 nt, synthetic construct
  source 1..20: mol_type = other RNA
ttagggaaacaatgggggca

SEQ ID NO: 1069 - RNA, 20 nt, synthetic construct
  source 1..20: mol_type = other RNA
gtttcataatgtgagaaaaa

SEQ ID NO: 1070 - RNA, 20 nt, synthetic construct
  source 1..20: mol_type = other RNA
gttttataatgtgagaaaaa

SEQ ID NO: 1071 - RNA, 20 nt, synthetic construct
  source 1..20: mol_type = other RNA
attatgaaactcttaaaatc

SEQ ID NO: 1072 - RNA, 20 nt, synthetic construct
  source 1..20: mol_type = other RNA
attataaaactcttaaaatc

SEQ ID NO: 1073 - RNA, 20 nt, synthetic construct
  source 1..20: mol_type = other RNA
agcattatatattcaactac

SEQ ID NO: 1074 - RNA, 20 nt, synthetic construct
  source 1..20: mol_type = other RNA
accattatatattcaactac

SEQ ID NO: 1075 - RNA, 20 nt, synthetic construct
  source 1..20: mol_type = other RNA
caacgggcagttgcagcagc

SEQ ID NO: 1076 - RNA, 20 nt, synthetic construct
  source 1..20: mol_type = other RNA
caacgggcagttacagcagc

SEQ ID NO: 1077 - RNA, 20 nt, synthetic construct
  source 1..20: mol_type = other RNA
gcagttgcagcagctggact

SEQ ID NO: 1078 - RNA, 20 nt, synthetic construct
  source 1..20: mol_type = other RNA
gcagttacagcagctggact

SEQ ID NO: 1079 - RNA, 20 nt, synthetic construct
  source 1..20: mol_type = other RNA
tcagcttctgggcaggccat

SEQ ID NO: 1080 - RNA, 20 nt, synthetic construct
  source 1..20: mol_type = other RNA
tcagcttctgggcaggccac

SEQ ID NO: 1081 - RNA, 20 nt, synthetic construct
  source 1..20: mol_type = other RNA
cagcttctgggcaggccata

SEQ ID NO: 1082 - RNA, 20 nt, synthetic construct
  source 1..20: mol_type = other RNA
cagcttctgggcaggccaca

SEQ ID NO: 1083 - RNA, 20 nt, synthetic construct
  source 1..20: mol_type = other RNA
tagggaacttaaaatcatga

SEQ ID NO: 1084 - RNA, 20 nt, synthetic construct
  source 1..20: mol_type = other RNA
cagggaacttaaaatcatga

SEQ ID NO: 1085 - RNA, 20 nt, synthetic construct
  source 1..20: mol_type = other RNA
tcatgattttaagttcccta

SEQ ID NO: 1086 - RNA, 20 nt, synthetic construct
  source 1..20: mol_type = other RNA
tcatgattttaagttccctg

SEQ ID NO: 1087 - RNA, 20 nt, synthetic construct
  source 1..20: mol_type = other RNA
ttttgattttacaggttcat

SEQ ID NO: 1088 - RNA, 20 nt, synthetic construct
  source 1..20: mol_type = other RNA
ttttgattttacaggttcac

SEQ ID NO: 1089 - RNA, 20 nt, synthetic construct
  source 1..20: mol_type = other RNA
ttacaggttcataggtagaa

SEQ ID NO: 1090 - RNA, 20 nt, synthetic construct
  source 1..20: mol_type = other RNA
ttacaggttcacaggtagaa

SEQ ID NO: 1091 - RNA, 20 nt, synthetic construct
  source 1..20: mol_type = other RNA
tttacaggttcataggtaga

SEQ ID NO: 1092 - RNA, 20 nt, synthetic construct
  source 1..20: mol_type = other RNA
tttacaggttcacaggtaga

SEQ ID NO: 1093 - RNA, 20 nt, synthetic construct
  source 1..20: mol_type = other RNA
tgagtgggctcccaaaacct

SEQ ID NO: 1094 - RNA, 20 nt, synthetic construct
  source 1..20: mol_type = other RNA
tgagtgggctccgaaaacct

SEQ ID NO: 1095 - RNA, 20 nt, synthetic construct
  source 1..20: mol_type = other RNA
gagtgggctcccaaaacctt

SEQ ID NO: 1096 - RNA, 20 nt, synthetic construct
  source 1..20: mol_type = other RNA
gagtgggctccgaaaacctt

SEQ ID NO: 1097 - RNA, 20 nt, synthetic construct
  source 1..20: mol_type = other RNA
tgtcttgtcatagctctgct

SEQ ID NO: 1098 - RNA, 20 nt, synthetic construct
  source 1..20: mol_type = other RNA
tctcttgtcatagctctgct

SEQ ID NO: 1099 - RNA, 20 nt, synthetic construct
  source 1..20: mol_type = other RNA
atcactgggtatatacccaa

SEQ ID NO: 1100 - RNA, 20 nt, synthetic construct
  source 1..20: mol_type = other RNA
atccctgggtatatacccaa

SEQ ID NO: 1101 - RNA, 20 nt, synthetic construct
  source 1..20: mol_type = other RNA
tgacccagccatcccatcac

SEQ ID NO: 1102 - RNA, 20 nt, synthetic construct
  source 1..20: mol_type = other RNA
tgacccagccatcccatccc

SEQ ID NO: 1103 - RNA, 20 nt, synthetic construct
  source 1..20: mol_type = other RNA
gacccagccatcccatcact

SEQ ID NO: 1104 - RNA, 20 nt, synthetic construct
  source 1..20: mol_type = other RNA
gacccagccatcccatccct

SEQ ID NO: 1105 - RNA, 20 nt, synthetic construct
  source 1..20: mol_type = other RNA
tttgggtatatacccagtga

SEQ ID NO: 1106 - RNA, 20 nt, synthetic construct
  source 1..20: mol_type = other RNA
tttgggtatatacccaggga

SEQ ID NO: 1107 - RNA, 20 nt, synthetic construct
  source 1..20: mol_type = other RNA
tacccagtgatgggatggct

SEQ ID NO: 1108 - RNA, 20 nt, synthetic construct
  source 1..20: mol_type = other RNA
tacccagggatgggatggct

SEQ ID NO: 1109 - RNA, 20 nt, synthetic construct
  source 1..20: mol_type = other RNA
atacccagtgatgggatggc

SEQ ID NO: 1110 - RNA, 20 nt, synthetic construct
  source 1..20: mol_type = other RNA
atacccagggatgggatggc

SEQ ID NO: 1111 - RNA, 20 nt, synthetic construct
  source 1..20: mol_type = other RNA
gtatatacccagtgatggga

SEQ ID NO: 1112 - RNA, 20 nt, synthetic construct
  source 1..20: mol_type = other RNA
gtatatacccagggatggga

SEQ ID NO: 1113 - RNA, 20 nt, synthetic construct
  source 1..20: mol_type = other RNA
ttgggtatatacccagtgat

SEQ ID NO: 1114 - RNA, 20 nt, synthetic construct
  source 1..20: mol_type = other RNA
ttgggtatatacccagggat

SEQ ID NO: 1115 - RNA, 20 nt, synthetic construct
  source 1..20: mol_type = other RNA
tattcacaatagcaaagact

SEQ ID NO: 1116 - RNA, 20 nt, synthetic construct
  source 1..20: mol_type = other RNA
tgttcacaatagcaaagact

SEQ ID NO: 1117 - RNA, 20 nt, synthetic construct
  source 1..20: mol_type = other RNA
tcacactctggggactgttg

SEQ ID NO: 1118 - RNA, 20 nt, synthetic construct
  source 1..20: mol_type = other RNA
tcacactccggggactgttg

SEQ ID NO: 1119 - RNA, 20 nt, synthetic construct
  source 1..20: mol_type = other RNA
cacactctggggactgttgt

SEQ ID NO: 1120 - RNA, 20 nt, synthetic construct
  source 1..20: mol_type = other RNA
cacactccggggactgttgt

SEQ ID NO: 1121 - RNA, 20 nt, synthetic construct
  source 1..20: mol_type = other RNA
acactctggggactgttgtg

SEQ ID NO: 1122 - RNA, 20 nt, synthetic construct
  source 1..20: mol_type = other RNA
acactccggggactgttgtg

SEQ ID NO: 1123 - RNA, 20 nt, synthetic construct
  source 1..20: mol_type = other RNA
ctctggggactgttgtgggg

SEQ ID NO: 1124 - RNA, 20 nt, synthetic construct
  source 1..20: mol_type = other RNA
ctccggggactgttgtgggg

SEQ ID NO: 1125 - RNA, 20 nt, synthetic construct
  source 1..20: mol_type = other RNA
tctggggactgttgtggggt

SEQ ID NO: 1126 - RNA, 20 nt, synthetic construct
  source 1..20: mol_type = other RNA
tccggggactgttgtggggt

SEQ ID NO: 1127 - RNA, 20 nt, synthetic construct
  source 1..20: mol_type = other RNA
ctggggactgttgtggggtg

SEQ ID NO: 1128 - RNA, 20 nt, synthetic construct
  source 1..20: mol_type = other RNA
ccggggactgttgtggggtg

SEQ ID NO: 1129 - RNA, 20 nt, synthetic construct
  source 1..20: mol_type = other RNA
tggggactgttgtggggtgg

SEQ ID NO: 1130 - RNA, 20 nt, synthetic construct
  source 1..20: mol_type = other RNA
cggggactgttgtggggtgg

SEQ ID NO: 1131 - RNA, 20 nt, synthetic construct
  source 1..20: mol_type = other RNA
gaaggggaacatcacactct

SEQ ID NO: 1132 - RNA, 20 nt, synthetic construct
  source 1..20: mol_type = other RNA
gaaggggaacatcacactcc

SEQ ID NO: 1133 - RNA, 20 nt, synthetic construct
  source 1..20: mol_type = other RNA
aaggggaacatcacactctg

SEQ ID NO: 1134 - RNA, 20 nt, synthetic construct
  source 1..20: mol_type = other RNA
aaggggaacatcacactccg

SEQ ID NO: 1135 - RNA, 20 nt, synthetic construct
  source 1..20: mol_type = other RNA
gaaagactcataggactaac

SEQ ID NO: 1136 - RNA, 20 nt, synthetic construct
  source 1..20: mol_type = other RNA
gaaagactcataggactaat

SEQ ID NO: 1137 - RNA, 20 nt, synthetic construct
  source 1..20: mol_type = other RNA
gtgctcatgcctatgagcat

SEQ ID NO: 1138 - RNA, 20 nt, synthetic construct
  source 1..20: mol_type = other RNA
gtgctcatgcctatgaacat

SEQ ID NO: 1139 - RNA, 20 nt, synthetic construct
  source 1..20: mol_type = other RNA
ttctaatagcctatgctcat

SEQ ID NO: 1140 - RNA, 20 nt, synthetic construct
  source 1..20: mol_type = other RNA
ttctaatagcctatgttcat

SEQ ID NO: 1141 - RNA, 20 nt, synthetic construct
  source 1..20: mol_type = other RNA
ttcacagatccctagaacag

SEQ ID NO: 1142 - RNA, 20 nt, synthetic construct
  source 1..20: mol_type = other RNA
ttcacagatacctagaacag

SEQ ID NO: 1143 - RNA, 20 nt, synthetic construct
  source 1..20: mol_type = other RNA
tgactttggaaccggctaac

SEQ ID NO: 1144 - RNA, 20 nt, synthetic construct
  source 1..20: mol_type = other RNA
tgactttggaacctgctaac

SEQ ID NO: 1145 - RNA, 20 nt, synthetic construct
  source 1..20: mol_type = other RNA
ggaacagagagagggaaagt

SEQ ID NO: 1146 - RNA, 20 nt, synthetic construct
  source 1..20: mol_type = other RNA
ggaacagagagagggaaagg

SEQ ID NO: 1147 - RNA, 20 nt, synthetic construct
  source 1..20: mol_type = other RNA
gaacagagagagggaaagtg

SEQ ID NO: 1148 - RNA, 20 nt, synthetic construct
  source 1..20: mol_type = other RNA
gaacagagagagggaaaggg

SEQ ID NO: 1149 - RNA, 20 nt, synthetic construct
  source 1..20: mol_type = other RNA
aacagagagagggaaagtgg

SEQ ID NO: 1150 - RNA, 20 nt, synthetic construct
  source 1..20: mol_type = other RNA
aacagagagagggaaagggg

SEQ ID NO: 1151 - RNA, 20 nt, synthetic construct
  source 1..20: mol_type = other RNA
agagagagggaaagtggggg

SEQ ID NO: 1152 - RNA, 20 nt, synthetic construct
  source 1..20: mol_type = other RNA
agagagagggaaaggggggg

SEQ ID NO: 1153 - RNA, 20 nt, synthetic construct
  source 1..20: mol_type = other RNA
aggctccatctccaacgttg

SEQ ID NO: 1154 - RNA, 20 nt, synthetic construct
  source 1..20: mol_type = other RNA
aggctccatctccaacgctg

SEQ ID NO: 1155 - RNA, 20 nt, synthetic construct
  source 1..20: mol_type = other RNA
ccaggctccatctccaacgt

SEQ ID NO: 1156 - RNA, 20 nt, synthetic construct
  source 1..20: mol_type = other RNA
ccaggctccatctccaacgc

SEQ ID NO: 1157 - RNA, 20 nt, synthetic construct
  source 1..20: mol_type = other RNA
caggctccatctccaacgtt

SEQ ID NO: 1158 - RNA, 20 nt, synthetic construct
  source 1..20: mol_type = other RNA
caggctccatctccaacgct

SEQ ID NO: 1159 - RNA, 20 nt, synthetic construct
  source 1..20: mol_type = other RNA
tcagttgcaatccccaacgt

SEQ ID NO: 1160 - RNA, 20 nt, synthetic construct
  source 1..20: mol_type = other RNA
tcagttgcaatccccagcgt

SEQ ID NO: 1161 - RNA, 20 nt, synthetic construct
  source 1..20: mol_type = other RNA
gcaatccccaacgttggaga

SEQ ID NO: 1162 - RNA, 20 nt, synthetic construct
  source 1..20: mol_type = other RNA
gcaatccccagcgttggaga

SEQ ID NO: 1163 - RNA, 20 nt, synthetic construct
  source 1..20: mol_type = other RNA
ccaacgttggagatggagcc

SEQ ID NO: 1164 - RNA, 20 nt, synthetic construct
  source 1..20: mol_type = other RNA
ccagcgttggagatggagcc

SEQ ID NO: 1165 - RNA, 20 nt, synthetic construct
  source 1..20: mol_type = other RNA
aatcttatcacatacataaa

SEQ ID NO: 1166 - RNA, 20 nt, synthetic construct
  source 1..20: mol_type = other RNA
aatcttatcgcatacataaa

SEQ ID NO: 1167 - RNA, 20 nt, synthetic construct
  source 1..20: mol_type = other RNA
tttagaataagactggaact

SEQ ID NO: 1168 - RNA, 20 nt, synthetic construct
  source 1..20: mol_type = other RNA
tttagagtaagactggaact

SEQ ID NO: 1169 - RNA, 20 nt, synthetic construct
  source 1..20: mol_type = other RNA
tccccagtttagaataagac

SEQ ID NO: 1170 - RNA, 20 nt, synthetic construct
  source 1..20: mol_type = other RNA
tccccagtttagagtaagac

SEQ ID NO: 1171 - RNA, 20 nt, synthetic construct
  source 1..20: mol_type = other RNA
tccagtcttattctaaactg

SEQ ID NO: 1172 - RNA, 20 nt, synthetic construct
  source 1..20: mol_type = other RNA
tccagtcttactctaaactg

SEQ ID NO: 1173 - RNA, 20 nt, synthetic construct
  source 1..20: mol_type = other RNA
ttccagtcttattctaaact

SEQ ID NO: 1174 - RNA, 20 nt, synthetic construct
  source 1..20: mol_type = other RNA
ttccagtcttactctaaact

SEQ ID NO: 1175 - RNA, 20 nt, synthetic construct
  source 1..20: mol_type = other RNA
gttccagtcttattctaaac

SEQ ID NO: 1176 - RNA, 20 nt, synthetic construct
  source 1..20: mol_type = other RNA
gttccagtcttactctaaac

SEQ ID NO: 1177 - RNA, 20 nt, synthetic construct
  source 1..20: mol_type = other RNA
gacacagcaggaagtttatc

SEQ ID NO: 1178 - RNA, 20 nt, synthetic construct
  source 1..20: mol_type = other RNA
gacacagcaggaaggttatc

SEQ ID NO: 1179 - RNA, 20 nt, synthetic construct
  source 1..20: mol_type = other RNA
ttcactcttgttgtccaggc

SEQ ID NO: 1180 - RNA, 20 nt, synthetic construct
  source 1..20: mol_type = other RNA
ttcactcttgttgtccaagc

SEQ ID NO: 1181 - RNA, 20 nt, synthetic construct
  source 1..20: mol_type = other RNA
ttgtccaggctggagtgcaa

SEQ ID NO: 1182 - RNA, 20 nt, synthetic construct
  source 1..20: mol_type = other RNA
ttgtccaagctggagtgcaa

SEQ ID NO: 1183 - RNA, 20 nt, synthetic construct
  source 1..20: mol_type = other RNA
cgtgccattgcactccagcc

SEQ ID NO: 1184 - RNA, 20 nt, synthetic construct
  source 1..20: mol_type = other RNA
cgtgccattgcactccagct

SEQ ID NO: 1185 - RNA, 20 nt, synthetic construct
  source 1..20: mol_type = other RNA
tcccgaagtgctgagattac

SEQ ID NO: 1186 - RNA, 20 nt, synthetic construct
  source 1..20: mol_type = other RNA
tcccaaagtgctgagattac

SEQ ID NO: 1187 - RNA, 20 nt, synthetic construct
  source 1..20: mol_type = other RNA
ctcagcacttcgggaggcca

SEQ ID NO: 1188 - RNA, 20 nt, synthetic construct
  source 1..20: mol_type = other RNA
ctcagcactttgggaggcca

SEQ ID NO: 1189 - RNA, 20 nt, synthetic construct
  source 1..20: mol_type = other RNA
tgtaatctcagcacttcggg

SEQ ID NO: 1190 - RNA, 20 nt, synthetic construct
  source 1..20: mol_type = other RNA
tgtaatctcagcactttggg

SEQ ID NO: 1191 - RNA, 20 nt, synthetic construct
  source 1..20: mol_type = other RNA
acctgtaatctcagcacttc

SEQ ID NO: 1192 - RNA, 20 nt, synthetic construct
  source 1..20: mol_type = other RNA
acctgtaatctcagcacttt

SEQ ID NO: 1193 - RNA, 20 nt, synthetic construct
  source 1..20: mol_type = other RNA
gcacttcgggaggccaagga

SEQ ID NO: 1194 - RNA, 20 nt, synthetic construct
  source 1..20: mol_type = other RNA
gcactttgggaggccaagga

SEQ ID NO: 1195 - RNA, 20 nt, synthetic construct
  source 1..20: mol_type = other RNA
agcacttcgggaggccaagg

SEQ ID NO: 1196 - RNA, 20 nt, synthetic construct
  source 1..20: mol_type = other RNA
agcactttgggaggccaagg

SEQ ID NO: 1197 - RNA, 20 nt, synthetic construct
  source 1..20: mol_type = other RNA
tgccttaatcattacggact

SEQ ID NO: 1198 - RNA, 20 nt, synthetic construct
  source 1..20: mol_type = other RNA
tgcctcaatcattacggact

SEQ ID NO: 1199 - RNA, 20 nt, synthetic construct
  source 1..20: mol_type = other RNA
tgtggttgccttaatcatta

SEQ ID NO: 1200 - RNA, 20 nt, synthetic construct
  source 1..20: mol_type = other RNA
tgtggttgcctcaatcatta

SEQ ID NO: 1201 - RNA, 20 nt, synthetic construct
  source 1..20: mol_type = other RNA
cttaatcattacggactagg

SEQ ID NO: 1202 - RNA, 20 nt, synthetic construct
  source 1..20: mol_type = other RNA
ctcaatcattacggactagg

SEQ ID NO: 1203 - RNA, 20 nt, synthetic construct
  source 1..20: mol_type = other RNA
cgcctagtccgtaatgatta

SEQ ID NO: 1204 - RNA, 20 nt, synthetic construct
  source 1..20: mol_type = other RNA
cgcctagtccgtaatgattg

SEQ ID NO: 1205 - RNA, 20 nt, synthetic construct
  source 1..20: mol_type = other RNA
tgccttaatcattacggact

SEQ ID NO: 1206 - RNA, 20 nt, synthetic construct
  source 1..20: mol_type = other RNA
tgccttaatcattacagact

SEQ ID NO: 1207 - RNA, 20 nt, synthetic construct
  source 1..20: mol_type = other RNA
cttaatcattacggactagg

SEQ ID NO: 1208 - RNA, 20 nt, synthetic construct
  source 1..20: mol_type = other RNA
cttaatcattacagactagg

SEQ ID NO: 1209 - RNA, 20 nt, synthetic construct
  source 1..20: mol_type = other RNA
cgcctagtccgtaatgatta

SEQ ID NO: 1210 - RNA, 20 nt, synthetic construct
  source 1..20: mol_type = other RNA
cgcctagtctgtaatgatta

SEQ ID NO: 1211 - RNA, 20 nt, synthetic construct
  source 1..20: mol_type = other RNA
ggggtttcaccatgttagtc

SEQ ID NO: 1212 - RNA, 20 nt, synthetic construct
  source 1..20: mol_type = other RNA
ggagtttcaccatgttagtc

SEQ ID NO: 1213 - RNA, 20 nt, synthetic construct
  source 1..20: mol_type = other RNA
ttgtatttttagtagagatg

SEQ ID NO: 1214 - RNA, 20 nt, synthetic construct
  source 1..20: mol_type = other RNA
ttgtatttttagtagagatg

SEQ ID NO: 1215 - RNA, 20 nt, synthetic construct
  source 1..20: mol_type = other RNA
tttgtatttttagtagagat

SEQ ID NO: 1216 - RNA, 20 nt, synthetic construct
  source 1..20: mol_type = other RNA
tttgtatttttagtagagat

SEQ ID NO: 1217 - RNA, 20 nt, synthetic construct
  source 1..20: mol_type = other RNA
ttttgtatttttagtagaga

SEQ ID NO: 1218 - RNA, 20 nt, synthetic construct
  source 1..20: mol_type = other RNA
ttttgtatttttagtagaga

SEQ ID NO: 1219 - RNA, 20 nt, synthetic construct
  source 1..20: mol_type = other RNA
gaatacagagttctggggag

SEQ ID NO: 1220 - RNA, 20 nt, synthetic construct
  source 1..20: mol_type = other RNA
gaatacagagttcttgggag

SEQ ID NO: 1221 - RNA, 20 nt, synthetic construct
  source 1..20: mol_type = other RNA
gcctggaatacagagttctg

SEQ ID NO: 1222 - RNA, 20 nt, synthetic construct
  source 1..20: mol_type = other RNA
gcctggaatacagagttctt

SEQ ID NO: 1223 - RNA, 20 nt, synthetic construct
  source 1..20: mol_type = other RNA
tccccagaactctgtattcc

SEQ ID NO: 1224 - RNA, 20 nt, synthetic construct
  source 1..20: mol_type = other RNA
tcccaagaactctgtattcc

SEQ ID NO: 1225 - RNA, 20 nt, synthetic construct
  source 1..20: mol_type = other RNA
aaagctaggccagttgctca

SEQ ID NO: 1226 - RNA, 20 nt, synthetic construct
  source 1..20: mol_type = other RNA
aaagctaggccggttgctca

SEQ ID NO: 1227 - RNA, 20 nt, synthetic construct
  source 1..20: mol_type = other RNA
taaagctaggccagttgctc

SEQ ID NO: 1228 - RNA, 20 nt, synthetic construct
  source 1..20: mol_type = other RNA
taaagctaggccggttgctc

SEQ ID NO: 1229 - RNA, 20 nt, synthetic construct
  source 1..20: mol_type = other RNA
agagaagcttgaaagtcaag

SEQ ID NO: 1230 - RNA, 20 nt, synthetic construct
  source 1..20: mol_type = other RNA
agagaagcttcaaagtcaag

SEQ ID NO: 1231 - RNA, 20 nt, synthetic construct
  source 1..20: mol_type = other RNA
tggagaggtgaggacatttc

SEQ ID NO: 1232 - RNA, 20 nt, synthetic construct
  source 1..20: mol_type = other RNA
tggagaggtggggacatttc

SEQ ID NO: 1233 - RNA, 20 nt, synthetic construct
  source 1..20: mol_type = other RNA
caatttcctcctcttgaaaa

SEQ ID NO: 1234 - RNA, 20 nt, synthetic construct
  source 1..20: mol_type = other RNA
caaattcctcctcttgaaaa

SEQ ID NO: 1235 - RNA, 20 nt, synthetic construct
  source 1..20: mol_type = other RNA
ttttcaagaggaggaaattg

SEQ ID NO: 1236 - RNA, 20 nt, synthetic construct
  source 1..20: mol_type = other RNA
ttttcaagaggaggaatttg

SEQ ID NO: 1237 - RNA, 20 nt, synthetic construct
  source 1..20: mol_type = other RNA
aaaaaaaaaaagctcttacc

SEQ ID NO: 1238 - RNA, 20 nt, synthetic construct
  source 1..20: mol_type = other RNA
aaaaaaaaaaagctcttacc

SEQ ID NO: 1239 - RNA, 20 nt, synthetic construct
  source 1..20: mol_type = other RNA
aaaagctcttacctggttgc

SEQ ID NO: 1240 - RNA, 20 nt, synthetic construct
  source 1..20: mol_type = other RNA
aaaagctcttacctggttgc

SEQ ID NO: 1241 - RNA, 20 nt, synthetic construct
  source 1..20: mol_type = other RNA
aaaaaaaaaaagctcttacc

SEQ ID NO: 1242 - RNA, 20 nt, synthetic construct
  source 1..20: mol_type = other RNA
aaaaaaaaaaagctcttacc

SEQ ID NO: 1243 - RNA, 20 nt, synthetic construct
  source 1..20: mol_type = other RNA
aaaagctcttacctggttgc

SEQ ID NO: 1244 - RNA, 20 nt, synthetic construct
  source 1..20: mol_type = other RNA
aaaagctcttacctggttgc

SEQ ID NO: 1245 - RNA, 20 nt, synthetic construct
  source 1..20: mol_type = other RNA
tttttttttaaatcaacata

SEQ ID NO: 1246 - RNA, 20 nt, synthetic construct
  source 1..20: mol_type = other RNA
ttttttttaaaatcaacata

SEQ ID NO: 1247 - RNA, 20 nt, synthetic construct
  source 1..20: mol_type = other RNA
aaacaactgatagtcgtcag

SEQ ID NO: 1248 - RNA, 20 nt, synthetic construct
  source 1..20: mol_type = other RNA
aaacaactgataatcgtcag

SEQ ID NO: 1249 - RNA, 20 nt, synthetic construct
  source 1..20: mol_type = other RNA
atgcctaaataatgtcattg

SEQ ID NO: 1250 - RNA, 20 nt, synthetic construct
  source 1..20: mol_type = other RNA
atgcctaaataatgtcatcg

SEQ ID NO: 1251 - RNA, 20 nt, synthetic construct
  source 1..20: mol_type = other RNA
tttccacaatgacattattt

SEQ ID NO: 1252 - RNA, 20 nt, synthetic construct
  source 1..20: mol_type = other RNA
tttccacgatgacattattt

SEQ ID NO: 1253 - RNA, 20 nt, synthetic construct
  source 1..20: mol_type = other RNA
gcagaaagtgacagtgagac

SEQ ID NO: 1254 - RNA, 20 nt, synthetic construct
  source 1..20: mol_type = other RNA
gcagaaagtgactgtgagac

SEQ ID NO: 1255 - RNA, 20 nt, synthetic construct
  source 1..20: mol_type = other RNA
agtgacagtgagactggaaa

SEQ ID NO: 1256 - RNA, 20 nt, synthetic construct
  source 1..20: mol_type = other RNA
agtgactgtgagactggaaa

SEQ ID NO: 1257 - RNA, 20 nt, synthetic construct
  source 1..20: mol_type = other RNA
aagtgacagtgagactggaa

SEQ ID NO: 1258 - RNA, 20 nt, synthetic construct
  source 1..20: mol_type = other RNA
aagtgactgtgagactggaa

SEQ ID NO: 1259 - RNA, 20 nt, synthetic construct
  source 1..20: mol_type = other RNA
cagtgagactggaaagggat

SEQ ID NO: 1260 - RNA, 20 nt, synthetic construct
  source 1..20: mol_type = other RNA
ctgtgagactggaaagggat

SEQ ID NO: 1261 - RNA, 20 nt, synthetic construct
  source 1..20: mol_type = other RNA
tcactctcactctgttgccc

SEQ ID NO: 1262 - RNA, 20 nt, synthetic construct
  source 1..20: mol_type = other RNA
tcattctcactctgttgccc

SEQ ID NO: 1263 - RNA, 20 nt, synthetic construct
  source 1..20: mol_type = other RNA
gtgagagtgagactctttct

SEQ ID NO: 1264 - RNA, 20 nt, synthetic construct
  source 1..20: mol_type = other RNA
gtgagaatgagactctttct

SEQ ID NO: 1265 - RNA, 20 nt, synthetic construct
  source 1..20: mol_type = other RNA
aactctgggggaaaagaagt

SEQ ID NO: 1266 - RNA, 20 nt, synthetic construct
  source 1..20: mol_type = other RNA
aactctgggggaaaaaaagt

SEQ ID NO: 1267 - RNA, 20 nt, synthetic construct
  source 1..20: mol_type = other RNA
cttcttttcccccagagttc

SEQ ID NO: 1268 - RNA, 20 nt, synthetic construct
  source 1..20: mol_type = other RNA
ctttttttcccccagagttc

SEQ ID NO: 1269 - RNA, 20 nt, synthetic construct
  source 1..20: mol_type = other RNA
gatgaatgaatcttgtttac

SEQ ID NO: 1270 - RNA, 20 nt, synthetic construct
  source 1..20: mol_type = other RNA
gatgaatgaattttgtttac

SEQ ID NO: 1271 - RNA, 20 nt, synthetic construct
  source 1..20: mol_type = other RNA
atgaatgaatcttgtttaca

SEQ ID NO: 1272 - RNA, 20 nt, synthetic construct
  source 1..20: mol_type = other RNA
atgaatgaattttgtttaca

SEQ ID NO: 1273 - RNA, 20 nt, synthetic construct
  source 1..20: mol_type = other RNA
agattcattcatctcaaatt

SEQ ID NO: 1274 - RNA, 20 nt, synthetic construct
  source 1..20: mol_type = other RNA
aaattcattcatctcaaatt

SEQ ID NO: 1275 - RNA, 20 nt, synthetic construct
  source 1..20: mol_type = other RNA
aagattcattcatctcaaat

SEQ ID NO: 1276 - RNA, 20 nt, synthetic construct
  source 1..20: mol_type = other RNA
aaaattcattcatctcaaat

SEQ ID NO: 1277 - RNA, 20 nt, synthetic construct
  source 1..20: mol_type = other RNA
gagaagtttttatctggtga

SEQ ID NO: 1278 - RNA, 20 nt, synthetic construct
  source 1..20: mol_type = other RNA
gagaagttcttatctggtga

SEQ ID NO: 1279 - RNA, 20 nt, synthetic construct
  source 1..20: mol_type = other RNA
ctctgtgagaagtttttatc

SEQ ID NO: 1280 - RNA, 20 nt, synthetic construct
  source 1..20: mol_type = other RNA
ctctgtgagaagttcttatc

SEQ ID NO: 1281 - RNA, 20 nt, synthetic construct
  source 1..20: mol_type = other RNA
agaagaaatgtctatgaaag

SEQ ID NO: 1282 - RNA, 20 nt, synthetic construct
  source 1..20: mol_type = other RNA
agaaaaaatgtctatgaaag

SEQ ID NO: 1283 - RNA, 20 nt, synthetic construct
  source 1..20: mol_type = other RNA
gacatttcttctgcatgggt

SEQ ID NO: 1284 - RNA, 20 nt, synthetic construct
  source 1..20: mol_type = other RNA
gacattttttctgcatgggt

SEQ ID NO: 1285 - RNA, 20 nt, synthetic construct
  source 1..20: mol_type = other RNA
catagacatttcttctgcat

SEQ ID NO: 1286 - RNA, 20 nt, synthetic construct
  source 1..20: mol_type = other RNA
catagacattttttctgcat

SEQ ID NO: 1287 - RNA, 20 nt, synthetic construct
  source 1..20: mol_type = other RNA
tcatagacatttcttctgca

SEQ ID NO: 1288 - RNA, 20 nt, synthetic construct
  source 1..20: mol_type = other RNA
tcatagacattttttctgca

SEQ ID NO: 1289 - RNA, 20 nt, synthetic construct
  source 1..20: mol_type = other RNA
tgtctatgaaagcggtcaaa

SEQ ID NO: 1290 - RNA, 20 nt, synthetic construct
  source 1..20: mol_type = other RNA
tgtctgtgaaagcggtcaaa

SEQ ID NO: 1291 - RNA, 20 nt, synthetic construct
  source 1..20: mol_type = other RNA
atgtctatgaaagcggtcaa

SEQ ID NO: 1292 - RNA, 20 nt, synthetic construct
  source 1..20: mol_type = other RNA
atgtctgtgaaagcggtcaa

SEQ ID NO: 1293 - RNA, 20 nt, synthetic construct
  source 1..20: mol_type = other RNA
agaagaaatgtctatgaaag

SEQ ID NO: 1294 - RNA, 20 nt, synthetic construct
  source 1..20: mol_type = other RNA
agaagaaatgtctgtgaaag

SEQ ID NO: 1295 - RNA, 20 nt, synthetic construct
  source 1..20: mol_type = other RNA
catagacatttcttctgcat

SEQ ID NO: 1296 - RNA, 20 nt, synthetic construct
  source 1..20: mol_type = other RNA
cacagacatttcttctgcat

SEQ ID NO: 1297 - RNA, 20 nt, synthetic construct
  source 1..20: mol_type = other RNA
tcatagacatttcttctgca

SEQ ID NO: 1298 - RNA, 20 nt, synthetic construct
  source 1..20: mol_type = other RNA
tcacagacatttcttctgca

SEQ ID NO: 1299 - RNA, 20 nt, synthetic construct
  source 1..20: mol_type = other RNA
aatgagtagctctgcaaaag

SEQ ID NO: 1300 - RNA, 20 nt, synthetic construct
  source 1..20: mol_type = other RNA
aatgagtagctttgcaaaag

SEQ ID NO: 1301 - RNA, 20 nt, synthetic construct
  source 1..20: mol_type = other RNA
tctgcaaaagtggattttcc

SEQ ID NO: 1302 - RNA, 20 nt, synthetic construct
  source 1..20: mol_type = other RNA
tttgcaaaagtggattttcc

SEQ ID NO: 1303 - RNA, 20 nt, synthetic construct
  source 1..20: mol_type = other RNA
ctactcattttttgataatt

SEQ ID NO: 1304 - RNA, 20 nt, synthetic construct
  source 1..20: mol_type = other RNA
ctactcatttttcgataatt

SEQ ID NO: 1305 - RNA, 20 nt, synthetic construct
  source 1..20: mol_type = other RNA
gctactcattttttgataat

SEQ ID NO: 1306 - RNA, 20 nt, synthetic construct
  source 1..20: mol_type = other RNA
gctactcatttttcgataat

SEQ ID NO: 1307 - RNA, 20 nt, synthetic construct
  source 1..20: mol_type = other RNA
aagacatacaaatggcaaac

SEQ ID NO: 1308 - RNA, 20 nt, synthetic construct
  source 1..20: mol_type = other RNA
aagacgtacaaatggcaaac

SEQ ID NO: 1309 - RNA, 20 nt, synthetic construct
  source 1..20: mol_type = other RNA
ttcaagagaagacatacaaa

SEQ ID NO: 1310 - RNA, 20 nt, synthetic construct
  source 1..20: mol_type = other RNA
ttcaagagaagacgtacaaa

SEQ ID NO: 1311 - RNA, 20 nt, synthetic construct
  source 1..20: mol_type = other RNA
aagacatacaaatggcaaac

SEQ ID NO: 1312 - RNA, 20 nt, synthetic construct
  source 1..20: mol_type = other RNA
aagacatacaagtggcaaac

SEQ ID NO: 1313 - RNA, 20 nt, synthetic construct
  source 1..20: mol_type = other RNA
ttcaagagaagacatacaaa

SEQ ID NO: 1314 - RNA, 20 nt, synthetic construct
  source 1..20: mol_type = other RNA
ttcaagagaagacatacaag

SEQ ID NO: 1315 - RNA, 20 nt, synthetic construct
  source 1..20: mol_type = other RNA
tatgtgttctgcctccccta

SEQ ID NO: 1316 - RNA, 20 nt, synthetic construct
  source 1..20: mol_type = other RNA
tacgtgttctgcctccccta

SEQ ID NO: 1317 - RNA, 20 nt, synthetic construct
  source 1..20: mol_type = other RNA
ctctttttttttttttttga

SEQ ID NO: 1318 - RNA, 22 nt, synthetic construct
  source 1..22: mol_type = other RNA
cctcctttttttttttttttga

SEQ ID NO: 1319 - RNA, 20 nt, synthetic construct
  source 1..20: mol_type = other RNA
ctctttttttttttttttga

SEQ ID NO: 1320 - RNA, 22 nt, synthetic construct
  source 1..22: mol_type = other RNA
cctcttttttttttttttttga

SEQ ID NO: 1321 - RNA, 20 nt, synthetic construct
  source 1..20: mol_type = other RNA
cgtcaaaaaaaaaaaaaaag

SEQ ID NO: 1322 - RNA, 20 nt, synthetic construct
  source 1..20: mol_type = other RNA
gtcaaaaaaaaaaaaaaaag

SEQ ID NO: 1323 - RNA, 20 nt, synthetic construct
  source 1..20: mol_type = other RNA
ctctttttttttttttttga

SEQ ID NO: 1324 - RNA, 24 nt, synthetic construct
  source 1..24: mol_type = other RNA
tcctctttttttttttttttttga

SEQ ID NO: 1325 - RNA, 20 nt, synthetic construct
  source 1..20: mol_type = other RNA
cgtcaaaaaaaaaaaaaaag

SEQ ID NO: 1326 - RNA, 20 nt, synthetic construct
  source 1..20: mol_type = other RNA
tcaaaaaaaaaaaaaaaaag

SEQ ID NO: 1327 - RNA, 20 nt, synthetic construct
  source 1..20: mol_type = other RNA
ttttatttattaattgccta

SEQ ID NO: 1328 - RNA, 20 nt, synthetic construct
  source 1..20: mol_type = other RNA
cccccccccccccccccccc

SEQ ID NO: 1329 - RNA, 20 nt, synthetic construct
  source 1..20: mol_type = other RNA
ggtttctttcttctgtagtg

SEQ ID NO: 1330 - RNA, 20 nt, synthetic construct
  source 1..20: mol_type = other RNA
cccccccccccccccccccc

SEQ ID NO: 1331 - RNA, 20 nt, synthetic construct
  source 1..20: mol_type = other RNA
cccagctattcaggaggttg

SEQ ID NO: 1332 - RNA, 20 nt, synthetic construct
  source 1..20: mol_type = other RNA
cccccccccccccccccccc

SEQ ID NO: 1333 - RNA, 20 nt, synthetic construct
  source 1..20: mol_type = other RNA
ctcccaaagtgctgggatta

SEQ ID NO: 1334 - RNA, 20 nt, synthetic construct
  source 1..20: mol_type = other RNA
gggggggggggggggggggg

SEQ ID NO: 1335 - RNA, 20 nt, synthetic construct
  source 1..20: mol_type = other RNA
ctttcttgaggatgtagcca

SEQ ID NO: 1336 - RNA, 20 nt, synthetic construct
  source 1..20: mol_type = other RNA
cccccccccccccccccccc

SEQ ID NO: 1337 - RNA, 20 nt, synthetic construct
  source 1..20: mol_type = other RNA
aaatgtgaaagctggtcaag

SEQ ID NO: 1338 - RNA, 20 nt, synthetic construct
  source 1..20: mol_type = other RNA
gggggggggggggggggggg

SEQ ID NO: 1339 - RNA, 20 nt, synthetic construct
  source 1..20: mol_type = other RNA
tctgggcaaacagaaaaaaa

SEQ ID NO: 1340 - RNA, 20 nt, synthetic construct
  source 1..20: mol_type = other RNA
gggggggggggggggggggg

SEQ ID NO: 1341 - RNA, 20 nt, synthetic construct
  source 1..20: mol_type = other RNA
ggtggttctgcccaaggttt

SEQ ID NO: 1342 - RNA, 20 nt, synthetic construct
  source 1..20: mol_type = other RNA
cccccccccccccccccccc

SEQ ID NO: 1343 - RNA, 20 nt, synthetic construct
  source 1..20: mol_type = other RNA
tgcattgttcctctgttcta

SEQ ID NO: 1344 - RNA, 20 nt, synthetic construct
  source 1..20: mol_type = other RNA
cccccccccccccccccccc

SEQ ID NO: 1345 - RNA, 20 nt, synthetic construct
  source 1..20: mol_type = other RNA
tgtagaagtgactttggaac

SEQ ID NO: 1346 - RNA, 20 nt, synthetic construct
  source 1..20: mol_type = other RNA
cccccccccccccccccccc

SEQ ID NO: 1347 - RNA, 20 nt, synthetic construct
  source 1..20: mol_type = other RNA
agagtttcactcttgttgtc

SEQ ID NO: 1348 - RNA, 20 nt, synthetic construct
  source 1..20: mol_type = other RNA
gggggggggggggggggggg

SEQ ID NO: 1349 - RNA, 20 nt, synthetic construct
  source 1..20: mol_type = other RNA
atgtggttgccttaatcatt

SEQ ID NO: 1350 - RNA, 20 nt, synthetic construct
  source 1..20: mol_type = other RNA
gggggggggggggggggggg

SEQ ID NO: 1351 - RNA, 20 nt, synthetic construct
  source 1..20: mol_type = other RNA
ttgtatttttagtagagatg

SEQ ID NO: 1352 - RNA, 20 nt, synthetic construct
  source 1..20: mol_type = other RNA
cccccccccccccccccccc

SEQ ID NO: 1353 - RNA, 20 nt, synthetic construct
  source 1..20: mol_type = other RNA
tttgtatttttagtagagat

SEQ ID NO: 1354 - RNA, 20 nt, synthetic construct
  source 1..20: mol_type = other RNA
cccccccccccccccccccc

SEQ ID NO: 1355 - RNA, 20 nt, synthetic construct
  source 1..20: mol_type = other RNA
aaggcctggaatacagagtt

SEQ ID NO: 1356 - RNA, 20 nt, synthetic construct
  source 1..20: mol_type = other RNA
gggggggggggggggggggg

SEQ ID NO: 1357 - RNA, 20 nt, synthetic construct
  source 1..20: mol_type = other RNA
ttacattttgggggcaaata

SEQ ID NO: 1358 - RNA, 20 nt, synthetic construct
  source 1..20: mol_type = other RNA
gggggggggggggggggggg

SEQ ID NO: 1359 - RNA, 20 nt, synthetic construct
  source 1..20: mol_type = other RNA
agggactggtttcatgacta

SEQ ID NO: 1360 - RNA, 20 nt, synthetic construct
  source 1..20: mol_type = other RNA
cccccccccccccccccccc

SEQ ID NO: 1361 - RNA, 20 nt, synthetic construct
  source 1..20: mol_type = other RNA
gggggggggggggggggggg

SEQ ID NO: 1362 - RNA, 20 nt, synthetic construct
  source 1..20: mol_type = other RNA
tatattaatattataataat

SEQ ID NO: 1363 - RNA, 20 nt, synthetic construct
  source 1..20: mol_type = other RNA
gggggggggggggggggggg

SEQ ID NO: 1364 - RNA, 20 nt, synthetic construct
  source 1..20: mol_type = other RNA
atgtacaaaggtttttcttc

SEQ ID NO: 1365 - RNA, 20 nt, synthetic construct
  source 1..20: mol_type = other RNA
gggggggggggggggggggg

SEQ ID NO: 1366 - RNA, 20 nt, synthetic construct
  source 1..20: mol_type = other RNA
cctcctgggttcaagcgatt

SEQ ID NO: 1367 - RNA, 20 nt, synthetic construct
  source 1..20: mol_type = other RNA
gggggggggggggggggggg

SEQ ID NO: 1368 - RNA, 20 nt, synthetic construct
  source 1..20: mol_type = other RNA
ctcactgcaacctacacctc

SEQ ID NO: 1369 - RNA, 20 nt, synthetic construct
  source 1..20: mol_type = other RNA
gggggggggggggggggggg

SEQ ID NO: 1370 - RNA, 20 nt, synthetic construct
  source 1..20: mol_type = other RNA
tacttttgcagggggtggag

SEQ ID NO: 1371 - RNA, 20 nt, synthetic construct
  source 1..20: mol_type = other RNA
gggggggggggggggggggg

SEQ ID NO: 1372 - RNA, 20 nt, synthetic construct
  source 1..20: mol_type = other RNA
ggcaaaggacatgaaaattt

SEQ ID NO: 1373 - RNA, 20 nt, synthetic construct
  source 1..20: mol_type = other RNA
gggggggggggggggggggg

SEQ ID NO: 1374 - RNA, 20 nt, synthetic construct
  source 1..20: mol_type = other RNA
ctttcctaatcagttggggt

SEQ ID NO: 1375 - RNA, 20 nt, synthetic construct
  source 1..20: mol_type = other RNA
gggggggggggggggggggg

SEQ ID NO: 1376 - RNA, 20 nt, synthetic construct
  source 1..20: mol_type = other RNA
ttctcagacttatttgacca

SEQ ID NO: 1377 - RNA, 20 nt, synthetic construct
  source 1..20: mol_type = other RNA
gggggggggggggggggggg

SEQ ID NO: 1378 - RNA, 20 nt, synthetic construct
  source 1..20: mol_type = other RNA
aagaaaagtgcaaaatcctt

SEQ ID NO: 1379 - RNA, 20 nt, synthetic construct
  source 1..20: mol_type = other RNA
gggggggggggggggggggg

SEQ ID NO: 1380 - RNA, 20 nt, synthetic construct
  source 1..20: mol_type = other RNA
cacattaggtactattacta

SEQ ID NO: 1381 - RNA, 20 nt, synthetic construct
  source 1..20: mol_type = other RNA
gggggggggggggggggggg

SEQ ID NO: 1382 - RNA, 20 nt, synthetic construct
  source 1..20: mol_type = other RNA
gcacacagttttgtttcaaa

SEQ ID NO: 1383 - RNA, 20 nt, synthetic construct
  source 1..20: mol_type = other RNA
gggggggggggggggggggg

SEQ ID NO: 1384 - RNA, 20 nt, synthetic construct
  source 1..20: mol_type = other RNA
atttgctaaagtggctaata

SEQ ID NO: 1385 - RNA, 20 nt, synthetic construct
  source 1..20: mol_type = other RNA
gggggggggggggggggggg

SEQ ID NO: 1386 - RNA, 20 nt, synthetic construct
  source 1..20: mol_type = other RNA
atcctttagtaacctccaca

SEQ ID NO: 1387 - RNA, 20 nt, synthetic construct
  source 1..20: mol_type = other RNA
gggggggggggggggggggg

SEQ ID NO: 1388 - RNA, 20 nt, synthetic construct
  source 1..20: mol_type = other RNA
ttctaagtttcctcaacttg

SEQ ID NO: 1389 - RNA, 20 nt, synthetic construct
  source 1..20: mol_type = other RNA
gggggggggggggggggggg

SEQ ID NO: 1390 - RNA, 20 nt, synthetic construct
  source 1..20: mol_type = other RNA
ttatatattcaactacagga

SEQ ID NO: 1391 - RNA, 20 nt, synthetic construct
  source 1..20: mol_type = other RNA
gggggggggggggggggggg

SEQ ID NO: 1392 - RNA, 20 nt, synthetic construct
  source 1..20: mol_type = other RNA
tgggtatatacccaaaggac

SEQ ID NO: 1393 - RNA, 20 nt, synthetic construct
  source 1..20: mol_type = other RNA
gggggggggggggggggggg

SEQ ID NO: 1394 - RNA, 20 nt, synthetic construct
  source 1..20: mol_type = other RNA
gggtatatacccaaaggact

SEQ ID NO: 1395 - RNA, 20 nt, synthetic construct
  source 1..20: mol_type = other RNA
gggggggggggggggggggg

SEQ ID NO: 1396 - RNA, 20 nt, synthetic construct
  source 1..20: mol_type = other RNA
gggactgttgtggggtgggg

SEQ ID NO: 1397 - RNA, 20 nt, synthetic construct
  source 1..20: mol_type = other RNA
gggggggggggggggggggg

SEQ ID NO: 1398 - RNA, 20 nt, synthetic construct
  source 1..20: mol_type = other RNA
ggaggaacagagagagggaa

SEQ ID NO: 1399 - RNA, 20 nt, synthetic construct
  source 1..20: mol_type = other RNA
gggggggggggggggggggg

SEQ ID NO: 1400 - RNA, 20 nt, synthetic construct
  source 1..20: mol_type = other RNA
gaggaacagagagagggaaa

SEQ ID NO: 1401 - RNA, 20 nt, synthetic construct
  source 1..20: mol_type = other RNA
gggggggggggggggggggg

SEQ ID NO: 1402 - RNA, 20 nt, synthetic construct
  source 1..20: mol_type = other RNA
tcctgctgtgtcaaaaatgc

SEQ ID NO: 1403 - RNA, 20 nt, synthetic construct
  source 1..20: mol_type = other RNA
gggggggggggggggggggg

SEQ ID NO: 1404 - RNA, 20 nt, synthetic construct
  source 1..20: mol_type = other RNA
gcctagctttacccctctac

SEQ ID NO: 1405 - RNA, 20 nt, synthetic construct
  source 1..20: mol_type = other RNA
gggggggggggggggggggg

SEQ ID NO: 1406 - RNA, 20 nt, synthetic construct
  source 1..20: mol_type = other RNA
acctctccattttgtgtgag

SEQ ID NO: 1407 - RNA, 20 nt, synthetic construct
  source 1..20: mol_type = other RNA
gggggggggggggggggggg

SEQ ID NO: 1408 - RNA, 20 nt, synthetic construct
  source 1..20: mol_type = other RNA
cctctccattttgtgtgagc

SEQ ID NO: 1409 - RNA, 20 nt, synthetic construct
  source 1..20: mol_type = other RNA
gggggggggggggggggggg

SEQ ID NO: 1410 - RNA, 20 nt, synthetic construct
  source 1..20: mol_type = other RNA
tacattataaatcaggagac

SEQ ID NO: 1411 - RNA, 20 nt, synthetic construct
  source 1..20: mol_type = other RNA
ttggtatttagaaaggtggt